SEQUENCE LISTING (94 sequences)

SEQ ID NO: 1 - protein, 375 aa, Saccharomyces cerevisiae
MDSEVAALVIDNGSGMCKAGFAGDDAPRAVFPSIVGRPRHQGIMVGMGQKDSYVGDEAQSKRGILTLRYPIEHGIVTNWDDMEKIWHHTFYNELRVAPEEHPVLLTEAPMNPKSNREKMTQIMFETFNVPAFYVSIQAVLSLYSSGRTTGIVLDSGDGVTHVVPIYAGFSLPHAILRIDLAGRDLTDYLMKILSERGYSFSTTAEREIVRDIKEKLCYVALDFEQEMQTAAQSSSIEKSYELPDGQVITIGNERFRAPEALFHPSVLGLESAGIDQTTYNSIMKCDVDVRKELYGNIVMSGGTTMFPGIAERMQKEITALAPSSMKVKIIAPPERKYSVWIGGSILASLTTFQQMWISKQEYDESGPSIVHHKCF

SEQ ID NO: 2 - DNA, 1125 bp, Saccharomyces cerevisiae
atggattctggggttgctgctttggttattgataacggttctggtatgtgtaaagccggttttgccggtgacgacgctcctcgtgctgtcttcccatctatcgtcggtagaccaagacaccaaggtatcatggtcggtatgggtcaaaaagactcctacgttggtgatgaagctcaatccaagagaggtatcttgactttacgttacccaattgaacacggtattgtcaccaactgggacgatatggaaaagatctggcatcataccttctacaacgaattgagagttgccccagaagaacaccctgttcttttgactgaagctccaatgaaccctaaatcaaacagagaaaagatgactcaaattatgtttgaaactttcaacgttccagccttctacgtttccatccaagccgttttgtccttgtactcttccggtagaactactggtattgttttggattccggtgatggtgttactcacgtcgttccaatttacgctggtttctctctacctcacgccattttgagaatcgatttggccggtagagatttgactgactacttgatgaagatcttgagtgaacgtggttactctttctccaccactgctgaaagagaaattgtccgtgacatcaaggaaaaactatgttacgtcgccttggacttcgaacaagaaatgcaaaccgctgctcaatcttcttcaattgaaaaatcctacgaacttccagatggtcaagtcatcactattggtaacgaaagattcagagccccagaagctttgttccatccttctgttttgggtttggaatctgccggtattgaccaaactacttacaactccatcatgaagtgtgatgtcgatgtccgtaaggaattatacggtaacatcgttatgtccggtggtaccaccatgttcccaggtattgccgaaagaatgcaaaaggaaatcaccgctttggctccatcttccatgaaggtcaagatcattgctcctccagaaagaaagtactccgtctggattggtggttctatcttggcttctttgactaccttccaacaaatgtggatctcaaaacaagaatacgacgaaagtggtccatctatcgttcaccacaagtgtttc

SEQ ID NO: 3 - protein, 1357 aa, Saccharomyces cerevisiae
MNVYDDVLDATVVSHSLATHFTTSDYEELLVVRTNILSVYRPTRDGKLYLTDEFKFHGLITDIGLIPQKDSPLSCLLLCTGVAKISILKFNTLTNSIDTLSLHYYEGKFKGKSLVELAKISTLRMDPGSSCALLFNNDIIAFLPFHVNKNDDDEEEEDEDENIDDSELIHSMNQKSQGTNTFNKRKRTKLGDKFTAPSVVLVASELYEGAKNIIDIQFLKNFTKPTIALLYQPKLVWAGNTTISKLPTQYVILTLNIQPAESATKIESTTIAFVKELPWDLHTIVPVSNGAIIVGTNELAFLDNTGVLQSTVLLNSFADKELQKTKIINNSSLEIMFREKNTTSIWIPSSKSKNGGSNNDETLLLMDLKSNIYYIQMEAEGRLLIKFDIFKLPIVNDLLKENSNPKCITRLNATNSNKNMDLFIGFGSGNALVLRLNNLKSTIETREAHNPSSGTNSLMDINDDDDEEMDDLYADEAPENGLTTNDSKGTVETVQPFDIELLSSLRNVGPITSLTVGKVSSIDDVVKGLPNPNKNEYSLVATSGNGSGSHLTVIQTSVQPEIELALKFISITQIWNLKIKGRDRYLITTDSTKSRSDIYESDNNFKLHKGGRLRRDATTVYISMFGEEKRIIQVTTNHLYLYDTHFRRLTTIKFDYEVIHVSVMDPYILVTVSRGDIKIFELEEKNKRKLLKVDLPEILNEMVITSGLILKSNMCNEFLIGLSKSQEEQLLFTFVTADNQIIFFTKDHNDRIFQLNGVDQLNESLYISTYQLGDEIVPDPSIKQVMINKLGHDNKEEYLTILTFGGEIYQYRKLPQRRSRFYRNVTRNDLAITGAPDNAYAKGVSSIERIMHYFPDYNGYSVIFVTGSVPYILIKEDDSTPKIFKFGNIPLVSVTPWSERSVMCVDDIKNARVYTLTTDNMYYGNKLPLKQIKISNVLDDYKTLQKLVYHERAQLFLVSYCKRVPYEALGEDGEKVIGYDENVPHAEGFQSGILLINPKSWKVIDKIDFPKNSVVNEMRSSMIQINSKTKRKREYIIAGVANATTEDTPPTGAFHIYDVIEVVPEPGKPDTNYKLKEIFQEEVSGTVSTVCEVSGRFMISQSQKVLVRDIQEDNSVIPVAFLDIPVFVTDSKSFGNLLIIGDAMQGFQFIGFDAEPYRMISLGRSMSKFQTMSLEFLVNGGDMYFAATDADRNVHVLKYAPDEPNSLSGQRLVHCSSFTLHSTNSCMMLLPRNEEFGSPQVPSFQNVGGQVDGSVFKIVPLSEEKYRRLYVIQQQIIDRELQLGGLNPRMERLANDFYQMGHSMRPMLDFNVIRRFCGLAIDRRKSIAQKAGRHAHFEAWRDIINIEFSMRSLCQGK

SEQ ID NO: 4 - DNA, 2000 bp, Saccharomyces cerevisiae
atgaatgtatatgatgatgtgcttgacgctactgttgtgtctcattcattagcaacacattttactacttcagattatgaggagctcttagtggtgcgaacaaatatcctttctgtctacaggcctactagggatggtaaactgtatttgactgatgagtttaagtttcatggcttgatcaccgatataggcctcattccccaaaaagacagtccgttaagttgcctgcttttatgcacaggcgtcgctaaaatctctattttaaagttcaacacgctaacaaactcaatcgataccctaagcctgcattactacgaaggaaagttcaagggtaaatcgttggtagagctggccaaaatttccaccttgagaatggatcctggaagttcttgtgccttactgttcaataatgacattatcgcttttctacccttccatgtcaataaaaatgacgacgatgaggaggaagaagatgaggacgagaacatagacgatagtgaattaatccatagcatgaatcaaaaatcccaagggacgaatactttcaataagaggaagagaaccaagttaggtgacaagttcacggctccaagcgtggtattggtagcgagtgagttgtacgaaggcgccaaaaatatcatagatattcagttcttgaaaaattttactaaaccaacgatagcgctcttataccaaccaaagcttgtttgggcaggaaacaccaccatttcaaaactccctacacaatacgttatactcacattaaatatccagcctgcagaaagtgccaccaagattgaatcaaccacaatagcatttgtcaaggaactgccttgggatctacacaccattgtacctgtttcgaatggtgctatcattgtgggaaccaatgaactggcatttctagataatactggcgttttacaatcgacagtattattaaactcatttgctgataaagagttacagaaaacaaaaattatcaacaattcttcattggaaattatgttcagggagaaaaacactacgtcaatttggataccttcgtccaagagcaaaaacggaggaagtaataatgacgaaacactactacttatggacttgaaatccaatatatactatatccaaatggaagcagagggcaggttactgattaaattcgatattttcaagctacctatagtcaatgatcttttgaaggagaactccaatccaaaatgtataacacgtttgaatgccaccaattcaaacaaaaatatggacttatttattgggtttggttcaggaaatgctttggttctcagattgaataatttgaaatctaccattgaaacaagagaagcacacaatccatcttcaggtacaaatagtttgatggatatcaatgacgacgatgatgaggaaatggacgatttatacgccgatgaagctcctgaaaatggactaacaactaatgattccaaaggcactgtggaaactgttcaacctttcgatatcgaactgttatcatctctaaggaatgttggtcccatcacatcgttaactgtaggtaaggtatcttccattgatgatgtggtaaaaggactaccaaatccaaacaaaaacgagtattcgctagttgccacatcgggaaatggttcaggttctcatttgaccgtcatacagactagtgttcaaccggaaattgaattagcattgaaatttatcagtataacacaaatctggaatctgaagatcaaaggaagagataggtacttaataactactgattccacaaaatcccggagtgacatatatgaaagtgacaataacttcaaacttcataagggcggccgtttaagaagagatgctactacagtttacatttcaatgtttggtgaagaaaaaagaataatacaagtcaccaccaatcacttatatttatatgatacacattttagacgtctcaccacaattaaatttgactacgaagttattcatgtttccgtcatggatccgta

SEQ ID NO: 5 - protein, 859 aa, Saccharomyces cerevisiae
MTYKYNCCDDGSGTTVGSVVRFDNVTLLIDPGWNPSKVSYEQCIKYWEKVIPEIDVIILSQPTIECLGAHSLLYYNFTSHFISRIQVYATLPVINLGRVSTIDSYASAGVIGPYDTNKLDLEDIEISFDHIVPLKYSQLVDLRSRYDGLTLLAYNAGVCPGGSIWCISTYSEKLVYAKRWNHTRDNILNAASILDATGKPLSTLMRPSAIITTLDRFGSSQPFKKRSKIFKDTLKKGLSSDGSVIIPVDMSGKFLDLFTQVHELLFESTKINAHTQVPVLILSYARGRTLTYAKSMLEWLSPSLLKTWENRNNTSPFEIGSRIKIIAPNELSKYPGSKICFVSEVGALINEVIIKVGNSEKTTLILTKPSFECASSLDKILEIVEQDERNWKTFPEDGKSFLCDNYISIDTIKEEPLSKEETEAFKVQLKEKKRDRNKKILLVKRESKKLANGNAIIDDTNGERAMRNQDILVENVNGVPPIDHIMGGDEDDDEEEENDNLLNLLKDNSEKSAAKKNTEVPVDIIIQPSAASKHKMFPFNPAKIKKDDYGTVVDFTMFLPDDSDNVNQNSRKRPLKDGAKTTSPVNEEDNKNEEEDGYNMSDPISKRSKHRASRYSGFSGTGEAENFDNLDYLKIDKTLSKRTISTVNVQLKCSVVILNLQSLVDQRSASIIWPSLKSRKIVLSAPKQIQNEEITAKLIKKNIEVVNMPLNKIVEFSTTIKTLDISIDSNLDNLLKWQRISDSYTVATVVGRLVKESLPQVNNHQKTASRSKLVLKPLHGSSRSHKTGALSIGDVRLAQLKKLLTEKNYIAEFKGEGTLVINEKVAVRKINDAETIIDGTPSELFDTVKKLVTDMLAKI

SEQ ID NO: 6 - DNA, 2000 bp, Saccharomyces cerevisiae
atgacttataaatacaattgctgtgatgatggatcaggaaccaccgttggttcagtggtacgatttgataacgtcaccttgttaatcgatcccggttggaatccatctaaagtatcgtatgagcaatgcatcaagtactgggaaaaagtgataccggaaattgatgtaataatactatcacaaccaacaattgaatgtttaggtgctcattcccttctgtactataattttacctcgcatttcatatccagaattcaagtttacgcaactcttcctgttatcaacttagggagagtttccaccatagattcatatgcatctgcaggtgttataggtccatatgacacaaataaactcgatcttgaagatattgagatatcttttgatcatattgtacctttgaagtactcgcaattggtagatctacgatcaagatacgatggattgactttactggcgtacaatgctggtgtgtgtccaggtggttctatttggtgcattagcacatactcagagaaattggtttatgcaaagcgatggaaccacactagagataacatactaaatgctgcttctatattggacgccactggtaagcccttatcaactttgatgaggccctctgcaataattactactttggacaggtttggctcctctcaaccgtttaaaaaacgttcaaaaatctttaaagatacattaaagaaaggtttgagttcggatgggtccgttataataccggtagatatgagtggcaaatttctggacctgttcacacaagttcatgagttattatttgaaagcacaaagatcaatgcgcacacacaagtacctgtacttattctgtcttacgcgagaggaagaactctaacatatgccaaatcaatgctcgagtggctgtccccttcactacttaagacatgggaaaacagaaataatacttctccatttgaaattggatcacggatcaaaatcatcgcaccaaacgaattgagtaaatatcctggttcaaagatttgcttcgtatctgaggtaggagctttgattaatgaagtgataattaaagttggtaattctgagaagacaaccttgattttgaccaagccgagctttgaatgtgcgtcgtctttagacaagatactagaaattgttgaacaagatgaaagaaattggaaaacatttcctgaagatggtaaatcattcctctgcgataattacatttcaatagacaccataaaagaagaacccttgagtaaagaggaaacagaggcgtttaaagtacagcttaaagaaaagaaaagggacagaaataagaaaattctactggtgaaaagagagtctaagaaactggctaacggcaacgcaatcatagatgataccaacggagagagggcaatgagaaatcaagacattttagtggaaaacgtcaatggagtaccaccgattgatcatattatggggggtgacgaagatgatgatgaagaggaagaaaacgacaatttgttgaatttactcaaggataactctgagaaatcagcagcgaaaaaaaatacggaagttcctgtagacattataattcaaccaagtgccgcttcaaaacataaaatgttcccatttaaccccgctaaaatcaagaaagatgattatggtactgttgtagattttacgatgtttttacctgatgattctgacaatgttaatcaaaatagcaggaaaagacctctcaaagacggtgctaaaaccacgagccctgtaaacgaagaagataacaaaaatgaagaagaagatggttataatatgagtgatccaattagcaaaaggagcaaacaccgtgcttctcgttactcgggattttctggaaccggagaagcagaaaattttgataaccttgactatttaaagatagacaaaacgctctccaaaagaacaatctcaactgtaaatgtccaattgaaatgctccgtagtaatcctaaatttacagagtttagttgatcaaag

SEQ ID NO: 7 - protein, 372 aa, Saccharomyces cerevisiae
MKCRVWSEARVYTNINKQRTEEYWDYENTVIDWSTNTKDYEIENKVGRGKYSEVFQGVKLDSKVKIVIKMLKPVKKKKIKREIKILTDLSNEKVPPTTLPFQKDQYYTNQKEDVLKFIRPYIFDQPHNGHANIIHLFDIIKDPISKTPALVFEYVDNVDFRILYPKLTDLEIRFYMFELLKALDYCHSMGIMHRDVKPHNVMIDHKNKKLRLIDWGLAEFYHVNMEYNVRVASRFFKGPELLVDYRMYDYSLDLWSFGTMLASMIFKREPFFHGTSNTDQLVKIVKVLGTSDFEKYLLKYEITLPREFYDMDQYIRKPWHRFINDGNKHLSGNDEIIDLIDNLLRYDHQERLTAKEAMGHPWFAPIREQIEK

SEQ ID NO: 8 - DNA, 1119 bp, Saccharomyces cerevisiae
atgaaatgcagggtatggtcagaggctcgtgtttatacgaatatcaataaacaaagaaccgaggaatattgggattatgaaaatactgtaattgattggtccacaaatacaaaggactatgaaattgaaaataaagttgggcgaggaaaatactccgaggttttccaaggtgtcaaattagactctaaagttaaaattgttattaagatgttgaaaccagttaaaaagaagaagatcaagagagaaatcaaaatcttaacggatttgtctaacgaaaaagtgcctccaacgactttgccatttcaaaaagatcaatattacacaaatcaaaaggaggacgttttaaaattcatcaggccctatatctttgatcagccacacaatggtcatgcaaacataattcatctatttgatataataaaggatcccatctcaaaaactccggctttggtcttcgaatacgtagataatgtggacttccgtattctttaccctaaattaaccgatctcgaaattaggttttatatgtttgagttattaaaggccttagactattgtcattcaatgggaataatgcatagagatgttaaacctcataacgtaatgattgatcataagaataaaaaattgcgattgatagattgggggcttgctgaattttatcatgttaatatggaatacaatgttcgtgttgcgtcgaggttttttaagggtcctgaactactagttgactacagaatgtatgattattctttagacttgtggtcgtttgggacaatgttggcttctatgatctttaaaagagagccatttttccatggaacgagtaacacagaccagcttgtcaagatcgtcaaagtacttggtacaagcgattttgagaaatacctgttgaagtatgaaattaccttaccgagagaattttacgatatggaccaatacatcagaaagccttggcatagattcatcaatgatggtaataaacatttaagcggcaacgatgaaattattgaccttattgacaatcttttgagatatgatcatcaagaaagattaactgctaaggaagcgatgggacacccgtggtttgccccaataagggaacaaattgaaaaataa

SEQ ID NO: 9 - protein, 445 aa, Saccharomyces cerevisiae
MASLPGIDEHTTSEELITGDNEWHKLVIPKGSDWQIDLKAEGKLIVKVNSGIVEIFGTELAVDDEYTFQNWKFPIYAVEETELLWKCPDLTTNTITVKPNHTMKYIYNLHFMLEKIRMSNFEGPRVVIVGGSQTGKTSLSRTLCSYALKFNAYQPLYINLDPQQPIFTVPGCISATPISDILDAQLPTWGQSLTSGATLLHNKQPMVKNFGLERINENKDLYLECISQLGQVVGQRLHLDPQVRRSGCIVDTPSISQLDENLAELHHIIEKLNVNIMLVLCSETDPLWEKVKKTFGPELGNNNIFFIPKLDGVSAVDDVYKRSLQRTSIREYFYGSLDTALSPYAIGVDYEDLTIWKPSNVFDNEVGRVELFPVTITPSNLQHAIIAITFAERRADQATVIKSPILGFALITEVNEKRRKLRVLLPVPGRLPSKAMILTSYRYLE

SEQ ID NO: 10 - DNA, 1338 bp, Saccharomyces cerevisiae
atggcaagtctacctggtattgatgagcatactacctctgaagaattgataactggtgataacgaatggcacaagttggttattcccaagggcagtgattggcaaattgatcttaaagcagagggaaaactgatagtcaaggtaaactcgggcatagttgagatatttggcaccgagttggcagtagatgatgagtacacttttcagaactggaagtttcccatatacgctgtcgaagaaacagaattattatggaaatgtcctgatttaactacgaatacaataactgtcaagcctaaccatactatgaaatatatttataatctacacttcatgttggagaagatacgaatgtctaactttgaaggccctcgagtggtgattgttggtggttcgcaaaccgggaagacttccctatcgaggacactatgttcttacgctttgaaattcaacgcttaccaaccactatacatcaatcttgaccctcaacagcccatttttactgttcctggatgcatatctgccaccccaatatcagatatacttgatgcacaactacccacttggggtcagagtctcactagtggtgccacactactacataataagcagccaatggtgaaaaattttggcctggaaaggattaatgagaacaaagatctataccttgagtgtataagccagttaggtcaagtagtaggtcaaaggttacatttggatcctcaagtcaggagatcagggtgcattgtcgatacgccatcaatatcacaactggatgaaaatttggctgaactgcaccatatcattgagaaactcaatgttaacattatgctagtactatgttctgaaacggatcctctttgggagaaagtaaaaaagacatttggtccagaattgggaaataataatatttttttcattcctaaattggatggtgtatccgcagttgatgatgtatacaaaagatccttgcagaggacatccataagagagtacttttacggctctcttgatacagccttgagtccttatgctattggtgttgattatgaggatttaactatttggaaacctagcaatgttttcgataacgaggtcggtagggtggaactgttccccgtcactataactccaagtaacctacagcacgctattatagccataacgttcgcagaaagaagggcagatcaggcaacagtaataaaatcgcctattttaggattcgctttgattacagaagttaatgaaaaaaggcgtaaattaagggttttactacctgtcccaggccgacttcccagcaaggcgatgattctaacttcatatagatatttagagtaa

SEQ ID NO: 11 - protein, 842 aa, Saccharomyces cerevisiae
MVAFTVDQMRSLMDKVTNVRNMSVIAHVDHGKSTLTDSLVQRAGIISAAKAGEARFTDTRKDEQERGITIKSTAISLYSEMSDEDVKEIKQKTDGNSFLINLIDSPGHVDFSSEVTAALRVTDGALVVVDTIEGVCVQTETVLRQALGERIKPVVVINKVDRALLELQVSKEDLYQTFARTVESVNVIVSTYADEVLGDVQVYPARGTVAFGSGLHGWAFTIRQFATRYAKKFGVDKAKMMDRLWGDSFFNPKTKKWTNKDTDAEGKPLERAFNMFILDPIFRLFTAIMNFKKDEIPVLLEKLEIVLKGDEKDLEGKALLKVVMRKFLPAADALLEMIVLHLPSPVTAQAYRAEQLYEGPADDANCIAIKNCDPKADLMLYVSKMVPTSDKGRFYAFGRVFAGTVKSGQKVRIQGPNYVPGKKDDLFIKAIQRVVLMMGRFVEPIDDCPAGNIIGLVGIDQFLLKTGTLTTSETAHNMKVMKFSVSPVVQVAVEVKNANDLPKLVEGLKRLSKSDPCVLTYMSESGEHIVAGTGELHLEICLQDLEHDHAGVPLKISPPVVAYRETVESESSQTALSKSPNKHNRIYLKAEPIDEEVSLAIENGIINPRDDFKARARIMADDYGWDVTDARKIWCFGPDGNGPNLVIDQTKAVQYLHEIKDSVVAAFQWATKEGPIFGEEMRSVRVNILDVTLHADAIHRGGGQIIPTMRRATYAGFLLADPKIQEPVFLVEIQCPEQAVGGIYSVLNKKRGQVVSEEQRPGTPLFTVKAYLPVNESFGFTGELRQATGGQAFPQMVFDHWSTLGSDPLDPTSKAGEIVLAARKRHGMKEEVPGWQEYYDKL

SEQ ID NO: 12 - DNA, 2529 bp, Saccharomyces cerevisiae
atggttgctttcactgttgaccaaatgcgttctttaatggacaaagttaccaatgtgcgtaacatgtccgttattgctcacgtcgatcatggtaagtccactttgaccgattccttggtccaaagagccggtattatttccgctgctaaggctggtgaagctcgtttcaccgataccagaaaggatgaacaagaaagaggtatcactatcaagtctaccgctatttctctatactctgaaatgtctgacgaagatgtcaaggaaatcaagcaaaagaccgacggtaactccttcttgatcaacttgatcgactctccaggtcacgttgacttctcctctgaagttactgccgctttacgtgtcactgacggtgctttggttgtcgtcgacaccattgaaggtgtctgtgtccaaaccgaaactgttttgagacaagctttgggtgagagaatcaagcctgttgttgttatcaacaaggtcgacagagctttgttggaattgcaagtttctaaggaagatttataccaaacctttgccagaactgttgaatccgttaacgtcatcgtttccacctacgccgatgaagttttgggtgatgtccaagtttacccagccagaggtaccgttgccttcggttccggtttgcacggttgggctttcactatccgtcaattcgccaccagatatgctaagaaattcggtgtcgacaaggccaagatgatggacagattatggggtgactctttcttcaacccaaagaccaagaagtggaccaacaaggacactgatgctgaaggtaagccattggaaagagctttcaacatgttcatcttggacccaatcttcagattattcactgctatcatgaacttcaagaaagatgaaattccagttttgctagaaaagttggaaattgtcttgaagggtgacgaaaaggacttggaaggtaaggccttgttgaaggttgttatgagaaagttcttgccagctgccgatgccttattggaaatgattgtcttgcacttgccatctccagtcactgctcaagcctacagagctgaacaattatacgaaggtccagctgacgatgccaactgtattgctatcaagaactgtgatccaaaggctgatttgatgttgtacgtctccaagatggtgccaacctctgataagggtagattctacgccttcggtagagtttttgccggtactgttaagtccggtcaaaaggtcagaatccaaggtccaaactacgttccaggtaagaaggacgatttgttcatcaaggccattcaaagagttgttttgatgatgggtagatttgtcgaaccaatcgatgactgtccagccggtaacattatcggtttagtcggtatcgatcaattcttgttgaagactggtactttgaccaccagtgaaactgctcacaacatgaaggtcatgaaattctctgtctctccagttgtgcaagtcgctgtcgaagtcaagaacgctaacgacttaccaaaattggtcgaaggtttgaagagattgtccaagtctgatccatgtgtcttgacctatatgtctgaatccggtgaacatatcgttgctggtaccggtgaattgcatttggaaatttgtttgcaagatttggaacacgaccacgctggtgttccattgaagatctccccaccagttgtcgcttacagagaaactgttgaaagtgaatcttctcaaactgctttgtccaagtctccaaacaagcataacagaatctacttgaaggctgaaccaattgacgaagaagtctctttggctattgaaaacggtatcatcaacccaagagatgatttcaaggccagagctagaatcatggctgacgactacggttgggatgtcaccgatgccagaaagatctggtgtttcggtccagacggtaacggtccaaacttggttattgaccaaactaaggctgtccaatacttgcacgaaatcaaggattccgttgttgctgctttccaatgggctaccaaggaaggtccaattttcggtgaagaaatgagatctgtcagagttaacattttggatgttactttacatgccgatgctatccacagaggtggtggtcaaatcatcccaaccatgagaagagctacttacgctggtttcttgttggctgatccaaagatccaagaaccagttttcttggtcgaaattcaatgtccagaacaagccgtcggtggtatctactccgtcttaaacaagaagagaggtcaagtcgtttctgaagaacaaagaccaggtactccattgtttaccgtcaaggcctacttgccagttaacgaatctttcggtttcactggtgaattgagacaagctactggtggtcaagctttcccacaaatggttttcgaccattggtccactttaggttctgacccattggacccaacctctaaggctggtgaaattgttcttgctgctcgtaagagacacggtatgaaggaagaagttccaggctggcaagaatattacgacaaattgtaa

SEQ ID NO: 13 - protein, 437 aa, Saccharomyces cerevisiae
MAVSKVYARSVYDSRGNPTVEVELTTEKGVFRSIVPSGASTGVHEALEMRDEDKSKWMGKGVMNAVNNVNNVIAAAFVKANLDVKDQKAVDDFLLSLDGTANKSKLGANAILGVSMAAARAAAAEKNVPLYQHLADLSKSKTSPYVLPVPFLNVLNGGSHAGGALALQEFMIAPTGAKTFAEAMRIGSEVYHNLKSLTKKRYGASAGNVGDEGGVAPNIQTAEEALDLIVDAIKAAGHDGKVKIGLDCASSEFFKDGKYDLDFKNPESDKSKWLTGVELADMYHSLMKRYPIVSIEDPFAEDDWEAWSHFFKTAGIQIVADDLTVTNPARIATAIEKKAADALLLKVNQIGTLSESIKAAQDSFAANWGVMVSHRSGETEDTFIADLVVGLRTGQIKTGAPARSERLAKLNQLLRIEEELGDKAVYAGENFHHGDKL

SEQ ID NO: 14 - DNA, 1314 bp, Saccharomyces cerevisiae
atggctgtctctaaagtttacgctagatccgtctacgactcccgtggtaacccaaccgtcgaagtcgaattaaccaccgaaaagggtgttttcagatccattgttccatctggtgcctccaccggtgtccacgaagctttggaaatgagagatgaagacaaatccaagtggatgggtaagggtgttatgaacgctgtcaacaacgtcaacaacgtcattgctgctgctttcgtcaaggccaacctagatgttaaggaccaaaaggccgtcgatgacttcttgttgtctttggatggtaccgccaacaagtccaagttgggtgctaacgctatcttgggtgtctccatggccgctgctagagccgctgctgctgaaaagaacgtcccattgtaccaacatttggctgacttgtctaagtccaagacctctccatacgttttgccagttccattcttgaacgttttgaacggtggttcccacgctggtggtgctttggctttgcaagaattcatgattgctccaactggtgctaagaccttcgctgaagccatgagaattggttccgaagtttaccacaacttgaagtctttgaccaagaagagatacggtgcttctgccggtaacgtcggtgacgaaggtggtgttgctccaaacattcaaaccgctgaagaagctttggacttgattgttgacgctatcaaggctgctggtcacgacggtaaggtcaagatcggtttggactgtgcttcctctgaattcttcaaggacggtaagtacgacttggacttcaagaacccagaatctgacaaatccaagtggttgactggtgtcgaattagctgacatgtaccactccttgatgaagagatacccaattgtctccatcgaagatccatttgctgaagatgactgggaagcttggtctcacttcttcaagaccgctggtatccaaattgttgctgatgacttgactgtcaccaacccagctagaattgctaccgccatcgaaaagaaggctgctgacgctttgttgttgaaggttaaccaaatcggtaccttgtctgaatccatcaaggctgctcaagactctttcgctgccaactggggtgttatggtttcccacagatctggtgaaactgaagacactttcattgctgacttggttgtcggtttgagaactggtcaaatcaagactggtgctccagctagatccgaaagattggctaagttgaaccaattgttgagaatcgaagaagaattgggtgacaaggctgtctacgccggtgaaaacttccaccacggtgacaagttgtaa

SEQ ID NO: 15 - protein, 312 aa, Saccharomyces cerevisiae
MDSQPVDVDNIIDRLLEVRGSKPGQQVDLEENEIRYLCSKARSIFIKQPILLELEAPIKICGDIHGQYYDLLRLFEYGGFPPESNYLFLGDYVDRGKQSLETICLLLAYKIKYPENFFILRGNHECASINRIYGFYDECKRRYNIKLWKTFTDCFNCLPIAAIIDEKIFCMHGGLSPDLNSMEQIRRVMRPTDIPDVGLLCDLLWSDPDKDIVGWSENDRGVSFTFGPDVVNRFLQKQDMELICRAHQVVEDGYEFFSKRQLVTLFSAPNYCGEFDNAGAMMSVDESLLCSFQILKPAQKSLPRQAGGRKKK

SEQ ID NO: 16 - DNA, 939 bp, Saccharomyces cerevisiae
atggactcacaaccagttgacgttgataatatcatcgatagattattggaagtaagaggatctaaacctggtcaacaagttgatctagaagaaaatgaaatcagatacttatgttcgaaagccagatctatattcataaagcaacccattttactagagttagaagccccaattaaaatatgtggtgacattcatgggcaatactatgatttactacgtctatttgagtacggtggattcccgccagaatctaattatctatttttgggtgattatgtcgaccgtggtaaacaatccttagagactatttgtctattactggcttacaaaattaagtatccagaaaactttttcattttaagagggaaccatgaatgtgcttccattaatagaatttacgggttttatgatgaatgtaagagacgttataatatcaaactttggaaaactttcacggattgtttcaattgtttaccaattgctgcaattattgatgagaaaatcttctgtatgcatggtggtctctcaccagatttgaatagtatggaacagatcagaagggtgatgaggccaacagatattcccgacgttggcttattatgtgacttattgtggtcagatccagataaagatatcgtaggttggagtgaaaatgatagaggtgtttctttcacttttggtcctgatgtagtgaacagatttttacagaaacaagatatggagttgatttgcagggcccatcaagttgtggaagatggttatgaattctttagtaaaagacaattggtgacacttttcagtgctccgaattattgtggtgaatttgataacgctggtgcaatgatgagtgttgatgaaagtttattatgttcttttcaaattttaaagccagcccaaaaaagtctaccaaggcaagctgggggtagaaagaaaaaataa

SEQ ID NO: 17 - protein, 247 aa, Saccharomyces cerevisiae
MPKLVLVRHGQSEWNEKNLFTGWVDVKLSAKGQQEAARAGELLKEKKVYPDVLYTSKLSRAIQTANIALEKADRLWIPVNRSWRLNERHYGDLQGKDKAETLKKFGEEKFNTYRRSFDVPPPPIDASSPFSQKGDERYKYVDPNVLPETESLALVIDRLLPYWQDVIAKDLLSGKTVMIAAHGNSLRGLVKHLEGISDADIAKLNIPTGIPLVFELDENLKPSKPSYYLDPEAAAAGAAAVANQGKK

SEQ ID NO: 18 - DNA, 744 bp, Saccharomyces cerevisiae
atgccaaagttagttttagttagacacggtcaatccgaatggaacgaaaagaacttattcaccggttgggttgatgttaaattgtctgccaagggtcaacaagaagccgctagagccggtgaattgttgaaggaaaagaaggtctacccagacgtcttgtacacttccaagttgtccagagctatccaaactgctaacattgctttggaaaaggctgacagattatggattccagtcaacagatcctggagattgaacgaaagacattacggtgacttacaaggtaaggacaaggctgaaactttgaagaagttcggtgaagaaaaattcaacacctacagaagatccttcgatgttccacctcccccaatcgacgcttcttctccattctctcaaaagggtgatgaaagatacaagtacgttgacccaaatgtcttgccagaaactgaatctttggctttggtcattgacagattgttgccatactggcaagatgtcattgccaaggacttgttgagtggtaagaccgtcatgatcgccgctcacggtaactccttgagaggtttggttaagcacttggaaggtatctctgatgctgacattgctaagttgaacatcccaactggtattccattggtcttcgaattggacgaaaacttgaagccatctaagccatcttactacttggacccagaagctgccgctgctggtgccgctgctgttgccaaccaaggtaagaaataa

SEQ ID NO: 19 - protein, 327 aa, Saccharomyces cerevisiae
MSSSEDEDDKFLYGSDSELALPSSKRSRDDEADAGASSNPDIVKRQKFDSPVEETPATARDDRSDEDIYSDSSDDDSDSDLEVIISLGPDPTRLDAKLLDSYSTAATSSSKDVISVATDVSNTITKTSDERLITEGEANQGVTATTVKATESDGNVPKAMTGSIDLDKEGIFDSVGITTIDPEVLKEKPWRQPGANLSDYFNYGFNEFTWMEYLHRQEKLQQDYNPRRILMGLLSLQQQGKLNSANDTDSNLGNIIDNNNNVNNANMSNLNSNMGNSMSGTPNPPAPPMHPSFPPLPMFGSFPPFPMPGMMPPMNQQPNQNQNQNSK

SEQ ID NO: 20 - DNA, 984 bp, Saccharomyces cerevisiae
atgagctccagtgaagacgaagacgacaagttcttgtatggttccgactccgaattagcactaccttcatctaaacgatcaagagatgatgaagcagacgcaggtgcgtccagtaatcctgatatagttaaaaggcaaaaattcgactctcccgtggaagaaactccagctactgccagagatgatcgttctgatgaagatatctactctgactcctcagatgacgatagtgattctgacctagaggttatcataagtctgggtcctgaccctactaggttagatgcaaaactactcgattcttattctaccgcagcgacatcttcaagcaaagacgtaattagcgtagctacagatgtatccaataccatcacaaagacatcagatgaaagactaataacagaaggagaagcaaatcaaggtgtaacggcaacgaccgtaaaagctacagagagcgatggaaatgtaccgaaagcaatgactggttctatagacctggataaagagggaatctttgatagtgttggcataacgacaatagatcctgaagtattaaaggagaaaccctggaggcaaccgggggccaacttaagtgattatttcaattacggttttaacgaatttacctggatggagtatttacatagacaggaaaaactacaacaagattataatcctaggaggatcctaatgggcctattatccctccaacagcaagggaagttgaattccgcgaatgatacagactcaaacctcggtaatataattgataacaacaacaacgtaaacaatgcaaatatgtctaatctgaacagtaatatgggtaatagtatgtctggaacaccaaaccctcccgctccaccaatgcatccaagcttcccacccttacctatgtttggtagctttccaccattccccatgccaggtatgatgccacccatgaaccaacagcctaatcaaaatcaaaatcaaaattcgaaatga

SEQ ID NO: 21 - protein, 103 aa, Saccharomyces cerevisiae
MSGRGKGGKGLGKGGAKRHRKILRDNIQGITKPAIRRLARRGGVKRISGLIYEEVRAVLKSFLESVIRDSVTYTEHAKRKTVTSLDVVYALKRQGRTLYGFGG

SEQ ID NO: 22 - DNA, 312 bp, Saccharomyces cerevisiae
atgtccggtagaggtaaaggtggtaaaggtctaggaaaaggtggtgccaagcgtcacagaaagattctaagagataacattcaaggtatcactaagccagctatcagaagattagctagaagaggtggtgtcaagcgtatttctggtttgatctacgaagaagtcagagccgtcttgaaatccttcttggaatccgtcatcagggactctgttacttacactgaacacgccaagagaaagactgttacttctttggatgttgtttatgctttgaagagacaaggtagaaccttatatggtttcggtggttaa

SEQ ID NO: 23 - protein, 132 aa, Saccharomyces cerevisiae
MSGGKGGKAGSAAKASQSRSAKAGLTFPVGRVHRLLRRGNYAQRIGSGAPVYLTAVLEYLAAEILELAGNAARDNKKTRIIPRHLQLAIRNDDELNKLLGNVTIAQGGVLPNIHQNLLPKKSAKATKASQEL

SEQ ID NO: 24 - DNA, 399 bp, Saccharomyces cerevisiae
atgtccggtggtaaaggtggtaaagctggttcagctgctaaagcttctcaatctagatctgctaaggctggtttgacattcccagtcggtagagtgcacagattgctaagaagaggtaactacgcccaaagaattggttctggtgctccagtctacttgactgctgtcttggaatatttggccgctgaaattttagaattagctggtaatgctgctagggataacaagaagaccagaattattccaagacatttgcaattggctatcagaaatgatgacgaattgaacaagctattgggtaacgttaccattgcccaaggtggtgttttgccaaacatccatcaaaacttgttgccaaagaagtctgccaaggctaccaaggcttctcaagaattataa

SEQ ID NO: 25 - protein, 705 aa, Saccharomyces cerevisiae
MAGETFEFQAEITQLMSLIINTVYSNKEIFLRELISNASDALDKIRYQALSDPKQLETEPDLFIRITPKPEEKVLEIRDSGIGMTKAELINNLGTIAKSGTKAFMEALSAGADVSMIGQFGVGFYSLFLVADRVQVISKNNEDEQYIWESNAGGSFTVTLDEVNERIGRGTVLRLFLKDDQLEYLEEKRIKEVIKRHSEFVAYPIQLLVTKEVEKEVPIPEEEKKDEEKKDEDDKKPKLEEVDEEEEEKKPKTKKVKEEVQELEELNKTKPLWTRNPSDITQEEYNAFYKSISNDWEDPLYVKHFSVEGQLEFRAILFIPKRAPFDLFESKKKKNNIKLYVRRVFITDEAEDLIPEWLSFVKGVVDSEDLPLNLSREMLQQNKIMKVIRKNIVKKLIEAFNEIAEDSEQFDKFYSAFAKNIKLGVHEDTQNRAALAKLLRYNSTKSVDELTSLTDYVTRMPEHQKNIYYITGESLKAVEKSPFLDALKAKNFEVLFLTDPIDEYAFTQLKEFEGKTLVDITKDFELEETDEEKAEREKEIKEYEPLTKALKDILGDQVEKVVVSYKLLDAPAAIRTGQFGWSANMERIMKAQALRDSSMSSYMSSKKTFEISPKSPIIKELKKRVDEGGAQDKTVKDLTNLLFETALLTSGFSLEEPTSFASRINRLISLGLNIDEDEETETAPEASTEAPVEEVPADTEMEEVD

SEQ ID NO: 26 - DNA, 2000 bp, Saccharomyces cerevisiae
atggctggtgaaacttttgaatttcaagctgaaatcactcagttgatgagtttgatcatcaacactgtctattctaacaaggaaattttcttgagagaactgatctctaacgcctccgatgctttagacaaaattagataccaagctttgtctgatccaaagcaattggaaaccgaaccagatttgttcattagaatcaccccaaaaccagaagaaaaagttttggaaatcagagattctggtattggtatgaccaaggctgaattgattaacaatttgggtaccattgctaagtctggtactaaagctttcatggaagctctatctgctggtgccgatgtatccatgattggtcaattcggtgttggtttttactctttattcttagtcgccgacagagttcaagttatttccaagaacaatgaggacgaacaatatatttgggaatctaatgccggtggttctttcaccgttactttggacgaagttaacgaaagaattggtagaggtaccgtcttgagattattcttgaaagatgaccaattggagtacttggaagaaaagagaattaaagaagtcatcaagagacattctgaattcgttgcttaccctatccaacttctagtcaccaaggaagtcgaaaaggaagttccaattccagaagaagaaaagaaagacgaggaaaagaaggatgaagatgacaagaaaccaaaattggaagaagtcgatgaagaagaagaagaaaagaagccaaaaaccaaaaaagttaaagaagaggttcaagaattagaagagttgaacaagactaagccattatggactagaaacccatctgatatcactcaagaggaatacaatgctttctataagtctatttctaacgactgggaagacccattgtacgttaagcatttctctgttgaaggtcaattggaatttagagctatcttgttcattccaaagagagcaccattcgacttatttgagagtaagaagaagaagaacaatatcaagttgtacgttcgtcgtgtcttcatcactgatgaagctgaagacttgattccagagtggttatctttcgtcaagggtgttgttgactctgaagatttaccattgaatttgtccagagaaatgttacaacaaaataagattatgaaggttattagaaagaatattgtcaagaaattgattgaagccttcaacgaaatcgctgaagactccgagcaatttgacaaattttactctgccttcgctaagaacattaagctgggtgtacatgaggacactcaaaacagagctgctttagctaagttgctacgttacaattctactaaatctgtcgatgaattgacttccttgactgattacgttactagaatgccagaacaccaaaagaacatctattacatcaccggtgaatctctaaaggcagtcgaaaagtctccattcttggacgccttgaaggctaagaactttgaagttttgttcttgaccgacccaattgatgaatacgctttcactcaattgaaggaattcgagggtaaaactttggttgacattactaaagatttcgaattggaagaaacagacgaagaaaaagctgaaagagagaaggagatcaaagaatacgaaccattgaccaaggccttgaaggatatcttgggtgaccaagtggagaaggttgttgtttcttacaaattgctagatgctccagctgccatcagaactggtcaattcggctggtctgctaacatggaaagaatcatgaaggctcaagccttgagagactcttccatgtcctcctacatgtcttccaagaagactttcgaaatttctccaaaatctccaattattaaggaattgaaaaagagagttgatgagggtggtgcacaagataagaccgtcaaagatttgactaacttattattcgagaccgctttgttgacttctggtttcagtttggaagaaccaacttcttttgcatcaagaataaatagatt

SEQ ID NO: 27 - protein, 523 aa, Saccharomyces cerevisiae
MAAIRDYKTALDFTKSLPRPDGLSVQELMDSKIRGGLTYNDFLILPGLVDFASSEVSLQTKLTRNITLNIPLVSSPMDTVTESEMATFMALLGGIGFIHHNCTPEDQADMVRRVKNYENGFINNPIVISPTTTVGEAKSMKEKYGFAGFPVTTDGKRNAKLVGVITSRDIQFVEDNSLLVQDVMTKNPVTGAQGITLSEGNEILKKIKKGRLLVVDEKGNLVSMLSRTDLMKNQNYPLASKSANTKQLLCGASIGTMDADKERLRLLVKAGLDVVILDSSQGNSIFELNMLKWVKESFPGLEVIAGNVVTREQAANLIAAGADGLRIGMGTGSICITQEVMACGRPQGTAVYNVCEFANQFGVPCMADGGVQNIGHITKALALGSSTVMMGGMLAGTTESPGEYFYQDGKRLKAYRGMGSIDAMQKTGTKGNASTSRYFSESDSVLVAQGVSGAVVDKGSIKKFIPYLYNGLQHSCQDIGCRSLTLLKNNVQRGKVRFEFRTASAQLEGGVHNLHSYEKRLHN

SEQ ID NO: 28 - DNA, 1572 bp, Saccharomyces cerevisiae
atggccgccattagagactacaagaccgcactagactttaccaagagcctaccaagaccggatggtttgtcagtgcaggaattgatggactccaagatcagaggtgggttgacttataacgattttttaatcttaccaggtttagtcgattttgcgtcctctgaagttagcctacagaccaagctaaccaggaatattactttaaatattccattagtttcctctccaatggacacggtgacagagtcagaaatggccacttttatggctctgttgggtggtatcggtttcattcaccataactgtaccccagaggaccaagctgacatggtcagaagagtcaagaactatgaaaatgggtttattaacaaccctatagtgatttctccaactacgaccgttggtgaagctaagagcatgaaggaaaagtatggatttgcaggcttccctgtcacgacagatggaaagagaaatgcaaagttggtgggtgtcatcacctctcgtgatatacaattcgttgaggacaactctttactcgttcaggatgtcatgaccaaaaaccctgttaccggcgcacaaggtatcacattatcagaaggtaacgaaattctaaagaaaatcaaaaagggtaggctattggttgttgatgaaaagggtaacttagtttctatgctttcccgaactgatttaatgaaaaatcagaactacccattagcgtccaaatctgccaacaccaagcaactgttatgtggtgcttctattgggactatggacgctgataaagaaagactaagattattggtaaaagctgggttggatgtcgtcatattggattcatcccaaggaaactctatcttcgaattgaacatgctcaagtgggtcaaagagagtttcccaggtctggaagtcatcgctggtaacgttgtcaccagggaacaagctgccaatttgattgctgccggtgcggacggtttgagaattggtatgggaactggctctatttgtattacccaagaagttatggcttgtggtaggccacaaggtacagccgtctacaatgtgtgtgaatttgctaaccaattcggtgttccatgtatggctgatggtggtgttcaaaacattggtcatattaccaaagctttggctcttggttcttctactgttatgatgggtggtatgttggccggtactaccgaatcaccaggtgaatatttctatcaagatggtaaaagattgaaggcgtatcgtggtatgggctccattgacgccatgcaaaagactggtaccaaaggtaatgcatctacctcccgttacttttccgaatcagacagtgttttggtcgcacaaggtgtctccggtgctgtcgttgacaaaggatccattaagaaatttattccatatttgtacaatggtttacaacattcctgtcaagacatcggctgtaggtcgctaactttattaaagaataatgttcaaaggggtaaagttagatttgaattcagaaccgcttctgctcaactagaaggtggcgttcataatttacattcttacgaaaagcgtttacataactga

SEQ ID NO: 29 - protein, 524 aa, Saccharomyces cerevisiae
MSAAPLDYKKALEHLKTYSSKDGLSVQELMDSTTRGGLTYNDFLVLPGLVNFPSSAVSLQTKLTKKITLNTPFVSSPMDTVTEADMAIYMALLGGIGFIHHNCTPKEQASMVKKVKMFENGFINSPIVISPTTTVGEVKVMKRKFGFSGFPVTEDGKCPGKLVGLVTSRDIQFLEDDSLVVSEVMTKNPVTGIKGITLKEGNEILKQTKKGKLLIVDDNGNLVSMLSRADLMKNQNYPLASKSATTKQLLCGAAIGTIEADKERLRLLVEAGLDVVILDSSQGNSVFQLNMIKWIKETFPDLEIIAGNVATREQAANLIAAGADGLRIGMGSGSICITQEVMACGRPQGTAVYNVCQFANQFGVPCMADGGVQNIGHITKALALGSSTVMMGGMLAGTTESPGEYFYKDGKRLKAYRGMGSIDAMQKTGNKGNASTSRYFSESDSVLVAQGVSGAVVDKGSIKKFIPYLYNGLQHSCQDIGCESLTSLKENVQNGEVRFEFRTASAQLEGGVHNLHSYEKRLYN

SEQ ID NO: 30 - DNA, 1575 bp, Saccharomyces cerevisiae
atgagtgctgctccattggattacaaaaaggctttagaacatttgaagacatacagttccaaggacggtctttctgtccaggaattgatggactccacaactagaggtgggttgacctacaatgattttttggtcttaccaggcttggtcaatttcccatcttctgctgtcagtttgcaaaccaaattgaccaagaaaatcactttgaacactccttttgtctcttctcctatggacacagtcactgaagctgatatggctatttatatggctttattgggtggtattggtttcatccatcacaactgtactccaaaggaacaggcttccatggtcaagaaagttaaaatgtttgaaaacggtttcatcaattctccaatagtaatttctccaaccaccactgttggtgaagttaaggttatgaagagaaagtttggtttctctggcttcccagttactgaagacggtaagtgtccaggtaagttagttgggttagtcacctctcgtgatatacaattcttagaagatgattctttggttgtttctgaagtcatgactaaaaatccagttactggcatcaagggtattactttgaaagaaggtaatgaaatcctaaaacaaaccaagaaaggtaaattgcttatcgttgatgataacggtaacctcgtttccatgttgtcaagagcggatttgatgaagaatcaaaactacccgttagcttctaaatccgccaccaccaagcaattgctatgtggtgctgcaattggtactatcgaagctgataaggaaagattaagactattagtcgaagcaggtttggatgttgttatcttagattcctctcaaggtaactctgttttccaattgaacatgatcaaatggattaaagaaactttcccagatttggaaatcattgctggtaacgttgccaccagagaacaagctgctaacttgattgctgccggtgccgatggtttaagaattggtatgggttctgggtctatttgtatcactcaagaagttatggcttgtggtagaccacaaggtacagctgtctacaacgtttgtcaatttgctaaccaatttggcgttccatgtatggctgatggtggtgtccaaaacattggccacatcaccaaggctttggcccttggttcctccaccgtcatgatgggtggtatgttagctggtactaccgagtcaccaggtgaatacttctacaaggatggtaagagattgaaggcttatcgtggtatggggtccattgacgccatgcaaaagaccggtaacaagggtaatgcctctacttctcgttatttctctgagtcagacagtgtcttggttgcacaaggtgtttctggggctgtcgtagacaaaggttccatcaagaagtttattccatatttgtacaacggtttacaacattcttgtcaagacattggttgcgaatctctaacttcattgaaagagaatgttcaaaatggtgaagttagatttgaattcagaaccgcttctgctcaattagaaggtggtgtccataatttgcactcctatgaaaaacgtctatacaattga

SEQ ID NO: 31 - protein, 767 aa, Saccharomyces cerevisiae
MVQSAVLGFPRIGPNRELKKATEGYWNGKITVDELFKVGKDLRTQNWKLQKEAGVDIIPSNDFSFYDQVLDLSLLFNVIPDRYTKYDLSPIDTLFAMGRGLQRKATETEKAVDVTALEMVKWFDSNYHYVRPTFSKTTQFKLNGQKPVDEFLEAKELGIHTRPVLLGPVSYLFLGKADKDSLDLEPLSLLEQLLPLYTEILSKLASAGATEVQIDEPVLVLDLPANAQAAIKKAYTYFGEQSNLPKITLATYFGTVVPNLDAIKGLPVAALHVDFVRAPEQFDEVVAAIGNKQTLSVGIVDGRNIWKNDFKKSSAIVNKAIEKLGADRVVVATSSSLLHTPVDLNNETKLDAEIKGFFSFATQKLDEVVVITKNVSGQDVAAALEANAKSVESRGKSKFIHDAAVKARVASIDEKMSTRAAPFEQRLPEQQKVFNLPLFPTTTIGSFPQTKDIRINRNKFNKGTISAEEYEKFINSEIEKVIRFQEEIGLDVLVHGEPERNDMVQYFGEQINGYAFTVNGWVQSYGSRYVRPPIIVGDLSRPKAMSVKESVYAQSITSKPVKGMLTGPITCLRWSFPRDDVDQKTQAMQLALALRDEVNDLEAAGIKVIQVDEPALREGLPLREGTERSAYYTWAAEAFRVATSGVANKTQIHSHFCYSDLDPNHIKALDADVVSIEFSKKDDANYIAEFKNYPNHIGLGLFDIHSPRIPSKDEFIAKISTILKSYPAEKFWVNPDCGLKTRGWEETRLSLTHMVEAAKYFREQYKN

SEQ ID NO: 32 - DNA, 2304 bp, Saccharomyces cerevisiae
atggttcaatctgctgtcttagggttcccaagaatcggtccaaacagagaattaaagaaggccactgaaggttactggaacggtaaaatcactgtcgatgaattattcaaggtcggtaaggatttgagaactcaaaactggaagttgcaaaaggaggctggtgttgatatcatcccatccaatgacttctccttttacgaccaagttttggatttgtctttgttgttcaatgtcattccagaccgttacactaagtacgatctatctccaatcgacactttgtttgctatgggtagaggtttacaaagaaaggccactgaaactgaaaaggctgtcgacgtcactgctttggaaatggttaaatggttcgactctaactaccattacgttagaccaactttctccaagaccactcaatttaagttgaacggccaaaagccagttgacgaatttttggaagccaaggagttaggtattcatactagacctgtcttgttgggtccagtttcttacttattcttgggtaaggctgacaaggattctctagatttggaaccattgtccctattggaacaattgttgcctctatacactgaaatcctatctaaattggcttctgctggtgccactgaagttcaaattgacgaacctgtcttagttttggacttgcctgccaacgcccaagccgccattaagaaggcttacacttacttcggtgaacaaagcaatctaccaaagattactttggctacttacttcggtaccgttgtccctaacttagacgccatcaagggcttgccagttgctgccttacacgttgactttgttagagctccagaacaatttgatgaagtcgttgccgccattggtaacaaacaaaccttgtccgttggtattgttgatggtagaaacatttggaagaatgatttcaagaagtcttccgctatcgttaacaaggctattgaaaagttgggtgctgacagagtcgttgttgccacttcttcttctctattgcacacaccagttgatttgaacaacgaaaccaagttggacgctgaaatcaagggctttttctctttcgccactcaaaaattggatgaagttgttgtgatcaccaagaacgtttccggtcaagacgttgctgctgccctagaagctaacgctaaatctgttgaatccagaggtaaatccaagtttatccacgatgctgccgttaaggccagagttgcctctatcgacgaaaaaatgtctactagagcagctccatttgaacaaagattgcctgaacaacaaaaagtcttcaacttgccattgttcccaacaacaactattggttccttccctcaaaccaaggacatcagaattaacagaaacaaattcaacaagggcaccatctctgctgaagaatatgaaaaattcatcaattctgaaattgaaaaggtcatcagattccaagaagaaattggtttggatgtcttagtccacggtgaaccagaaagaaacgatatggttcaatacttcggtgaacaaatcaacggttatgctttcactgttaacggttgggttcaatcttacggttccagatatgtcagaccaccaattattgttggtgacttgtccagaccaaaggctatgtccgtcaaggaatctgtttacgctcaatccatcacttctaagccagtaaagggtatgttgactggtccaattacctgtttgagatggtctttcccaagagacgatgtcgaccaaaaaactcaagctatgcaattagctttggctttgagagatgaagtcaatgatttggaagctgccggtatcaaggttatccaagttgatgaaccagctttaagagaaggtttaccattgagagaaggtactgagagatctgcttactacacctgggctgccgaagctttcagagttgctacttctggtgttgctaacaagactcaaatacactctcatttctgttactctgacttggatccaaaccatatcaaggctttggatgctgatgttgtttccatcgaattctctaagaaggacgatgctaactacattgctgaattcaaaaactatccaaaccacattggtctaggtttattcgatattcattctccaagaattccatcaaaggatgaatttatcgccaagatttcaaccatcttgaagagctacccagctgaaaagttctgggttaacccagattgtggtttgaagactagaggctgggaagaaactagattgtctttgactcatatggtcgaagccgccaagtacttccgtgaacaatacaagaattaa

SEQ ID NO: 33 - protein, 577 aa, Saccharomyces cerevisiae
MADITDKTAEQLENLNIQDDQKQAATGSESQSVENSSASLYVGDLEPSVSEAHLYDIFSPIGSVSSIRVCRDAITKTSLGYAYVNFNDHEAGRKAIEQLNYTPIKGRLCRIMWSQRDPSLRKKGSGNIFIKNLHPDIDNKALYDTFSVFGDILSSKIATDENGKSKGFGFVHFEEEGAAKEAIDALNGMLLNGQEIYVAPHLSRKERDSQLEETKAHYTNLYVKNINSETTDEQFQELFAKFGPIVSASLEKDADGKLKGFGFVNYEKHEDAVKAVEALNDSELNGEKLYVGRAQKKNERMHVLKKQYEAYRLEKMAKYQGVNLFVKNLDDSVDDEKLEEEFAPYGTITSAKVMRTENGKSKGFGFVCFSTPEEATKAITEKNQQIVAGKPLYVAIAQRKDVRRSQLAQQIQARNQMRYQQATAAAAAAAAGMPGQFMPPMFYGVMPPRGVPFNGPNPQQMNPMGGMPKNGMPPQFRNGPVYGVPPQGGFPRNANDNNQFYQQKQRQALGEQLYKKVSAKTSNEEAAGKITGMILDLPPQEVFPLLESDELFEQHYKEASAAYESFKKEQEQQTEQA

SEQ ID NO: 34 - DNA, 1734 bp, Saccharomyces cerevisiae
atggctgatattactgataagacagctgaacaattggaaaacttgaatattcaagatgaccaaaagcaagccgccactggttcagaaagccaatctgttgaaaactcttctgcatcattatatgttggtgacttagaaccttctgtttccgaagcccacttatatgatatcttctctccaatcggttcagtctcctccattcgtgtctgtcgtgatgccatcactaagacctctttgggctatgcttatgttaactttaacgaccatgaagccggcagaaaagcaattgagcaattgaactacactccaatcaagggtagattatgccgtattatgtggtctcaacgtgacccatcattgagaaagaagggttctggtaacatctttatcaagaacttgcaccctgatattgacaacaaggctttgtatgacactttctctgtgtttggtgacatcttgtccagcaagattgccaccgacgaaaacggaaaatccaagggttttgggtttgttcacttcgaagaagaaggtgctgccaaggaagctattgatgctttgaatggtatgctgttgaacggtcaagaaatttatgttgctcctcacttgtccagaaaggaacgtgactctcaattggaagagactaaggcacattacactaacctttatgtgaaaaacatcaactccgaaactactgacgaacaattccaagaattgtttgccaaatttggtccaattgtttctgcctctttggaaaaggatgctgatggaaaattgaagggtttcgggtttgttaactacgaaaagcatgaagacgctgtgaaagctgttgaagctttgaatgactctgaactaaatggagaaaagttatacgttggtcgtgcccaaaagaagaatgaacgtatgcatgtcttgaagaagcaatacgaagcttacagattggaaaaaatggccaagtaccaaggtgttaatttgtttgttaagaacttagatgacagcgttgatgacgaaaagttggaagaagaatttgctccatatggtactatcacttctgcaaaggttatgagaaccgaaaacggtaagtctaagggttttggttttgtttgtttctcaactccagaggaagctactaaggccattacagaaaagaaccaacaaattgttgctggtaagccattatacgttgccattgctcaaagaaaagacgtaagacgttctcaattggctcaacaaatccaagccagaaatcaaatgagataccagcaagctactgctgccgctgccgccgccgctgccggtatgccaggtcaattcatgcctccaatgttctatggtgttatgccaccaagaggtgttccattcaacggtccaaacccacaacaaatgaacccaatgggcggtatgccaaagaacggcatgccacctcaatttagaaatggtccggtttacggcgtccccccacaaggtggtttcccaagaaatgccaacgataacaaccaattttatcaacaaaagcaaagacaagctttgggtgaacaattatacaagaaggtttctgctaagacttcaaatgaagaagcagctggtaaaattactggtatgattttggatttgccacctcaagaggtcttcccattgttggaaagtgatgaattgttcgaacaacactacaaagaagcttctgctgcctatgagtctttcaaaaaggagcaagaacaacaaactgagcaagcttaa

SEQ ID NO: 35 - protein, 568 aa, Saccharomyces cerevisiae
MSSQKVFGITGPVSTVGATAAENKLNDSLIQELKKEGSFETEQETANRVQVLKILQELAQRFVYEVSKKKNMSDGMARDAGGKIFTYGSYRLGVHGPGSDIDTLVVVPKHVTREDFFTVFDSLLRERKELDEIAPVPDAFVPIIKIKFSGISIDLICARLDQPQVPLSLTLSDKNLLRNLDEKDLRALNGTRVTDEILELVPKPNVFRIALRAIKLWAQRRAVYANIFGFPGGVAWAMLVARICQLYPNACSAVILNRFFIILSEWNWPQPVILKPIEDGPLQVRVWNPKIYAQDRSHRMPVITPAYPSMCATHNITESTKKVILQEFVRGVQITNDIFSNKKSWANLFEKNDFFFRYKFYLEITAYTRGSDEQHLKWSGLVESKVRLLVMKLEVLAGIKIAHPFTKPFESSYCCPTEDDYEMIQDKYGSHKTETALNALKLVTDENKEEESIKDAPKAYLSTMYIGLDFNIENKKEKVDIHIPCTEFVNLCRSFNEDYGDHKVFNLALRFVKGYDLPDEVFDENEKRPSKKSKRKNLDARHETVKRSKSDAASGDNINGTTAAVDVN

SEQ ID NO: 36 - DNA, 1707 bp, Saccharomyces cerevisiae
atgagctctcaaaaggtttttggtattactggacctgtttccaccgtgggcgccacagcagcagaaaataaattaaatgatagtttaatccaagaactgaaaaaggaaggatcgttcgaaacagagcaagaaactgccaatagggtacaagtgttgaaaatattgcaggaattggcacaaagatttgtttatgaagtatcgaagaagaaaaatatgtcagacgggatggcaagggatgctggtgggaagatttttacgtatgggtcctatagactaggagtccatgggcctggtagtgatatcgatactttggtagttgttccaaaacatgtaactcgggaagatttttttacggtatttgattcactactgagagagaggaaggaactggatgaaattgcacctgtacctgatgcgtttgtcccgattatcaagataaagttcagtggtatttctatcgatttaatctgtgcacgtctagaccaacctcaagtgcctttatccttgactttatcagataaaaatctactgcgaaatctagacgagaaggacttgagagctttgaatggtaccagagtaacagatgagatattagaactggtaccaaagccgaatgttttcagaatcgctttaagagctattaagctatgggcccaaagaagggctgtttatgctaatatttttggttttcctggtggtgtggcttgggccatgctagtggctagaatttgtcaactataccctaacgcctgtagcgcagttatattgaacagatttttcatcattttgtcggaatggaattggccacaacctgttatcttgaaaccaattgaggatggcccgttacaagttcgtgtatggaatccaaagatatatgcccaagacaggtctcatagaatgcccgtcattacaccagcttatccatcaatgtgtgctacccataacatcacggaatctactaaaaaagtcattttacaggaattcgtaagaggcgttcaaattacgaatgatattttttccaataagaagtcctgggccaatttattcgaaaaaaacgattttttctttcgatacaagttctatttagaaattactgcatatacaaggggcagtgacgagcagcatttaaaatggagtggtcttgttgaaagtaaggtaaggcttctagttatgaaactggaggtgttagctggaataaaaattgcacatcctttcaccaaaccctttgaaagtagttattgttgtccaaccgaggatgactatgaaatgattcaagacaaatacggtagtcataaaactgagacagcactgaacgcccttaaactggtaacagatgaaaataaagaggaagaaagtattaaagatgcaccaaaggcatatttaagcaccatgtacataggccttgactttaatattgaaaacaaaaaggaaaaagttgacattcacattccctgcactgaatttgtgaatttatgtcgaagtttcaatgaggattatggtgaccacaaagtattcaatctagccctccgcttcgtaaagggttacgatttgccagatgaagttttcgatgaaaatgaaaagagaccatcaaagaagagtaaaaggaagaatttagatgctagacatgaaaccgtgaagagatctaaatcagatgctgcttcaggtgacaacatcaatggcacaaccgcagctgttgacgtaaactaa

SEQ ID NO: 37 - protein, 626 aa, Saccharomyces cerevisiae
MDHDTEVIVKDFNSILEELTFNSRPIITTLTKLAEENISCAQYFVDAIESRIEKCMPKQKLYAFYALDSICKNVGSPYTIYFSRNLFNLYKRTYLLVDNTTRTKLINMFKLWLNPNDTGLPLFEGSALEKIEQFLIKASALHQKNLQAMLPTPTVPLLLRDIDKLTCLTSERLKNQPNDEKLKMKLLVLSQLKQELKREKLTLNALKQVQMQLRQVFSQDQQVLQERMRYHELQQQQQQQQQQQQQQQQQQQQYHETKDMVGSYTQNSNSAIPLFGNNSDTTNQQNSLSSSLFGNISGVESFQEIEKKKSLNKINNLYASLKAEGLIYTPPKESIVTLYKKLNGHSNYSLDSHEKQLMKNLPKIPLLNDILSDCKAYFATVNIDVLNNPSLQLSEQTLLQENPIVQNNLIHLLYRSKPNKCSVCGKRFGNSESEKLLQNEHLDWHFRINTRIKGSQNTANTGISNSNLNTTTTRKNIQSRNWYLSDSQWAAFKDDEITSTKHKNDYTDPHANKNIDKSALNIHADENDEGSVDNTLGSDRSNELEIRGKYVVVPETSQDMAFKCPICKETVTGVYDEESGEWVWKNTIEVNGKYFHSTCYHETSQNSSKSNSGKVGLDDLKKLVTK

SEQ ID NO: 38 - DNA, 1881 bp, Saccharomyces cerevisiae
atggatcacgacacagaagttatagtcaaggatttcaacagtattcttgaagaattaacttttaactcgagacccattattaccactctcacgaagttagcagaagaaaacatttcgtgtgctcaatattttgttgacgccattgagagtagaattgaaaaatgtatgcctaagcagaagctatacgctttttatgccttggactccatttgtaagaatgttggcagcccatacacaatctacttcagtagaaatttgtttaacttgtacaaaagaacatacttattggtggacaacaccacaagaacaaaactcatcaatatgtttaagctttggttgaatcctaatgacaccggcctgcctctgtttgaaggctcagcactggagaaaattgaacagtttttaataaaggccagtgctctccaccagaaaaacctgcaagcaatgttaccaactccgactgttcccctcctattaagagacattgataaattaacttgtttaactagtgaaaggctaaagaatcaaccaaatgatgaaaaattaaaaatgaaactgttggttttatctcagttgaaacaagaactgaaaagagaaaaactaactctcaatgctctgaaacaagttcaaatgcaactaaggcaggtcttctcacaggaccaacaagttttgcaggaaagaatgaggtatcacgaacttcagcagcaacagcaacaacagcagcaacagcaacaacaacaacagcagcagcagcagcagtatcacgaaactaaggatatggtcggttcttacacacaaaactctaattcagcgattccattgtttggaaataattcagatacaacgaaccaacagaattctttatcttcctctctttttggtaatatttctggcgttgaatcttttcaagaaattgagaaaaaaaagtccctaaacaaaatcaataacctctatgcatctcttaaggctgaagggctcatatatactccaccaaaagaatccatcgtaacgctttataaaaagctaaatggacactcaaactactctcttgattcacacgaaaaacaattgatgaagaacctgccaaagatacccttattaaatgatattctttctgactgcaaagcttattttgccacggttaacatcgacgttttgaataacccatccctgcagctgtccgaacaaactcttcttcaagaaaatcccatcgtccaaaataatcttattcacttattataccgatccaagccaaataaatgtagcgtgtgtggtaagagatttggcaactcagaaagtgaaaaactactacaaaatgagcatttggactggcatttcagaataaatacaagaattaagggatcacaaaacacagcaaatacaggcataagcaactctaacttaaatacaactaccacaaggaaaaatattcaatcaagaaactggtatttgagtgactctcaatgggcagccttcaaagatgatgaaattacctccacaaagcataaaaatgattatactgatccgcatgccaataaaaatatcgataaaagtgccctaaatattcacgctgatgaaaatgacgaaggctctgtagacaacactttaggaagtgatagaagtaacgagttggaaatacggggaaaatatgttgttgtaccagaaacttcacaagatatggccttcaaatgtccaatttgtaaggaaaccgttactggcgtttatgatgaagaatctggtgaatgggtttggaaaaatactattgaagtgaatggcaaatatttccactcgacatgttatcatgaaacgtcgcaaaattcaagcaaatctaatagtggcaaggtcggtttggatgacttaaagaaattggtcacaaaataa

SEQ ID NO: 39 - protein, 563 aa, Saccharomyces cerevisiae
MSEITLGKYLFERLKQVNVNTVFGLPGDFNLSLLDKIYEVEGMRWAGNANELNAAYAADGYARIKGMSCIITTFGVGELSALNGIAGSYAEHVGVLHVVGVPSISAQAKQLLLHHTLGNGDFTVFHRMSANISETTAMITDIATAPAEIDRCIRTTYVTQRPVYLGLPANLVDLNVPAKLLQTPIDMSLKPNDAESEKEVIDTILALVKDAKNPVILADACCSRHDVKAETKKLIDLTQFPAFVTPMGKGSIDEQHPRYGGVYVGTLSKPEVKEAVESADLILSVGALLSDFNTGSFSYSYKTKNIVEFHSDHMKIRNATFPGVQMKFVLQKLLTTIADAAKGYKPVAVPARTPANAAVPASTPLKQEWMWNQLGNFLQEGDVVIAETGTSAFGINQTTFPNNTYGISQVLWGSIGFTTGATLGAAFAAEEIDPKKRVILFIGDGSLQLTVQEISTMIRWGLKPYLFVLNNDGYTIEKLIHGPKAQYNEIQGWDHLSLLPTFGAKDYETHRVATTGEWDKLTQDKSFNDNSKIRMIEIMLPVFDAPQNLVEQAKLTAATNAKQ

SEQ ID NO: 40 - DNA, 1692 bp, Saccharomyces cerevisiae
atgtctgaaattactttgggtaaatatttgttcgaaagattaaagcaagtcaacgttaacaccgttttcggtttgccaggtgacttcaacttgtccttgttggacaagatctacgaagttgaaggtatgagatgggctggtaacgccaacgaattgaacgctgcttacgccgctgatggttacgctcgtatcaagggtatgtcttgtatcatcaccaccttcggtgtcggtgaattgtctgctttgaacggtattgccggttcttacgctgaacacgtcggtgttttgcacgttgttggtgtcccatccatctctgctcaagctaagcaattgttgttgcaccacaccttgggtaacggtgacttcactgttttccacagaatgtctgccaacatttctgaaaccactgctatgatcactgacattgctaccgccccagctgaaattgacagatgtatcagaaccacttacgtcacccaaagaccagtctacttaggtttgccagctaacttggtcgacttgaacgtcccagctaagttgttgcaaactccaattgacatgtctttgaagccaaacgatgctgaatccgaaaaggaagtcattgacaccatcttggctttggtcaaggatgctaagaacccagttatcttggctgatgcttgttgttccagacacgacgtcaaggctgaaactaagaagttgattgacttgactcaattcccagctttcgtcaccccaatgggtaagggttccattgacgaacaacacccaagatacggtggtgtttacgtcggtaccttgtccaagccagaagttaaggaagccgttgaatctgctgacttgattttgtctgtcggtgctttgttgtctgatttcaacaccggttctttctcttactcttacaagaccaagaacattgtcgaattccactccgaccacatgaagatcagaaacgccactttcccaggtgtccaaatgaaattcgttttgcaaaagttgttgaccactattgctgacgccgctaagggttacaagccagttgctgtcccagctagaactccagctaacgctgctgtcccagcttctaccccattgaagcaagaatggatgtggaaccaattgggtaacttcttgcaagaaggtgatgttgtcattgctgaaaccggtacctccgctttcggtatcaaccaaaccactttcccaaacaacacctacggtatctctcaagtcttatggggttccattggtttcaccactggtgctaccttgggtgctgctttcgctgctgaagaaattgatccaaagaagagagttatcttattcattggtgacggttctttgcaattgactgttcaagaaatctccaccatgatcagatggggcttgaagccatacttgttcgtcttgaacaacgatggttacaccattgaaaagttgattcacggtccaaaggctcaatacaacgaaattcaaggttgggaccacctatccttgttgccaactttcggtgctaaggactatgaaacccacagagtcgctaccaccggtgaatgggacaagttgacccaagacaagtctttcaacgacaactctaagatcagaatgattgaaatcatgttgccagtcttcgatgctccacaaaacttggttgaacaagctaagttgactgctgctaccaacgctaagcaataa

SEQ ID NO: 41 - protein, 987 aa, Saccharomyces cerevisiae
MQSQDSCYGVAFRSIITNDEALFKKTIHFYHTLGFATVKDFNKFKHGENSLLSSGTSQDSLREVWLESFKLSEVDASGFRIPQQEATNKAQSQGALLKIRLVMSAPIDETFDTNETATITYFSTDLNKIVEKFPKQAEKLSDTLVFLKDPMGNNITFSGLANATDSAPTSKDAFLEATSEDEIISRASSDASDLLRQTLGSSQKKKKIAVMTSGGDSPGMNAAVRAVVRTGIHFGCDVFAVYEGYEGLLRGGKYLKKMAWEDVRGWLSEGGTLIGTARSMEFRKREGRRQAAGNLISQGIDALVVCGGDGSLTGADLFRHEWPSLVDELVAEGRFTKEEVAPYKNLSIVGLVGSIDNDMSGTDSTIGAYSALERICEMVDYIDATAKSHSRAFVVEVMGRHCGWLALMAGIATGADYIFIPERAVPHGKWQDELKEVCQRHRSKGRRNNTIIVAEGALDDQLNPVTANDVKDALIELGLDTKVTILGHVQRGGTAVAHDRWLATLQGVDAVKAVLEFTPETPSPLIGILENKIIRMPLVESVKLTKSVATAIENKDFDKAISLRDTEFIELYENFLSTTVKDDGSELLPVSDRLNIGIVHVGAPSAALNAATRAATLYCLSHGHKPYAIMNGFSGLIQTGEVKELSWIDVENWHNLGGSEIGTNRSVASEDLGTIAYYFQKNKLDGLIILGGFEGFRSLKQLRDGRTQHPIFNIPMCLIPATVSNNVPGTEYSLGVDTCLNALVNYTDDIKQSASATRRRVFVCEVQGGHSGYIASFTGLITGAVSVYTPEKKIDLASIREDITLLKENFRHDKGENRNGKLLVRNEQASSVYSTQLLADIISEASKGKFGVRTAIPGHVQQGGVPSSKDRVTASRFAVKCIKFIEQWNKKNEASPNTDAKVLRFKFDTHGEKVPTVEHEDDSAAVICVNGSHVSFKPIANLWENETNVELRKGFEVHWAEYNKIGDILSGRLKLRAEVAALAAENK

SEQ ID NO: 42 - DNA, 2000 bp, Saccharomyces cerevisiae
gatcacagtttttcgaggatagcactgatctatacaataaaaggagctattttcatcataatgaaagagatgtatttgtattcattttcgcacttgccaccattttactaattgtttgctcatgttatgttacgcctctatatcgtatgcatcacaagatgggaacttggaattggtcctcggcgttagattgcgccttcattggtgccttctcaattgaatttatcgtgaaaacagtagctgacggatttatatattctccaaatgcttacctgaggaatccatggaactttattgatttttgtgtcctaatctcaatgtggattaatttaattgcatacctaaaaaacaatggaaatttgtctaggattttcaagggattgacagccctgagggccctcagatgcctcacgatcagtaacacagctcgtcaaacatttaacctagttatgtttgatggtttaaataaaatttttgaagctgggttgatttcactcagtttgctatttccatttacagtttggggcttaagcatttttaaaggccgtttaggtacttgcaatgacggaagtttgggccgtgcagattgttacaatgaatattcaaattccgtttttcaatgggatatcatgtctccaagggtttaccagcaaccatatcttcatttggactctttcgcaagcgcttttagttcattataccaaatcatttctttggaaggatgggttgatttgttggaaaatatgatgaatagttcaggaataggtacacccgctacggtaatgggttcagcagggaatgctttattcctcgttctgtttaattttttaagtatggttttcatcctgaacttgtttgtttcattcattgttaacaaccaagcaaggacaacaggaagcgcttactttaccattgaggaaaaggcgtggctggaatcccagaaacttttatctcaggccaagccaaaagctatcccaaatttaattgagttatcaagagttaggcaatttttctatcaacttgcagtggagaaaaaaaatttctactacgcatcgtttcttcaggtagtactttatttgcacataatcatgctcctgagtcgaagctacaatccaggaaacttgataggttatcaaggtgtttattttatgttttccactagtgtttttttaattcaagaggcacttcacatgtgcggtgaaggaccaagattatattttaggcaaaaatggaacagcatacgactcagtatcataattatagcctttattatgaacgctgtagcattccacgttccagcctctcactattggttccacaatataaaggggtttttcctgttagtgatatttttgtttattattcctcaaaatgacacactaactgaactattagaaaccgcaatggcaagcttaccgcctattctatcattgacctacacttggggggttttatttttagtatatgctattgctttgaatcaaatcttcggcctaacaaggttagggagtaatacgaccgataacataaattttagaactgtaatcaaatccatgattgttctgtttagatgtagttttggtgagggctggaattatatcatggccgacctaactgtgtcagaaccttattgctcctctgatgataattcaacctatacggactgtggatcagagacatatgcctatttgttattaatgtcgtggaatattatttccatgtatatttttgtgaatatgtttgtttcgttgattattggtaatttcagttatgtttaccgtagcggtggatctcgctctggcatcaacagatcggagataaaaaaatacattgaagcttggtccaaatttgatactgatggaactggcgagcttgagctgtcctacctcccaagaataatgcattcatttgacggtcctctttcatttaaaatttgggaaggtagattgacaataaaaagtctagtcgagaactacatggaggttaacccagatgatcca

SEQ ID NO: 43 - protein, 465 aa, Saccharomyces cerevisiae
MDGHNQNQYQNQNQIQQSQQPPLKKYVTQRRSVDVSSPYINLYYNRRHGLPNLVVEPETSYTIDIMPPNAYRGRDRVINLPSKFTHLSSNKVKHVIPAIQWTPEGRRLVVATYSGEFSLWNASSFTFETLMQAHDSAVTTMKYSHDSDWMISGDADGMIKIWQPNFSMVKEIDAAHTESIRDMAFSSNDSKFVTCSDDNILKIWNFSNGKQERVLSGHHWDVKSCDWHPEMGLIASASKDNLVKLWDPRSGNCISSILKFKHTVLKTRFQPTKGNLLMAISKDKSCRVFDIRYSMKELMCVRDETDYMTLEWHPINESMFTLACYDGSLKHFDLLQNLNEPILTIPYAHDKCITSLSYNPVGHIFATAAKDRTIRFWTRARPIDPNAYDDPTYNNKKINGWFFGINNDINAVREKSEFGAAPPPPATLEPHALPNMNGFINKKPRQEIPGIDSNIKSSTLPGLSI

SEQ ID NO: 44 - DNA, 1398 bp, Saccharomyces cerevisiae
atggacgggcataatcagaaccagtaccagaatcagaatcaaattcagcagtcacaacaaccacctctgaagaaatatgtgacacagagacgatcggtagacgtgagttctccgtatattaacctatattacaacagacgacatggattaccgaacctagtggtagagccagaaacttcatacacaatagatataatgccgcctaatgcctacagaggtcgagatcgagtcataaatttgcccagcaaatttacgcatttaagctcgaataaagtgaaacatgtgatacccgccatccaatggaccccagaaggcagaagacttgtagtagcaacttatagcggagaattttctctatggaacgcatcttcgtttacgtttgaaaccctcatgcaagcacatgattcggctgtgactacaatgaaatattctcacgacagtgattggatgattagtggggacgcagatggaatgattaagatatggcaaccaaactttagtatggttaaagaaattgatgctgcacacacggaaagtattagggatatggcattcagtagtaacgactcgaaatttgttacctgttcagatgataatatcttgaagatttggaacttcagcaatggcaaacaagaaagagtattatcggggcaccactgggacgtgaaaagttgtgattggcatcccgagatggggttaattgcctccgcatctaaggacaatttagtaaaactgtgggatccgcgttccggaaactgcatatcatcaattctgaagttcaaacatactgtcttgaagacgcgatttcaacccaccaagggaaatctattaatggcgatttcaaaagataaatcgtgtcgtgtgttcgacattagatacagtatgaaggaactaatgtgtgtaagagatgaaacggattatatgacactagaatggcacccaataaatgaatctatgttcactctggcgtgctatgacggctcgctaaagcattttgatcttttacaaaatctaaacgaacctattttgactattccatacgcccatgataagtgtatcacctcgttatcatataatccagtaggacatatatttgccacagcagctaaggatagaaccataagattttggactagagcgaggccaattgatcctaatgcatatgatgatccaacttataacaataagaagataaatggatggttttttggcatcaataacgacatcaatgctgtgagagagaagagtgaattcggtgcggccccgccaccaccagctacacttgaaccacatgcgttaccaaacatgaatggattcatcaacaagaaaccgcgtcaagaaatcccaggcatcgattcaaacatcaagagttccacattgccaggtttaagcatataa

SEQ ID NO: 45 - protein, 785 aa, Saccharomyces cerevisiae [sequence omitted]

SEQ ID NO: 46 - DNA, 2000 bp, Saccharomyces cerevisiae
atgtcatctgcagagatggaacaattgttacaggccaagacactggccatgcacaacaatccaacggagatgctgcccaaggtgctcgaaactacggcatccatgtaccacaacggtaatctcagcaagctgaagttgcctttggccaagttttttacacagttagttctagacgtggtgtcgatggactctccaattgcgaatactgagagaccgtttattgctgctcaatatctgccactacttcttgctatggcgcaatccaccgcggacgtactagtgtacaagaatatcgtgcttattatgtgcgcttcatacccgctggtgttggatctggttgctaagacatcaaaccaggaaatgtttgatcagttgtgtatgctgaagaagttcgtgctctcgcactggagaactgcatatcctttgcgtgccaccgttgacgatgaaacggatgtcgaacaatggctggcgcagattgaccaaaatatcggcgtgaaattagcgaccatcaagttcatatctgaggtcgtgctgtcgcaaactaaatcacccagcggcaacgagattaattcatctaccatcccggataaccaccctgtgttgaacaaaccggctttggagagcgaggctaagaggcttcttgatatgttgctaaactacctaattgaggaacagtacatggtctcgtccgttttcattggtatcatcaattctttatccttcgtcatcaaaagaaggccgcagacaacaataagaattctttccgggctgttgcgtttcaacgtcgacgccaagtttcccctagagggcaagtctgacttgaactacaaactatccaagagatttgttgaaagggcgtacaagaactttgtgcaatttgggctaaaaaatcaaatcattacaaaatccctctcatccggatcagggtcatcgatctactccaagctgaccaagatttctcaaactttacacgttattggcgaagagaccaagagcaagggaattttgaacttcgacccttccaagggcaatagcaagaaaacgttgtccaggcaggacaaactaaaatacatctcactatggaaaaggcaattatccgcgttattgtctactctaggggtgtccacaaagacccccacgcctgtgtccgcacctgcaacgggctcttcaaccgaaaacatgcttgatcaactgaagatattgcaaaaatacaccctcaacaaggcttcacaccagggcaatacttttttcaacaactcacccaaaccaatcagcaacacctactcatctgtgtactcattgatgaacagttcgaactccaaccaggatgtgacccagctacccaatgacatacttatcaagctgtccacagaggccatcttgcaaatggacagcacgaaactgatcaccggattgtctatcgttgcttcgaggtacacggatttaatgaatacgtacatcaattctgtaccgtcctcgtcatcatcaaagaggaaatccgacgatgatgacgacggcaacgacaatgaagaagttggaaacgatggcccaacggctaatagcaagaaaatcaaaatggaaacagaaccactagcggaggaaccagaggagcccgaagacgatgaccgaatgcagaagatgcttcaagaagaggaaagcgcccaagaaatctcaggagatgccaacaaatcaacttctgccattaaggagatcgcacccccctttgaacctgactcattgacgcaggatgaaaaactaaagtacctctcaaagctgaccaagaaactgtttgaattatccggtcgccaggatactacccgggccaaatcttcgtcttcctcctccatattactggacgatgacgactcctcgtcatggttacacgtcttaatcagattggttacgagaggaatcgaagcacaagaggccagtgacctgattcgtgaagaactgcttggcttcttcatccaggatttcgagcaacgtgtcagtctgatcat

SEQ ID NO: 47 - protein, 533 aa, Saccharomyces cerevisiae
MSAPVPQLVNISHALQASTIQQIRLDMVDFNKDCKLSSIQLARIDKYIDSLQAALNQFTKDNLHIERKEKNVTEADIQLYSGLKSMYLDYLNQLIKLKHEKQHHSTPPIANDVSLDFFVNQLPKFSPEERKNYIDNLILNKNSHNRLSKMDGLVDAVINLCVLDTSVAENVRSYMKLLDTLGFQKGSNSTGTKANLKKKLASSKAKIKDSEKEKEKEKDKSKVKMKTKLKPSPLLNNDDKNSSPSPTASTSSMKKLKSGLFNKNEAKSTESLPTSSKKKLSFSKYLNKDDADMTKLGTKRSIDVDFKVNPEASTVASNIISSSTSGSSTTTVATPASSEEPLKKKTKISVQDSNVQSILRNGKPKKARISSIKFLDDSQLIKVYGDDLPNQGLQVSPTQLKKILKPFKEGEPKEIILFEDMSIKLKPLDLMFLKNTNSDDYMDISETKGGPIHCETRTPLIYRKNFNHFNPDLNKRPPREPIEFDLNGNTNSTPTIAKAFGKNSLLLRKDRGGLPYKHVPIVKRNKYPPRPVH

SEQ ID NO: 48 - DNA, 1602 bp, Saccharomyces cerevisiae
atgtcagcacctgttcctcaattagtaaatatttctcatgccttgcaagcttccactatccagcaaattcgtttggatatggtagattttaataaagattgcaaattatcttccattcaactggcaagaattgacaagtatatcgattctttgcaagcggctttgaatcagtttaccaaagataacttgcacatagaacggaaggaaaagaacgtgactgaagcagatatacagctgtattctggcttaaaatcaatgtatctagattatttgaaccagctaattaaactaaagcatgagaaacaacaccactctacaccgcccatcgctaatgatgtctccctggacttcttcgttaaccaattgcctaagttttctcccgaggaaaggaaaaattacattgacaatttaatcctgaataaaaatagccataatcgattatctaaaatggatggtctggtagatgcagtcatcaatttatgcgtcttggatacttccgttgctgaaaatgtacgatcctatatgaaattattagatacattgggtttccaaaagggttcgaatagtactggtacaaaggccaacctcaaaaagaaattggctagttcgaaagcaaagataaaggattcagaaaaagaaaaggagaaggagaaggataaatcaaaagtcaagatgaaaactaaattaaaaccttctcctttgctcaataacgatgacaaaaattcttctccatcgcctactgcatctacctcttcaatgaagaaattgaagtcgggtttattcaataaaaatgaagctaagtctacagaatctctacctacttcttccaagaaaaaactatcattttctaaatatctgaacaaggatgacgcagatatgaccaagcttgggactaaacggtcaatagatgtggatttcaaagtcaaccccgaagcatccacggtggcttctaatatcatatcttcgtcaacgtcaggatcgtcaaccacaacggtagcgactcctgcttcttcagaagagcccttaaaaaaaaaaaccaaaatatccgtgcaagactctaatgtacaatcgattttgagaaatggtaaaccgaaaaaagcacgcataagtagcatcaaatttttggatgattcccaactaataaaagtttacggtgacgatctaccgaaccaagggctacaagtttctcctactcaattgaaaaaaattctgaaaccattcaaggagggggaaccgaaggaaattatattgttcgaggatatgtcaatcaaattaaaacctcttgatttgatgtttctgaagaacacaaacagtgatgactatatggatatatccgagactaaaggtggcccaatacattgtgaaacaaggaccccgttgatctatagaaaaaatttcaatcatttcaacccggacttgaataaaaggccgccaagagaacccatagaattcgacttaaatggaaatacgaactcaaccccgactatagcaaaggctttcggtaaaaatagtttattactaaggaaggacagaggtggtttgccatacaagcatgtccccatagtaaaaagaaataaatatcctccaagaccagtacactaa

SEQ ID NO: 49 - protein, 677 aa, Saccharomyces cerevisiae
MSSSTTPDLLYPSADKVAEPSDNIHGDELRLRERIKDNPTNILSYFQLIQYLETQESYAKVREVYEQFHNTFPFYSPAWTLQLKGELARDEFETVEKILAQCLSGKLENNDLSLWSTYLDYIRRKNNLITGGQEARAVIVKAFQLVMQKCAIFEPKSSSFWNEYLNFLEQWKPFNKWEEQQRIDMLREFYKKMLCVPFDNLEKMWNRYTQWEQEINSLTARKFIGELSAEYMKARSLYQEWLNVTNGLKRASPINLRTANKKNIPQPGTSDSNIQQLQIWLNWIKWERENKLMLSEDMLSQRISYVYKQGIQYMIFSAEMWYDYSMYISENSDRQNILYTALLANPDSPSLTFKLSECYELDNDSESVSNCFDKCTQTLLSQYKKIASDVNSGEDNNTEYEQELLYKQREKLTFVFCVYMNTMKRISGLSAARTVFGKCRKLKRILTHDVYVENAYLEFQNQNDYKTAFKVLELGLKYFQNDGVYINKYLDFLIFLNKDSQIKTLFETSVEKVQDLTQLKEIYKKMISYESKFGNLNNVYSLEKRFFERFPQENLIEVFTSRYQIQNSNLIKKLELTYMYNEEEDSYFSSGNGDGHHGSYNMSSSDRKRLMEETGNNGNFSNKKFKRDSELPTEVLDLLSVIPKRQYFNTNLLDAQKLVNFLNDQVEIPTVESTKSG

SEQ ID NO: 50 - DNA, 2000 bp, Saccharomyces cerevisiae
atgtccagctctacgactcctgatttactatatccctctgcggacaaagtcgcagagcctagtgacaatatacatggagatgaactacgacttagagaaaggattaaagacaatcccacgaatattttatcatacttccagcttattcaatatttggaaactcaagagtcatacgctaaggtgagagaagtatacgagcaatttcataacacattcccgttttattcacctgcgtggactttgcaactaaagggtgaattggcaagagatgaatttgagactgttgagaagattttggctcaatgtctttctggcaagttggaaaataatgacctatctctttggtcaacatatttggactacatacgcagaaaaaacaacttaattactggtggacaagaggcgagagctgttattgtcaaggcattccaactagttatgcaaaagtgtgcaatttttgaacccaaatcatcttctttttggaacgaatatctcaattttttagagcagtggaagccattcaacaaatgggaggagcaacagcgaattgacatgctcagagaattctacaagaaaatgctatgtgttccttttgataatctagaaaaaatgtggaatagatacactcaatgggaacaagaaataaattccctaacagccagaaaatttattggcgagttatcagccgaatacatgaaagcccgttccttataccaggaatggttgaacgttactaatggattgaaaagggcatctccaattaatctgcgcacagcaaacaagaaaaacataccacaaccaggtacctcagactcaaacattcagcagttacagatttggttgaattggataaaatgggaaagggagaataagttgatgcttagtgaagatatgctatcacaaagaatcagttacgtttataaacaaggtattcaatacatgatattttctgctgaaatgtggtacgattattcaatgtatatatctgaaaattcggatcgacaaaatatcttatatactgcgttattagctaatcccgactcaccttctcttacattcaagttatccgaatgctacgaactggataatgattctgaaagtgtttctaactgttttgacaagtgcactcaaactttactatcgcagtataaaaagatcgcctccgatgtaaattcgggtgaagataataacacagagtatgaacaagagctgctatacaaacagagggaaaaattaacattcgtgttttgcgtgtatatgaatacgatgaaaagaatatcaggactatccgcagcacgtactgtatttggtaaatgtcgtaaactgaagcgtatattaacacatgacgtctacgtggaaaatgcatatttagaatttcaaaatcaaaacgattataagactgcttttaaggttttagaattgggtttaaaatacttccaaaacgatggagtttatatcaacaaatacttagattttttaatatttttaaataaggattcgcagatcaaaaccttatttgaaacatcagtggaaaaagtgcaagatttaacccagctgaaggaaatatacaagaaaatgataagttatgaatcgaaattcggtaacttaaacaacgtttattctctagagaaaagatttttcgaacggttcccccaagaaaatttgattgaagttttcacaagtcgttatcaaattcaaaactccaacttaataaagaaattagagttaacttatatgtataatgaggaagaagacagttacttttcttctggaaacggggatggccatcatggctcttacaatatgagttcgtcagatagaaagagactaatggaggaaactggaaacaatggaaacttctccaataagaaattcaaaagagactcagagcttccaacagaggttcttgatttattgagcgttataccaaaacgtcaatattttaatacaaatttactcgatgcgcagaaattggtgaattttttaaatgatcaagtaga

SEQ ID NO: 51 - protein, 296 aa, Saccharomyces cerevisiae
MNRQSGVNAGVQNNPPSRVVYLGSIPYDQTEEQILDLCSNVGPVINLKMMFDPQTGRSKGYAFIEFRDLESSASAVRNLNGYQLGSRFLKCGYSSNSDISGVSQQQQQQYNNINGNNNNNGNNNNNSNGPDFQNSGNANFLSQKFPELPSGIDVNINMTTPAMMISSELAKKPKEVQLKFLQKFQEWTRAHPEDAVSLLELCPQLSFVTAELLLTNGICKVDDLIPLASRPQEEASATNNNSVNEVVDPAVLNKQKELLKQVLQLNDSQISILPDDERMAIWDLKQKALRGEFGAF

SEQ ID NO: 52 - DNA, 891 bp, Saccharomyces cerevisiae
atgaataggcagagcggtgtgaatgcgggggtacagaacaacccaccatcccgagtggtgtatctgggttctataccatacgatcaaacagaggagcagatacttgatttatgtagtaatgttgggcccgtgatcaatttgaaaatgatgttcgacccccaaactggtaggtcgaaagggtacgcgtttattgaatttagagatttagagtccagtgccagcgcagtacgtaatttgaatggataccaattaggctctaggtttttgaaatgcggttactccagcaatagtgatatatcgggagtttcacaacagcaacaacaacagtacaacaacattaatgggaacaataacaacaatggaaataataataataatagtaatgggccggactttcaaaacagcggaaatgccaattttctaagtcaaaagtttccagaattgccctctggtatcgacgttaacataaacatgaccacccctgctatgatgatatcgagcgaactagctaaaaaaccgaaagaggtgcagttgaaatttttacaaaaattccaagaatggacaagagcgcatcctgaagatgctgtttcgctattagagctgtgtccacagttgagttttgttacggctgaattattgctaacgaatgggatatgtaaagtggatgatttgatcccgttagcttccaggccgcaagaagaggcatcggctacgaataacaatagcgtgaacgaggtggtggatccagctgtgcttaacaaacagaaagaactactgaaacaggtgttacaactgaatgacagtcaaatttctatcttgcccgatgatgaaaggatggctatttgggacttaaaacaaaaagcattaaggggagaatttggtgcattttga

SEQ ID NO: 53 - protein, 297 aa, Saccharomyces cerevisiae
MVVIANAHNELIHDAVLDYYGKRLATCSSDKTIKIFEVEGETHKLIDTLTGHEGPVWRVDWAHPKFGTILASCSYDGKVLIWKEENGRWSQIAVHAVHSASVNSVQWAPHEYGPLLLVASSDGKVSVVEFKENGTTSPIIIDAHAIGVNSASWAPATIEEDGEHNGTKESRKFVTGGADNLVKIWKYNSDAQTYVLESTLEGHSDWVRDVAWSPTVLLRSYLASVSQDRTCIIWTQDNEQGPWKKTLLKEEKFPDVLWRASWSLSGNVLALSGGDNKVTLWKENLEGKWEPAGEVHQ

SEQ ID NO: 54 - DNA, 894 bp, Saccharomyces cerevisiae
atggtcgtcatagctaatgcgcacaacgaattaatccatgacgctgttctagactattatgggaagcgccttgcaacctgctcttctgacaagacaatcaagatctttgaagtcgaaggagaaacacacaagttaatagacacgttgactggccacgaaggcccagtttggcgtgttgattgggcacatcctaaattcggaaccattttggcatcgtgttcttatgatggtaaagtgttgatttggaaggaagaaaacggtagatggtctcaaattgccgttcatgctgtccactctgcttctgtcaactctgttcaatgggctcctcatgaatatggccccctactgctggttgcttcctctgatggtaaggtctccgtagtagagttcaaagaaaacggtactacttccccaataatcatcgatgctcatgccattggcgttaactctgcttcttgggctccagctaccatcgaagaagatggtgaacacaacggtactaaagaatctcgcaagtttgttactgggggtgctgacaatttggtaaagatttggaagtacaattcagatgcccaaacttatgttctggaaagcaccttagaaggtcacagcgattgggttagagacgtagcatggtcacctactgttcttctacgttcttatttggccagtgtttctcaagatcgcacctgtattatttggactcaagacaatgaacaaggcccatggaaaaaaactttattaaaagaagaaaaattcccagatgttttatggagagccagttggtctttgtcaggtaatgtactagctctttccggtggcgataataaagttactttatggaaggaaaatcttgagggtaaatgggaacccgctggtgaagttcatcagtga

SEQ ID NO: 55 - protein, 1273 aa, Saccharomyces cerevisiae
MVKLAEFSRTATFAWSHDKIPLLVSGTVSGTVDANFSTDSSLELWSLLAADSEKPIASLQVDSKFNDLDWSHNNKIIAGALDNGSLELYSTNEANNAINSMARFSNHSSSVKTVKFNAKQDNVLASGGNNGEIFIWDMNKCTESPSNYTPLTPGQSMSSVDEVISLAWNQSLAHVFASAGSSNFASIWDLKAKKEVIHLSYTSPNSGIKQQLSVVEWHPKNSTRVATATGSDNDPSILIWDLRNANTPLQTLNQGHQKGILSLDWCHQDEHLLLSSGRDNTVLLWNPESAEQLSQFPARGNWCFKTKFAPEAPDLFACASFDNKIEVQTLQNLTNTLDEQETETKQQESETDFWNNVSREESKEKPSVFHLQAPTWYGEPSPAAHWAFGGKLVQITPDGKGVSITNPKISGLESNTTLSEALKTKDFKPLINQRLVKVIDDVNEEDWNLLEKLSMDGTEEFLKEALAFDNDESDAQDDANNEKEDDGEEFFQQIETNFQPEGDFSLSGNIEQTISKNLVSGNIKSAVKNSLENDLLMEAMVIALDSNNERLKESVKNAYFAKYGSKSSLSRILYSISKREVDDLVENLDVSQWKFISKAIQNLYPNDIAQRNEMLIKLGDRLKENGHRQDSLTLYLAAGSLDKVASIWLSEFPDLEDKLKKDNKTIYEAHSECLTEFIERFTVFSNFINGSSTINNEQLIAKFLEFINLTTSTGNFELATEFLNSLPSDNEEVKTEKARVLIASGKSLPAQNPATATTSKAKYTNAKTNKNVPVLPTPGMPSTTSIPSMQAPFYGMTPGASANALPPKPYVPATTTSAPVHTEGKYAPPSQPSMASPFVNKTNSSTRLNSFAPPPNPYATATVPATNVSTTSIPQNTFAPIQPGMPIMGDYNAQSSSIPSQPPINAVSGQTPHLNRKANDGWNDLPLKVKEKPSRAKAVSVAPPNILSTPTPLNGIPANAASTMPPPPLSRAPSSVSMVSPPPLHKNSRVPSLVATSESPRASISNPYAPPQSSQQFPIGTISTANQTSNTAQVASSNPYAPPPQQRVATPLSGGVPPAPLPKASNPYAPTATTQPNGSSYPPTGPYTNNHTMTSPPPVFNKPPTGPPPISMKKRSNKLASIEQNPSQGATYPPTLSSSASPLQPSQPPTLASQVNTSAENVSHEIPADQQPIVDFLKEELARVTPLTPKEYSKQLKDCDKRLKILFYHLEKQDLLTQPTIDCLHDLVALMKEKKYKEAMVIHANIATNHAQEGGNWLTGVKRLIGIAEATLN

SEQ ID NO: 56 - DNA, 2000 bp, Saccharomyces cerevisiae
atggtcaaacttgctgagttttctcgaacagccacgtttgcgtggtcacatgataaaattccattattggtctctggtaccgtatctggtacggtggatgctaatttctccactgattcatctctagaattgtggtcattgttggctgctgattcggagaagcctattgcttccttgcaagtggattccaaattcaatgatttggattggtctcataataacaagattattgctggtgctctggataacggtagtttggaattgtactccaccaatgaagcaaacaacgctatcaactccatggccagatttagcaaccattcttcctctgtgaagacggtaaagtttaacgcaaagcaagacaacgttcttgcttcgggtggtaacaacggtgaaatttttatttgggacatgaataaatgcactgaatcgccctccaattatactccattgacaccgggtcaatcgatgtcgtccgttgacgaggtcatttccctagcatggaaccaatctttggcccatgtttttgcatctgccgggtcgtctaatttcgcatctatttgggatttgaaggctaagaaggaagtcattcatctaagttacacttcacctaattcaggtatcaagcaacagctgtccgttgttgaatggcacccaaaaaactccacaagagtggcaacggctactggtagcgataatgatccatctatcctgatctgggatttaagaaacgccaacacaccattgcagactttaaatcaaggccatcaaaagggtattttgtcattagattggtgtcatcaggacgaacatctattattgtccagtggtagagataataccgttcttctatggaaccctgagtcagccgaacaactgtcccaattcccagctcgtggaaactggtgttttaagaccaaatttgcaccagaggctccagacctatttgcttgtgcctcctttgataacaaaattgaggtacagactttgcaaaatctcacaaacactttggatgagcaagaaaccgaaactaagcagcaagaatctgaaacagatttttggaataatgtttcccgagaggaatcaaaagagaagccatctgttttccatttacaagccccaacttggtatggggaaccatctcccgcagctcattgggctttcggtggtaaattggttcaaattactccagatggtaaaggtgtatctataacaaacccaaaaatttcaggcttagaatcaaacactactttgagtgaagcgttgaaaactaaggatttcaaaccattaataaatcaaagactggtcaaagttattgatgacgttaatgaagaagattggaatttattggaaaagttatcaatggacggtactgaggagttcttgaaagaggctcttgcattcgacaacgatgaatcagatgcacaagacgatgccaacaatgagaaagaagacgatggggaagaattctttcaacaaattgaaaccaatttccaacccgagggcgatttctccttgtctggtaatatcgaacaaactatttccaagaacttggtttctggcaacattaagagcgctgtgaaaaattctctagagaatgacttactaatggaggccatggtgatcgcattagattcaaataacgaaagattaaaggaaagtgtcaagaatgcctattttgcgaagtatggatctaaatcatcgctctcgaggatactatactccatttctaagagggaagtagatgatttggttgaaaatttggatgtctctcagtggaagtttatctctaaagcaattcaaaacttatatccaaatgatatcgcccagaggaatgaaatgttgattaaattgggagacaggttaaaggaaaatggtcatagacaagattctttgactttgtacttggctgccggatcattagataaggtggcttcaatttggttatcagaatttccagatttggaggataaattgaagaaagataataagacaattta

SEQ ID NO: 57 - protein, 649 aa, Saccharomyces cerevisiae
MSRAVGIDLGTTYSCVAHFSNDRVEIIANDQGNRTTPSYVAFTDTERLIGDAAKNQAAINPHNTVFDAKRLIGRKFDDPEVTTDAKHFPFKVISRDGKPVVQVEYKGETKTFTPEEISSMVLSKMKETAENYLGTTVNDAVVTVPAYFNDSQRQATKDAGTIAGMNVLRIINEPTAAAIAYGLDKKGRAEHNVLIFDLGGGTFDVSLLSIDEGVFEVKATAGDTHLGGEDFDNRLVNHLATEFKRKTKKDISNNQRSLRRLRTAAERAKRALSSSSQTSIEIDSLFEGMDFYTSLTRARFEELCADLFRSTLEPVEKVLKDSKLDKSQIDEIVLVGGSTRIPKIQKLVSDFFNGKEPNRSINPDEAVAYGAAVQAAILTGDQSTKTQDLLLLDVAPLSLGIETAGGIMTKLIPRNSTIPTKKSETFSTYADNQPGVLIQVFEGERTRTKDNNLLGKFELSGIPPAPRGVPQIDVTFDIDANGILNVSALEKGTGKSNKITITNDKGRLSKDDIDRMVSEAEKYRADDEREAERVQAKNQLESYAFTLKNTINEASFKEKVGEDDAKRLETASQETIDWLDASQAASTDEYKDRQKELEGIANPIMTKFYGAGAGAGPGAGESGGFPGSMPNSGATGGGEDTGPTVEEVD

SEQ ID NO: 58 - DNA, 1950 bp, Saccharomyces cerevisiae
atgtctagagcagttggtattgatttgggaacaacttactcgtgtgttgctcatttttccaatgatagggtagagataattgcaaatgatcaaggtaataggaccactccatcgtatgtggctttcacagacaccgaaagattaattggtgacgccgccaaaaatcaagctgcaatcaatcctcataatacagtttttgatgcaaagcggttaattggtcgtaaatttgatgatcctgaagtgacgacagatgccaagcacttccctttcaaagttatatccagagatggtaaacctgtagtgcaagtagaatataagggtgaaacgaaaacatttacgcctgaggaaatttcttccatggttttaagcaaaatgaaggaaactgctgagaactatttgggaactacggtcaatgatgctgttgtaactgttcctgcatatttcaatgattctcaaagacaagccactaaggatgcaggaactattgcagggatgaacgttttacgtattatcaatgaacccactgcagcagcaattgcttatggcttggataagaaaggcagggctgagcacaatgtcctgatttttgatttgggtggtggtacttttgacgtctctttactttcaattgatgagggtgtttttgaggttaaggctaccgcaggagacactcatttaggtggtgaagattttgataataggttggtgaaccatttagccactgaattcaaaaggaaaacgaaaaaggacatctctaataatcaaagatcgttaagaagattgagaactgcggcagaaagagctaagagagcgctttcttcctcatctcaaacctcgatcgagatcgattctttatttgaaggtatggatttctacacttcgttaacaagggcaaggtttgaagagctatgtgctgatttattcagatccacattggaaccagtagaaaaggttcttaaagattcgaagctggacaagtcccaaattgatgagattgtgttagtcggtggatctaccagaatcccaaagattcagaaattagtttctgacttcttcaatggcaaagagcctaatcgttctatcaacccggatgaggctgttgcttatggtgcagccgttcaagctgccattttaaccggcgatcaatcaacaaagacacaagatttactattattggatgttgcgccattgtccctaggaattgaaactgcaggcggcataatgactaagctaattcctagaaactcaacgattccaacaaagaaatcggaaaccttctctacctatgcagataatcaacctggtgttttaattcaagtctttgaaggtgaaagaacaagaacaaaggataataacttacttggtaaattcgaattaagtggcattccgcctgctcccagaggtgtgcctcaaattgatgttacctttgatatcgacgctaatggtattcttaatgtgtctgctttggaaaagggtactggtaagagtaacaaaatcacgatcactaacgataaaggtaggctctcgaaggatgatattgataggatggtttctgaagctgaaaaatatagggctgacgatgaaagggaggcagaacgagttcaggctaagaatcagcttgaatcgtatgcatttactttgaagaataccataaacgaagcaagtttcaaagagaaagtaggtgaagatgatgcaaagagattagaaacagcgtctcaggaaaccattgactggttagatgcatcgcaggcagcctctacggacgaatataaggatagacaaaaggagttggaaggcattgccaatccaataatgacgaaattttacggtgctggtgccggcgcaggtcctggagcgggggaatccggtggattccccggatccatgcccaactcgggtgctacgggaggtggagaagatacaggtccaacagtggaagaggttgattga

SEQ ID NO: 59 - protein, 206 aa, Saccharomyces cerevisiae
MPSHRNSNLKFCTVCASNNNRSMESHKVLQEAGYNVSSYGTGSAVRLPGLSIDKPNVYSFGTPYNDIYNDLLSQSADRYKSNGLLQMLDRNRRLKKAPEKWQESTKVFDFVFTCEERCFDAVCEDLMNRGGKLNKIVHVINVDIKDDDENAKIGSKAILELADMLNDKIEQCEKDDIPFEDCIMDILTEWQSSHSQLPSLYAPSYY

SEQ ID NO: 60 - DNA, 621 bp, Saccharomyces cerevisiae
atgcctagtcatcgcaattcaaacttgaagttttgcacagtttgtgcatcaaacaacaatcgttcaatggaatcgcataaagtcctgcaagaagcaggctataatgttagctcttacggaacaggttcagctgtgagactgcctggtctatcgatagataagcctaatgtgtactcatttggtacaccctataatgatatatataatgatcttttatcacaatcagcagaccgttacaagtcgaacggtttattgcaaatgctggatcgtaatagaagactcaaaaaagcacctgaaaaatggcaagaaagtacaaaagtcttcgacttcgttttcacttgtgaagagagatgttttgatgccgtttgtgaagatttgatgaatagaggtgggaaattaaacaaaatagtgcatgtaattaatgttgacattaaagatgatgatgaaaatgctaaaattggtagcaaagctatattggaattagctgatatgctcaatgataaaatagaacaatgtgaaaaagatgacattccctttgaagattgtataatggacattttaactgagtggcaaagctcacattctcaactaccgtcattatacgctccttcatattactaa

SEQ ID NO: 61 - protein, 516 aa, Saccharomyces cerevisiae
MSTQQQSYTIWSPQDTVKDVAESLGLENINDDVLKALAMDVEYRILEIIEQAVKFKRHSKRDVLTTDDVSKALRVLNVEPLYGYYDGSEVNKAVSFSKVNTSGGQSVYYLDEEEVDFDRLINEPLPQVPRLPTFTTHWLAVEGVQPAIIQNPNLNDIRVSQPPFIRGAIVTALNDNSLQTPVTSTTASASVTDTGASQHLSNVKPGQNTEVKPLVKHVLSKELQIYFNKVISTLTAKSQADEAAQHMKQAALTSLRTDSGLHQLVPYFIQFIAEQITQNLSDLQLLTTILEMIYSLLSNTSIFLDPYIHSLMPSILTLLLAKKLGGSPKDDSPQEIHEFLERTNALRDFAASLLDYVLKKFPQAYKSLKPRVTRTLLKTFLDINRVFGTYYGCLKGVSVLEGESIRFFLGNLNNWARLVFNESGITLDNIEEHLNDDSNPTRTKFTKEETQILVDTVISALLVLKKDLPDLYEGKGEKVTDEDKEKLLERCGVTIGFHILKRDDAKELISAIFFGE

SEQ ID NO: 62 - DNA, 1551 bp, Saccharomyces cerevisiae
atgtctacacagcagcaatcttacacaatttggtctccacaagatactgttaaagatgtcgctgagtcgcttgggttggaaaatatcaacgatgacgtgttgaaagcacttgctatggacgttgaataccgtattctagagattattgaacaggcagtcaagtttaaaagacactctaaaagagatgttttaactacagacgatgtatcgaaggccctgcgtgttctgaatgtggaacctttatatggatattatgatggctctgaagttaataaagctgtatcatttagtaaggttaacacaagtggaggacagtcggtttactacctggatgaggaagaagtagattttgatagattaataaatgagccattaccgcaagtgcctcgtctaccaacttttactacacattggctagcagttgaaggggttcaacctgccataatccaaaatccaaatttgaatgatataagggtatcccaaccaccatttattagaggtgctattgtcactgctttgaatgataacagcctccaaacacctgtaacgtcgacgacagcaagtgcttctgtgacggatacaggcgcctctcaacacctgtctaatgttaaaccaggacagaatactgaagtcaaacctttagtgaaacacgttttatccaaagaattacagatttatttcaataaggtcatttcaaccttgacagcgaagagtcaagctgatgaagctgcacagcatatgaaacaagcagctttgacttcactacgaactgatagtggcctgcaccaactggttccatattttattcaatttattgccgaacaaatcacacaaaatctttccgatttacaattgctaacaacaattttggaaatgatttactctttactaagcaatacttctattttcctggacccttatattcattcgttaatgccttccattttaaccttgctattagcaaaaaaacttggtggatcaccaaaagatgattcgccgcaagaaattcatgaatttttagaaagaacgaatgcactacgtgattttgccgcatctttgttggactatgtattgaaaaaatttcctcaagcatacaaatccctgaagccaagagtaactaggacactgctaaagacatttttggacataaaccgtgtttttgggacttactatgggtgcttaaagggtgtatcggtactagaaggtgaatccatcagattcttcttaggaaacctaaataattgggcgcgcttggtattcaatgaaagcggaataactctagacaatatagaggaacatttgaacgatgactccaatccaactaggaccaaatttactaaagaagaaactcaaatcttggttgatactgtaattagtgcattgttagttctaaaaaaggatttaccagatttgtacgagggtaaaggtgaaaaagttacggatgaggataaggaaaaattgctagagaggtgtggtgtcactattggatttcacattttgaaaagagacgacgcaaaagaattaattagtgcgatattttttggcgaatag

SEQ ID NO: 63 - protein, 914 aa, Saccharomyces cerevisiae
MTDQRGPPPPHPQQANGYKKFPPHDNQYSGANNSQPNNHYNENLYSAREPHNNKQYQSKNGKYGTNKYNNRNNSQGNAQYYNNRFNNGYRLNNNDYNPAMLPGMQWPANYYAPQMYYIPQQMVPVASPPYTHQPLNTNPEPPSTPKTTKIEITTKTGERLNLKKFHEEKKASKGEEKNDGVEQKSKSGTPFEKEATPVLPANEAVKDTLTETSNEKSTSEAENTKRLFLEQVRLRKAAMERKKNGLISETEKKQETSNHDNTDTTKPNSVIESEPIKEAPKPTGEANEVVIDGKSGASVKTPQHVTGSVTKSVTFNEPENESSSQDVDELVKDDDTTEISDTTGGKTVNKSDDETINSVITTEENTVKETEPSTSDIEMPTVSQLLETLGKAQPISDIYEFAYPENVERPDIKYKKPSVKYTYGPTFLLQFKDKLKFRPDPAWVEAVSSKIVIPPHIARNKPKDSGRFGGDFRSPSMRGMDHTSSSRVSSKRRSKRMGDDRRSNRGYTSRKDREKAAEKAEEQAPKEEIAPLVPSANRWIPKSRVKKTEKKLAPDGKTELFDKEEVERKMKSLLNKLTLEMFDSISSEILDIANQSKWEDDGETLKIVIEQIFHKACDEPHWSSMYAQLCGKVVKDLDPNIKDKENEGKNGPKLVLHYLVARCHEEFEKGWADKLPAGEDGNPLEPEMMSDEYYIAAAAKRRGLGLVRFIGYLYCLNLLTGKMMFECFRRLMKDLNNDPSEETLESVIELLNTVGEQFEHDKFVTPQATLEGSVLLDNLFMLLQHIIDGGTISNRIKFKLIDVKELREIKHWNSAKKDAGPKTIQQIHQEEEQLRQKKNSQRSNSRFNNHNQSNSNRYSSNRRNMQNTQRDSFASTKTGSFRNNQRNARKVEEVSQAPRANMFDALMNNDGDSD

SEQ ID NO: 64 - DNA, 2000 bp, Saccharomyces cerevisiae
atgactgaccaaagaggtccaccgcccccacacccgcagcaagccaatggctacaagaaatttcctcctcatgataaccaatactctggagccaataatagtcagccaaataaccactacaatgaaaatctttacagtgcaagggaacctcacaataacaagcaataccagtcgaaaaatggcaaatacgggacaaataaatacaataatcgtaataatagtcaaggaaatgcacagtactacaataacagattcaataatggctacagactgaataacaacgactataacccggcaatgttgccaggtatgcaatggccagctaactactatgctcctcagatgtattatataccccaacaaatggttccagttgcttctccaccatatacacaccaaccactcaacactaatcccgaacctccctctacacctaaaactacaaaaatagaaatcactacgaaaactggtgaacgtttaaatttgaaaaaattccacgaagaaaagaaagcttcgaaaggtgaagagaagaatgatggggtagagcagaaatctaaatcaggaaccccttttgaaaaggaggcaactcctgtactacctgctaatgaagcagttaaagatacgttaaccgaaacatctaatgaaaaatctacctctgaagcggaaaatactaagagattatttttagagcaggtgagattgcgcaaagctgccatggaaagaaagaaaaacggtttaatctccgaaactgagaagaagcaagaaacttcaaatcatgataatactgatacaactaagcccaactcggttattgaatctgaacctattaaggaagcgccgaaaccaacgggagaagctaatgaagttgtgattgatggaaaatcaggggcaagtgtcaaaactccacaacatgtaactggaagtgtaactaaatccgtgacttttaacgaacccgagaatgagagcagttctcaggatgttgatgagcttgttaaagatgatgatactactgaaatttcggatacgactggtggaaaaactgtaaataaaagtgacgacgaaacaataaattccgtaatcaccacagaggagaacacagtgaaggagacagaaccttccacttccgatattgagatgccaactgtatctcaactacttgaaactttaggtaaggctcaaccaatttctgacatatatgagtttgcctatccggaaaatgttgaaaggcctgatatcaaatacaagaaaccaagcgttaagtacacttatggccctaccttcttactgcaattcaaagataaactaaaattcaggcctgatcctgcgtgggttgaagctgtatcctcgaaaattgttatacctcctcatatagccagaaataaaccaaaagatagtggcagatttggaggcgatttcagaagtccatctatgcgcggtatggaccatacttccagttcaagagtttcatcgaaaagaagatcaaagagaatgggtgacgacagaagatctaatagaggatatacctccagaaaagatcgcgaaaaagctgcagaaaaggcagaagagcaagctccaaaggaagaaatcgccccgttggttccgagtgctaatagatggatacctaaatcaagggttaaaaaaacagaaaagaagttagctcctgacggcaaaactgaattatttgacaaggaagaagtagaacgaaaaatgaagtctctattaaacaagttaactttagaaatgtttgattcaatctcctctgagattttggatatagcaaaccaatcaaagtgggaagatgatggcgaaacattgaaaatagttattgaacaaattttccacaaggcttgtgatgaacctcattggtcttcaatgtatgctcagttatgtggcaaagtggtcaaagaccttgacccaaacattaaagataaagagaacgaaggaaagaatggaccaaagcttgtgttgcattatttagtggcaagatgtcacgaagaatt

SEQ ID NO: 65 - protein, 680 aa, Saccharomyces cerevisiae
MTQFTDIDKLAVSTIRILAVDTVSKANSGHPGAPLGMAPAAHVLWSQMRMNPTNPDWINRDRFVLSNGHAVALLYSMLHLTGYDLSIEDLKQFRQLGSRTPGHPEFELPGVEVTTGPLGQGISNAVGMAMAQANLAATYNKPGFTLSDNYTYVFLGDGCLQEGISSEASSLAGHLKLGNLIAIYDDNKITIDGATSISFDEDVAKRYEAYGWEVLYVENGNEDLAGIAKAIAQAKLSKDKPTLIKMTTTIGYGSLHAGSHSVHGAPLKADDVKQLKSKFGFNPDKSFVVPQEVYDHYQKTILKPGVEANNKWNKLFSEYQKKFPELGAELARRLSGQLPANWESKLPTYTAKDSAVATRKLSETVLEDVYNQLPELIGGSADLTPSNLTRWKEALDFQPPSSGSGNYSGRYIRYGIREHAMGAIMNGISAFGANYKPYGGTFLNFVSYAAGAVRLSALSGHPVIWVATHDSIGVGEDGPTHQPIETLAHFRSLPNIQVWRPADGNEVSAAYKNSLESKHTPSIIALSRQNLPQLEGSSIESASKGGYVLQDVANPDIILVATGSEVSLSVEAAKTLAAKNIKARVVSLPDFFTFDKQPLEYRLSVLPDNVPIMSVEVLATTCWGKYAHQSFGIDRFGASGKAPEVFKFFGFTPEGVAERAQKTIAFYKGDKLISPLKKAF

SEQ ID NO: 66 - DNA, 2000 bp, Saccharomyces cerevisiae
atgactcaattcactgacattgataagctagccgtctccaccataagaattttggctgtggacaccgtatccaaggccaactcaggtcacccaggtgctccattgggtatggcaccagctgcacacgttctatggagtcaaatgcgcatgaacccaaccaacccagactggatcaacagagatagatttgtcttgtctaacggtcacgcggtcgctttgttgtattctatgctacatttgactggttacgatctgtctattgaagacttgaaacagttcagacagttgggttccagaacaccaggtcatcctgaatttgagttgccaggtgttgaagttactaccggtccattaggtcaaggtatctccaacgctgttggtatggccatggctcaagctaacctggctgccacttacaacaagccgggctttaccttgtctgacaactacacctatgttttcttgggtgacggttgtttgcaagaaggtatttcttcagaagcttcctccttggctggtcatttgaaattgggtaacttgattgccatctacgatgacaacaagatcactatcgatggtgctaccagtatctcattcgatgaagatgttgctaagagatacgaagcctacggttgggaagttttgtacgtagaaaatggtaacgaagatctagccggtattgccaaggctattgctcaagctaagttatccaaggacaaaccaactttgatcaaaatgaccacaaccattggttacggttccttgcatgccggctctcactctgtgcacggtgccccattgaaagcagatgatgttaaacaactaaagagcaaattcggtttcaacccagacaagtcctttgttgttccacaagaagtttacgaccactaccaaaagacaattttaaagccaggtgtcgaagccaacaacaagtggaacaagttgttcagcgaataccaaaagaaattcccagaattaggtgctgaattggctagaagattgagcggccaactacccgcaaattgggaatctaagttgccaacttacaccgccaaggactctgccgtggccactagaaaattatcagaaactgttcttgaggatgtttacaatcaattgccagagttgattggtggttctgccgatttaacaccttctaacttgaccagatggaaggaagcccttgacttccaacctccttcttccggttcaggtaactactctggtagatacattaggtacggtattagagaacacgctatgggtgccataatgaacggtatttcagctttcggtgccaactacaaaccatacggtggtactttcttgaacttcgtttcttatgctgctggtgccgttagattgtccgctttgtctggccacccagttatttgggttgctacacatgactctatcggtgtcggtgaagatggtccaacacatcaacctattgaaactttagcacacttcagatccctaccaaacattcaagtttggagaccagctgatggtaacgaagtttctgccgcctacaagaactctttagaatccaagcatactccaagtatcattgctttgtccagacaaaacttgccacaattggaaggtagctctattgaaagcgcttctaagggtggttacgtactacaagatgttgctaacccagatattattttagtggctactggttccgaagtgtctttgagtgttgaagctgctaagactttggccgcaaagaacatcaaggctcgtgttgtttctctaccagatttcttcacttttgacaaacaacccctagaatacagactatcagtcttaccagacaacgttccaatcatgtctgttgaagttttggctaccacatgttggggcaaatacgctcatcaatccttcggtattgacagatttggtgcctccggtaaggcaccagaagtcttcaagttcttcggtttcaccccagaaggtgttgctgaaagagctcaaaagaccattgcattcta

SEQ ID NO: 67 - protein, 196 aa, Saccharomyces cerevisiae
MVAQVQKQAPTFKKTAVVDGVFDEVSLDKYKGKYVVLAFIPLAFTFVCPTEIIAFSEAAKKFEEQGAQVLFASTDSEYSLLAWTNIPRKEGGLGPINIPLLADTNHSLSRDYGVLIEEEGVALRGLFIIDPKGVIRHITINDLPVGRNVDEALRLVEAFQWTDKNGTVLPCNWTPGAATIKPTVEDSKEYFEAANK

SEQ ID NO: 68 - DNA, 591 bp, Saccharomyces cerevisiae
atggtcgctcaagttcaaaagcaagctccaacttttaagaaaactgccgtcgtcgacggtgtctttgacgaagtctccttggacaaatacaagggtaagtacgttgtcctagcctttattccattggccttcactttcgtctgtccaaccgaaatcattgctttctcagaagctgctaagaaattcgaagaacaaggcgctcaagttcttttcgcctccactgactccgaatactcccttttggcatggaccaatatcccaagaaaggaaggtggtttgggcccaatcaacattccattgttggctgacaccaaccactctttgtccagagactatggtgtcttgatcgaagaagaaggtgtcgccttgagaggtttgttcatcatcgacccaaagggtgtcattagacacatcaccattaacgatttgccagtcggtagaaacgttgacgaagccttgagattggttgaagccttccaatggaccgacaagaacggtactgtcttgccatgtaactggactccaggtgctgctaccatcaagccaaccgttgaagactccaaggaatacttcgaagctgccaacaaataa

SEQ ID NO: 69 - protein, 291 aa, Saccharomyces cerevisiae
MNSILDRNVRSSETTLIKPESEFDNWLSDENDGASHINVNKDSSSVLSASSSTWFEPLENIISSASSSSIGSPIEDQFISSNNEESALFPTDQFFSNPSSYSHSPEVSSSIKREEDDNALSLADFEPASLQLMPNMINTDNNDDSTPLKNEIELNDSFIKTNLDAKETKKRAPRKRLTPFQKQAHNKIEKRYRININTKIARLQQIIPWVASEQTAFEVGDSVKKQDEDGAETAATTPLPSAAATSTKLNKSMILEKAVDYILYLQNNERLYEMEVQRLKSEIDTLKQDQK

SEQ ID NO: 70 - DNA, 876 bp, Saccharomyces cerevisiae
atgaactctattttagacagaaatgttagatctagcgaaactactttaattaaacctgaatctgaatttgataattggttgtcggatgaaaatgacggagctagtcatatcaacgtcaacaaggactcctcgtcagttctttctgcatcttcttccacatggttcgaaccattggaaaacattatctcctctgcatccagctcctcgataggctctccaatcgaagaccagtttatatcttccaacaacgaggaatctgctctttttccaacagatcagtttttcagtaatccttcctcatactcgcattctcccgaggttagcagctcgataaaaagagaagaggatgacaatgccctttcgttggcagattttgaaccggcttctttgcaattaatgcctaacatgataaatactgataataatgacgatagtaccccacttaagaatgaaatcgagctaaacgactcgtttataaaaacaaatctagatgctaaggaaacgaaaaagagggctccaagaaaaagactgacccccttccaaaagcaagctcacaacaagattgaaaaacgctacagaataaacatcaacacaaagattgcaagactgcagcagattatcccatgggtagcaagtgaacaaacagctttcgaagtaggtgattctgtaaaaaaacaggacgaagacggcgcagaaactgccgctactactcctcttccatctgccgctgctacaagcacgaagctaaataaaagcatgatcctagaaaaagctgttgactatattctatatctacaaaataacgaacgactatacgaaatggaagttcaaaggttgaaaagtgaaatcgacactttgaaacaagaccaaaaataa

SEQ ID NO: 71 - protein, 362 aa, Saccharomyces cerevisiae
MINNPKVDSVAEKPKAVTSKQSEQAASPEPTPAPPVSRNQYPITFNLTSTAPFHLHDRHRYLQEQDLYKCASRDSLSSLQQLAHTPNGSTRKKYIVEDQSPYSSENPVIVTSSYNHTVCTNYLRPRMQFTGYQISGYKRYQVTVNLKTVDLPKKDCTSLSPHLSGFLSIRGLTNQHPEISTYFEAYAVNHKELGFLSSSWKDEPVLNEFKATDQTDLEHWINFPSFRQLFLMSQKNGLNSTDDNGTTNAAKKLPPQQLPTTPSADAGNISRIFSQEKQFDNYLNERFIFMKWKEKFLVPDALLMEGVDGASYDGFYYIVHDQVTGNIQGFYYHQDAEKFQQLELVPSLKNKVESSDCSFEFA

SEQ ID NO: 72 - DNA, 1089 bp, Saccharomyces cerevisiae
atgatcaataatcctaaggtagacagtgtagcggagaaacccaaagctgtgacatcaaagcagtcggagcaagcggcttcgccagaaccaacaccagccccccctgtttctagaaatcagtatccgatcacgttcaacttgacttcaaccgcaccctttcatcttcatgaccgtcatcgctacttacaagagcaagatctttacaagtgcgcttctagggattcgttgtcttccctgcagcaactcgcccatacacctaatgggtcgacaaggaagaaatatattgttgaggaccaatctccctatagttcggaaaatccagtcattgtgacctcttcctataaccatacggtttgcacaaactacttaagaccaagaatgcagtttacagggtaccagatatcaggatacaaacgctatcaggtaacagttaacttaaagactgtagatttgccaaagaaagattgcacgtcgctgtcccctcatttatctggatttttgtcgataagaggactcacgaaccaacacccggaaatcagtacatattttgaagcctacgcggtaaaccacaaggaattagggttcttgtcctctagctggaaagatgaacctgttttaaacgaattcaaagccacagaccaaacagacttagaacactggataaatttcccctcctttagacagcttttcctgatgagccaaaaaaacggtctcaactcaactgacgacaatggcactacaaatgcagccaagaagttgcctccacagcagcttcctactacaccaagcgcagacgctggtaacatatcaagaatttttagccaagagaaacaatttgacaactacttgaacgaacggtttatatttatgaaatggaaggaaaaatttttggtaccagatgccctattaatggaaggtgtagacggcgcatcttatgatgggttttattacattgtccatgatcaagttaccgggaacattcaagggttttactatcatcaagatgctgaaaagttccaacagctggaattagtaccatctttgaaaaataaagtcgagtccagtgattgttcttttgagtttgcttga

SEQ ID NO: 73 - protein, 822 aa, Saccharomyces cerevisiae
MLEGTVDYDPLEDITNILFSKESLNNIDELISITRSYKKQLQEDILKEENELKEHPKNSAEIEASLRKVFQDFKETQDVSASTELTISNLTEGISYLDIAKKNLTHSLTLFQNLKILTDSYIQCNELLSQGSFKKMVSPYKIMCSLAENTFISYKSLDEINYLLSSISRLKGDTLSKIKQNYNALFSGGNISEHDTALTMELREGACELLDCDTSTRAQMIDWCLDKLLFEMKEIFRVDDEAGSLENLSRRYIYFKKILNNFNSKFADYFLKDWEMAVRLTTTFYHITHKDLQTLLKREFKDKNPSIDLFMTALQSTLDFEKYIDVRFSKKIKEPKLSSCFEPYLTLWVSHQNQMMEKKFLSYMSEPKYPSNETESLVLPSSADLFRTYRSVLTQTLELIDNNANDSILTSLANFFSRWLQTYSQKILLPLLLPDNIEVQDKLEAAKYTVLLINTADYCATTIDQLEDKLSEFSGNREKLANSFTKTKNIYDDLLAKGTSFLLNRVIPLDLNFVWREFINNDWSNAAIEDYSRYMVTLKSVLKMPALTDASIKQQQEQPSTLAFILSQFNRDVYKWNFLDKVIDIITTNFVSNTIRLLQPVPPFSLAGSKRKFETRTVVNIGEQLLLDLELLKEIFHTLPESVSNDSDLRENTSYKRVKRHADNNIDQLLKFIKLLMAPLDSADDYYETYSKLTNNNPDSAVWSFVLALKGIPWDLALWKKLWSAYNLETDDTDEGSRPDSNRDLFIFKWDKVLLGQFENNLARMQDPNWSKFVRQDLKISPPVMKRIVSTPQIQQQKEEQKKQSLSVKDFVSHSRFFNRGT

SEQ ID NO: 74 - DNA, 2000 bp, Saccharomyces cerevisiae
atgctggaaggtacggtagattatgacccgctggaagatattaccaatatacttttttcaaaagaatccctgaacaacatagatgaactgatcagtattaccagaagctacaaaaagcaattgcaagaggatattctcaaagaagagaatgaattgaaggaacaccctaaaaattccgctgaaatagaggcttctctgaggaaagttttccaagatttcaaagaaactcaagatgtctcagcctccaccgagttgacgatatcgaatctgacagaaggtatctcgtacctggacattgccaagaaaaacctcacccactctttgactcttttccaaaatttaaagatattgacagacagttacatacaatgcaatgaattactctcacagggctcattcaaaaaaatggtgtccccttataagataatgtgttcgcttgctgaaaacacattcatctcttacaaatcattggacgagataaactatttgttgagctccatttcaagactgaaaggagacactttgtccaaaattaaacaaaactacaatgcgctcttttccggcggcaatatctcagagcatgatacagcactcactatggaattgcgcgaaggtgcctgcgagctactcgactgcgatacaagtacgagagcccagatgatagattggtgtttggacaaacttctcttcgaaatgaaagagatatttagggtcgacgatgaagccggatccctagaaaatttatcgagaagatacatttacttcaaaaaaattcttaataacttcaattcaaagttcgcagactatttcttaaaagactgggaaatggcagtcagattgaccaccactttttatcacattacacacaaggaccttcagacacttctgaaaagggaattcaaagacaagaacccttccattgatctattcatgacagcattacaatcgacgctagatttcgaaaaatacatcgacgtacgattttcaaaaaaaattaaggaaccaaaactaagttcctgcttcgaaccttatttgactttatgggtgtctcaccaaaaccaaatgatggaaaagaaatttctttcttatatgagtgagccgaagtacccatctaatgaaacagaatctctcgtgttaccctcgagtgcagaccttttcaggacatatcgttccgtactgactcagaccttagagctcattgataataatgccaatgatagcatattgacttcattggcaaattttttcagtagatggcttcaaacttactcacaaaaaattcttcttcctttactgctgcccgacaatattgaagtccaggataagctagaagctgccaagtataccgttttattgatcaatactgcagattattgtgccacgactatagatcaattggaggataaattatctgaattcagcggtaatcgtgaaaagctggcaaacagttttacgaaaacgaaaaatatatacgacgatttactagcaaaaggaacttcttttctattaaaccgtgtcatacccttagatctaaattttgtatggagagagtttatcaacaatgattggtcaaatgctgcgatagaagattatagcaggtacatggtaaccctcaaatccgtacttaaaatgcccgcattaacagatgcctctattaaacaacagcaagagcaaccttcgactttggcatttattttgtcgcaattcaatagagatgtttataagtggaatttcttggataaggtgattgatatcatcactacaaattttgtaagcaataccatccgccttctgcagcccgttccacccttttccctggcgggcagcaaaaggaaatttgaaaccagaactgttgtcaacattggcgagcagcttctccttgatttagaattgctgaaggagatttttcacactttaccagaaagtgtaagtaacgattctgacttgcgagaaaatacctcttataagagggtgaaaagacatgcagacaataatataga

SEQ ID NO: 75 - protein, 779 aa, Saccharomyces cerevisiae
MERTNTTTFKFFSLGGSNEVGRSCHILQYKGKTVMLDAGIHPAYQGLASLPFYDEFDLSKVDILLISHFHLDHAASLPYVMQRTNFQGRVFMTHPTKAIYRWLLRDFVRVTSIGSSSSSMGTKDEGLFSDEDLVDSFDKIETVDYHSTVDVNGIKFTAFHAGHVLGAAMFQIEIAGLRVLFTGDYSREVDRHLNSAEVPPLSSNVLIVESTFGTATHEPRLNRERKLTQLIHSTVMRGGRVLLPVFALGRAQEIMLILDEYWSQHADELGGGQVPIFYASNLAKKCMSVFQTYVNMMNDDIRKKFRDSQTNPFIFKNISYLRNLEDFQDFGPSVMLASPGMLQSGLSRDLLERWCPEDKNLVLITGYSIEGTMAKFIMLEPDTIPSINNPEITIPRRCQVEEISFAAHVDFQENLEFIEKISAPNIILVHGEANPMGRLKSALLSNFASLKGTDNEVHVFNPRNCVEVDLEFQGVKVAKAVGNIVNEIYKEENVEIKEEIAAKIEPIKEENEDNLDSQAEKGLVDEEEHKDIVVSGILVSDDKNFELDFLSLSDLREHHPDLSTTILRERQSVRVNCKKELIYWHILQMFGEAEVLQDDDRVTNQEPKVKEESKDNLTNTGKLILQIMGDIKLTIVNTLAVVEWTQDLMNDTVADSIIAILMNVDSAPASVKLSSHSCDDHDHNNVQSNAQGKIDEVERVKQISRLFKEQFGDCFTLFLNKDEYASNKEETITGVVTIGKSTAKIDFNNMKILECNSNPLKGRVESLLNIGGNLVTPLC

SEQ ID NO: 76 - DNA, 2000 bp, Saccharomyces cerevisiae
atggagcgaacaaatacaacaacattcaaatttttttcattgggaggaagtaacgaagttggacgatcatgtcatatattgcaatataaaggtaaaacagtgatgctcgacgcaggaattcatccggcatatcaaggattagcttcattacctttttacgatgaatttgatctttccaaagtcgatatcttactgatatcacatttccatttagaccatgccgcttcacttccgtatgtgatgcaacggactaactttcaaggcagagttttcatgacacatccaaccaaagccatttacagatggctactgcgagattttgtaagagttactagtataggttcttcatcctcctctatggggactaaagacgaaggtctattctcagatgaggatttagttgattctttcgataaaattgaaacggtggactatcattctactgttgacgtcaatggtatcaaatttacggcattccatgcaggccatgtattgggtgcagcgatgtttcaaatagagattgctggccttagggtgttattcacaggtgactattcgagagaagtagatcgtcacttaaattctgctgaggtgcctccactttcatccaacgtattaattgtggaatctacctttggtaccgctactcacgagccccgtttaaatagagaaaggaagctaacccaactaatccattccacagtgatgagaggaggccgtgttctactgcctgtttttgctttagggagagctcaagaaatcatgcttatactggatgagtactggtctcaacatgctgatgaactcggtggtggacaagtcccaatattttatgcatcaaatttggcaaaaaaatgtatgagcgtttttcaaacctatgtaaatatgatgaatgatgacattagaaaaaaatttagagactctcagactaatcccttcatattcaaaaatatatcttatcttagaaacttggaggatttccaagattttggtcccagtgtgatgttggcctcaccaggtatgctgcaaagtgggttatcaagagatttacttgaaaggtggtgccctgaagataaaaatctagtactaatcaccggttactccatcgaaggaacaatggcgaaatttattatgcttgagccagatacaataccttccataaataatccggaaataaccattccaagacgttgtcaagttgaagaaatctcctttgccgcacacgttgacttccaggaaaatttagaatttattgaaaagattagtgcgccaaatatcatccttgttcatggagaagccaatcccatgggccgtttgaaatctgcattgttatccaatttcgcgtctttaaagggtacagataatgaagtccatgtttttaatcctagaaactgtgttgaagtagatcttgaatttcaaggtgtcaaggttgcaaaagctgtgggaaatattgtgaacgaaatatataaagaagaaaacgtagagataaaggaagaaattgcggctaaaattgaacctataaaggaagaaaacgaagacaacttggattctcaagcagaaaaaggtttggttgatgaagaggaacacaaagacatagtcgtttctgggattctagtttcagatgacaaaaatttcgaattagacttcctttctttgtctgatttaagagagcaccatcccgatctttctacaacgatattaagagagcgccagtcagttcgtgtaaattgtaaaaaggagctaatttactggcacattttacaaatgtttggagaggctgaagttcttcaagatgatgatagagtaacaaatcaagaaccaaaggttaaagaagagtcaaaagacaatctgaccaatacaggtaaattgattctacagataatgggtgatattaagttaactattgttaatactctagccgttgtggaatggactcaagatttaatgaatgacactgtagcagactccattattgctatacttatgaatgtggattcagc

SEQ ID NO: 77 - protein, 208 aa, Saccharomyces cerevisiae
MSLIHPDTAKYPFKFEPFLRQEYSFSLDPDRPICEFYNSREGPKSCPRGPLCPKKHVLPIFQNKIVCRHWLRGLCKKNDQCEYLHEYNLRKMPECVFFSKNGYCTQSPDCQYLHIDPASKIPKCENYEMGFCPLGSSCPRRHIKKVFCQRYMTGFCPLGKDECDMEHPQFIIPDEGSKLRIKRDDEINTRKMDEEKERRLNAIINGEV

SEQ ID NO: 78 - DNA, 627 bp, Saccharomyces cerevisiae
atgagcctaattcaccccgatacagcaaaatatccttttaaatttgaacctttcctcaggcaagagtattcgttttcactcgatcctgacagacctatttgtgaattttacaattctagagaaggccctaaatcatgtccgaggggaccgttatgtccaaaaaagcatgtgttaccaatatttcagaataaaattgtttgtagacattggcttcgagggttgtgcaaaaagaatgaccaatgtgaatacttacatgaatacaatcttcgaaaaatgcctgaatgtgtcttcttcagcaaaaacgggtactgtacacaaagtccagattgtcaatatctacacatagatcccgctagcaagataccaaaatgtgaaaattacgaaatgggattctgtcctctggggagttcttgtcctagacggcatattaagaaggttttctgtcaaagatacatgaccggattttgtcctttagggaaggatgaatgtgatatggaacatccacagttcataatcccagatgaaggtagtaaattaagaattaagagagacgatgagataaataccaggaaaatggatgaagaaaaggaaaggcgtttaaacgcaattataaacggtgaagtttga

SEQ ID NO: 79 - protein, 107 aa, Saccharomyces cerevisiae
MVKGKTFLKRICPEETLNEETKQEVSVGFDKMRTLLRSRESGMTFSQGPKLASCQSVINASSEKTAWTQLVFRKSKMKTYTKSVHVIFIAMGEGEDESVDMNVGISY

SEQ ID NO: 80 - DNA, 324 bp, Saccharomyces cerevisiae
atggtaaagggtaaaacgtttctgaaaagaatctgtccggaagaaacgttaaacgaagaaactaagcaggaagtttcggtagggttcgataagatgagaaccctgttgcggtctcgagaatcagggatgactttctcccaaggacctaagttagccagttgccaatcagtgataaatgcatcatctgaaaaaacggcttggacacaactcgtgtttaggaagagtaaaatgaagacgtacaccaagtctgtacacgttatcttcattgctatgggggaaggggaggatgaaagtgttgatatgaatgtaggtattagttattaa

SEQ ID NO: 81 - protein, 425 aa, Saccharomyces cerevisiae
MTDPRRRTGRHFLTPENLSSTLQITNLPPEWNQDIITSVVAGSGPVIDIKAKNDPRTGKLTGVLFDYLTSKDCKRAWEILNRIENFPVKIEQIIPPNYKDHLRETANKNSQKQVLQLNRDSYPFEAGLELPFEMVTEVPIPRRPPPPQAANNTNSVSNNTNIQFPDILSKASKHLPSFQDGSIIAPDKISQNLSKIPPLQLIEIISNLKILSNQENIQKSQLESFLDTNSDITISVTQALLEMGFIDYSVVTKVLKSQVGEAPSLLSSNNTSNSNTPVSVIRNNTPLHVPSNEVSNNPNNMPLNVAMPMPMSTPPFIPLPLQQQPFGFAPPGPFMPPAQGPSMGQPVLANQLGQVQQQNISSTEGPSNANKANDSGTINMAKLQLLPENQQDMIKQVLTLTPAQIQSLPSDQQLMVENFRKEYII

SEQ ID NO: 82 - DNA, 1278 bp, Saccharomyces cerevisiae
atgacagatcccagaagaagaacaggccgtcatttcttgacaccggagaatttatcctctacattacaaatcacaaacttacctccagaatggaaccaagatataattacttcggtcgtggccggttctggtccagttatagatataaaagctaagaatgacccgagaactggtaaactaaccggtgtactgttcgattatttgactagtaaagattgtaaacgcgcttgggaaattttaaatagaattgaaaactttcccgtaaagatagagcaaataatcccaccaaattataaggaccatcttagagaaacagcaaataaaaattctcaaaagcaggtattacaacttaatagagattcgtaccccttcgaggcgggtttggagctacctttcgaaatggtgacagaagtccccattcctaggcgaccaccgccaccacaggctgcaaataacacaaactctgtatcaaataacacaaacattcaattccccgacatactaagtaaagcatctaaacacttgccaagtttccaagatggctcgattattgcaccagacaaaatttcacaaaatttaagtaaaattccgccgttgcaacttattgaaattatatcaaatttgaaaatattatcaaaccaagaaaacatccaaaaatcgcaattagaatctttcttagatactaacagtgatatcacaatatcagtgacccaagccctactagaaatgggatttatagactacagcgtggtgactaaagtgttgaaatcccaagttggcgaggccccatctttgctttcgagtaataacacaagtaattcgaacacccccgtaagcgtaattagaaataacactccgttgcatgtaccttctaatgaagtcagcaacaatcctaacaatatgccactgaacgtagctatgccaatgcctatgtcgacaccaccatttatccctttacctctgcaacaacaaccgttcggttttgcgccaccgggccctttcatgcctccagctcaaggcccctccatgggacagcctgtgttggcaaatcaactcggccaggtccagcaacaaaatataagttctacagaaggaccctctaacgcgaataaagcgaatgacagcggcaccattaatatggcgaaactgcaattactacctgaaaaccaacaagatatgatcaaacaagttcttactttgacacctgcccagatccaaagtttaccaagtgaccagcaacttatggtggaaaactttagaaaagaatatataatctaa

SEQ ID NO: 83 - protein, 1592 aa, Saccharomyces cerevisiae
MGTDPLIIRNNGSFWEVDDFTRLGRTQLLSYYLPLAIIASIGIFALCRSGLSRYVRSAECDLVNEYLFGAQEERKEDNSIERLLRNSNTQANYVNVKKQGRILKLRHFDITTIDVKQIDAKNHGGLTFSRPSTSDHLRKSSEIVLMSLQIIGLSFLRVTKINIELTNRDVTTLLLFWLILLSLSILRVYKRSTNLWAICFTAHTTIWISTWIPIRSVYIGNIDDVPSQIFYIFEFVITSTLQPIKLTSPIKDNSSIIYVRDDHTSPSREHISSILSCITWSWITNFIWEAQKNTIKLKDIWGLSMEDYSIFILKGFTRRNKHINNLTLALFESFKTYLLIGMLWVLVNSIVNLLPTILMKRFLEIVDNPNRSSSCMNLAWLYIIGMFICRLTLAICNSQGQFVSDKICLRIRAILIGEIYAKGLRRRLFTSPKTSSDSDSISANLGTIINLISIDSFKVSELANYLYVTVQAVIMIIVVVGLLFNFLGVSAFAGISIILVMFPLNFLLANLLGKFQKQTLKCTDQRISKLNECLQNIRIVKYFAWERNIINEIKSIRQKELRSLLKKSLVWSVTSFLWFVTPTLVTGVTFAICTFVQHEDLNAPLAFTTLSLFTLLKTPLDQLSNMLSFINQSKVSLKRISDFLRMDDTEKYNQLTISPDKNKIEFKNATLTWNENDSDMNAFKLCGLNIKFQIGKLNLILGSTGSGKSALLLGLLGELNLISGSIIVPSLEPKHDLIPDCEGLTNSFAYCSQSAWLLNDTVKNNIIFDNFYNEDRYNKVIDACGLKRDLEILPAGDLTEIGEKGITLSGGQKQRISLARAVYSSAKHVLLDDCLSAVDSHTAVWIYENCITGPLMKNRTCILVTHNVSLTLRNAHFAIVLENGKVKNQGTITELQSKGLFKEKYVQLSSRDSINEKNANRLKAPRKNDSQKIEPVTENINFDANFVNDGQLIEEEEKSNGAISPDVYKWYLKFFGGFKALTALFALYITAQILFISQSWWIRHWVNDTNVRINAPGFAMDTLPLKGMTDSSKNKHNAFYYLTVYFLIGIIQAMLGGFKTMMTFLSGMRASRKIFNNLLDLVLHAQIRFFDVTPVGRIMNRFSKDIEGVDQELIPYLEVTIFCLIQCASIIFLITVITPRFLTVAVIVFVLYFFVGKWYLTASRELKRLDSITKSPIFQHFSETLVGVCTIRAFGDERRFILENMNKIDQNNRAFFYLSVTVKWFSFRVDMIGAFIVLASGSFILLNIANIDSGLAGISLTYAILFTDGALWLVRLYSTFEMNMNSVERLKEYSSIEQENYLGHDEGRILLLNEPSWPKDGEIEIENLSLRYAPNLPPVIRNVSFKVDPQSKIGIVGRTGAGKSTIITALFRLLEPITGCIKIDGQDISKIDLVTLRRSITIIPQDPILFAGTIKSNVDPYDEYDEKKIFKALSQVNLISSHEFEEVLNSEERFNSTHNKFLNLHTEIAEGGLNLSQGERQLLFIARSLLREPKIILLDEATSSIDYDSDHLIQGIIRSEFNKSTILTIAHRLRSVIDYDRIIVMDAGEVKEYDRPSELLKDERGIFYSMCRDSGGLELLKQIAKQSSKMMK

SEQ ID NO: 84 - DNA, 2000 bp, Saccharomyces cerevisiae
atgggaacggatccccttattatccgaaataatggttcattttgggaagttgatgattttactcgtttaggaagaactcagctattgagctactatttaccattggctatcatagcctcaattggcattttcgcactttgtcgcagtggattatctcgttatgtaagatctgccgagtgcgatttagtgaacgaatatctatttggcgcacaagaagagagaaaagaagataatagtatagaaagacttctacggaactcaaatacccaagccaattacgtcaacgtcaaaaagcaaggaaggattttgaaacttagacattttgatataacaactatagatgtcaagcaaatcgatgctaaaaatcatggtggactaacgtttagtagaccgtctactagtgaccacttaagaaaatcatctgaaattgtattaatgtctttacaaataattggcctttcctttttaagagtaacaaaaatcaatattgaattaacgaacagagatgttacaactttactattattttggttaatactactttccctaagtatcttaagagtttacaagcgttcaacgaatctttgggccatctgttttactgcccatacaactatttggatttcaacctggattccaattcgttcggtctatattggtaatatcgatgatgtaccctcacagatattttacatctttgaattcgtaattacttcaaccttacagccaataaagctcacttcaccgattaaagacaactcatccatcatctacgttagagacgaccatacgtctccttcgagggaacacatatcctcaattttaagttgcattacttggagctggattaccaattttatatgggaggcccaaaagaacactattaagttaaaggatatttggggcttatcaatggaagactatagcattttcattctaaaagggtttaccaggagaaacaagcacattaataatttgacgctagcactgtttgaatctttcaaaacatatttactcataggaatgttatgggttctggtgaacagtattgtaaaccttcttcccacaattttaatgaaaagatttttagaaattgtggataacccaaaccgttcctcatcatgcatgaatttggcgtggctttatattattggtatgttcatttgtagattgacattagcaatttgtaattcccaaggtcaatttgtttctgataagatttgtttaagaataagagccatactcataggagaaatttatgcaaaaggcttacgtaggaggctgtttacatctccaaaaaccagctctgattcagatagtatctccgcaaaccttggtaccataattaatctcatttctattgactcatttaaggtatcggaactagcaaactacctttatgtgacagttcaggcagtaattatgataatagttgttgtaggactacttttcaactttttaggtgtttcagcttttgcaggaatttcaattatcttagtgatgttcccattgaatttcttgttagcgaatttgttaggtaagtttcaaaagcaaacactgaaatgtactgaccaaagaatctcaaaattgaacgagtgcttacagaacataagaattgtcaaatattttgcttgggaaaggaatattataaatgaaatcaaatcaataaggcaaaaggaattaagatccttattaaaaaaatctttggtgtggtccgtaacttcttttctttggttcgtgacaccgaccttggtgacaggtgtcactttcgccatctgtacatttgttcaacatgaagatttgaatgccccgcttgctttcactactttgtcactcttcactttgttaaagacacccctggatcaattatcaaatatgctaagtttcataaatcaatcaaaagtctctctaaaaagaataagcgattttttaaggatggacgatacagaaaaatataatcaactaaccatatctccagacaaaaataaaattgaatttaa

SEQ ID NO: 85 - protein, 329 aa, Saccharomyces cerevisiae
MTTVSINKPNLLKFKHVKSFQPQEKDCGPVTSLNFDDNGQFLLTSSSNDTMQLYSATNCKFLDTIASKKYGCHSAIFTHAQNECIYSSTMKNFDIKYLNLETNQYLRYFSGHGALVNDLKMNPVNDTFLSSSYDESVRLWDLKISKPQVIIPSLVPNCIAYDPSGLVFALGNPENFEIGLYNLKKIQEGPFLIIKINDATFSQWNKLEFSNNGKYLLVGSSIGKHLIFDAFTGQQLFELIGTRAFPMREFLDSGSACFTPDGEFVLGTDYDGRIAIWNHSDSISNKVLRPQGFIPCVSHETCPRSIAFNPKYSMFVTADETVDFYVYDE

SEQ ID NO: 86 - DNA, 990 bp, Saccharomyces cerevisiae
atgaccaccgtgtccatcaataagcccaacctgctgaaattcaagcatgttaaaagctttcaacctcaagaaaaagactgcggacccgtaacctcattgaatttcgacgataatggccagtttctactgacctcttcttccaacgatacaatgcaattgtacagtgccacgaactgcaaattcttggacactatagcctctaagaaatatggctgtcactccgctatctttacgcacgcacaaaacgaatgtatctattcctctacaatgaaaaattttgacattaaataccttaatctggaaacaaaccaatatctaagatatttttccggtcatggcgccctagtgaatgatttgaagatgaaccccgtgaacgatacgtttctatcgtcgtcatacgatgaatccgttaggctttgggatttgaagatctctaaaccgcaagttattataccaagtctcgtaccaaattgtatcgcatatgatccaagtggccttgtattcgcattggggaacccagagaatttcgaaatagggctatataatctgaaaaaaattcaggagggtcctttcttgataattaaaattaatgatgcgactttcagtcaatggaataaattagaattttctaacaatggaaagtatttattagttggctcctcgataggaaagcatttaatttttgacgcattcacaggtcaacaattattcgaactaataggaacaagggccttcccgatgagagaatttctagattctggatctgcttgtttcacaccagatggtgaattcgtccttggaaccgattatgacggtaggattgccatttggaatcattctgattcaataagtaacaaagtattaaggccgcaagggttcattccctgtgtttctcatgagacctgccccaggtcaattgcattcaaccctaaatattcgatgtttgttaccgcagacgaaacagtagatttttacgtgtacgatgaatga

SEQ ID NO: 87 - protein, 220 aa, Saccharomyces cerevisiae
MSAGDISAINIKSVKKNRRRKKRRTADVSSSDSSSSDPSSESEKEEIQNGAIEEHVGENGKSDHVFSKGNDEDKQEDIAIEVSDVELTDEESKDLKLNSKEVIDDLTKISLSKIPEPTKSQNKEGFMNASKIAENIKLAREEYNELAENFVPKGKDKTKLREEYLNLLFENYGDDINRLRAAPDFTNKSLSILADALQEGIGMFDIGELELVLKNKEMEN

SEQ ID NO: 88 - DNA, 663 bp, Saccharomyces cerevisiae
atgtcggcaggtgatatatcagccataaatatcaagtctgtcaaaaaaaacagaaggaggaagaagagaagaacagctgatgtttcatcatcagattcttcatcatcggatccatcatcagaaagtgaaaaggaggaaatccaaaatggggccatcgaagaacacgttggagaaaatggtaaaagtgatcatgttttctcaaaaggtaatgacgaagacaaacaagaggacattgcaatagaagtttcggatgtcgagcttacagacgaagaaagcaaggatttgaagttaaattcaaaagaagtgatagacgatttaaccaaaatttctttgagcaagatcccagagcctacgaaatctcaaaacaaggagggttttatgaatgcatcgaaaattgccgaaaatatcaagcttgcgagagaagaatacaatgaattggcagaaaactttgtgcccaaagggaaagacaagacaaagttaagggaagaatacttaaatttactttttgaaaactacggtgatgatatcaatcgtcttagagctgccccggatttcacgaataaatcactatccattttggcagatgctctgcaggaaggcataggaatgtttgatattggtgaactagaattggtcttgaaaaataaagaaatggagaactga

SEQ ID NO: 89 - protein, 441 aa, Saccharomyces cerevisiae
MSSTIFYRFKSQRNTSRILFDGTGLTVFDLKREIIQENKLGDGTDFQLKIYNPDTEEEYDDDAFVIPRSTSVIVKRSPAIKSFSVHSRLKGNVGAAALGNATRYVTGRPRVLQKRQHTATTTANVSGTTEEERIASMFATQENQWEQTQEEMSAATPVFFKSQTNKNSAQENEGPPPPGYMCYRCGGRDHWIKNCPTNSDPNFEGKRIRRTTGIPKKFLKSIEIDPETMTPEEMAQRKIMITDEGKFVVQVEDKQSWEDYQRKRENRQIDGDETIWRKGHFKDLPDDLKCPLTGGLLRQPVKTSKCCNIDFSKEALENALVESDFVCPNCETRDILLDSLVPDQDKEKEVETFLKKQEELHGSSKDGNQPETKKMKLMDPTGTAGLNNNTSLPTSVNNGGTPVPPVPLPFGIPPFPMFPMPFMPPTATITNPHQADASPKK

SEQ ID NO: 90 - DNA, 1326 bp, Saccharomyces cerevisiae
atgagtagcacgatattttaccgctttaagtctcaacgaaacacatcaagaattttatttgatggtaccggcctgacagtatttgatttgaaaagggaaattattcaagagaacaaactaggtgacggcacagatttccaattaaaaatttacaacccagatacagaagaggaatacgacgatgatgcctttgttatacctagatctactagtgtcatagtaaaaagatctccagcaattaaatcattctccgtacacagtcgacttaaagggaatgtgggagcagcagctcttgggaacgcaacaaggtatgttactggtaggccaagagtgttgcaaaagagacaacacactgctacaaccactgctaatgttagtggtacaacggaagaagaaagaattgctagtatgtttgccacacaagaaaatcaatgggaacaaacgcaagaagaaatgtctgcagccacacctgtttttttcaagtcacagacgaataagaattctgcacaagaaaacgaaggcccaccgccaccaggttatatgtgctatcgttgtgggggtagagaccactggattaaaaattgtccaactaacagcgatccaaattttgaaggaaaaagaatcagaagaaccacaggtattccaaagaagtttttaaaatccattgaaatagatcccgagacaatgacaccggaagagatggctcagcgaaagattatgattacggacgaaggcaagttcgtggtacaagttgaagacaaacaatcatgggaagactaccaaaggaaaagagagaaccgtcaaattgatggtgatgaaaccatttggagaaaaggccatttcaaagatcttcctgacgatttaaaatgtcccttgacaggtggtcttttgaggcagccggtaaagacaagcaagtgctgtaacatagatttctcaaaagaggcgctggaaaatgcactggtagagagcgactttgtatgccccaattgcgaaacccgcgatatccttctcgattctttagtacccgaccaggacaaggaaaaggaggtcgaaacgtttttgaagaaacaagaggaactacacggaagctctaaagatggcaaccagccagaaactaagaaaatgaagttgatggatccaactggcaccgctggcttgaacaacaataccagccttccaacttctgtaaataacggcggtacgccagtgccaccagtaccgttacctttcggtatacctcctttccccatgtttccaatgcccttcatgcctccaacggctactatcacaaatcctcatcaagctgacgcaagccctaagaaatga

SEQ ID NO: 91 - protein, 159 aa, Saccharomyces cerevisiae
MSRMPSSFDVTERDLDDMTFGERIIYHCKKQPLVPIGCLLTTGAVILAAQNVRLGNKWKAQYYFRWRVGLQAATLVALVAGSFIYGTSGKELKAKEEQLKEKAKMREKLWIQELERREEETEARRKRAELARMKTLENEEEIKNLEKELSDLENKLGKK

SEQ ID NO: 92 - DNA, 480 bp, Saccharomyces cerevisiae
atgtcacgcatgccatctagtttcgatgttacggagagggatttggatgatatgacctttggcgaaaggattatataccattgtaagaaacagccattggtacccattgggtgcttgctgactacaggagctgtcattctggctgctcaaaatgttcgtcttggtaataaatggaaagctcagtactacttccgttggcgtgtgggtctacaagcggccacactagtcgcactagtcgcaggttcatttatctatgggacttctggtaaggaactgaaggcgaaggaggaacaattgaaggagaaagccaagatgagagaaaagttatggatccaagagctggagagaagggaggaagaaacggaggcaaggagaaaaagagccgaattggcaagaatgaagacccttgagaacgaagaggaaatcaagaacttagaaaaggaactaagcgacctggaaaataagcttggaaagaagtaa

SEQ ID NO: 93 - protein, 188 aa, Saccharomyces cerevisiae
MDLPKDKSDRTHQRINLNNSGTDRTNDLYLHIVQTFGCIETTATENATKLLMLGDVEVEISASSVSIEWTQKSMISQTIADSIVIMIIGLCASDKNVLSESELKERNHNVWKIQELQNLFREQFGDSFSIDEGIGKKENVKNGSVTIGKSKATIDFSTMKLIDCNSNPLKGRVESILSIGQKLTTPLC

SEQ ID NO: 94 - DNA, 567 bp, Saccharomyces cerevisiae
atggatttacccaaggacaaaagtgacagaactcatcaaagaattaatctaaataacagtgggacagatcgaactaatgatttgtaccttcatattgtccaaacgttcggttgcatagaaacaactgcaacggaaaatgccacgaaactgttaatgctgggtgacgtcgaagtagaaatatctgcgagcagcgtttcaattgagtggacacagaagtcaatgataagccaaacaattgccgatagtatagtaataatgatcatcggtttgtgtgcaagcgacaagaacgtgctatctgaatcagaattgaaagagagaaaccataacgtttggaagatccaagaattgcaaaatctgtttcgagaacaatttggagacagttttagcatcgatgaaggaataggaaaaaaagaaaatgtaaagaatggtagcgtcaccataggcaagagtaaagccacgatcgatttctccaccatgaagctgattgattgtaattcgaacccactaaagggaagagtggagagcatactaagcattggccagaaattaacaactccattgtgctga

(19) United States
(12) Patent Application Publication
(10) Pub. No.: US 2004/0167066 A1
(43) Pub. Date: Aug. 26, 2004

(54) Cleavage and polyadenylation complex of precursor mrna

(76) Inventors: Martina Marzioch, Heidelberg (DE); Anne-Claude Gavin, Leimen (DE); Andreas Bauer, Heidelberg (DE); Jorg Schultz, Heidelberg (DE); Miro Brajenovic, Heidelberg (DE); Paola Grandi, Heidelberg (DE)

Correspondence Address: LEYDIG VOIT & MAYER, LTD, TWO PRUDENTIAL PLAZA, SUITE 4900, 180 NORTH STETSON AVENUE, CHICAGO, IL, 60601-6780, US

(21) Appl. No.: 10/477,369

(22) PCT Filed: May 15, 2002
(86) PCT No.: PCT/EP02/05359

(30) Foreign Application Priority Data
May 15, 2001 (EP) 01111774.4
Dec. 20, 2001 (EP) 01130253.6

(51) Int. Cl. A61K 38/17; C12Q 1/68; C07H 21/04; C12N 9/22

(52) U.S. Cl. 514/12; 435/6; 435/69.1; 435/199; 435/320.1; 435/325; 536/23.2

(57) ABSTRACT

The present invention relates to novel components of the cleavage/polyadenylation machinery of precursor mRNA as well as to the complex containing the new components and its use. The complex is obtained by using one component thereof as a bait and isolating a highly organized complex consisting of at least 13 distinct proteins.

### 1. FIELD OF THE INVENTION

The present invention relates to components of the cleavage/polyadenylation machinery of pre-cursor mRNA, the complete protein complex, uses of said components and complex as well as to methods of preparing same.

### 2. BACKGROUND OF THE INVENTION

Polyadenylation of precursor mRNA (pre-mRNAs) is an obligatory step in the maturation of most eukaryotic transcripts. The addition of poly(A) (polyadenosine) tails promote transcription termination and export of the mRNA from the nucleus. Furthermore, the poly(A) tails have the function to increase the efficiency of translation initiation and to help to stabilize mRNAs. Polyadenylation occurs posttranscriptionally in the nucleus of eukaryotic cells in two tightly coupled steps: the endonucleolytic cleavage of the precursor and the addition of a poly(A) tail.

In the yeast Saccharomyces cerevisiae, the pre-mRNA 3′-end processing signals are not as well conserved, as in mammalian cells (see below). In addition to the cleavage and polyadenylation site, two cis-acting elements, called the efficiency element and the positioning element, are found upstream of the cleavage site. Efficiency elements contain the sequence UAUAUA (or close variants thereof) and are often repeated. The sequence AAUAAA and several related sequences can function as a positioning element.

Fractionation of yeast extracts led to the separation of protein factors that are required for mRNA 3′-end formation in vitro. The cleavage reaction requires cleavage factors I and II (CF I and CF II), whereas polyadenylation involves CF I, polyadenylation factor I (PF I) and poly(A) polymerase (Pap1).

CF I can be separated into two activities, CF IA and CF IB. CF IA is needed for both processing steps and is a heterotetrameric protein with subunits of 38, 50, 70 and 76 kDa that are encoded by the RNA5, CLP1, PCF1 1 and RNA14 genes. Rna14 shares significant sequence similarity to the 77 kDa subunit of mammalian cleavage stimulation factor (CstF) and Rna15 contains a RNA-binding domain homologous to that of the 64 kDa subunit of CstF.

In addition to the above mentioned four CFI subunits, Pab1 (poly(A) binding protein) was identified in purified CFI fractions. Both biochemical and genetic data indicate an involvement of Pab1 in poly(A) length control. CF IB consists of a single protein called Nab4/Hrp1 and is required for cleavage site selection and polyadenylation. A multiprotein complex which has CFII-PF I (=CPF) activity consists of nine polypeptides: Pap1 (poly(A) polymerase), Pta1, Pfs1, Pfs2, Fip1, Cft1/Yhh1, Cft2/Ydh1, Ysh1/Brr5, and Yth1. Pap1, a 64 kDa protein, was the first component of the yeast 3′-end formation complex to be purified to homogeneity. Pta1 is a 90 kDa protein which is required for both cleavage and polyadenylation of mRNA precursors. Pfs2 is a 53 kDa protein that contains seven WD40 repeats. Pfs2 has been shown to directly interact with subunits of CFII-PF1 and CFIA and is thought to function in the assembly and stabilization of the 3′-end processing complex. Fip1 has been demonstrated to physically interact with Pap1, Yth1 and Rna14 and it is believed that it tethers Pap1 to its substrate during polyadenylation. Cft1/Yhh1, Cft2/Ydh1, Ysh1/Brr5, and Yth1 are the counterparts of the four subunits of the mammalian cleavage and polyadenylation specificity factor, CPSF160, CPSF100, CPSF73 and CPSF30, respectively.

Furthermore TIF4632 has been found to interact with Pab1 (see Table 1)

For the mammalian system, various data have been presented which have given evidence both for a conserved mechanism and also showed some differences between the yeast and the mammalian structures.

The composition and function of the mammalian complex based on the data to date is as follows:

The cleavage and polyadenylation factor (CPSF) is composed of 4 subunits: CPSF160 (involved in mRNA and poly(A) polymerase (PAP) binding), CPSF100, CPSF 73 and CPSF30 (involved in mRNA and PABII binding).

CPSF binds the AAUAAA hexanucleotides. CPSF links the mRNA 3′-end processing to the transcription. CPSF exists as a stable complex with the transcription factor TFIID complex. The 160 kDa subunit of CPSF binds to several hTAFII. TFIID recruits CPSF to the RNA polymerase II pre-initiation complex. Upon transcriptional activation CPSF dissociates from TFII and associates with the elongating RNA pol II (CTD carboxy-terminal domain of the largest subunit of the RNA polymerase II). CPSF is thought to travel with RNA pol II until they reach the polyadenylation site, where CPSF can bind the AAUAAA element. CPSF is required for the termination of transcription.

The interaction between CPSF and the AAUAAA element is weak and not so specific. The binding of CPSF to the hexanucleotide is greatly enhanced by a 2nd component of the poly-adenylation machinery, the cleavage stimulation factor (CstF), which binds the G-U rich motif. CstF also binds the RNA pol II through its 50 kDa subunit (CstF50). Furthermore, CstF50 binds another component of the transcriptional machinery: BRCA1 associated RING domain protein (BARD1). BARD1 also interacts with RNA pol II. BARD1-CstF50 interaction inhibits polyadenylation in vitro and may prevent inappropriate mRNA processing during transcription. CstF is composed of 3 subunits: CstF64 (binds mRNA and symplekin (yeast homolog: Pta1), CstF77 (binds CPSF160, CstF64, CstF50) and CstF50 (binds RNA pol II and BARDL). The co-operative binding of CPSF and CstF to the polyadenylation site forms a ternary complex, which functions to recruit the other components of the polyadenylation machinery to the cleavage site: the two cleavage factors (CFIm and CFIIm) and the poly(A) polymerase (PAP).

CFIm is an heterodimer of 4 subunits 72, 68, 59, 25 components: one essential, CFIImA and one stimulatory, CFIIB. CFIImA contains hPCF11p and hClp1p (binds cPSF and CF I). CF IImB contains no factors previously shown to be involved in 3′-end processing and may be a new 3′-end processing factor. Although the identity of the proteins that perform the cleavage step is still unknown, it is well established that both CFIm and CFIIm are required. The reaction products of the cleavage suggest that a metal ion is involved. Surprisingly, PAP (but not its catalytic activity) is required for the cleavage.

After the cleavage step CstF, CFIm and CFIIm are dispensable. PAP bound to CPSF (through its 160 kD subunit) can start polyadenylating the cleaved 3′-end, but at that step, the process is very inefficient. The poly(A) binding protein II (PAB II) can bind the nascent poly(A) chain as soon as it reaches a minimal length of 10 poly(A). PAB II also interacts with the CPSF30. The binding of PAB II greatly stabilizes PAP at the 3′-end of the mRNA, supporting the progressive synthesis of a long poly(A) tail. In the nucleus, the length of the poly(A) tail is restricted to about 250 poly(A). This size restriction is probably achieved through stoichiometric binding of multiple PAB II. It is not yet known how the incorporation of a certain amount of PAB II in the complex terminates processive elongation.

CstF is part of the mammalian 3′-end processing complex and is a heterotrimeric protein with subunits of 77, 64 and 50 kDa. CstF-50 has been shown to interact with the BRCA1-associated protein BARD1 and this interaction suppresses the nuclear mRNA polyadenylation machinery in vivo. In a recent study it was found that treatment of cells with DNA damage-inducing agents causes a transient, but specific, inhibition of mRNA 3′-end processing in cell extracts. This inhibition reflects the BARD1/CstF interaction and involves enhanced formation of a CstF/BARD1/BRCA1 complex. A tumor-associated germline mutation in BARD1 decreases binding to CstF-50 and renders the protein inactive in polyadenylation inhibition. These results support the existence of a link between mRNA 3′-end formation and DNA repair/tumor suppression. The in vivo function of these interactions may be to inhibit the cleavage and polyadenylation of pre-mRNAs on polymerase molecules that are stalled at sites of DNA repair.

Cleavage stimulation factor (CstF) is one of the multiple factors required for mRNA polyadenylation in mammalians. CstF-64 may play a role in regulating gene expression and cell growth in B cells. The concentration of one CstF subunit (CstF-64) increases during activation of B cells, and this is sufficient to switch IgM heavy chain mRNA expression from membrane-bound to secreted form. Reduction in CstF-64 causes reversible cell cycle arrest in G0/G1 phase, while depletion results in apoptotic cell death.

In contrast to what is observed in yeast, the sequence elements in mammals, which specify the site of cleavage and polyadenylation, flank the site of endonucleolytic attack. One is the hexanucleotide AAUAAA found 10-30 bases upstream of the cleavage/polyadenylation site. The second is a G-U-rich motif located 20-40 bases downstream of the cleavage/polyadenylation site. These two elements and their spacing determine the site of cleavage/polyadenylation and also the strength of the polyadenylation signal.

Some other elements, like sequences upstream of the AAUAAA (upstream sequence elements, USEs) play regulatory roles.

A schematic presentation of the motifs underlying mammalian polyadenylation and yeast polyadenylation are shown in FIG. 1. A review on the formation of mRNA 3′-ends in eukaryotes is given in Zhao, Hyman and Moore in Microbiology and Molecular Biology Reviews, 1999, pp. 405-445. A comparison of mammalian and yeast pre-mRNA 3′-end processing is also given in Keller and Minvielle-Sebastia in Nucleus and gene expression in Current Opinion in Cell Biology, 1997, Vol. 9, pp. 329-336.

There are diseases which involve defects in the function of the polyadenylation machinery.

Many viruses interact directly with components of the mRNA processing machinery.

The herpes simplex virus type 1 (HSV-1) immediate early (alpha) protein ICP27 is an essential regulatory protein that is involved in the shutoff of host protein synthesis. It affects mRNA processing at the level of both polyadenylation and splicing. During polyadenylation, ICP27 appears to stimulate 3′ mRNA processing at selected poly(A) sites. The opposite effect occurs on host cell splicing. That is, during HSV-1 infection, an inhibition in host cell splicing requires ICP27 expression. This contributes to the shutoff of host protein synthesis by decreasing levels of spliced cellular mRNAs available for translation. A redistribution of splicing factors regulated by ICP27 has also been seen.

Epstein-Barr virus BMLF1 gene product EB2 seems to affect mRNA nuclear export of intronless mRNAs and pre-mRNA 3′ processing. EB2 contains an Arg-X-Pro tripeptide repeated eight times, similar to that described as an mRNA-binding domain in the herpes simplex virus type 1 protein US11.

Interestingly, both viruses have been found to precede the onset of lymphomas. Influenza A virus NS1A protein binds the 30 kDa subunit of the cleavage and polyadenylation specificity factor (CPSF), NS1 protein (NS1A protein) via its effector domain targets the poly(A)-binding protein II (PABII) of the cellular 3′-end processing machinery. In vitro the NS1A protein binds the PABII protein, and in vivo causes PABII protein molecules to relocalize from nuclear speckles to a uniform distribution throughout the nucleoplasm. In vitro the NS1A protein inhibits the ability of PABII to stimulate the processive synthesis of long poly(A) tails catalyzed by poly(A) polymerase (PAP). Such inhibition also occurs in vivo in influenza virus-infected cells. Consequently, although the NS1A protein also binds the 30 kDa subunit of the cleavage and polyadenylation specificity factor (CPSF), 3′ cleavage of some cellular pre-mRNAs still occurs in virus-infected cells, followed by the PAP-catalyzed addition of short poly(A) tails. Subsequent elongation of these short poly(A) tails is blocked because the NS1A protein inhibits PABII function. The NS1 effector domain functionally interacts with the cellular 30 kDa subunit of CPSF, an essential component of the 3′ end processing machinery of cellular pre-mRNAs.

Metachromatic leukodystrophy (MLD) is a lysosomal storage disorder caused by the deficiency of arylsulfatase A (ASA). A substantial ASA deficiency has also been described in clinically healthy persons, a condition for which the term pseudodeficiency was introduced. The mutations characteristic for the pseudodeficiency (PD) allele have been identified. Sequence analysis revealed two A-G transitions. One of them changes the first polyadenylation signal downstream of the stop codon from AATAAC to AGTAAC. This causes a severe deficiency of a 2.1-kilobase (kb) mRNA species. The deficiency of the 2.1-kb RNA species provides an explanation for the diminished synthesis of ASA seen in pseudodeficiency fibroblasts.

MLD patients have been identified who are homozygous for the ASA-PD allele and it is thought that the allele might play a role in the development and progression of disease.

There is a tight link between cell cycle control and polyadenylation machinery suggesting an important role of this machinery in the development of cancer. Cyclin-dependent enzymes seem to regulate the activity of the polyadenylation machinery. The amounts of some factors of the mRNA 3′ processing machinery (CstF) increase in mitotically active cells in phases of the cell cycle preceding DNA synthesis. The amount of the 64-kDa subunit CstF-64 increases 5-fold during the G0 to S phase transition and concomitant proliferation induced by serum in 3T6 fi-broblasts. The increase in CstF-64 is associated with the G0 to S phase transition. Cdc2-cyclin B phosphorylates PAP at the Ser-Thr-rich region.

However, as it seems now, most diseases associated with defects in mRNA processing are caused by mutations in cis-acting elements that disrupt sequences essential for pre-mRNA splicing. These can be canonical sequences at the intron-exon border or located within an exon. They directly affect the expression of a single mutated gene. Approximately 15% of the nucleotide substitutions that cause human diseases disrupt pre-mRNA splicing. Thus these diseases do not seem to be directly caused by alterations in the polyadenyation/cleavage-machinery.

However, since recently evidence for a number of interrelationships between polyadenylation/cleavage and splicing is accumulating (for review see Zhao, Hyman and Moore in Microbiology and Molecular Biology Reviews, 1999, pp. 405-445), it might very well be that alterations in the 3′-end processing machinery contribute to the etiology of these diseases.

Examples of diseases caused by incorrect splicing are mentioned below:

Amyotrophic lateral sclerosis (ALS) is a neurodegenerative disease involving degeneration of cortical motor neurons and spinal/bulbar motor neurons. In the sporadic form of the disease, the neuron degeneration is caused by excessive extracellular glutamate. The glutamate transporter functional in the CNS is the astrocyte EAAT2 which is altered in ALS. The pre-mRNA for EAAT2 is aberrantly spliced in the brain regions affected. The reason for this is still unknown, but the defect lies probably in one or a few auxiliary splicing factors that regulate the splicing of a sub-set of pre-mRNA in these cells. The factors have not yet been identified.

The human papillomavirus (HPV) E2 protein plays an important role in transcriptional regulation of viral genes as well as in viral DNA replication. HPV-5 (an EV epidermodysplasia verruciformis-HPV) protein can specifically interact with cellular splicing factors including a set of prototypical SR proteins and two snRNP-associated proteins (Lai, Teh et al. 1999, J. Biol. Chem. 274, pages 11832-41). Interestingly all these three viruses have been associated with cancer progression. Papillomavirus infection precedes cervical cancer, whereas EBV and HSV-8 have been described in association with lymphomas.

In hepatocellular carcinoma, there is a defect in mRNA splicing. In this disease, there are anti-nuclear antibodies to a 64 kD protein, which has splicing factor motifs. A defect in the regulatory subunit 3 of the protein phosphatase 1(PP1) has been found in haematological malignancies and in lung, ovarian, colorectal and gastric cancers. Low PP1 activity has been observed also in acute myelogenous leukaemia.

Heterogeneous nuclear ribonucleoproteins (hnRNP) associate with pre-mRNA and have a role in RNA processing and splice site selection. HnRNP A2 shows a marked overexpression in lung cancer and brain tumours and has thus been used as a biomarker for these tumor types.

The development of antinuclear antibodies (ANA) in malignancies has been described but its mechanism is still not understood. A great diversity of ANA specificities is found in hepatocellular carcinoma. In hepatoma sera antibodies co-localize with non-snRNP splicing factor SC35, suggesting that the antigenic targets might be involved in mRNA splicing. Hepatocellular carcinoma has a significantly higher frequency of ANA than chronic hepatitis C, chronic hepatitis B, alcoholic liver cirrhosis or healthy donors.

In some autoimmune diseases, a possible link has been detected to a preceding virus infection, like Epstein-Barr virus in SLE. Furthermore it seems that even vaccination is potentially dangerous: a candidate for cytomegalovirus CMV vaccine is glycoprotein gB (UL55). Immunization with an adenovirus-gB construct (Ad-gB) not only induces a significant anti-viral response, but a significant IgG auto-antibody response (p>0.005) to the U1-70 kDa spliceosome protein. Auto-antibodies to U1-70 kDa are part of the anti-ribonucleoprotein response seen in systemic lupus erythematosus and mixed connective tissue disease.

At least two molecules which are also part of the complex are known to be inhibited by natural toxins or treatment against various diseases.

Protein phosphatase 1 is inhibited by several natural product toxins.

The marine toxins include the cyanobacteria-derived cyclic heptapeptide microcystin-LR and the polyether fatty acid okadaic acid from dinoflagellate sources. They bind to a common site on PP1. The dephosphorylation of PP1 is inhibited (among other serine/threonine phosphatases PP2A, PP2B, PP2C and PP5/T/K/H ) by Fumonisin B1 (FB1), a mycotoxin produced by the fungus Fusarium moniliforme. This is a common contaminant of corn, and is suspected to be a cause of human esophageal cancer. FB1 is hepatotoxic and hepatocarcinogenic in rats, although the mechanisms involved have not been clarified.

Viral proteins are able to interfere with PP1 activity:

The transcription factor EBNA2 of the Epstein-Barr virus induces the expression of LMP1 onco-gene in human B-cells. EBNA2A from an EBV-immortalized B-cell line co-immunopurifies with a PP1-like protein. A PP1-like activity in nuclear extracts from EBV-immortalized B-cell line can be inhibited by a GST-EBNA2A fusion product.

Poly(A)polymerase (PAP) is affected by anticancer drugs and is inhibited by some antiviral agents.

Anticancer drugs:

Most anticancer drugs act through the mechanism of apoptosis. Apoptosis may be regulated at all levels of gene expression including the addition of the poly(A) tail to the 3′ end of mRNAs. Drug combinations are more effective than single drugs and various chemotherapeutic strategies have therefore been developed. Dimethylsulfoxide (DMSO) in combination with interferon (IFN) results in pronounced PAP dephosphorylation, activity reduction and apoptosis of HeLa cells.

Purine and pyrimidine analogues often affect PAP activity. They are potentially useful agents for chemotherapy of cancer diseases. The anticancer drugs 5-Fluorouracil (5-FU), interferon and tamoxifen mediate both partial dephosphorylation and inactivation of poly(A) polymerase (PAP).

PAP (from isolated hepatic nuclei) is inhibited by cordycepin 5′-triphosphate. The nucleoside analogue cordycepin is a therapeutic agent for TdT+(terminal deoxynucleotidyl transferase positive) leukemia. In the presence of an adenosine deaminase inhibitor, deoxycoformycin (dCF), cordycepin is cytotoxic to leukemic TdT+ cells. A cordycepin analog of (2′-5′) oligo(A) which can be synthesized enzymatically from cordycepin 5′-triphosphate and the core cordycepin analog can replace human fibroblast interferon in preventing the transformation of human lymphocytes after infection with Epstein-Barr virus B95-8 (EBV). The core cordycepin analog is not cytotoxic to uninfected lymphocytes and proliferating lymphoblasts.

Not only is PAP affected by anticancer drugs, but it has a possible use as a tumor marker involved in cell commitment and/or induction of apoptosis and could be used to evaluate tumor cell sensitivity to anticancer treatment.

Antiviral drugs:

Ara-ATP (arabinofuranosyladenosine triphosphate) is an antiherpetic drug that inhibits herpes simplex virus replication. It inhibits poly(A) polymerase activity by competing with ATP. It blocks both cleavage and polyadenylation reactions by interacting with the ATP-binding site on poly(A) polymerase, the activity of which is essential for the cleavage reaction.

Purine and pyrimidine analogues are also used as antiviral agents. As an example, the most extensively used drug against HSV is idoxyuridine, the 5′-amino analog of thymidine.

A decrease in herpes simplex virus transcription and perturbation of RNA polyadenylation is induced by 5′-amino-5′-deoxythymidine (AdThd).

The cleavage stimulation factor (CSTF):

Treatment with hydroxyurea or ultraviolet light strongly, but transiently, inhibits 3′ cleavage. This is accompanied by increased amounts of a CstF/BARD1/BRCA1 complex, though the amount of these proteins remains the same.

Despite the large body of information already available from the prior art concerning the cleavage/polyaderiylation machinery of precursor mRNA up to now not all components of the machinery are known not to speak of the composition of the complex as a whole.

### 3. SUMMARY OF THE INVENTION

An object of the present invention was to identify the components of the cleavage/polyadenylation machinery of precursor mRNA and provide new components of the cleavage/polyadenylation machinery to provide the machinery and to provide new targets for therapy.

By applying the process according to the invention to the isolation of the polyadenylation/cleavage machinery from yeast 32 new components could be identified which are Act1 (SEQ ID:1), Cka1 (SEQ ID:7), Eft2 (SEQ ID 11), Eno2 (SEQ ID: 13), Glc7 (SEQ ID:15), Gpm1 (SEQ ID:17), Hhf2 (SEQ ID:21), Hta1 (SEQ ID:23), Hsc82 (SEQ ID:25), Imd2 (SEQ ID:27), Imd4 (SEQ ID:29), Met6 (SEQ ID:31), Pdc1 (SEQ ID:39), Pfk1 (SEQ ID:41), Ref2 (SEQ ID:47), Sec13 (SEQ ID:53), Sec31 (SEQ ID:55), Ssa3 (SEQ ID:57), Ssu72 (SEQ ID: 59), Taf60 (SEQ ID:61), Tkl1 (SEQ ID:65), Tsa1 (SEQ ID: 67), Tye7 (SEQ ID: 69), Vid24 (SEQ ID:71), Vps3 (SEQ ID: 73), Ycl046w (SEQ ID: 79), Ygr156w (SEQ ID: 81), Yhl035c (SEQ ID:83), Ykl018w (SEQ ID:85), Ylr221c (SEQ ID: 87), Yml030w (SEQ ID:91) and Yor179c (SEQ ID:93).

Said object is further achieved by the characterisation of Ycl046w (SEQ ID: 79), Ygr156w (SEQ ID: 81), Yhl035c (SEQ ID:83), Ykl018w (SEQ ID:85), Ylr221c (SEQ ID: 87), Yml030w (SEQ ID:91) and Yor179c (SEQ ID:93) as components of the cleavage/polyadenylation machinery.

The invention thus relates to:

An isolated complex selected from complex (I) and comprising

(a) a first protein, or a functionally active fragment or functionally active derivative thereof, which first protein is selected from the group of proteins in Table 1, column A, or a mammalian homolog thereof, or a variant of said protein encoded by a nucleic acid that hybridizes to the nucleic acid of said protein or its complement under low stringency conditions; and

(b) a second protein, or a functionally active fragment or functionally active derivative thereof, which second protein is selected from the group of proteins in Table 1, column B, or a mammalian homolog thereof, or a variant of said protein encoded by a nucleic acid that hybridizes to the nucleic acid of said protein or its complement under low stringency conditions, wherein said first protein and said second protein are members of a native cellular Polyadenylation-complex, and wherein said low stringency conditions comprise hybridization in a buffer comprising 35% formamide, 5×SSC, 50 mM Tris-HCl (pH 7.5), 5 mM EDTA, 0.02% PVP, 0.02% Ficoll, 0.2% BSA, 100 ug/ml denatured salmon sperm DNA, and 10% (wt/vol) dextran sulfate for 18-20 hours at 40° C., washing in a buffer consisting of 2×SSC, 25 mM Tris-HCl (pH 7.4), 5 mM EDTA, and 0.1% SDS for 1.5 hours at 55° C., and washing in a buffer consisting of 2×SSC, 25 mM Tris-HCl (pH 7.4), 5 mM EDTA, and 0.1% SDS for 1.5 hours at 60° C. and a complex (II) comprising at least two second proteins.

Furthermore, the invention relates to an isolated complex comprising all proteins in column C of table 1, or the mammalian homologs of those proteins, or variants of said proteins encoded by nucleic acid that hybridises to the nucleic acid of any of said proteins or its complements under low stringency conditions, wherein proteins are members of a native cellular complex, and wherein said low stringency conditions comprise hybridization in a buffer comprising 35% formamide, 5×SSC, 50 mM Tris-HCl (pH 7.5), 5 mM EDTA, 0.02% PVP, 0.02% Ficoll, 0.2% BSA, 100 ug/ml denatured salmon sperm DNA, and 10% (wt/vol) dextran sulfate for 18-20 hours at 40° C., washing in a buffer consisting of 2×SSC, 25 mM Tris-HCl (pH 7.4), 5 mM EDTA, and 0.1% SDS for 1.5 hours at 55° C., and washing in a buffer consisting of 2×SSC, 25 mM Tris-HCl (pH 7.4), 5 mM EDTA, and 0.1% SDS for 1.5 hours at 60° C.

Furthermore, the invention relates to an isolated complex that comprises all but 1,2,3,4, 5, 6, 7, 8, 9, 10, 11, 12, 13, 14, 15, 16, 17,18,19,20,21,22,23,24,25,26,27 or 28 of all proteins in column C of table 1.

Furthermore, the invention relates to the complex as described above comprising a functionally active derivative of said first protein and/or a functionally active derivative of said second protein, wherein the functionally active derivative is a fusion protein comprising said first protein or said second protein fused to an amino acid sequence different from the first protein or second protein, respectively.

In a preferred embodiment of the present invention, the protein components of the complex are vertebrate homologs of the yeast proteins, or a mixture of yeast and vertebrate homolog proteins. In a more preferred embodiment, the protein components of the complex are mammalian homologs of the yeast proteins, or a mixture of yeast and mammalian homolog proteins. In particular aspects,n the native component proteins, or derivatives or fragments of the complex are obtained from a mammal such as mouse, rat, pig, cow, dog, monkey, human, sheep or horse. In another preferred embodiment, the protein components of the complex are human homologs of the yeast proteins, or a mixture of yeast and human homolog proteins. In yet another preferred embodiment, the protein components of the complex are a mixture of yeast, vertebrate, mammalian and/or human proteins.

Furthermore, the invention relates to a complex as described aboveof claim that is involved in the 3′ end processing activity. Such a complex might also exist as a module or subcomplex of a larger physiological protein complex or assembly.

Furthermore, the invention relates to a complex as described above comprising a fragment of said first protein and/or a fragment of said second protein, which fragment binds to another protein component of said complex.

Furthermore, the invention relates to a complex as described above, wherein the functionally active derivative is a fusion protein comprising said first protein or said second protein preferentially fused to an affinity tag or label.

It is further directed to complexes comprising a fusion protein which comprises a component of the complex or a fragment thereof linked via a covalent bond to an amino acid sequence different from said component protein, as well as nucleic acids encoding the protein, fusions and fragments thereof. For example, the non-component protein portion of the fusion protein, which can be added to the N-terminal, the C-terminal or inserted into the amino acid sequence of the complex component can comprise a few amino acids, which provide an epitope that is used as a target for affinity purification of the fusion protein and/or complex.

Furthermore the invention relates to a process for processing RNA comprising the step of bringing into contact any of the complexes described above with RNA, such that RNA is processed.

Furthermore, the invention relates to an antibody or a fragment of said antibody containing the binding domain thereof, which binds the complex as described above of claim and which does not bind the first protein when uncomplexed or the second protein when uncomplexed.

Furthermore, the invention relates to a pharmaceutical composition comprising the protein complex as described above and a pharmaceutically acceptable carrier.

Moreover, the present invention provides a process for the identification and/or preparation of an effector of a composition according to the invention which process comprises the steps of bringing into contact the composition of the invention or of a component thereof with a compound, a mixture of compounds or a library of compounds and determining whether the compounds or certain compounds of the mixture or library bind to the composition of the invention and/or a component thereof and/or affects the biological activity of such a composition or component and then optionally further purifying the compound positively tested as effector by such a process.

A major application of the composition according to the invention results in the identification of an active agent capable of binding thereto. Hence, the compositions of the invention are useful tools in screening for new pharmaceutical drugs.

Furthermore, the invention relates to a method for screening for a molecule that modulates directly or indirectly the function, activity, composition or formation of the complex as described above comprising the steps of:

(a) exposing said complex, or a cell or organism containing said complex to one or more candidate molecules; and

(b) determining the amount of, the 3′ end processing activity for mRNA of, or protein components of, said complex, wherein a change in said amount, activity, or protein components relative to said amount, activity or protein components in the absence of said candidate molecules indicates that the molecules modulate function, activity or composition of said complex.

Furthermore, the invention relates to a method as described above, wherein the amount of said complex is determined.

Furthermore, the invention relates to a method as described above, wherein the activity of said complex is determined.

Furthermore, the invention relates to a method as described above, wherein said determining step comprises isolating from the cell or organism said complex to produce said isolated complex and contacting said isolating complex with the substrate under conditions conducive to binding to the complex.

Furthermore, the invention relates to a method as described above, wherein the protein components of said complex are determined.

Furthermore, the invention relates to a method as described above, wherein said determining step comprises determining whether any of the proteins listed in column B of table 1 of said complex or the mammalian homologs thereof, or variant of said proteins encoded by a nucleic acid that hybridises to the nucleic acids of any of said proteins or its complements under low stringency conditions, is present in the complex, wherein said low stringency conditions comprise hybridization in a buffer comprising 35% formamide, 5×SSC, 50 mM Tris-HCl (pH 7.5), 5 mM EDTA, 0.02% PVP, 0.02% Ficoll, 0.2% BSA, 100 ug/ml denatured salmon sperm DNA, and 10% (wt/vol) dextran sulfate for 18-20 hours at 40° C., washing in a buffer consisting of 2×SSC, 25 mM Tris-HCl (pH 7.4), 5 mM EDTA, and 0.1% SDS for 1.5 hours at 55° C., and washing in a buffer consisting of 2×SSC, 25 mM Tris-HCl (pH 7.4), 5 mM EDTA, and 0.1% SDS for 1.5 hours at 60° C.

Furthermore, the invention relates to a method as described above, wherein said method is a method of screening for a drug for treatment or prevention of diseases and disorders, preferably diseases or disorders such as infectious diseases; viral infections such as herpes simplex infections, Epstein-Barr-infections, influenza; metabolic disease such as metachromatic leukodystrophy; neurodegenerative disorders such as amyotrophic lateral sclerosis and cancer.

Furthermore, the invention relates to a method for screening for a molecule that binds the complex as described above comprising the following steps:

(a) exposing said complex, or a cell or organism containing said complex, to one or more candidate molecules; and

(b) determining whether said complex is bound by any of said candidate molecules.

Furthermore, the invention relates to a method for diagnosing or screening for the presence of a disease or disorder or a predisposition for developing a disease or disorder in a subject, which disease or disorder is characterized by an aberrant amount of, the 3′ end processing activity for mRNA biochemical activity of, or component composition or formation of, the complex as described above, comprising determining the amount of, the 3′ end processing activity for mRNA of, or protein components of, said complex in a sample derived from a subject, wherein a difference in said amount, activity, or protein components of, said complex in an analogous sample from a subject not having the disease or disorder or predisposition indicates the presence in the subject of the disease or disorder or predisposition.

Furthermore, the invention relates to a method as described above, wherein the amount of said complex is determined.

Furthermore, the invention relates to a method as described above, wherein the activity of said complex is determined.

Furthermore, the invention relates to a method as described above, wherein said determining step comprises isolating from the cell or organism said complex to produce said isolated complex and contacting said isolating complex with the substrate under conditions conducive to binding to the complex.

Furthermore, the invention relates to a method as described above, wherein the protein components of said complex are determined.

Furthermore, the invention relates to a method as described above, wherein said determining step comprises determining whether any of the proteins listed in column B of table 1 of said complex or the mammalian homologs thereof, or variant of said proteins encoded by a nucleic acid that hybridises to the nucleic acids of any of said proteins or its complements under low stringency conditions, is present in the complex, wherein said low stringency conditions comprise hybridization in a buffer comprising 35% formamide, 5×SSC, 50 mM Tris-HCl (pH 7.5), 5 mM EDTA, 0.02% PVP, 0.02% Ficoll, 0.2% BSA, 100 ug/ml denatured salmon sperm DNA, and 10% (wt/vol) dextran sulfate for 18-20 hours at 40° C., washing in a buffer consisting of 2×SSC, 25 mM Tris-HCl (pH 7.4), 5 mM EDTA, and 0.1% SDS for 1.5 hours at 55° C., and washing in a buffer consisting of 2×SSC, 25 mM Tris-HCl (pH 7.4), 5 mM EDTA, and 0.1% SDS for 1.5 hours at 60° C.

Furthermore, the invention relates to a method for treating or preventing a disease or disorder characterized by an aberrant amount of, the 3′ end processing activity for mRNA of, or component composition or formation of, the complex as described above, comprising administering to a subject in need of such treatment or prevention a therapeutically effective amount of one or more molecules that modulate the amount of, the 3′ end processing activity for mRNA of, or protein components or formation of, said complex.

Furthermore, the invention relates to a method as described above, wherein said disease or disorder involves decreased levels of the amount or activity of said complex. Furthermore, the invention relates to a method as described above, wherein said disease or disorder involves increased levels of the amount or activity of said complex.

Furthermore, the invention relates to the use of a molecule that modulates the amount of, the 3′ end processing activity for mRNA of, or protein components or formation of the complex as described above for the manufacture of a medicament for the treatment or prevention of a disease or disorder, preferably diseases or disorders such as infectious diseases; viral infections such as herpes simplex infections, Epstein-Barr-infections, influenza; metabolic disease such as metachromatic leukodystrophy; neurodegenerative disorders such as amyotrophic lateral sclerosis; cancer

Furthermore, the invention relates to a kit comprising in one or more containers

(a) an isolated first protein, or a functionally active fragment or functionally active derivative thereof selected from the proteins in column A of table 1 of a given complex or a mammalian homolog thereof, or a variant of said protein encoded by a nucleic acid that hybridises to the nucleic acid of said protein or its complement under low stringency conditions; and

(b) an isolated second protein, or a functionally active fragment or functionally active derivative thereof selected from the proteins in column B of table 1 of a given complex or a mammalian homolog thereof, or a variant of said protein encoded by a nucleic acid that hybridises to the nucleic acid of said protein or its complement under low stringency conditions, wherein said first and said second protein are members of a native cellular complex, and wherein said low stringency conditions comprise hybridization in a buffer comprising 35% formamide, 5×SSC, 50 mM Tris-HCl (pH 7.5), 5 mM EDTA, 0.02% PVP, 0.02% Ficoll, 0.2% BSA, 100 ug/ml denatured salmon sperm DNA, and 10% (wt/vol) dextran sulfate for 18-20 hours at 40° C., washing in a buffer consisting of 2×SSC, 25 mM Tris-HCl (pH 7.4), 5 mM EDTA, and 0.1% SDS for 1.5 hours at 55° C., and washing in a buffer consisting of 2×SSC, 25 mM Tris-HCl (pH 7.4), 5 mM EDTA, and 0.1% SDS for 1.5 hours at 60° C.

Furthermore, the invention relates to a kit comprising in a container the isolated complex as described above or the antibody as described above, optionally together with further reagents and working instructions. The further reagents may be, for example, buffers, substrates for enzymes but also carrier material such as beads, filters, microarrays and other solid carries. The working instructions may indicate how to use the ingredients of the kit in order to perform a desired assay.

Furthermore, the invention relates to such kits for use in processing of RNA and for use in the diagnosis, prognosis and screening in or for the diseases mentioned above.

Furthermore, the invention relates to a complex as described above, or the antibody or fragment as described above, for use in a method of diagnosing a disease or disorder, preferably the diseases or disorders as mentioned above.

Furthermore, the invention relates to a method for the production of a pharmaceutical composition comprising carrying out the method as described above to identify a molecule that modulates the function, activity or formation of said complex, and further comprising mixing the identified molecule with a pharmaceutically acceptable carrier.

Furthermore, the invention relates to a process for preparing complex as described above and optionally the components thereof comprising the following steps: expressing such a protein in a target cell, isolating the protein complex which is attached to the tagged protein, and optionally disassociating the protein complex and isolating the individual complex members.

Furthermore, the invention relates to the process as described above characterized in that the tagged protein comprises two different tags which allow two separate affinity purification steps.

Furthermore, the invention relates to the process as described above, characterized in that two tags are separated by a cleavage site for a protease.

Furthermore, the invention relates to a component of the said complex obtainable by a process as described above.

The present invention further relates to a composition, preferably a protein complex, which is obtainable by the method comprising the following steps: tagging a protein as defined above, i.e. a protein which forms part of a protein complex, with a moiety, preferably an amino acid sequence, that allows affinity purification of the tagged protein and expressing such protein in a target cell and isolating the protein complex which is attached to the tagged protein. The details of such purification are described in WO 00/09716 and in Rigaut, G. et al. (1999), Nature Biotechnology, Vol. 17 (10): 1030-1032 and further herein below. The tagging can essentially be performed with any moiety which is capable of providing a specific interaction with a further moiety, e.g. in the sense of a ligand receptor interaction, antigen antibody interaction or the like. The tagged protein can also be expressed in an amount in the target cell which comes close to the physiological concentration in order to avoid a complex formation merely due to high concentration of the expressed protein but not reflecting the natural occurring complex.

In a further preferred embodiment, the composition is obtained by using a tagged protein which comprises two different tags which allow two different affinity purification steps. This measure allows a higher degree of purification of the composition in question. In a further preferred embodiment the tagged protein comprises two tags that are separated by a cleavage site for a protease. This allows a step-by-step purification on affinity columns.

Furthermore, the invention relates to a complex as described above and/or protein thereof as a target for an active agent of a pharmaceutical, preferably a drug target in the treatment or prevention of disease or disorder, preferably diseases or disorders as mentioned above.

Furthermore, the invention relates to the Ycl046w (SEQ ID: 59), Ygr156w (SEQ ID: 61), Yhl035c (SEQ ID:63), Ykl018w (SEQ ID:179), Ylr221c (SEQ ID: 67), Yml030w (SEQ ID:69), and Yor17c (SEQ ID:71), the mammalian homologs/orthologs of said proteins and functionally active fragments and derivatives of said proteins and the mammalian homologs thereof carrying one or more amino acid substitutions, deletions and/or additions and the nucleic acid encoding said proteins or said homologs, orthologs and functionally active fragments and derivatives thereof.

Such a nucleic acid may be used for example to express a desired tagged protein in a given cell for the isolation of a complex or component according to the invention. Such a nucleic acid may also be used for the identification and isolation of genes from other organisms by cross species hybridization.

The present invention further relates to a construct, preferably a vector construct, which comprises a nucleic acid as described above. Such constructs may comprise expression controlling elements such as promoters, enhancers and terminators in order to express the nucleic acids in a given host cell, preferably under conditions which resemble the physiological concentrations.

The present invention further relates to a host cell containing a construct as defined above.

Such a host cell can be, e.g., any eukaryotic cell such as yeast, plant or mammalian, whereas human cells are preferred. Such host cells may form the starting material for isolation of a complex according to the present invention.

Animal models and methods of screening for modulators (i.e., agonists, and antagonists) of the amount of, activity of, or protein component composition of, a complex of the present invention are also provided.

### 3.1 Definitions

The term “mammalian homolog” or “homologous gene products” as used herein means a component protein of the cleavage/polyadenylation machinery of a mammal which performs the same function as the corresponding yeast protein. Such homologs are also termed “orthologue gene products”. The algorithm for the detection of orthologue gene pairs from yeast and mammalian and human uses the whole genome of these organisms. First, pairwise best hits are retrieved, using a full Smith-Waterman alignment of predicted proteins. To further improve reliability, these pairs are clustered with pairwise best hits involving Drosophila melanogaster and C. elegans proteins. Such analysis is given, e.g., in Nature, 2001, 409:860-921. The mammalian homologs of the yeast proteins according to the invention can either be isolated based on the sequence homology of the yeast genes to the mammalian genes by cloning the respective gene applying conventional technology and expressing the protein from such gene, or by isolating the mammalian proteins by isolating the analogous complex according to methods commonly known in the art, and as described in Section 6, infra.

The term “protein complex machinery” as used herein means a complex of proteins in the cell that is able to perform one or more functions of the wild type protein complex. The protein complex may or may not include and/or be associated with other molecules such as nucleic acid, such as RNA or DNA, or lipids.

As used herein, the term “percent identity” means the number of identical residues as defined by an optimal alignment using the Smith-Waterman algorithm divided by the length of the overlap multiplied by 100. The alignment is performed by the search program (W. R. Pearson, 1991, Genomics 11:635-650) with the constraint to align the maximum of both sequences.

As used herein, the term “derivatives” or analogs of component proteins a or “variants” include, but are not limited, to molecules comprising regions that are substantially homologous to the component proteins, in various embodiments, by at least 30%, 40%, 50%, 60%, 70%, 80%, 90% or 95% identity over an amino acid sequence of identical size or when compared to an aligned sequence in which the alignment is done by a computer homology program known in the art, or whose encoding nucleic acid is capable of hybridizing to a sequence encoding the component protein under stringent, moderately stringent, or nonstringent conditions. It means a protein which is the outcome of a modification of the naturally occurring protein, by amino acid substitutions, deletions and additios, respectively, which derivatives still exhibit the biological function of the naturally occurring protein although not necessarily to the same degree. The biological function of such proteins can e.g. be examined by available in vitro cleavage/polyadenylation assays as will be described below.

As used herein, the term “Therapeutics” includes, but are not limited to, a protein complex of the present invention, the individual component proteins, and analogs and derivatives (including fragments) of the foregoing (e.g., as described hereinabove); antibodies thereto (as described hereinabove); nucleic acids encoding the component protein, and analogs or derivatives, thereof (e.g., as described hereinabove); component protein antisense nucleic acids, and agents that modulate complex formation and/or activity (i.e., agonists and antagonists).

“Target for therapeutic drug” means that the respective protein (target) can bind the active ingredient of a pharmaceutical composition and thereby changes its biological activity in response to the drug binding.

“Effector of the cleavage/polyadenylation of precursor mRNA” means a compound that is capable of binding to a member of the cleavage/polyadenylation machinery thereby altering the cleavage/polyadenylation activity of the complex. This altering can be a reduction or increase in cleavage/polyadenylation activity.

The terms “polyadenylation complex”, “cleavage/polyadenylation machinery” and “cleavage/polyadenylation complex” are used interchangeably herein.

### 4. BRIEF DESCRIPTION OF THE DRAWINGS

FIG. :1 shows elements of mammalian and yeast mRNA, respectively, which are involved in polyadenylation/cleavage of precursor mRNA

FIG. 2 shows a schematic representation of the gene targeting procedure. The TAP cassette is inserted at the C-terminus of a given yeast ORF by homologous recombination, generating the TAP-tagged fusion protein.

FIG. 3. showns the protein pattern obtained by separation of the members of the polyadenylation-complex of yeast using Pta1 as a bait using TAP. Protein bands for Cft1, Cft2, Ysh1, Rna14, Pab1, Pcf11, Ref2, Pap1, Clp1, Ykl059c, Pfs2, Ygr156w, Fip1, Rna15, YKL018w, Glc7, Yth1, Ssu72, YOR179c and Pta1 (in bold) are labeled. (Further proteins identified as components of the yeast complex as described in the EXAMPLES-section (infra) are not stated in the figure)

FIG. 4 shows the protein pattern obtained by the separation of the members of the polyadenylation-complex in some of the reverse tagging-experiments and re-purification of a selection of the novel interactors. The baits using TAP used for the different experiments are given on top of each gel picture. The band constituing the protein used as the bait in the respective experiments is indicated by an arrow. Previously known members of the complex are listed in bold letters. (Note: only experiments using Cft1, Cft2, Pap1, Ref2, Ykl059c, Pfs2, YOR179c and Pta1 as a bait are shown and only the proteins bands of Cft1, Cft2, Ysh1, Rna14, Pab1, Ref2, Clp1, Ygr156w, Fip1, Glc7, Yht1, Yor179c, Pta1, Pcf11, Pab1, Ykl059c, Pfs2, Rna15, Ykl018w and Ssu72 are labelled).

### 5. DETAILED DESCRIPTION OF THE INVENTION

The present invention relates to components of the cleavage/polyadenylation machinery of pre-cursor mRNA, the complete protein complex, uses of said components and complex as well as to methods of preparing same.

This is further described below. Also a description of the newly identified components of the cleavage/polyadenylation machinery is given below.

In more detail, the present invention relates to the following embodiments: An isolated complex selected from complex (I) and comprising

(a) a first protein, or a functionally active fragment or functionally active derivative thereof, which first protein is selected from the group of proteins in Table 1, column A, or a mammalian homolog thereof, or a variant of said protein encoded by a nucleic acid that hybridizes to the nucleic acid of said protein or its complement under low stringency conditions; and

(b) a second protein, or a functionally active fragment or functionally active derivative thereof, which second protein is selected from the group of proteins in Table 1, column B, or a mammalian homolog thereof, or a variant of said protein encoded by a nucleic acid that hybridizes to the nucleic acid of said protein or its complement under low stringency conditions, wherein said first protein and said second protein are members of a native cellular Polyadenylation-complex, and wherein said low stringency conditions comprise hybridization in a buffer comprising 35% formamide, 5×SSC, 50 mM Tris-HCl (pH 7.5), 5 mM EDTA, 0.02% PVP, 0.02% Ficoll, 0.2% BSA, 100 ug/ml denatured salmon sperm DNA, and 10% (wt/vol) dextran sulfate for 18-20 hours at 40° C., washing in a buffer consisting of 2×SSC, 25 mM Tris-HCl (pH 7.4), 5 mM EDTA, and 0.1% SDS for 1.5 hours at 55° C., and washing in a buffer consisting of 2×SSC, 25 mM Tris-HCl (pH 7.4), 5 mM EDTA, and 0.1% SDS for 1.5 hours at 60° C., and a complex (II) comprising at least two second proteins.

The present invention further relates to a new protein complex which is useful for cleaving and/or polyadenylating a nucleic acid and which complex comprises at least one of the components according to the invention. Such a complex can be isolated from a natural source by applying the process according to the invention or can be reconstituted from the different components made available by the present invention.

Furthermore, the invention relates to an isolated complex comprising all proteins in column C of table 1, or the mammalian homologs of those proteins, or variants of said proteins encoded by nucleic acid that hybridises to the nucleic acid of any of said proteins or its complements under low stringency conditions, wherein proteins are members of a native cellular complex, and wherein said low stringency conditions comprise hybridization in a buffer comprising 35% formamide, 5×SSC, 50 mM Tris-HCl (pH 7.5), 5 mM EDTA, 0.02% PVP, 0.02% Ficoll, 0.2% BSA, 100 ug/ml denatured salmon sperm DNA, and 10% (wt/vol) dextran sulfate for 18-20 hours at 40° C., washing in a buffer consisting of 2×SSC, 25 mM Tris-HCl (pH 7.4), 5 mM EDTA, and 0.1% SDS for 1.5 hours at 55° C., and washing in a buffer consisting of 2×SSC, 25 mM Tris-HCl (pH 7.4), 5 mM EDTA, and 0.1% SDS for 1.5 hours at 60° C.

Furthermore, the invention relates to an isolated complex that comprises all but 1,2,3,4, 5, 6, 7, 8, 9, 10, 11, 12, 13, 14, 15, 16, 17, 18,19,20,21,22,23,24,25,26,27 or 28 of all proteins in column C of table 1.

Furthermore, the invention relates to the complex as described above comprising a functionally active derivative of said first protein and/or a functionally active derivative of said second protein, wherein the functionally active derivative is a fusion protein comprising said first protein or said second protein fused to an amino acid sequence different from the first protein or second protein, respectively.

The present invention further relates to a fusion protein comprising a component according to the invention. The fusion part, which can be added to the N-terminal, the C-terminal or into the amino acid sequence of the component according to the invention may comprise a few amino acids only e.g. at least five, which amino acids for example provide an epitope which is then be used as a target for affinity purification of the protein and the complex, respectively. Such a type of added amino acid is also termed “tag” throughout the present specification (optionally, the fusion protein may comprise even more than one such fusion partner).

In a preferred embodiment of the present invention, the protein components of the complex are vertebrate homologs of the yeast proteins, or a mixture of yeast and vertebrate homolog proteins. In a more preferred embodiment, the protein components of the complex are mammalian homologs of the yeast proteins, or a mixture of yeast and mammalian homolog proteins. In particular aspects,n the native component proteins, or derivatives or fragments of the complex are obtained from a mammal such as mouse, rat, pig, cow, dog, monkey, human, sheep or horse. In another preferred embodiment, the protein components of the complex are human homologs of the yeast proteins, or a mixture of yeast and human homolog proteins. In yet another preferred embodiment, the protein components of the complex are a mixture of yeast, vertebrate, mammalian and/or human proteins.

The mammalian homologs or “orthologues” of the yeast proteins according to the invention can either be isolated based on the sequence homology of the yeast genes to the mammalian genes by cloning the respective gene applying conventional technology and expressing the protein from such gene or by isolating the mammalian proteins according to the process of the invention as explained in more detail below.

The derivatives of the proteins according to the invention can be produced e.g. by recombinant DNA technology applying the standard technology to modify the amino acid sequence of a given protein via the modification of the underlying gene using e.g. site directed mutagenesis, etc.

A protein that shows a certain degree of identity to the naturally occurring proteins from mammals and/or yeast, respectively, can also be prepared e.g. by applying recombinant DNA technology as described above for the derivatives according to the invention. Alternatively, such protein can be isolated from natural sources by applying the process of the invention.

Furthermore, the invention relates to a complex as described above that is involved in the 3′ end processing activity. Such a complex might also exist as a module or subcomplex of a larger physiological protein complex or assembly.

Furthermore, the invention relates to a complex as described above comprising a fragment of said first protein and/or a fragment of said second protein, which fragment binds to another protein component of said complex.

Furthermore, the invention relates to a complex as described above, wherein the functionally active derivative is a fusion protein comprising said first protein or said second protein preferentially fused to an affinity tag or label.

It is further directed to complexes comprising a fusion protein which comprises a component of the complex or a fragment thereof linked via a covalent bond to an amino acid sequence different from said component protein, as well as nucleic acids encoding the protein, fusions and fragments thereof. For example, the non-component protein portion of the fusion protein, which can be added to the N-terminal, the C-terminal or inserted into the amino acid sequence of the complex component can comprise a few amino acids, which provide an epitope that is used as a target for affinity purification of the fusion protein and/or complex.

Furthermore the invention relates to a process for processing RNA comprising the step of bringing into contact any of the complexes described above with RNA, such that RNA is processed.

Furthermore, the invention relates to an antibody or a fragment of said antibody containing the binding domain thereof, which binds the complex as described above of claim and which does not bind the first protein when uncomplexed or the second protein when uncomplexed.

Furthermore, the invention relates to a pharmaceutical composition comprising the protein complex as described above and a pharmaceutically acceptable carrier.

Moreover, the present invention provides a process for the identification and/or preparation of an effector of a composition according to the invention which process comprises the steps of bringing into contact the composition of the invention or of a component thereof with a compound, a mixture of compounds or a library of compounds and determining whether the compounds or certain compounds of the mixture or library bind to the composition of the invention and/or a component thereof and/or affects the biological activity of such a composition or component and then optionally further purifying the compound positively tested as effector by such a process.

A major application of the composition according to the invention results in the identification of an active agent capable of binding thereto. Hence, the compositions of the invention are useful tools in screening for new pharmaceutical drugs.

Furthermore, the invention relates to a method for screening for a molecule that modulates directly or indirectly the function, activity, composition or formation of the complex as described above comprising the steps of:

(a) exposing said complex, or a cell or organism containing said complex to one or more candidate molecules; and

(b) determining the amount of, the 3′ end processing activity for mRNA of, or protein components of, said complex, wherein a change in said amount, activity, or protein components relative to said amount, activity or protein components in the absence of said candidate molecules indicates that the molecules modulate function, activity or composition of said complex.

Furthermore, the invention relates to a method as described above, wherein the amount of said complex is determined.

Furthermore, the invention relates to a method as described above, wherein the activity of said complex is determined.

Furthermore, the invention relates to a method as described above, wherein said determining step comprises isolating from the cell or organism said complex to produce said isolated complex and contacting said isolating complex with the substrate under conditions conducive to binding to the complex.

Furthermore, the invention relates to a method as described above, wherein the protein components of said complex are determined.

Furthermore, the invention relates to a method as described above, wherein said determining step comprises determining whether any of the proteins listed in column B of table 1 of said complex or the mammalian homologs thereof, or variant of said proteins encoded by a nucleic acid that hybridises to the nucleic acids of any of said proteins or its complements under low stringency conditions, is present in the complex, wherein said low stringency conditions comprise hybridization in a buffer comprising 35% formamide, 5×SSC, 50 mM Tris-HCl (pH 7.5), 5 mM EDTA, 0.02% PVP, 0.02% Ficoll, 0.2% BSA, 100 ug/ml denatured salmon sperm DNA, and 10% (wt/vol) dextran sulfate for 18-20 hours at 40° C., washing in a buffer consisting of 2×SSC, 25 mM Tris-HCl (pH 7.4), 5 mM EDTA, and 0.1% SDS for 1.5 hours at 55° C., and washing in a buffer consisting of 2×SSC, 25 mM Tris-HCl (pH 7.4), 5 mM EDTA, and 0.1% SDS for 1.5 hours at 60° C.

Furthermore, the invention relates to a method as described above, wherein said method is a method of screening for a drug for treatment or prevention of diseases and disorders, preferably diseases or disorders such as infectious diseases; viral infections such as herpes simplex infections, Epstein-Barr-infections, influenza; metabolic disease such as metachromatic leukodystrophy; neurodegenerative disorders such as amyotrophic lateral sclerosis and cancer.

Furthermore, the invention relates to a method for screening for a molecule that binds the complex as described above comprising the following steps:

(a) exposing said complex, or a cell or organism containing said complex, to one or more candidate molecules; and

(b) determining whether said complex is bound by any of said candidate molecules.

Furthermore, the invention relates to a method for diagnosing or screening for the presence of a disease or disorder or a predisposition for developing a disease or disorder in a subject, which disease or disorder is characterized by an aberrant amount of, the 3′ end processing activity for mRNA biochemical activity of, or component composition or formation of, the complex as described above, comprising determining the amount of, the 3′ end processing activity for mRNA of, or protein components of, said complex in a sample derived from a subject, wherein a difference in said amount, activity, or protein components of, said complex in an analogous sample from a subject not having the disease or disorder or predisposition indicates the presence in the subject of the disease or disorder or predisposition.

Furthermore, the invention relates to a method as described above, wherein the amount of said complex is determined.

Furthermore, the invention relates to a method as described above, wherein the activity of said complex is determined.

Furthermore, the invention relates to a method as described above, wherein said determining step comprises isolating from the cell or organism said complex to produce said isolated complex and contacting said isolating complex with the substrate under conditions conducive to binding to the complex.

Furthermore, the invention relates to a method as described above, wherein the protein components of said complex are determined.

Furthermore, the invention relates to a method as described above, wherein said determining step comprises determining whether any of the proteins listed in column B of table 1 of said complex or the mammalian homologs thereof, or variant of said proteins encoded by a nucleic acid that hybridises to the nucleic acids of any of said proteins or its complements under low stringency conditions, is present in the complex, wherein said low stringency conditions comprise hybridization in a buffer comprising 35% formamide, 5×SSC, 50 mM Tris-HCl (pH 7.5), 5 mM EDTA, 0.02% PVP, 0.02% Ficoll, 0.2% BSA, 100 ug/ml denatured salmon sperm DNA, and 10% (wt/vol) dextran sulfate for 18-20 hours at 40° C., washing in a buffer consisting of 2×SSC, 25 mM Tris-HCl (pH 7.4), 5 mM EDTA, and 0.1% SDS for 1.5 hours at 55° C., and washing in a buffer consisting of 2×SSC, 25 mM Tris-HCl (pH 7.4), 5 mM EDTA, and 0.1% SDS for 1.5 hours at 60° C.

Furthermore, the invention relates to a method for treating or preventing a disease or disorder characterized by an aberrant amount of, the 3′ end processing activity for mRNA of, or component composition or formation of, the complex as described above, comprising administering to a subject in need of such treatment or prevention a therapeutically effective amount of one or more molecules that modulate the amount of, the 3′ end processing activity for mRNA of, or protein components or formation of, said complex.

Furthermore, the invention relates to a method as described above, wherein said disease or disorder involves decreased levels of the amount or activity of said complex. Furthermore, the invention relates to a method as described above, wherein said disease or disorder involves increased levels of the amount or activity of said complex.

Furthermore, the invention relates to the use of a molecule that modulates the amount of, the 3′ end processing activity for mRNA of, or protein components or formation of the complex as described above for the manufacture of a medicament for the treatment or prevention of a disease or disorder, preferably diseases or disorders such as infectious diseases; viral infections such as herpes simplex infections, Epstein-Barr-infections, influenza; metabolic disease such as metachromatic leukodystrophy; neurodegenerative disorders such as amyotrophic lateral sclerosis; cancer

The present invention further relates to the use of the products according to the invention in therapy wherein the products according to the invention are useful as a target for a therapeutic drug. It is known from the prior art that mRNA 3′-end processing is involved in viral growth, in the development of cancer and in certain neurodegenerative diseases. By having identified new components of the cleavage/polyadenylation machinery the present invention, hence, offers new targets for treating viral diseases, cancer and neurodegenerative diseases. By affecting the biological activity of the components of the invention and/or by affecting the complex as a whole the cleavage/polyadenylation activity thereof can be influenced depending by the needs of the patient to be treated.

The present invention further relates to a pharmaceutical composition comprising a product according to the invention. Such pharmaceutical composition contains beside the product according to the invention as active ingredient further excipients and additives as known by a skilled person. The present invention, hence, allows the identification of new effectors which affect the biological activity of the cleavage/polyadenylation machinery of precursor RNA. Said effectors than can be used to modify the cleavage/polyadenylation machinery in a cell by introducing an effector into a cell. Moreover, the mRNA processing activity of a given cell can also be affected by introducing a product according to the invention into such cell.

Furthermore, the invention relates to a kit comprising in one or more containers

(a) an isolated first protein, or a functionally active fragment or functionally active derivative thereof selected from the proteins in column A of table 1 of a given complex or a mammalian homolog thereof, or a variant of said protein encoded by a nucleic acid that hybridises to the nucleic acid of said protein or its complement under low stringency conditions; and

(b) an isolated second protein, or a functionally active fragment or functionally active derivative thereof selected from the proteins in column B of table 1 of a given complex or a mammalian homolog thereof, or a variant of said protein encoded by a nucleic acid that hybridises to the nucleic acid of said protein or its complement under low stringency conditions, wherein said first and said second protein are members of a native cellular complex, and wherein said low stringency conditions comprise hybridization in a buffer comprising 35% formamide, 5×SSC, 50 mM Tris-HCl (pH 7.5), 5 mM EDTA, 0.02% PVP, 0.02% Ficoll, 0.2% BSA, 100 ug/ml denatured salmon sperm DNA, and 10% (wt/vol) dextran sulfate for 18-20 hours at 40° C., washing in a buffer consisting of 2×SSC, 25 mM Tris-HCl (pH 7.4), 5 mM EDTA, and 0.1% SDS for 1.5 hours at 55° C., and washing in a buffer consisting of 2×SSC, 25 mM Tris-HCl (pH 7.4), 5 mM EDTA, and 0.1% SDS for 1.5 hours at 60° C.

Furthermore, the invention relates to a kit comprising in a container the isolated complex as described above or the antibody as described above, optionally together with further reagents and working instructions. The further reagents may be, for example, buffers, substrates for enzymes but also carrier material such as beads, filters, microarrays and other solid carries. The working instructions may indicate how to use the ingredients of the kit in order to perform a desired assay.

Furthermore, the invention relates to such kits for use in processing of RNA and for use in the diagnosis, prognosis and screening in or for the diseases mentioned above.

The present invention further relates to a kit for processing RNA which kit comprises a product according to the invention. Such a kit may contain e.g. expression vectors encoding the essential components of the cleavage/polyadenylation machinery which components after being expressed can be reconstituted in order to form a biologically active cleavage/polyadenylation complex. Such a kit preferably also contains the required buffers and reagents together with the working instructions.

The present invention further relates to a kit for the diagnosis of diseases of mammals which kit comprises a product according to the invention. As stated above the polyadenylation/cleavage machinery is involved in a large number of diseases. If said machinery activity is changed due to e.g. mutations of some components thereof and/or effectors, this may have severe implications on the affected organism. The kit according to the present invention containing the products according to the invention allows to examine as to whether the cleavage/polyadenylation machinery in a given sample might show some defects. Such a kit may be used to determine genetic defects of the genes encoding the components of the cleavage/polyadenylation machinery.

Furthermore, the invention relates to a complex as described above, or the antibody or fragment as described above, for use in a method of diagnosing a disease or disorder, preferably the diseases or disorders as mentioned above.

Furthermore, the invention relates to a method for the production of a pharmaceutical composition comprising carrying out the method as described above to identify a molecule that modulates the function, activity or formation of said complex, and further comprising mixing the identified molecule with a pharmaceutically acceptable carrier.

Furthermore, the invention relates to a process for preparing complex as described above and optionally the components thereof comprising the following steps: expressing such a protein in a target cell, isolating the protein complex which is attached to the tagged protein, and optionally disassociating the protein complex and isolating the individual complex members.

Furthermore, the invention relates to the process as described above characterized in that the tagged protein comprises two different tags which allow two separate affinity purification steps.

Furthermore, the invention relates to the process as described above, characterized in that two tags are separated by a cleavage site for a protease.

Furthermore, the invention relates to a component of the said complex obtainable by a process as described above.

The present invention further relates to a composition, preferably a protein complex, which is obtainable by the method comprising the following steps: tagging a protein as defined above, i.e. a protein which forms part of a protein complex, with a moiety, preferably an amino acid sequence, that allows affinity purification of the tagged protein and expressing such protein in a target cell and isolating the protein complex which is attached to the tagged protein. The details of such purification are described in WO 00/09716 and in Rigaut, G. et al. (1999), Nature Biotechnology, Vol. 17 (10): 1030-1032 and further herein below. The tagging can essentially be performed with any moiety which is capable of providing a specific interaction with a further moiety, e.g. in the sense of a ligand receptor interaction, antigen antibody interaction or the like. The tagged protein can also be expressed in an amount in the target cell which comes close to the physiological concentration in order to avoid a complex formation merely due to high-concentration of the expressed protein but not reflecting the natural occurring complex.

In a further preferred embodiment, the composition is obtained by using a tagged protein which comprises two different tags which allow two different affinity purification steps. This measure allows a higher degree of purification of the composition in question. In a further preferred embodiment the tagged protein comprises two tags that are separated by a cleavage site for a protease. This allows a step-by-step purification on affinity columns.

Furthermore, the invention relates to a complex as described above and/or protein thereof as a target for an active agent of a pharmaceutical, preferably a drug target in the treatment or prevention of disease or disorder, preferably diseases or disorders as mentioned above.

Furthermore, the invention relates to the Ycl046w (SEQ ID: 59), Ygr156w (SEQ ID: 61), Yhl035c (SEQ ID:63), Ykl018w (SEQ ID:179), Ylr221c (SEQ ID: 67), Yml030w (SEQ ID:69), and Yor17c (SEQ ID:71), the mammalian homologs/orthologs of said proteins and functionally active fragments and derivatives of said proteins and the mammalian homologs thereof carrying one or more amino acid substitutions, deletions and/or additions and the nucleic acid encoding said proteins or said homologs, orthologs and functionally active fragments and derivatives thereof.

Such a nucleic acid may be used for example to express a desired tagged protein in a given cell for the isolation of a complex or component according to the invention. Such a nucleic acid may also be used for the identification and isolation of genes from other organisms by cross species hybridization.

The component according to the invention is preferably a protein component which can be further modified e.g. by carbohydrate residues. The components can either be prepared by recombinant DNA technology based on the sequences provided by the present invention or can be isolated from a biological source by using the process according to the invention.

The present invention further relates to a fusion protein comprising a component according to the invention. The fusion part, which can be added to the N-terminal, the C-terminal or into the amino acid sequence of the component according to the invention may comprise a few amino acids only e.g. at least five, which amino acids for example provide an epitope which is then be used as a target for affinity purification of the protein and the complex, respectively. Such a type of added amino acid is also termed “tag” throughout the present specification (optionally, the fusion protein may comprise even more than one such fusion partner).

The present invention further relates to a construct, preferably a vector construct, which comprises a nucleic acid as described above. Such constructs may comprise expression controlling elements such as promoters, enhancers and terminators in order to express the nucleic acids in a given host cell, preferably under conditions which resemble the physiological concentrations.

The present invention further relates to a construct which comprises the nucleic acid according to the invention and at least one further nucleic acid which is normally not associated with the nucleic acid according to the invention. Such a construct is preferably a vector which preferably is capable of replicating in a given cell and contains the necessary transcription control elements for expressing the nucleic acid according to the invention in a given expression system. Moreover, such vector construct may contain selection markers.

The present invention further relates to a host cell containing a construct as defined above.

Such a host cell can be, e.g., any eukaryotic cell such as yeast, plant or mammalian, whereas human cells are preferred. Such host cells may form the starting material for isolation of a complex according to the present invention.

The present invention also relates to a host cell containing a nucleic acid according to the invention or a construct according to the invention. Such a host cell may contain an expression vector which encodes a component according to the invention which component may serve as a bait in order to isolate the further proteins of the complex and which at least partly interact with the bait. Host cells can be prokaryotic and eukaryotic cells, whereas mammalian host cells are preferred.

Animal models and methods of screening for modulators (i.e., agonists, and antagonists) of the amount of, activity of, or protein component composition of, a complex of the present invention are also provided.

Below is a more detailed list of the newly identified components of the polyadenylation complex (see also Tab. 1). The Accession-Number stated is the GenBank-Accession number for the protein.

Act1: Is a known and essential protein (GenBankAcc. No. BAA21512.1), which has been shown to be involved in Pol II transcription and has been found to be associated with histone acetylation. It serves as a structural protein.

Cka1: Is a known and non-essential protein (GenBank Acc. No. CAA86916.1), which has been found to be involved in Polymerase III transcription and has been found to be associated with the Casein kinase II complex.

Eft2: The translation elongation factor EF-2 is a known protein involved in protein synthesis (GenBank AAB64827.1)

Eno2: Is a known and essential protein (GenBank Acc. No. AAB68019.1). It has been shown to have lyase activity and is known to be involved in carbohydrate metabolism.

Glc7 (YER133w) is also a known protein (GenBank Acc. No. AAC03231.1). It is also an essential protein and is a Type I protein serine threonine phosphatase which has been implicated in distinct cellular roles, such as carbohydrate metabolism, meiosis, mitosis and cell polarity. Its occurrence in the cleavage/polyadenylation machinery has not been known before.

Gpm1: This protein is a phosphoglycerate mutase that converts 2-phosphoglyvcerate to 3-phosphoglycerate in glycolysis. It is an essential protein (GenBank: CAA81994.1)

Hhf2: Is a known and non-essential protein (GenBank Acc. No. CAA95892.1) which has been shown to be involved in DNA-binding. It has previously been linked to Histone octamer and the RNA polymerase I upstream activation factor.

Hta1: Is a known and non-essential protein (GenBank Acc. No. CAA88505.1) which has DNA-binding capability and has been shown to be involved in polymerase II transcription.

Hsc82: Is a non-essential protein so far being associated with protein folding. (GenBank Acc. No: CAA89919.1)

Imd2: Is an Inosine-5′-monophosphate dehydrogenase so far being associated with nucleotide metabolism. It is non-essential. (GenBank Acc.-No.: AAB69728.1)

Imd4: Is a non-essential protein with similiarity to Imd2 so far being associated with nucleotide metabolism (GenBank Acc-No.: CAA86719.1)

Met6: Is a homocysteine methyltransferase so far being associated with amino-acid metabolism (GenBank Acc.-No.: AAB64646.1)

Pdc1: Is a pyruvate decarboxylase isozymel so far being associated with carbohydrate metabolism (GenBank Acc.-No.: CAA97573.1)

Pfk1: Is a known protein (GenBank Acc. No. CAA97268.1) which has previously been described as part of the phosphofructokinase complex.

Ref2 (YDR195w) is a known protein (GenBank Acc. No. CAA88708.1). It is a non-essential gene product. It has been shown to be involved in 3′-end formation prior to the final polyadenylation step. However, Ref2 has never been identified before as a component of the 3′-end processing machinery. Ref2 has been shown to interact with Glc7, another new component of the cleavage/polyadenylation machinery.

Sec13: Is a known and essential protein (GenBank Acc. No AAB67426.1).

Sec31: Is a known and essential protein (GenBank Acc. No. CAA98772.1)

Ssa3: Is a known and non-essential protein (GenBank Acc. No. CAA84896.1) which so far has been implicated with protein folding/protein transport.

Ssu72 (YNL222w) is also a known protein (GenBank Acc. No. CAA96125.1) and is an essential phylogenetically conserved protein which has been shown to interact with the general transcription factor TFIIB (Sua7). TFIIB is an essential component of the RNA polymerase II (RNAP II) core transcriptional machinery. It is thought that this interaction plays a role in the mechanism of start site selection by RNAP II. The finding according to the present invention that Ssu72 is associated with Pta1 is likely to be relevant since it is believed that mRNA 3′-end formation is linked with other nuclear processes like transcription, capping and splicing. Furthermore, Ssu 72 has also been clearly identified in a “reverse tagging experiment” as explained herein below by using some of the Pta1 associated proteins as bait. However, when Ssu72 itself was used as a bait associated proteins were not found most likely due to the fact that the addition of a C-terminal tag renders Ssu72 non-functional.

Taf60: Is a known and essential protein (GenBank Acc. No. CAA96819.1) which has been shown to be involved in Polymerase II transcription.

Tkl1: Is a non-essential transketolase so far being associated with amino-acid metabolism and carbohydrate metabolism (GenBank Acc-No.: CAA89191.1)

Tsa1: Translation initiation factor elF5 which so far has been to shown to catalyze hydrolysis of GTP on the 40S ribosomal subunit-initiation complex followed by joining to 60S ribosomal subunit. (GenBank Acc.-No.: CAA92145.1)

Tye7: Is a known protein (GenBank Acc. No. CAA99671.1). It has been shown to be a basic helix-loop-helix transcription factor.

Vid24: Is a known and non-essential protein (GenBank Acc. No. CAA89320.1) which has previously been associated with protein degradation and vesicular transport.

Vps53: Is a known protein (GenBank Acc. No. CAA89320.1) which has been found to play a role in protein sorting.

YCL046w: Is a non-essential protein (GenBank Acc. No. CAA42371.1).

YGR156w is the protein product of an essential gene. This protein also contains a RNA binding motif. (GenBankAcc. No. CAA97170.1).

YHL035c: Is a known and non-essential protein (GenBank Acc. No. AAB65047.1). It is a member of the ATP-binding cassette superfamily.

YKL018w is also an essential protein containing a WD40 domain which is a typical protein binding domain. (GenBank Acc. No. CAA81853.1)

YLR221c: Is a protein of unknown function (GenBank Acc. No.AAB67410.1)

YML030w: Is a protein of unknown function (GenBank Acc. No. CAA86625.1)

YOR179c shows significant sequence similarity to Ysh1(GenBank Acc. No. CAA99388.1)

Two further proteins for which binary interactions with members of the polyadenylation complex as known so far have been shown before have also been purified with the complex:

YKL059c: is the product of an essential gene and is a zinc binding protein containing a C2HC Zinc finger. The presence of this domain predicts a RNA binding function of YKL059c. We believe the corresponding gene product is identical to Pfs1, a protein which has been mentioned in several publications, but which has never been annotated in the databases (for review see Keller, W. and Minvielle-Sebastia (1997). Curr Opin Cell Biol 11: 352-357). (GenBank Acc. No. CAA81896.1)

Tif4632: Is a known and non-essential protein (GenBank Acc. No. CAA96751.1) which has been shown to have an RNA-binding/translation factor activity and is involved in protein synthesis.

Tables:

Table 1: Composition of the Complex (Cleavage/polyadenylation machinery):

First column (‘Entry point’) lists the bait proteins (TAP-tag fusion proteins) that have been chosen for the isolation of the given complex. Note: in several cases, different baits have been used for validation in reverse tagging experiments.

Second column (‘Interactions’) briefly lists any known interactions between different members of the complex (Abbrevations: ‘2-hybrid’: interaction as identified in yeast-2-hybrid screens; ‘far-western’: interaction as identified in far-western experiments; ‘coipp’: interaction as identified by co-immunoprecipitation experiments; ‘high-throughput 2 hybrid’: interaction as identified by high-throughput yeast-2-hybrid screens; ‘copurification’: interaction as identified by copurification experiments; ‘immuno-affinity-columns’: interaction as identified in experiments using immuno-affinity columns; ‘in vitro binding’: interaction as identified in in-vitro-binding experiments. If a core complex has been known previously containing several of the identified proteins, the name of the complex is stated.

Third colum (‘Proteins found’) lists all proteins which have been identified in the particular complex.

Fourth column (‘COLUMN A, ‘Known components of the complex’) lists the components of the complex as found by Cellzome, which have been known to interact with other members of the complex as identified herein. (see also third column).

Firth column (‘COLUMN B, ‘Novel proteins’) lists the novel members of the complex as provided in the invention.

Sixth column (‘Column C, cleavage/polyadenylation machinery’): lists again all components of the cleavage/polyadenylation machinery as identified herein

Seventh column (COLUMN C, ‘Activity of the complex’): List the biochemical activities of the newly identified complex.

Eighth column (COLUMN D, ‘Proteins of unknown function’): Separately lists again the members of the newly identified complex which previously have not been annotated.

Ninth column (‘localization’) indicates the localization of the identified complex (Abbevations: c: cytoplasma; b: membrane; e: ER/Golgi/vesicles; m: mitochondria; n: nucleus; u: unknown)

Table 2: Individual Yeast Proteins of the Complexes

A) Table lists in alphabetical order all yeast proteins which have been identified as members of the complex presented herein. Furthermore, the SEQ ID of the proteins are listed as used herein. Further columns lists the Accession-Number of the respective sequences in MIPS, SWISS-PROT, SGD and Genbank. In addition, where applicable, the GenBank accession numbers of the respective orthologues in humans, C.elegans and Drosophila are listed.

B) Table lists again the proteins and SEQ ID as in part A. In addition, the table contains an overview about what has been previously reported on the protein, the biochemical function thereof and the cellular function thereof as stated in YPD (Constanzo, M. C. et al., 2001, Nucl. Acid Res, 29: 75-9; Hodges, P. E. et al., 1999, Nucl. Acids Res 27: 69-73).

Table 3: Medical Application of the Complex:

First column (‘Name of complex’) lists again the name of the complex as used herein.

Second column (‘Cellular role’) lists keyword on the cellular role of the complex

Third column (‘Medical applications’) lists disorder, diseases, disease areas etc. which are treatable and/or preventable and/or diagnosable etc. by therapeutics and methods interacting with/acting via the complex.

Table 4: Characterization of previously undescribed individual proteins of the complexes:

The table provides data on proteins which have not been annotated previously but which have now been linked to a functional complex as described in table 2. Names are listed on the left. In addition the table contains a list of motifs found by sequence analysis which has been part of the invention provided herein. Futhermore, the predicted known human orthologues are listed on the right (By SWISS-PROT Accession numbers). Used Abbrevations are listed at the end of the table. The function of the individual proteins as deduced from the association with the complex, the sequence analysis and the analysis of the predicted ortholgues is listed in the second column (‘Putative function’).

Table 5: Overview on Experimental Steps: The tables illustrates the construction of a yeast strain expressing a TAP-tagged bait in a high-throuphput fashion.

Table 6: Known and Novel Components of the yeast mRNA 3′-end processing machinery (the cleavage/polyadenylation complex): Top part of the table states the different known subcomponents of the polyadenylation complex, the function thereof, the proteins constituting the different subcomplexes as known so far (including their molecular weight and sequence motifs contained in the protein). Bottom part lists the novel components of the complex as provided herein

### 5.1. Protein Complexes

The protein complexes of the present invention and their component proteins are described in the Tables 1,2,3,4,6 (whereas Table 6 gives an overview on the construction of the yeast strains). The protein complexes and component proteins can be obtained by methods well known in the art for protein purification and recombinant protein expression. For example, the protein complexes of the present invention can be isolated using the TAP method described in Section 6, infra, and in WO 00/09716 and Rigaut et al., 1999, Nature Biotechnology 17:1030-1032, which are each incorporated by reference in their entirety. Additionally, the protein complexes can be isolated by immunoprecipitation of the component proteins and combining the immunoprecipitated proteins. The protein complexes can also be produced by recombinantly expressing the component proteins and combining the expressed proteins.

The nucleic and amino acid sequences of the component proteins of the protein complexes of the present invention are provided herein (SEQ ID NOS:1-2670), and can be obtained by any method known in the art, e.g., by PCR amplification using synthetic primers hybridizable to the 3′ and 5′ ends of each sequence, and/or by cloning from a cDNA or genomic library using an oligonucleotide specific for each nucleotide sequence.

Homologs (e.g., nucleic acids encoding component proteins from other species) or other related sequences (e.g., variants, paralogs) which are members of a native cellular protein complex can be obtained by low, moderate or high stringency hybridization with all or a portion of the particular nucleic acid sequence as a probe, using methods well known in the art for nucleic acid hybridization and cloning.

Exemplary moderately stringent hybridization conditions are as follows: prehybridization of filters containing DNA is carried out for 8 hours to overnight at 65° C. in buffer composed of 6×SSC, 50 mM Tris-HCl (pH 7.5), 1 mM EDTA, 0.02% PVP, 0.02% Ficoll, 0.02% BSA, and 500 μg/ml denatured salmon sperm DNA. Filters are hybridized for 48 hours at 65° C. in prehybridization mixture containing 100 μg/ml denatured salmon sperm DNA and 5-20×10⁶ cpm of ³²P-labeled probe. Washing of filters is done at 37° C. for 1 hour in a solution containing 2×SSC, 0.01% PVP, 0.01% Ficoll, and 0.01% BSA. This is followed by a wash in 0.1×SSC at 50° C. for 45 min before autoradiography. Alternatively, exemplary conditions of high stringency are as follows: e.g., hybridization to filter-bound DNA in 0.5 M NaHPO₄, 7% sodium dodecyl sulfate (SDS), 1 mM EDTA at 65° C., and washing in 0.1×SSC/0.1% SDS at 68° C. (Ausubel F. M. et al., eds., 1989, Current Protocols in Molecular Biology, Vol. 1, Green Publishing Associates, Inc., and John Wiley & sons, Inc., New York, at p. 2.10.3). Other conditions of high stringency which may be used are well known in the art. Exemplary low stringency hybridization conditions comprise hybridization in a buffer comprising 35% formamide, 5×SSC, 50 mM Tris-HCl (pH 7.5), 5 mM EDTA, 0.02% PVP, 0.02% Ficoll, 0.2% BSA, 100 μg/ml denatured salmon sperm DNA, and 10% (wt/vol) dextran sulfate for 18-20 hours at 40° C., washing in a buffer consisting of 2×SSC, 25 mM Tris-HCl (pH 7.4), 5 mM EDTA, and 0.1% SDS for 1.5 hours at 55° C., and washing in a buffer consisting of 2×SSC, 25 mM Tris-HCl (pH 7.4), 5 mM EDTA, and 0.1% SDS for 1.5 hours at 60° C.

For recombinant expression of one or more of the proteins, the nucleic acid containing all or a portion of the nucleotide sequence encoding the protein can be inserted into an appropriate expression vector, i.e., a vector that contains the necessary elements for the transcription and translation of the inserted protein coding sequence. The necessary transcriptional and translational signals can also be supplied by the native promoter of the component protein gene, and/or flanking regions.

A variety of host-vector systems may be utilized to express the protein coding sequence. These include but are not limited to mammalian cell systems infected with virus (e.g., vaccinia virus, adenovirus, etc.); insect cell systems infected with virus (e.g., baculovirus); microorganisms such as yeast containing yeast vectors; or bacteria transformed with bacteriophage, DNA, plasmid DNA, or cosmid DNA. The expression elements of vectors vary in their strengths and specificities. Depending on the host-vector system utilized, any one of a number of suitable transcription and translation elements may be used.

In a preferred embodiment, a complex of the present invention is obtained by expressing the entire coding sequences of the component proteins in the same cell, either under the control of the same promoter or separate promoters. In yet another embodiment, a derivative, fragment or homolog of a component protein is recombinantly expressed. Preferably the derivative, fragment or homolog of the protein forms a complex with the other components of the complex, and more preferably forms a complex that binds to an anti-complex antibody.

The present invention further relates to an antibody which reacts with a product according to the invention. Such an antibody might be used e.g. during purification of the machinery from a given source by affinity purification methods. Moreover, the antibody might be used in diagnosis in order to detect changes and/or modifications of a product according to the invention in a given sample.

Any method available in the art can be used for the insertion of DNA fragments into a vector to construct expression vectors containing a chimeric gene consisting of appropriate transcriptional/translational control signals and protein coding sequences. These methods may include in vitro recombinant DNA and synthetic techniques and in vivo recombinant techniques (genetic recombination). Expression of nucleic acid sequences encoding a component protein, or a derivative, fragment or homolog thereof, may be regulated by a second nucleic acid sequence so that the gene or fragment thereof is expressed in a host transformed with the recombinant DNA molecule(s). For example, expression of the proteins may be controlled by any promoter/enhancer known in the art. In a specific embodiment, the promoter is not native to the gene for the component protein. Promoters that may be used can be selected from among the many known in the art, and are chosen so as to be operative in the selected host cell.

In a specific embodiment, a vector is used that comprises a promoter operably linked to nucleic acid sequences encoding a component protein, or a fragment, derivative or homolog thereof, one or more origins of replication, and optionally, one or more selectable markers (e.g., an antibiotic resistance gene).

In another specific embodiment, an expression vector containing the coding sequence, or a portion thereof, of a component protein, either together or separately, is made by subcloning the gene sequences into the EcoRI restriction site of each of the three pGEX vectors (glutathione S-transferase expression vectors; Smith and Johnson, 1988, Gene 7:31-40). This allows for the expression of products in the correct reading frame.

Expression vectors containing the sequences of interest can be identified by three general approaches: (a) nucleic acid hybridization, (b) presence or absence of “marker” gene function, and (c) expression of the inserted sequences. In the first approach, coding sequences can be detected by nucleic acid hybridization to probes comprising sequences homologous and complementary to the inserted sequences. In the second approach, the recombinant vector/host system can be identified and selected based upon the presence or absence of certain “marker” functions (e.g., resistance to antibiotics, occlusion body formation in baculovirus, etc.) caused by insertion of the sequences of interest in the vector. For example, if a component protein gene, or portion thereof, is inserted within the marker gene sequence of the vector, recombinants containing the encoded protein or portion will be identified by the absence of the marker gene function (e.g., loss of beta-galactosidase activity). In the third approach, recombinant expression vectors can be identified by assaying for the component protein expressed by the recombinant vector. Such assays can be based, for example, on the physical or functional properties of the interacting species in in vitro assay systems, e.g., formation of a complex comprising the protein or binding to an anti-complex antibody.

Once recombinant component protein molecules are identified and the complexes or individual proteins isolated, several methods known in the art can be used to propagate them. Using a suitable host system and growth conditions, recombinant expression vectors can be propagated and amplified in quantity. As previously described, the expression vectors or derivatives which can be used include, but are not limited to, human or animal viruses such as vaccinia virus or adenovirus; insect viruses such as baculovirus, yeast vectors; bacteriophage vectors such as lambda phage; and plasmid and cosmid vectors.

In addition, a host cell strain may be chosen that modulates the expression of the inserted sequences, or modifies or processes the expressed proteins in the specific fashion desired. Expression from certain promoters can be elevated in the presence of certain inducers; thus expression of the genetically-engineered component proteins may be controlled. Furthermore, different host cells have characteristic and specific mechanisms for the translational and post-translational processing and modification (e.g., glycosylation, phosphorylation, etc.) of proteins. Appropriate cell lines or host systems can be chosen to ensure that the desired modification and processing of the foreign protein is achieved. For example, expression in a bacterial system can be used to produce an unglycosylated core protein, while expression in mammalian cells ensures “native” glycosylation of a heterologous protein. Furthermore, different vector/host expression systems may effect processing reactions to different extents.

In other specific embodiments, a component protein or a fragment, homolog or derivative thereof, may be expressed as fusion or chimeric protein product comprising the protein, fragment, homolog, or derivative joined via a peptide bond to a heterologous protein sequence of a different protein. Such chimeric products can be made by ligating the appropriate nucleic acid sequences encoding the desired amino acids to each other by methods known in the art, in the proper coding frame, and expressing the chimeric products in a suitable host by methods commonly known in the art. Alternatively, such a chimeric product can be made by protein synthetic techniques, e.g., by use of a peptide synthesizer. Chimeric genes comprising a portion of a component protein fused to any heterologous protein-encoding sequences may be constructed.

In particular, protein component derivatives can be made by altering their sequences by substitutions, additions or deletions that provide for functionally equivalent molecules. Due to the degeneracy of nucleotide coding sequences, other DNA sequences that encode substantially the same amino acid sequence as a component gene or cDNA can be used in the practice of the present invention. These include but are not limited to nucleotide sequences comprising all or portions of the component protein gene that are altered by the substitution of different codons that encode a functionally equivalent amino acid residue within the sequence, thus producing a silent change. Likewise, the derivatives of the invention include, but are not limited to, those containing, as a primary amino acid sequence, all or part of the amino acid sequence of a component protein, including altered sequences in which functionally equivalent amino acid residues are substituted for residues within the sequence resulting in a silent change. For example, one or more amino acid residues within the sequence can be substituted by another amino acid of a similar polarity that acts as a functional equivalent, resulting in a silent alteration. Substitutes for an amino acid within the sequence may be selected from other members of the class to which the amino acid belongs. For example, the nonpolar (hydrophobic) amino acids include alanine, leucine, isoleucine, valine, proline, phenylalanine, tryptophan and methionine. The polar neutral amino acids include glycine, serine, threonine, cysteine, tyrosine, asparagine, and glutamine. The positively charged (basic) amino acids include arginine, lysine and histidine. The negatively charged (acidic) amino acids include aspartic acid and glutamic acid.

In a specific embodiment, up to 1%, 2%, 5%, 10%, 15% or 20% of the total number of amino acids in the wild type protein are substituted or deleted; or 1, 2, 3, 4, 5, or 6 amino acids are inserted, substituted or deleted relative to the wild type protein.

In a specific embodiment of the invention, the nucleic acids encoding a protein component and protein components consisting of or comprising a fragment of or consisting of at least 6 (continuous) amino acids of the protein are provided. In other embodiments, the fragment consists of at least 10, 20, 30, 40, or 50 amino acids of the component protein. In specific embodiments, such fragments are not larger than 35, 100 or 200 amino acids. Derivatives or analogs of component proteins include, but are not limited, to molecules comprising regions that are substantially homologous to the component proteins, in various embodiments, by at least 30%, 40%, 50%, 60%, 70%, 80%, 90% or 95% identity over an amino acid sequence of identical size or when compared to an aligned sequence in which the alignment is done by a computer homology program known in the art, or whose encoding nucleic acid is capable of hybridizing to a sequence encoding the component protein under stringent, moderately stringent, or nonstringent conditions.

The protein component derivatives and analogs of the invention can be produced by various methods known in the art. The manipulations which result in their production can occur at the gene or protein level. For example, the cloned gene sequences can be modified by any of numerous strategies known in the art (Sambrook et al., 1989, Molecular Cloning, A Laboratory Manual, 2d Ed., Cold Spring Harbor Laboratory Press, Cold Spring Harbor, N.Y.). The sequences can be cleaved at appropriate sites with restriction endonuclease(s), followed by further enzymatic modification if desired, isolated, and ligated in vitro. In the production of the gene encoding a derivative, homolog or analog of a component protein, care should be taken to ensure that the modified gene retains the original translational reading frame, uninterrupted by translational stop signals, in the gene region where the desired activity is encoded.

Additionally, the encoding nucleic acid sequence can be mutated in vitro or in vivo, to create and/or destroy translation, initiation, and/or termination sequences, or to create variations in coding regions and/or form new restriction endonuclease sites or destroy pre-existing ones, to facilitate further in vitro modification. Any technique for mutagenesis known in the art can be used, including but not limited to, chemical mutagenesis and in vitro site-directed mutagenesis (Hutchinson et al., 1978, J. Biol. Chem 253:6551-6558), amplification with PCR primers containing a mutation, etc.

Once a recombinant cell expressing a component protein, or fragment or derivative thereof, is identified, the individual gene product or complex can be isolated and analyzed. This is achieved by assays based on the physical and/or functional properties of the protein or complex, including, but not limited to, radioactive labeling of the product followed by analysis by gel electrophoresis, immunoassay, cross-linking to marker-labeled product, etc.

The component proteins and complexes may be isolated and purified by standard methods known in the art (either from natural sources or recombinant host cells expressing the complexes or proteins), including but not restricted to column chromatography (e.g., ion exchange, affinity, gel exclusion, reversed-phase high pressure, fast protein liquid, etc.), differential centrifugation, differential solubility, or by any other standard technique used for the purification of proteins. Functional properties may be evaluated using any suitable assay known in the art.

Alternatively, once a component protein or its derivative, is identified, the amino acid sequence of the protein can be deduced from the nucleic acid sequence of the chimeric gene from which it was encoded. As a result, the protein or its derivative can be synthesized by standard chemical methods known in the art (e.g., Hunkapiller et al., 1984, Nature 310: 105-111).

Manipulations of component protein sequences may be made at the protein level. Included within the scope of the invention is a complex in which the component proteins or derivatives and analogs that are differentially modified during or after translation, e.g., by glycosylation, acetylation, phosphorylation, amidation, derivatization by known protecting/blocking groups, proteolytic cleavage, linkage to an antibody molecule or other cellular ligand, etc. Any of numerous chemical modifications may be carried out by known techniques, including but not limited to specific chemical cleavage by cyanogen bromide, trypsin, chymotrypsin, papain, V8 protease, NaBH₄, acetylation, formylation, oxidation, reduction, metabolic synthesis in the presence of tunicamycin, etc.

In specific embodiments, the amino acid sequences are modified to include a fluorescent label. In another specific embodiment, the protein sequences are modified to have a heterofunctional reagent; such heterofunctional reagents can be used to crosslink the members of the complex.

In addition, complexes of analogs and derivatives of component proteins can be chemically synthesized. For example, a peptide corresponding to a portion of a component protein, which comprises the desired domain or mediates the desired activity in vitro (e.g., complex formation) can be synthesized by use of a peptide synthesizer. Furthermore, if desired, non-classical amino acids or chemical amino acid analogs can be introduced as a substitution or addition into the protein sequence.

In cases where natural products are suspected of being mutant or are isolated from new species, the amino acid sequence of a component protein isolated from the natural source, as well as those expressed in vitro, or from synthesized expression vectors in vivo or in vitro, can be determined from analysis of the DNA sequence, or alternatively, by direct sequencing of the isolated protein. Such analysis can be performed by manual sequencing or through use of an automated amino acid sequenator.

The complexes can also be analyzed by hydrophilicity analysis (Hopp and Woods, 1981, Proc. Natl. Acad. Sci. USA 78:3824-3828). A hydrophilicity profile can be used to identify the hydrophobic and hydrophilic regions of the proteins, and help predict their orientation in designing substrates for experimental manipulation, such as in binding experiments, antibody synthesis, etc. Secondary structural analysis can also be done to identify regions of the component proteins, or their derivatives, that assume specific structures (Chou and Fasman, 1974, Biochemistry 13:222-23). Manipulation, translation, secondary structure prediction, hydrophilicity and hydrophobicity profile predictions, open reading frame prediction and plotting, and determination of sequence homologies, etc., can be accomplished using computer software programs available in the art.

Other methods of structural analysis including but not limited to X-ray crystallography (Engstrom, 1974 Biochem. Exp. Biol. 11:7-13), mass spectroscopy and gas chromatography (Methods in Protein Science, J. Wiley and Sons, New York, 1997), and computer modeling (Fletterick and Zoller, eds., 1986, Computer Graphics and Molecular Modeling, In: Current Communications in Molecular Biology, Cold Spring Harbor Laboratory, Cold Spring Harbor Press, New York) can also be employed.

### 5.2. Antibodies to Protein Complexes

According to the present invention, a protein complex of the present invention comprising a first protein, or a functionally active fragment or functionally active derivative thereof, selected from the group consisting of proteins listed in column A of table 1; and a second protein, or a functionally active fragment or functionally active derivative thereof, selected from the group consisting of proteins listed in column B of table 1, or a functionally active fragment or functionally active derivative thereof, can be used as an immunogen to generate antibodies which immunospecifically bind such immunogen. According to the present invention, also a protein complex of the present invention can be used as an immunogen to generate antibodies which immunospecifically bind to such immunogen comprising all proteins listed in column C of table 1

Such antibodies include, but are not limited to, polyclonal, monoclonal, chimeric, single chain, Fab fragments, and an Fab expression library. In a specific embodiment, antibodies to a complex comprising human protein components are produced. In another embodiment, a complex formed from a fragment of said first protein and a fragment of said second protein, which fragments contain the protein domain that interacts with the other member of the complex, are used as an immunogen for antibody production. In a preferred embodiment, the antibody specific for the complex in that the antibody does not bind the individual protein components of the complex.

Polyclonal antibodies can be prepared as described above by immunizing a suitable subject with a polypeptide of the invention as an immunogen. Preferred polyclonal antibody compositions are ones that have been selected for antibodies directed against a polypeptide or polypeptides of the invention. Particularly preferred polyclonal antibody preparations are ones that contain only antibodies directed against a polypeptide or polypeptides of the invention. Particularly preferred immunogen compositions are those that contain no other human proteins such as, for example, immunogen compositions made using a non-human host cell for recombinant expression of a polypeptide of the invention. In such a manner, the only human epitope or epitopes recognized by the resulting antibody compositions raised against this immunogen will be present as part of a polypeptide or polypeptides of the invention.

The antibody titer in the immunized subject can be monitored over time by standard techniques, such as with an enzyme linked immunosorbent assay (ELISA) using immobilized polypeptide. If desired, the antibody molecules can be isolated from the mammal (e.g., from the blood) and further purified by well-known techniques, such as protein A chromatography to obtain the IgG fraction. Alternatively, antibodies specific for a protein or polypeptide of the invention can be selected for (e.g., partially purified) or purified by, e.g., affinity chromatography. For example, a recombinantly expressed and purified (or partially purified) protein of the invention is produced as described herein, and covalently or non-covalently coupled to a solid support such as, for example, a chromatography column. The column can then be used to affinity purify antibodies specific for the proteins of the invention from a sample containing antibodies directed against a large number of different epitopes, thereby generating a substantially purified antibody composition, i.e., one that is substantially free of contaminating antibodies. By a substantially purified antibody composition is meant, in this context, that the antibody sample contains at most only 30% (by dry weight) of contaminating antibodies directed against epitopes other than those on the desired protein or polypeptide of the invention, and preferably at most 20%, yet more preferably at most 10%, and most preferably at most 5% (by dry weight) of the sample is contaminating antibodies. A purified antibody composition means that at least 99% of the antibodies in the composition are directed against the desired protein or polypeptide of the invention.

At an appropriate time after immunization, e.g., when the specific antibody titers are highest, antibody-producing cells can be obtained from the subject and used to prepare monoclonal antibodies by standard techniques, such as the hybridoma technique originally described by Kohler and Milstein, 1975, Nature 256:495-497, the human B cell hybridoma technique (Kozbor et al., 1983, Immunol. Today 4:72), the EBV-hybridoma technique (Cole et al., 1985, Monoclonal Antibodies and Cancer Therapy, Alan R. Liss, Inc., pp. 77-96) or trioma techniques. The technology for producing hybridomas is well known (see generally Current Protocols in Immunology (1994) Coligan et al. (eds.) John Wiley & Sons, Inc., New York, N.Y.). Hybridoma cells producing a monoclonal antibody of the invention are detected by screening the hybridoma culture supernatants for antibodies that bind the polypeptide of interest, e.g., using a standard ELISA assay.

Alternative to preparing monoclonal antibody-secreting hybridomas, a monoclonal antibody directed against a polypeptide of the invention can be identified and isolated by screening a recombinant combinatorial immunoglobulin library (e.g., an antibody phage display library) with the polypeptide of interest. Kits for generating and screening phage display libraries are commercially available (e.g., the Pharmacia Recombinant Phage Antibody System, Catalog No. 27-9400-01; and the Stratagene SurfZAP Phage Display Kit, Catalog No. 240612). Additionally, examples of methods and reagents particularly amenable for use in generating and screening antibody display library can be found in, for example, U.S. Pat. No. 5,223,409; PCT Publication No. WO 92/18619; PCT Publication No. WO 91/17271; PCT Publication No. WO 92/20791; PCT Publication No. WO 92/15679; PCT Publication No. WO 93/01288; PCT Publication No. WO 92/01047; PCT Publication No. WO 92/09690; PCT Publication No. WO 90/02809; Fuchs et al., 1991, Bio/Technology 9:1370-1372; Hay et al., 1992, Hum. Antibod. Hybridomas 3:81-85; Huse et al., 1989, Science 246:1275-1281; Griffiths et al., 1993, EMBO J. 12:725-734.

Additionally, recombinant antibodies, such as chimeric and humanized monoclonal antibodies, comprising both human and non-human portions, which can be made using standard recombinant DNA techniques, are within the scope of the invention. A chimeric antibody is a molecule in which different portions are derived from different animal species, such as those having a variable region derived from a murine mAb and a human immunoglobulin constant region. (See, e.g., Cabilly et al., U.S. Pat. No. 4,816,567; and Boss et al., U.S. Pat. No. 4,816,397, which are incorporated herein by reference in their entirety.) Humanized antibodies are antibody molecules from non-human species having one or more complementarily determining regions (CDRs) from the non-human species and a framework region from a human immunoglobulin molecule. (See, e.g., Queen, U.S. Pat. No. 5,585,089, which is incorporated herein by reference in its entirety.) Such chimeric and humanized monoclonal antibodies can be produced by recombinant DNA techniques known in the art, for example using methods described in PCT Publication No. WO 87/02671; European Patent Application 184,187; European Patent Application 171,496; European Patent Application 173,494; PCT Publication No. WO 86/01533; U.S. Pat. No. 4,816,567; European Patent Application 125,023; Better et al., 1988, Science 240:1041-1043; Liu et al., 1987, Proc. Natl. Acad. Sci. USA 84:3439-3443; Liu et al., 1987, J. Immunol. 139:3521-3526; Sun et al., 1987, Proc. Natl. Acad. Sci. USA 84:214-218; Nishimura et al., 1987, Canc. Res. 47:999-1005; Wood et al., 1985, Nature 314:446-449; and Shaw et al., 1988, J. Natl. Cancer Inst. 80:1553-1559); Morrison, 1985, Science 229:1202-1207; Oi et al., 1986, Bio/Techniques 4:214; U.S. Pat. No. 5,225,539; Jones et al., 1986, Nature 321:552-525; Verhoeyan et al., 1988, Science 239:1534; and Beidler et al., 1988, J. Immunol. 141:4053-4060.

Completely human antibodies are particularly desirable for therapeutic treatment of human patients. Such antibodies can be produced, for example, using transgenic mice which are incapable of expressing endogenous immunoglobulin heavy and light chains genes, but which can express human heavy and light chain genes. The transgenic mice are immunized in the normal fashion with a selected antigen, e.g., all or a portion of a polypeptide of the invention. Monoclonal antibodies directed against the antigen can be obtained using conventional hybridoma technology. The human immunoglobulin transgenes harbored by the transgenic mice rearrange during B cell differentiation, and subsequently undergo class switching and somatic mutation. Thus, using such a technique, it is possible to produce therapeutically useful IgG, IgA and IgE antibodies. For an overview of this technology for producing human antibodies, see Lonberg and Huszar, 1995, Int. Rev. Immunol. 13:65-93). For a detailed discussion of this technology for producing human antibodies and human monoclonal antibodies and protocols for producing such antibodies, see, e.g., U.S. Pat. No. 5,625,126; U.S. Pat. No. 5,633,425; U.S. Pat. No. 5,569,825; U.S. Pat. No. 5,661,016; and U.S. Pat. No. 5,545,806. In addition, companies such as Abgenix, Inc. (Freemont, Calif.), can be engaged to provide human antibodies directed against a selected antigen using technology similar to that described above.

Completely human antibodies which recognize a selected epitope can be generated using a technique referred to as “guided selection.” In this approach a selected non-human monoclonal antibody, e.g., a murine antibody, is used to guide the selection of a completely human antibody recognizing the same epitope. (Jespers et al., 1994, Bio/technology 12:899-903).

Antibody fragments that contain the idiotypes of the complex can be generated by techniques known in the art. For example, such fragments include, but are not limited to, the F(ab′)2 fragment which can be produced by pepsin digestion of the antibody molecule; the Fab′ fragment that can be generated by reducing the disulfide bridges of the F(ab′)2 fragment; the Fab fragment that can be generated by treating the antibody molecular with papain and a reducing agent; and Fv fragments.

In the production of antibodies, screening for the desired antibody can be accomplished by techniques known in the art, e.g., ELISA (enzyme-linked immunosorbent assay). To select antibodies specific to a particular domain of the complex, or a derivative thereof, one may assay generated hybridomas for a product that binds to the fragment of the complex, or a derivative thereof, that contains such a domain. For selection of an antibody that specifically binds a complex of the present, or a derivative, or homolog thereof, but which does not specifically bind to the individual proteins of the complex, or a derivative, or homolog thereof, one can select on the basis of positive binding to the complex and a lack of binding to the individual protein components.

Antibodies specific to a domain of the complex, or a derivative, or homolog thereof, are also provided.

The foregoing antibodies can be used in methods known in the art relating to the localization and/or quantification of the complexes of the invention, e.g., for imaging these proteins, measuring levels thereof in appropriate physiological samples (by immunoassay), in diagnostic methods, etc. This hold true also for a derivative, or homolog thereof of a complex.

In another embodiment of the invention (see infra), an antibody to a complex or a fragment of such antibodies containing the antibody binding domain, is a Therapeutic.

### 5.3. Diagnostic, Prognostic, and Screening Uses of Protein Complexes

The particular protein complexes of the present invention may be markers of normal physiological processes, and thus have diagnostic utility. Further, definition of particular groups of patients with elevations or deficiencies of a protein complex of the present invention, or wherein the protein complex has a change in protein component composition, can lead to new nosological classifications of diseases, furthering diagnostic ability.

Examples for diseases or disorders in which the complexes provided herein are involved and/or associated with are infectious diseases; viral infections such as herpes simplex infections, Epstein-Barr-infections, influenza; metabolic disease such as metachromatic leukodystrophy; neurodegenerative disorders such as amyotrophic lateral sclerosis and cancer.

Detecting levels of protein complexes, or individual component proteins that form the complexes, or detecting levels of the mRNAs encoding the components of the complex, may be used in diagnosis, prognosis, and/or staging to follow the course of a disease state, to follow a therapeutic response, etc.

A protein complex of the present invention and the individual components of the complex and a derivative, analog or subsequence thereof, encoding nucleic acids (and sequences complementary thereto), and anti-complex antibodies and antibodies directed against individual components that can form the complex, are useful in diagnostics. The foregoing molecules can be used in assays, such as immunoassays, to detect, prognose, diagnose, or monitor various conditions, diseases, and disorders characterized by aberrant levels of a complex or aberrant component composition of a complex, or monitor the treatment of such various conditions, diseases, and disorders.

In particular, such an immunoassay is carried out by a method comprising contacting a sample derived from a patient with an anti-complex antibody under conditions such that immunospecific binding can occur, and detecting or measuring the amount of any immunospecific binding by the antibody. In a specific aspect, such binding of antibody, in tissue sections, can be used to detect aberrant complex localization, or aberrant (e.g., high, low or absent) levels of a protein complex or complexes. In a specific embodiment, an antibody to the complex can be used to assay a patient tissue or serum sample for the presence of the complex, where an aberrant level of the complex is an indication of a diseased condition. By “aberrant levels” is meant increased or decreased levels relative to that present, or a standard level representing that present, in an analogous sample from a portion or fluid of the body, or from a subject not having the disorder.

The immunoassays which can be used include but are not limited to competitive and non-competitive assay systems using techniques such as Western blots, radioimmunoassays, ELISA (enzyme linked immunosorbent assay), “sandwich” immunoassays, immunoprecipitation assays, precipitin reactions, gel diffusion precipitin reactions, immunodiffusion assays, agglutination assays, complement-fixation assays, immunoradiometric assays, fluorescent immunoassays, protein A immunoassays, to name but a few known in the art.

Nucleic acids encoding the components of the protein complex and related nucleic acid sequences and subsequences, including complementary sequences, can be used in hybridization assays. The nucleic acid sequences, or subsequences thereof, comprising about at least 8 nucleotides, can be used as hybridization probes. Hybridization assays can be used to detect, prognose, diagnose, or monitor conditions, disorders, or disease states associated with aberrant levels of the mRNAs encoding the components of a complex as described, supra. In particular, such a hybridization assay is carried out by a method comprising contacting a sample containing nucleic acid with a nucleic acid probe capable of hybridizing to component protein coding DNA or RNA, under conditions such that hybridization can occur, and detecting or measuring any resulting hybridization.

In specific embodiments, diseases and disorders involving or characterized by aberrant levels of a protein complex or aberrant complex composition can be diagnosed, or its suspected presence can be screened for, or a predisposition to develop such disorders can be detected, by determining the component protein composition of the complex, or detecting aberrant levels of a member of the complex or un-complexed component proteins or encoding nucleic acids, or functional activity including, but not restricted to, binding to an interacting partner, or by detecting mutations in component protein RNA, DNA or protein (e.g., mutations such as translocations, truncations, changes in nucleotide or amino acid sequence relative to wild-type that cause increased or decreased expression or activity of a complex, and/or component protein. Such diseases and disorders include, but are not limited to, those described in Section 5.4 and its subsections.

By way of example, levels of a protein complex and the individual components of a complex can be detected by immunoassay, levels of component protein RNA or DNA can be detected by hybridization assays (e.g., Northern blots, dot blots, RNase protection assays), and binding of component proteins to each other (e.g., complex formation) can be measured by binding assays commonly known in the art. Translocations and point mutations in component protein genes can be detected by Southern blotting, RFLP analysis, PCR using primers that preferably generate a fragment spanning at least most of the gene by sequencing of genomic DNA or cDNA obtained from the patient, etc.

Assays well known in the art (e.g., assays described above such as immunoassays, nucleic acid hybridization assays, activity assays, etc.) can be used to determine whether one or more particular protein complexes are present at either increased or decreased levels, or are absent, in samples from patients suffering from a particular disease or disorder, or having a predisposition to develop such a disease or disorder, as compared to the levels in samples from subjects not having such a disease or disorder, or having a predisposition to develop such a disease or disorder. Additionally, these assays can be used to determine whether the ratio of the complex to the un-complexed components of the complex, is increased or decreased in samples from patients suffering from a particular disease or disorder, or having a predisposition to develop such a disease or disorder, as compared to the ratio in samples from subjects not having such a disease or disorder. In the event that levels of one or more particular protein complexes (i.e., complexes formed from component protein derivatives, homologs, fragments, or analogs) are determined to be increased in patients suffering from a particular disease or disorder, or having a predisposition to develop such a disease or disorder, then the particular disease or disorder, or predisposition for a disease or disorder, can be diagnosed, have prognosis defined for, be screened for, or be monitored by detecting increased levels of the one or more protein complexes, increased levels of the mRNA that encodes one or more members of the one or more particular protein complexes, or by detecting increased complex functional activity.

Accordingly, in a specific embodiment of the present invention, diseases and disorders involving increased levels of one or more protein complexes can be diagnosed, or their suspected presence can be screened for, or a predisposition to develop such disorders can be detected, by detecting increased levels of the one or more protein complexes, the mRNA encoding both members of the complex, or complex functional activity, or by detecting mutations in the component proteins that stabilize or enhance complex formation, e.g., mutations such as translocations in nucleic acids, truncations in the gene or protein, changes in nucleotide or amino acid sequence relative to wild-type, that stabilize or enhance complex formation.

In the event that levels of one or more particular protein complexes are determined to be decreased in patients suffering from a particular disease or disorder, or having a predisposition to develop such a disease or disorder, then the particular disease or disorder or predisposition for a disease or disorder can be diagnosed, have its prognosis determined, be screened for, or be monitored by detecting decreased levels of the one or more protein complexes, the mRNA that encodes one or more members of the particular one or more protein complexes, or by detecting decreased protein complex functional activity.

Accordingly, in a specific embodiment of the invention, diseases and disorders involving decreased levels of one or more protein complexes can be diagnosed, or their suspected presence can be screened for, or a predisposition to develop such disorders can be detected, by detecting decreased levels of the one or more protein complexes, the mRNA encoding one or more members of the one or more complexes, or complex functional activity, or by detecting mutations in the component proteins that decrease complex formation, e.g., mutations such as translocations in nucleic acids, truncations in the gene or protein, changes in nucleotide or amino acid sequence relative to wild-type, that decrease complex formation.

Accordingly, in a specific embodiment of the invention, diseases and disorders involving aberrant compositions of the complexes can be diagnosed, or their suspected presence can be screened for, or a predisposition to develop such disorders can be detected, by detecting the component proteins of one or more complexes, or the mRNA encoding the members of the one or more complexes.

The use of detection techniques, especially those involving antibodies against a protein complex, provides a method of detecting specific cells that express the complex or component proteins. Using such assays, specific cell types can be defined in which one or more particular protein complexes are expressed, and the presence of the complex or component proteins can be correlated with cell viability, state, health, etc.

Also embodied are methods to detect a protein complex of the present invention in cell culture models that express particular protein complexes or derivatives thereof, for the purpose of characterizing or preparing the complexes for harvest. This embodiment includes cell sorting of prokaryotes such as but not restricted to bacteria (Davey and Kell, 1996, Microbiol. Rev. 60:641-696), primary cultures and tissue specimens from eukaryotes, including mammalian species such as human (Steele et al., 1996, Clin. Obstet. Gynecol 39:801-813), and continuous cell cultures (Orfao and Ruiz-Arguelles, 1996, Clin. Biochem. 29:5-9). Such isolations can be used as methods of diagnosis, described, supra.

### 5.4. Therapeutic Uses of Protein Complexes

The present invention is directed to a method for treatment or prevention of various diseases and disorders by administration of a therapeutic compound (termed herein “Therapeutic”). Such “Therapeutics” include, but are not limited to, a protein complex of the present invention, the individual component proteins, and analogs and derivatives (including fragments) of the foregoing (e.g., as described hereinabove); antibodies thereto (as described hereinabove); nucleic acids encoding the component protein, and analogs or derivatives, thereof (e.g., as described hereinabove); component protein antisense nucleic acids, and agents that modulate complex formation and/or activity (i.e., agonists and antagonists).

The protein complexes, as identified herein, are implicated significantly in normal physiological processes such as RNA processing and modification.

Furthermore, the protein complexes as identified herein are implicated in processes which are implicated in or associated with pathological conditions.

Diseases and disorders which can be treated and/or prevented and/or diagnosed by Therapeutics interacting with any of the complexes provided herein are for example infectious diseases; viral infections such as herpes simplex infections, Epstein-Barr-infections, influenza; metabolic disease such as metachromatic leukodystrophy; neurodegenerative disorders such as amyotrophic lateral sclerosis and cancer.

These disorders are treated or prevented by administration of a Therapeutic that modulates (i.e. inhibits or promotes) protein complex activity or formation. Diseases or disorders associated with aberrant levels of complex activity or formation, or aberrant levels or activity of the component proteins, or aberrant complex composition, may be treated by administration of a Therapeutic that modulates complex formation or activity or by the administration of a protein complex.

Therapeutic may also be administered to modulate complex formation or activity or level thereof in a microbial organism such as yeast, fungi such as candida albicans causing an infectious disease in animals or humans.

Diseases and disorders characterized by increased (relative to a subject not suffering from the disease or disorder) complex levels or activity can be treated with Therapeutics that antagonize (i.e., reduce or inhibit) complex formation or activity. Therapeutics that can be used include, but are not limited to, the component proteins or an analog, derivative or fragment of the component protein; anti-complex antibodies (e.g., antibodies specific for the protein complex, or a fragment or derivative of the antibody containing the binding region thereof; nucleic acids encoding the component proteins; antisense nucleic acids complementary to nucleic acids encoding the component proteins; and nucleic acids encoding the component protein that are dysfunctional due to, e.g., a heterologous insertion within the protein coding sequence, that are used to “knockout” endogenous protein function by homologous recombination, see, e.g., Capecchi, 1989, Science 244:1288-1292. In one embodiment, a Therapeutic is 1, 2 or more antisense nucleic acids which are complementary to 1, 2, or more nucleic acids, respectfully, that encode component proteins of a complex.

In a specific embodiment of the present invention, a nucleic acid containing a portion of a component protein gene in which gene sequences flank (are both 5′ and 3′ to) a different gene sequence, is used as a component protein antagonist, or to promote component protein inactivation by homologous recombination (see also, Koller and Smithies, 1989, Proc. Natl. Acad. Sci. USA 86:8932-8935; Zijistra et al., 1989, Nature 342: 435-438). Additionally, mutants or derivatives of a component protein that has greater affinity for another component protein or the complex than wild type may be administered to compete with wild type protein for binding, thereby reducing the levels of complexes containing the wild type protein. Other Therapeutics that inhibit complex function can be identified by use of known convenient in vitro assays, e.g., based on their ability to inhibit complex formation, or as described in Section 5.5, infra.

In specific embodiments, Therapeutics that antagonize complex formation or activity are administered therapeutically, including prophylactically, (1) in diseases or disorders involving an increased (relative to normal or desired) level of a complex, for example, in patients where complexes are overactive or overexpressed; or (2) in diseases or disorders where an in vitro (or in vivo) assay (see infra) indicates the utility of antagonist administration. Increased levels of a complex can be readily detected, e.g., by quantifying protein and/or RNA, by obtaining a patient tissue sample (e.g., from biopsy tissue) and assaying it in vitro for RNA or protein levels, or structure and/or activity of the expressed complex (or the encoding mRNA). Many methods standard in the art can be thus employed including, but not limited to, immunoassays to detect complexes and/or visualize complexes (e.g., Western blot analysis, immunoprecipitation followed by sodium dodecyl sulfate polyacrylamide gel electrophoresis [SDS-PAGE], immunocytochemistry, etc.), and/or hybridization assays to detect concurrent expression of component protein mRNA (e.g., Northern assays, dot blot analysis, in situ hybridization, etc.).

A more specific embodiment of the present invention is directed to a method of reducing complex expression (i.e., expression of the protein components of the complex and/or formation of the complex) by targeting mRNAs that express the protein moieties. RNA therapeutics currently fall within three classes, antisense species, ribozymes, or RNA aptamers (Good et al., 1997, Gene Therapy 4:45-54).

Antisense oligonucleotides have been the most widely used. By way of example, but not limitation, antisense oligonucleotide methodology to reduce complex formation is presented below, infra. Ribozyme therapy involves the administration, induced expression, etc. of small RNA molecules with enzymatic ability to cleave, bind, or otherwise inactivate specific RNAs, to reduce or eliminate expression of particular proteins (Grassi and Marini, 1996, Annals of Medicine 28:499-510; Gibson, 1996, Cancer and Metastasis Reviews 15:287-299). RNA aptamers are specific RNA ligand proteins, such as for Tat and Rev RNA (Good et al., 1997, Gene Therapy 4:45-54) that can specifically inhibit their translation. Aptamers specific for component proteins can be identified by many methods well known in the art, for example, by affecting the formation of a complex in the protein-protein interaction assay described, infra.

In another embodiment, the activity or levels of a component protein are reduced by administration of another component protein, or the encoding nucleic acid, or an antibody that immunospecifically binds to the component protein, or a fragment or a derivative of the antibody containing the binding domain thereof.

In another aspect of the invention, diseases or disorders associated with increased levels of an component protein of the complex may be treated or prevented by administration of a Therapeutic that increases complex formation if the complex formation acts to reduce or inactivate the component protein through complex formation. Such diseases or disorders can be treated or prevented by administration of one component member of the complex, administration of antibodies or other molecules that stabilize the complex, etc.

Diseases and disorders associated with underexpression of a complex, or a component protein, are treated or prevented by administration of a Therapeutic that promotes (i.e., increases or supplies) complex levels and/or function, or individual component protein function. Examples of such a Therapeutic include but are not limited to a complex or a derivative, analog or fragment of the complex that are functionally active (e.g., able to form a complex), un-complexed component proteins and derivatives, analogs, and fragments of un-complexed component proteins, and nucleic acids encoding the members of a complex or functionally active derivatives or fragments of the members of the complex, e.g., for use in gene therapy. In a specific embodiment, a Therapeutic includes derivatives, homologs or fragments of a component protein that increase and/or stabilize complex formation. Examples of other agonists can be identified using in vitro assays or animal models, examples of which are described, infra.

In yet other specific embodiments of the present invention, Therapeutics that promote complex function are administered therapeutically, including prophylactically, (1) in diseases or disorders involving an absence or decreased (relative to normal or desired) level of a complex, for example, in patients where a complex, or the individual components necessary to form the complex, is lacking, genetically defective, biologically inactive or underactive, or under-expressed; or (2) in diseases or disorders wherein an in vitro or in vivo assay (see, infra) indicates the utility of complex agonist administration. The absence or decreased level of a complex, component protein or function can be readily detected, e.g., by obtaining a patient tissue sample (e.g., from biopsy tissue) and assaying it in vitro for RNA or protein levels, structure and/or activity of the expressed complex and/or the concurrent expression of mRNA encoding the two components of the complex. Many methods standard in the art can be thus employed, including but not limited to immunoassays to detect and/or visualize a complex, or the individual components of a complex (e.g., Western blot analysis, immunoprecipitation followed by sodium dodecyl sulfate polyacrylamide gel electrophoresis [SDS-PAGE], immunocytochemistry, etc.) and/or hybridization assays to detect expression of mRNAs encoding the individual protein components of a complex by detecting and/or visualizing component mRNA concurrently or separately using, e.g., Northern assays, dot blot analysis, in situ hybridization, etc.

In specific embodiments, the activity or levels of a component protein are increased by administration of another component protein of the same complex, or a derivative, homolog or analog thereof, a nucleic acid encoding the other component, or an agent that stabilizes or enhances the other component, or a fragment or derivative of such an agent.

Generally, administration of products of species origin or species reactivity (in the case of antibodies) that is the same species as that of the patient is preferred. Thus, in a preferred embodiment, a human complex, or derivative, homolog or analog thereof; nucleic acids encoding the members of the human complex or a derivative, homolog or analog thereof; an antibody to a human complex, or a derivative thereof; or other human agents that affect component proteins or the complex, are therapeutically or prophylactically administered to a human patient.

Preferably, suitable in vitro or in vivo assays are utilized to determine the effect of a specific Therapeutic and whether its administration is indicated for treatment of the affected tissue or individual.

In various specific embodiments, in vitro assays can be carried out with representative cells of cell types involved in a patient's disorder, to determine if a Therapeutic has a desired effect upon such cell types.

Compounds for use in therapy can be tested in suitable animal model systems prior to testing in humans, including, but not limited to, rats, mice, chicken, cows, monkeys, rabbits, etc. For in vivo testing, prior to administration to humans, any animal model system known in the art may be used. Additional descriptions and sources of Therapeutics that can be used according to the invention are found in Sections 5.1 to 5.3 and 5.7 herein.

### 5.4.1. Gene Therapy

In a specific embodiment of the present invention, nucleic acids comprising a sequence encoding the component proteins, or a functional derivative thereof, are administered to modulate complex activity or formation by way of gene therapy. Gene therapy refers to therapy performed by the administration of a nucleic acid to a subject. In this embodiment of the present invention, the nucleic acid expresses its encoded protein(s) that mediates a therapeutic effect by modulating complex activity or formation. Any of the methods for gene therapy available in the art can be used according to the present invention. Exemplary methods are described below.

For general reviews of the methods of gene therapy, see Goldspiel et al., 1993, Clinical Pharmacy 12:488-505; Wu and Wu, 1991, Biotherapy 3:87-95; Tolstoshev, 1993, Ann. Rev. Pharmacol. Toxicol. 32:573-596; Mulligan, 1993, Science 260:926-932; Morgan and Anderson, 1993, Ann. Rev. Biochem. 62:191-217; and May, 1993, TIBTECH 11:155-215. Methods commonly known in the art of recombinant DNA technology which can be used are described in Ausubel et al., eds., 1993, Current Protocols in Molecular Biology, John Wiley & Sons, NY; and Kriegler, 1990, Gene Transfer and Expression, A Laboratory Manual, Stockton Press, NY.

In a preferred aspect, the Therapeutic comprises a nucleic acid that is part of an expression vector that expresses one or more of the component proteins, or fragments or chimeric proteins thereof, in a suitable host. In particular, such a nucleic acid has a promoter operably linked to the protein coding region(s) (or, less preferably separate promoters linked to the separate coding regions separately), said promoter being inducible or constitutive, and optionally, tissue-specific. In another particular embodiment, a nucleic acid molecule is used in which the coding sequences, and any other desired sequences, are flanked by regions that promote homologous recombination at a desired site in the genome, thus providing for intra-chromosomal expression of the component protein nucleic acids (Koller and Smithies, 1989, Proc. Natl. Acad. Sci. USA 86:8932-8935; Zijlstra et al., 1989, Nature 342:435-438).

Delivery of the nucleic acid into a patient may be either direct, in which case the patient is directly exposed to the nucleic acid or nucleic acid-carrying vector, or indirect, in which case, cells are first transformed with the nucleic acid in vitro, then transplanted into the patient. These two approaches are known, respectively, as in vivo or ex vivo gene therapy.

In a specific embodiment, the nucleic acid is directly administered in vivo, where it is expressed to produce the encoded product. This can be accomplished by any of numerous methods known in the art, e.g., by constructing it as part of an appropriate nucleic acid expression vector and administering it so that it becomes intracellular, e.g., by infection using a defective or attenuated retroviral or other viral vector (U.S. Pat. No. 4,980,286), or by direct injection of naked DNA, or by use of microparticle bombardment (e.g., a gene gun; Biolistic, Dupont), or coating with lipids or cell-surface receptors, or through use of transfecting agents, by encapsulation in liposomes, microparticles, or microcapsules, or by administering it in linkage to a peptide that is known to enter the nucleus, or by administering it in linkage to a ligand subject to receptor-mediated endocytosis that can be used to target cell types specifically expressing the receptors (e.g., Wu and Wu, 1987, J. Biol. Chem. 262:4429-4432), etc. In another embodiment, a nucleic acid-ligand complex can be formed in which the ligand comprises a fusogenic viral peptide that disrupts endosomes, allowing the nucleic acid to avoid lysosomal degradation. In yet another embodiment, the nucleic acid can be targeted in vivo for cell specific uptake and expression, by targeting a specific receptor (see, e.g., International Patent Publications WO 92/06180; WO 92/22635; WO 92/20316; WO 93/14188; and WO 93/20221. Alternatively, the nucleic acid can be introduced intracellularly and incorporated within host cell DNA for expression, by homologous recombination (Koller and Smithies, 1989, Proc. Natl. Acad. Sci. USA 86:8932-8935; Zijlstra et al., 1989, Nature 342:435-438).

In a specific embodiment, a viral vector that contains the component protein encoding nucleic acids is used. For example, a retroviral vector can be used (Miller et al., 1993, Meth. Enzymol. 217:581-599). These retroviral vectors have been modified to delete retroviral sequences that are not necessary for packaging of the viral genome and integration into host cell DNA. The encoding nucleic acids to be used in gene therapy is/are cloned into the vector, which facilitates delivery of the gene into a patient. More detail about retroviral vectors can be found in Boesen et al., 1994, Biotherapy 6:291-302, which describes the use of a retroviral vector to deliver the mdr1 gene to hematopoetic stem cells in order to make the stem cells more resistant to chemotherapy. Other references illustrating the use of retroviral vectors in gene therapy are Clowes et al., 1994, J. Clin. Invest. 93:644-651; Kiem et al., 1994, Blood 83:1467-1473; Salmons and Gunzberg, 1993, Human Gene Therapy 4:129-141; and Grossman and Wilson, 1993, Curr. Opin. in Genetics and Devel. 3:110-114.

Adenoviruses are other viral vectors that can be used in gene therapy. Adenoviruses are especially attractive vehicles for delivering genes to respiratory epithelia. Adenoviruses naturally infect respiratory epithelia where they cause a mild disease. Other targets for adenovirus-based delivery systems are the liver, the central nervous system, endothelial cells and muscle. Adenoviruses have the advantage of being capable of infecting non-dividing cells. Kozarsky and Wilson, 1993, Current Opinion in Genetics and Development 3:499-503, discuss adenovirus-based gene therapy. The use of adenovirus vectors to transfer genes to the respiratory epithelia of rhesus monkeys has been demonstrated by Bout et al., 1994, Human Gene Therapy 5:3-10. Other instances of the use of adenoviruses in gene therapy can be found in Rosenfeld et al., 1991, Science 252:431-434; Rosenfeld et al., 1992, Cell 68:143-155; and Mastrangeli et al., 1993, J. Clin. Invest. 91:225-234.

Adeno-associated virus (AAV) has also been proposed for use in gene therapy (Walsh et al., 1993, Proc. Soc. Exp. Biol. Med. 204:289-300.

Another approach to gene therapy involves transferring a gene into cells in tissue culture by methods such as electroporation, lipofection, calcium phosphate-mediated transfection, or viral infection. Usually, the method of transfer includes the transfer of a selectable marker to the cells. The cells are then placed under selection to isolate those cells that have taken up and are expressing the transferred gene from these that have not. Those cells are then delivered to a patient.

In this embodiment, the nucleic acid is introduced into a cell prior to administration in vivo of the resulting recombinant cell. Such introduction can be carried out by any method known in the art including, but not limited to, transfection by electroporation, microinjection, infection with a viral or bacteriophage vector containing the nucleic acid sequences, cell fusion, chromosome-mediated gene transfer, microcell-mediated gene transfer, spheroplast fusion, etc. Numerous techniques are known in the art for the introduction of foreign genes into cells (see, e.g., Loeffler and Behr, 1993, Meth. Enzymol. 217:599-618; Cohen et al., 1993, Meth. Enzymol. 217:618-644; Cline, 1985, Pharmac. Ther. 29:69-92) and may be used in accordance with the present invention, provided that the necessary developmental and physiological functions of the recipient cells are not disrupted. The technique should provide for the stable transfer of the nucleic acid to the cell, so that the nucleic acid is expressible by the cell and preferably, is heritable and expressible by its cell progeny.

The resulting recombinant cells can be delivered to a patient by various methods known in the art. In a preferred embodiment, epithelial cells are injected, e.g., subcutaneously. In another embodiment, recombinant skin cells may be applied as a skin graft onto the patient. Recombinant blood cells (e.g., hematopoetic stem or progenitor cells) are preferably administered intravenously. The amount of cells envisioned for use depends on the desired effect, patient state, etc., and can be determined by one skilled in the art.

Cells into which a nucleic acid can be introduced for purposes of gene therapy encompass any desired, available cell type, and include but are not limited to epithelial cells, endothelial cells, keratinocytes, fibroblasts, muscle cells, hepatocytes, blood cells such as T lymphocytes, B lymphocytes, monocytes, macrophages, neutrophils, eosinophils, megakaryocytes, and granulocytes, various stem or progenitor cells, in particular hematopoetic stem or progenitor cells, e.g., as obtained from bone marrow, umbilical cord blood, peripheral blood, fetal liver, etc.

In a preferred embodiment, the cell used for gene therapy is autologous to the patient.

In an embodiment in which recombinant cells are used in gene therapy, a component protein encoding nucleic acid is/are introduced into the cells such that the gene or genes are expressible by the cells or their progeny, and the recombinant cells are then administered in vivo for therapeutic effect. In a specific embodiment, stem or progenitor cells are used. Any stem and/or progenitor cells which can be isolated and maintained in vitro can potentially be used in accordance with this embodiment of the present invention. Such stem cells include but are not limited to hematopoetic stem cells (HSCs), stem cells of epithelial tissues such as the skin and the lining of the gut, embryonic heart muscle cells, liver stem cells (International Patent Publication WO 94/08598), and neural stem cells (Stemple and Anderson, 1992, Cell 71:973-985).

Epithelial stem cells (ESCs), or keratinocytes, can be obtained from tissues such as the skin and the lining of the gut by known procedures (Rheinwald, 1980, Meth. Cell Biol. 2A:229). In stratified epithelial tissue such as the skin, renewal occurs by mitosis of stem cells within the germinal layer, the layer closest to the basal lamina. Similarly, stem cells within the lining of the gut provide for a rapid renewal rate of this tissue. ESCs or keratinocytes obtained from the skin or lining of the gut of a patient or donor can be grown in tissue culture (Rheinwald, 1980, Meth. Cell Bio. 2A:229; Pittelkow and Scott, 1986, Mayo Clinic Proc. 61:771). If the ESCs are provided by a donor, a method for suppression of host versus graft reactivity (e.g., irradiation, or drug or antibody administration to promote moderate immunosuppression) can also be used.

With respect to hematopoetic stem cells (HSCs), any technique that provides for the isolation, propagation, and maintenance in vitro of HSCs can be used in this embodiment of the invention. Techniques by which this may be accomplished include (a) the isolation and establishment of HSC cultures from bone marrow cells isolated from the future host, or a donor, or (b) the use of previously established long-term HSC cultures, which may be allogeneic or xenogeneic. Non-autologous HSCs are used preferably in conjunction with a method of suppressing transplantation immune reactions between the future host and patient. In a particular embodiment of the present invention, human bone marrow cells can be obtained from the posterior iliac crest by needle aspiration (see, e.g., Kodo et al., 1984, J. Clin. Invest. 73: 1377-1384). In a preferred embodiment of the present invention, the HSCs can be made highly enriched or in substantially pure form. This enrichment can be accomplished before, during, or after long-term culturing, and can be done by any technique known in the art. Long-term cultures of bone marrow cells can be established and maintained by using, for example, modified Dexter cell culture techniques (Dexter et al., 1977, J. Cell Physiol. 91:335) or Witlock-Witte culture techniques (Witlock and Witte, 1982, Proc. Natl. Acad. Sci. USA 79:3608-3612).

In a specific embodiment, the nucleic acid to be introduced for purposes of gene therapy comprises an inducible promoter operably linked to the coding region, such that expression of the nucleic acid is controllable by controlling the presence or absence of the appropriate inducer of transcription.

Additional methods can be adapted for use to deliver a nucleic acid encoding the component proteins, or functional derivatives thereof, e.g., as described in Section 5.1, supra.

5.4.2. Use of Anitsense Oligonucleotides for Suppression of Protein Complex Activity or Formation

In a specific embodiment of the present invention, protein complex activity and formation is inhibited by use of antisense nucleic acids for the component proteins of the complex, that inhibit transcription and/or translation of their complementary sequence. The present invention provides the therapeutic or prophylactic use of nucleic acids of at least six nucleotides that are antisense to a gene or cDNA encoding a component protein, or a portion thereof. An “antisense” nucleic acid as used herein refers to a nucleic acid capable of hybridizing to a sequence-specific portion of a component protein RNA (preferably mRNA) by virtue of some sequence complementarity. The antisense nucleic acid may be complementary to a coding and/or noncoding region of a component protein mRNA. Such antisense nucleic acids that inhibit complex formation or activity have utility as Therapeutics, and can be used in the treatment or prevention of disorders as described supra.

The antisense nucleic acids of the invention can be oligonucleotides that are double-stranded or single-stranded, RNA or DNA, or a modification or derivative thereof, which can be directly administered to a cell, or which can be produced intracellularly by transcription of exogenous, introduced sequences.

In another embodiment, the present invention is directed to a method for inhibiting the expression of component protein nucleic acid sequences, in a prokaryotic or eukaryotic cell, comprising providing the cell with an effective amount of a composition comprising an antisense nucleic acid of the component protein, or a derivative thereof, of the invention.

The antisense nucleic acids are of at least six nucleotides and are preferably oligonucleotides, ranging from 6 to about 200 nucleotides. In specific aspects, the oligonucleotide is at least 10 nucleotides, at least 15 nucleotides, at least 100 nucleotides, or at least 200 nucleotides. The oligonucleotides can be DNA or RNA or chimeric mixtures, or derivatives or modified versions thereof, and either single-stranded or double-stranded. The oligonucleotide can be modified at the base moiety, sugar moiety, or phosphate backbone. The oligonucleotide may include other appending groups such as peptides, agents facilitating transport across the cell membrane (see, e.g., Letsinger et al., 1989, Proc. Natl. Acad. Sci. U.S.A. 86:6553-6556; Lemaitre et al., 1987, Proc. Natl. Acad. Sci. 84:648-652; International Patent Publication No. WO 88/09810) or blood-brain barrier (see, e.g., International Patent Publication No. WO 89/10134), hybridization-triggered cleavage agents (see, e.g., Krol et al., 1988, BioTechniques 6:958-976), or intercalating agents (see, e.g., Zon, 1988, Pharm. Res. 5:539-549).

In a preferred aspect of the invention, an antisense oligonucleotide is provided, preferably as single-stranded DNA. The oligonucleotide may be modified at any position in its structure with constituents generally known in the art.

The antisense oligonucleotides may comprise at least one modified base moiety which is selected from the group including but not limited to 5-fluorouracil, 5-bromouracil, 5-chlorouracil, 5-iodouracil, hypoxanthine, xanthine, 4-acetylcytosine, 5-(carboxyhydroxylmethyl)uracil, 5-carboxymethylaminomethyl-2-thio-uridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5N-methoxycarboxymethyluracil, 5-methoxyuracil, 2-methyl-thio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine.

In another embodiment, the oligonucleotide comprises at least one modified sugar moiety selected from the group including, but not limited to, arabinose, 2-fluoroarabinose, xylulose, and hexose.

In yet another embodiment, the oligonucleotide comprises at least one modified phosphate backbone selected from the group consisting of a phosphorothioate, a phosphorodithioate, a phosphoramidothioate, a phosphoramidate, a phosphordiamidate, a methylphosphonate, an alkyl phosphotriester, and a formacetal, or an analog of the foregoing.

In yet another embodiment, the oligonucleotide is a 2-a-anomeric oligonucleotide. An a-anomeric oligonucleotide forms specific double-stranded hybrids with complementary RNA in which, contrary to the usual β-units, the strands run parallel to each other (Gautier et al., 1987, Nucl. Acids Res. 15:6625-6641).

The oligonucleotide may be conjugated to another molecule, e.g., a peptide, hybridization-triggered cross-linking agent, transport agent, hybridization-triggered cleavage agent, etc.

Oligonucleotides of the invention may be synthesized by standard methods known in the art, e.g., by use of an automated DNA synthesizer (such as are commercially available from Biosearch, Applied Biosystems, etc.). As examples, phosphorothioate oligo-nucleotides may be synthesized by the method of Stein et al. (1988, Nucl. Acids Res. 16:3209), methylphosphonate oligonucleotides can be prepared by use of controlled pore glass polymer supports (Sarin et al., 1988, Proc. Natl. Acad. Sci. U.S.A. 85:7448-7451), etc.

In a specific embodiment, the antisense oligonucleotides comprise catalytic RNAs, or ribozymes (see, e.g., International Patent Publication No. WO 90/11364; Sarver et al., 1990, Science 247:1222-1225). In another embodiment, the oligonucleotide is a 2′-0-methylribonucleotide (Inoue et al., 1987, Nucl. Acids Res. 15:6131-6148), or a chimeric RNA-DNA analog (Inoue et al., 1987, FEBS Lett. 215:327-330).

In an alternative embodiment, the antisense nucleic acids of the invention are produced intracellularly by transcription from an exogenous sequence. For example, a vector can be introduced in vivo such that it is taken up by a cell, within which cell the vector or a portion thereof is transcribed, producing an antisense nucleic acid (RNA) of the invention. Such a vector would contain a sequence encoding the component protein. Such a vector can remain episomal or become chromosomally integrated, as long as it can be transcribed to produce the desired antisense RNA. Such vectors can be constructed by recombinant DNA technology methods standard in the art. Vectors can be plasmid, viral, or others known in the art to be capable of replication and expression in mammalian cells. Expression of the sequences encoding the antisense RNAs can be by any promoter known in the art to act in mammalian, preferably human, cells. Such promoters can be inducible or constitutive. Such promoters include, but are not limited to, the SV40 early promoter region (Bemoist and Chambon, 1981, Nature 290:304-310), the promoter contained in the 3′ long terminal repeat of Rous sarcoma virus (Yamamoto et al., 1980, Cell 22:787-797), the herpes thymidine kinase promoter (Wagner et al., 1981, Proc. Natl. Acad. Sci. U.S.A. 78:1441-1445), the regulatory sequences of the metallothionein gene (Brinster et al., 1982, Nature 296:39-42), etc.

The antisense nucleic acids of the invention comprise a sequence complementary to at least a portion of an RNA transcript of a component protein gene, preferably a human gene. However, absolute complementarity, although preferred, is not required. A sequence “complementary to at least a portion of an RNA,” as referred to herein, means a sequence having sufficient complementarity to be able to hybridize with the RNA, forming a stable duplex; in the case of double-stranded antisense nucleic acids, a single strand of the duplex DNA may thus be tested, or triplex formation may be assayed. The ability to hybridize will depend on both the degree of complementarity and the length of the antisense nucleic acid. Generally, the longer the hybridizing nucleic acid, the more base mismatches with a component protein RNA it may contain and still form a stable duplex (or triplex, as the case may be). One skilled in the art can ascertain a tolerable. degree of mismatch by use of standard procedures to determine the melting point of the hybridized complex.

The component protein antisense nucleic acids can be used to treat (or prevent) disorders of a cell type that expresses, or preferably overexpresses, a protein complex.

Cell types that express or overexpress component protein RNA can be identified by various methods known in the art. Such methods include, but are not limited to, hybridization with component protein-specific nucleic acids (e.g., by Northern blot hybridization, dot blot hybridization, or in situ hybridization), or by observing the ability of RNA from the cell type to be translated in vitro into the component protein by immunohistochemistry, Western blot analysis, ELISA, etc. In a preferred aspect, primary tissue from a patient can be assayed for protein expression prior to treatment, e.g., by immunocytochemistry, in situ hybridization, or any number of methods to detect protein or mRNA expression.

Pharmaceutical compositions of the invention (see Section 5.7, infra), comprising an effective amount of a protein component antisense nucleic acid in a pharmaceutically acceptable carrier can be administered to a patient having a disease or disorder that is of a type that expresses or overexpresses a protein complex of the present invention.

The amount of antisense nucleic acid that will be effective in the treatment of a particular disorder or condition will depend on the nature of the disorder or condition, and can be determined by standard clinical techniques. Where possible, it is desirable to determine the antisense cytotoxicity in vitro, and then in useful animal model systems, prior to testing and use in humans.

In a specific embodiment, pharmaceutical compositions comprising antisense nucleic acids are administered via liposomes, microparticles, or microcapsules. In various embodiments of the invention, it may be useful to use such compositions to achieve sustained release of the antisense nucleic acids. In a specific embodiment, it may be desirable to utilize liposomes targeted via antibodies to specific identifiable central nervous system cell types (Leonetti et al., 1990, Proc. Natl. Acad. Sci. U.S.A. 87:2448-2451; Renneisen et al., 1990, J. Biol. Chem. 265:16337-16342).

### 5.5. Assays of Protein Complexes and Derivatives and Analogs Thereof

The functional activity of a protein complex of the present invention, or a derivative, fragment or analog thereof, can be assayed by various methods. Potential modulators (e.g., agonists and antagonists) of complex activity or formation, e.g., anti-complex antibodies and antisense nucleic acids, can be assayed for the ability to modulate complex activity or formation.

In one embodiment of the present invention, where one is assaying for the ability to bind or compete with a wild-type complex for binding to an anti-complex antibody, various immunoassays known in the art can be used, including but not limited to competitive and non-competitive assay systems using techniques such as radioimmunoassay, ELISA (enzyme linked immunosorbent assay), “sandwich” immunoassays, immunoradiometric assays, gel diffusion precipitin reactions, immunodiffusion assays, in situ immunoassays (using colloidal gold, enzyme or radioisotope labels), western blot analysis, precipitation reactions, agglutination assays (e.g., gel agglutination assays, hemagglutination assays), complement fixation assays, immunofluorescence assays, protein A assays, immunoelectrophoresis assays, etc. In one embodiment, antibody binding is detected by detecting a label on the primary antibody. In another embodiment, the primary antibody is detected by detecting binding of a secondary antibody or reagent to the primary antibody. In a further embodiment, the secondary antibody is labeled. Many means are known in the art for detecting binding in an immunoassay and are within the scope of the present invention.

The expression of the component protein genes (both endogenous and those expressed from cloned DNA containing the genes) can be detected using techniques known in the art, including but not limited to Southern hybridization (Southern, 1975, J. Mol. Biol. 98:503-517), northern hybridization (see, e.g., Freeman et al., 1983, Proc. Natl. Acad. Sci. USA 80:4094-4098), restriction endonuclease mapping (Sambrook et al., 1989, Molecular Cloning, A Laboratory Manual, 2^{nd} Ed. Cold Spring Harbor Laboratory Press, New York), RNase protection assays (Current Protocols in Molecular Biology, John Wiley and Sons, New York, 1997), DNA sequence analysis, and polymerase chain reaction amplification (PCR; U.S. Pat. Nos. 4,683,202, 4,683,195, and 4,889,818; Gyllenstein et al., 1988, Proc. Natl. Acad. Sci. USA 85:7652-7657; Ochman et al., 1988, Genetics 120:621-623; Loh et al., 1989, Science 243:217-220) followed by Southern hybridization with probes specific for the component protein genes, in various cell types. Methods of amplification other than PCR commonly known in the art can be employed. In one embodiment, Southern hybridization can be used to detect genetic linkage of component protein gene mutations to physiological or pathological states. Various cell types, at various stages of development, can be characterized for their expression of component proteins at the same time and in the same cells. The stringency of the hybridization conditions for northern or Southern blot analysis can be manipulated to ensure detection of nucleic acids with the desired degree of relatedness to the specific probes used. Modifications to these methods and other methods commonly known in the art can be used.

Derivatives (e.g., fragments), homologs and analogs of one component protein can be assayed for binding to another component protein in the same complex by any method known in the art, for example the modified yeast matrix mating test described in Section 5.6.1 infra, immunoprecipitation with an antibody that binds to the component protein complexed with other component proteins in the same complex, followed by size fractionation of the immunoprecipitated proteins (e.g., by denaturing or nondenaturing polyacrylamide gel electrophoresis), Western blot analysis, etc.

One embodiment of the invention provides a method for screening a derivative, homolog or analog of a component protein for biological activity comprising contacting said derivative, homolog or analog of the component protein with the other component proteins in the same complex; and detecting the formation of a complex between said derivative, homolog or analog of the component protein and the other component proteins; wherein detecting formation of said complex indicates that said derivative, homolog or analog of has biological (e.g., binding) activity.

The invention also provides methods of modulating the activity of a component protein that can participate in a protein complex by administration of a binding partner of that protein or derivative, homolog or analog thereof.

In a specific embodiment of the present invention, a protein complex of the present invention is administered to treat or prevent a disease or disorder, since the complex and/or component proteins have been implicated in the disease and disorder. Accordingly, a protein complex or a derivative, homolog, analog or fragment thereof, nucleic acids encoding the component proteins, anti-complex antibodies, and other modulators of protein complex activity, can be tested for activity in treating or preventing a disease or disorder in in vitro and in vivo assays.

In one embodiment, a Therapeutic of the invention can be assayed for activity in treating or preventing a disease by contacting cultured cells that exhibit an indicator of the disease in vitro, with the Therapeutic, and comparing the level of said indicator in the cells contacted with the Therapeutic, with said level of said indicator in cells not so contacted, wherein a lower level in said contacted cells indicates that the Therapeutic has activity in treating or preventing the disease.

In another embodiment of the invention, a Therapeutic of the invention can be assayed for activity in treating or preventing a disease by administering the Therapeutic to a test animal that is predisposed to develop symptoms of a disease, and measuring the change in said symptoms of the disease after administration of said Therapeutic, wherein a reduction in the severity of the symptoms of the disease or prevention of the symptoms of the disease indicates that the Therapeutic has activity in treating or preventing the disease. Such a test animal can be any one of a number of animal models known in the art for disease. These animal models are well known in the art. These animal models include, but are not limited to those which are listed in the section 5.6 (supra) as exemplary animald models to study any of the complexes provided in the invention.

### 5.6 Screening for Modulators of the Proetin Complexes

A complex of the present invention, the component proteins of the complex and nucleic acids encoding the component proteins, as well as derivatives and fragments of the amino and nucleic acids, can be used to screen for compounds that bind to, or modulate the amount of, activity of, or protein component composition of, said complex, and thus, have potential use as modulators, i.e., agonists or antagonists, of complex activity, and/or complex formation, i.e., the amount of complex formed, and/or protein component composition of the complex.

Thus, the present invention is also directed to methods for screening for molecules that bind to, or modulate the amount of, activity of, or protein component composition of, a complex of the present invention. In one embodiment of the invention, the method for screening for a molecule that modulates directly or indirectly the function, activity or formation of a complex of the present invention comprises exposing said complex, or a cell or organism containing the complex machinery, to one or more candidate molecules under conditions conducive to modulation; and determining the amount of, activity of, or identities of the protein components of, said complex, wherein a change in said amount, activity, or identities relative to said amount, activity or identities in the absence of said candidate molecules indicates that the molecules modulate function, activity or formation of said complex.

In another embodiment, the present invention further relates to a process for the identification and/or preparation of an effector of the cleavage/polyadenylation of precursor mRNA comprising the step of bringing into contact a product of any of claims 1 to 7 with a compound, a mixture or a library of compounds and determining whether the compound or a certain compound of the mixture or library binds to the product and/or effects the products biological activity and optionally further purifying the compound positively tested as effector.

In another embodiment, the present invention is directed to a method for screening for a molecule that binds a protein complex of the present invention comprising exposing said complex, or a cell or organism containing the complex machinery, to one or more candidate molecules; and determining whether said complex is bound by any of said candidate molecules. Such screening assays can be carried out using cell-free and cell-based methods that are commonly known in the art in vitro, in vivo or ex vivo. For example, an isolated complex can be employed, or a cell can be contacted with the candidate molecule and the complex can be isolated from such contacted cells and the isolated complex can be assayed for activity or component composition. In another example, a cell containing the complex can be contacted with the candidate molecule and the levels of the complex in the contacted cell can be measured. Additionally, such assays can be carried out in cells recombinantly expressing a component protein from column A of table 1 of a given row, or a functionally active fragment or functionally active derivative thereof, and a component protein from column B of table 1 of said row, or a functionally active fragment or functionally active derivative thereof. Additionally, such assays can also be carried out in cells recombinantly expressing all component proteins from the group of proteins in column C of table 1.

For example, assays can be carried out using recombinant cells expressing the protein components of a complex, to screen for molecules that bind to, or interfere with, or promote complex activity or formation. In preferred embodiments, polypeptide derivatives that have superior stabilities but retain the ability to form a complex (e.g., one or more component proteins modified to be resistant to proteolytic degradation in the binding assay buffers, or to be resistant to oxidative degradation), are used to screen for modulators of complex activity or formation. Such resistant molecules can be generated, e.g., by substitution of amino acids at proteolytic cleavage sites, the use of chemically derivatized amino acids at proteolytic susceptible sites, and the replacement of amino acid residues subject to oxidation, i.e. methionine and cysteine.

A particular aspect of the present invention relates to identifying molecules that inhibit or promote formation or degradation of a complex of the present invention, e.g., using the method described for isolating the complex and identifying members of the complex using the TAP assay described in Section 6, infra, and in WO 00/09716 and Rigaut et al., 1999, Nature Biotechnology 17:1030-1032, which are each incorporated by reference in their entirety.

In another embodiment of the invention, a modulator is identified by administering a candidate molecule to a transgenic non-human animal expressing the complex component proteins from promoters that are not the native promoters of the respective proteins, more preferably where the candidate molecule is also recombinantly expressed in the transgenic non-human animal. Alternatively, the method for identifying such a modulator can be carried out in vitro, preferably with a purified complex, and a purified candidate molecule.

Agents/molecules (candidate molecules) to be screened can be provided as mixtures of a limited number of specified compounds, or as compound libraries, peptide libraries and the like. Agents/molecules to be screened may also include all forms of antisera, antisense nucleic acids, etc., that can modulate complex activity or formation. Exemplary candidate molecules and libraries for screening are set forth in Section 5.6.1, infra.

Screening the libraries can be accomplished by any of a variety of commonly known methods. See, e.g., the following references, which disclose screening of peptide libraries: Parmley and Smith, 1989, Adv. Exp. Med. Biol. 251:215-218; Scott and Smith, 1990, Science 249:386-390; Fowlkes et al., 1992, BioTechniques 13:422-427; Oldenburg et al., 1992, Proc. Natl. Acad. Sci. USA 89:5393-5397; Yu et al., 1994, Cell 76:933-945; Staudt et al., 1988, Science 241:577-580; Bock et al., 1992, Nature 355:564-566; Tuerk et al., 1992, Proc. Natl. Acad. Sci. USA 89:6988-6992; Ellington et al., 1992, Nature 355:850-852; U.S. Pat. No. 5,096,815, U.S. Pat. No. 5,223,409, and U.S. Pat. No. 5,198,346, all to Ladner et al.; Rebar and Pabo, 1993, Science 263:671-673; and International Patent Publication No. WO 94/18318.

In a specific embodiment, screening can be carried out by contacting the library members with a complex immobilized on a solid phase, and harvesting those library members that bind to the protein (or encoding nucleic acid or derivative). Examples of such screening methods, termed “panning” techniques, are described by way of example in Parmley and Smith, 1988, Gene 73:305-318; Fowlkes et al., 1992, BioTechniques 13:422-427; International Patent Publication No. WO 94/18318; and in references cited hereinabove.

In a specific embodiment, fragments and/or analogs of protein components of a complex, especially peptidomimetics, are screened for activity as competitive or non-competitive inhibitors of complex formation (amount of complex or composition of complex) or activity in the cell, which thereby inhibit complex activity or formation in the cell.

In one embodiment, agents that modulate (i.e., antagonize or agonize) complex activity or formation can be screened for using a binding inhibition assay, wherein agents are screened for their ability to modulate formation of a complex under aqueous, or physiological, binding conditions in which complex formation occurs in the absence of the agent to be tested. Agents that interfere with the formation of complexes of the invention are identified as antagonists of complex formation. Agents that promote the formation of complexes are identified as agonists of complex formation. Agents that completely block the formation of complexes are identified as inhibitors of complex formation.

Methods for screening may involve labeling the component proteins of the complex with radioligands (e.g., ¹²⁵I or ³H), magnetic ligands (e.g., paramagnetic beads covalently attached to photobiotin acetate), fluorescent ligands (e.g., fluorescein or rhodamine), or enzyme ligands (e.g., luciferase or beta-galactosidase). The reactants that bind in solution can then be isolated by one of many techniques known in the art, including but not restricted to, co-immunoprecipitation of the labeled complex moiety using antisera against the unlabeled binding partner (or labeled binding partner with a distinguishable marker from that used on the second labeled complex moiety), immunoaffinity chromatography, size exclusion chromatography, and gradient density centrifugation. In a preferred embodiment, the labeled binding partner is a small fragment or peptidomimetic that is not retained by a commercially available filter. Upon binding, the labeled species is then unable to pass through the filter, providing for a simple assay of complex formation.

Methods commonly known in the art are used to label at least one of the component members of the complex. Suitable labeling methods include, but are not limited to, radiolabeling by incorporation of radiolabeled amino acids, e.g., ³H-leucine or ³⁵S-methionine, radiolabeling by post-translational iodination with ¹²⁵I or ¹³¹I using the chloramine T method, Bolton-Hunter reagents, etc., or labeling with ³²P using phosphorylase and inorganic radiolabeled phosphorous, biotin labeling with photobiotin acetate and sunlamp exposure, etc. In cases where one of the members of the complex is immobilized, e.g., as described infra, the free species is labeled. Where neither of the interacting species is immobilized, each can be labeled with a distinguishable marker such that isolation of both moieties can be followed to provide for more accurate quantification, and to distinguish the formation of homomeric from heteromeric complexes. Methods that utilize accessory proteins that bind to one of the modified interactants to improve the sensitivity of detection, increase the stability of the complex, etc., are provided.

Typical binding conditions are, for example, but not by way of limitation, in an aqueous salt solution of 10-250 mM NaCl, 5-50 mM Tris-HCl, pH 5-8, and 0.5% Triton X-100 or other detergent that improves specificity of interaction. Metal chelators and/or divalent cations may be added to improve binding and/or reduce proteolysis. Reaction temperatures may include 4, 10, 15, 22, 25, 35, or 42 degrees Celsius, and time of incubation is typically at least 15 seconds, but longer times are preferred to allow binding equilibrium to occur. Particular complexes can be assayed using routine protein binding assays to determine optimal binding conditions for reproducible binding.

The physical parameters of complex formation can be analyzed by quantification of complex formation using assay methods specific for the label used, e.g., liquid scintillation counting for radioactivity detection, enzyme activity for enzyme-labeled moieties, etc. The reaction results are then analyzed utilizing Scatchard analysis, Hill analysis, and other methods commonly known in the arts (see, e.g., Proteins, Structures, and Molecular Principles, 2^{nd} Edition (1993) Creighton, Ed., W.H. Freeman and Company, New York).

In a second common approach to binding assays, one of the binding species is immobilized on a filter, in a microtiter plate well, in a test tube, to a chromatography matrix, etc., either covalently or non-covalently. Proteins can be covalently immobilized using any method well known in the art, for example, but not limited to the method of Kadonaga and Tjian, 1986, Proc. Natl. Acad. Sci. USA 83:5889-5893, i.e., linkage to a cyanogen-bromide derivatized substrate such as CNBr-Sepharose 4B (Pharmacia). Where needed, the use of spacers can reduce steric hindrance by the substrate. Non-covalent attachment of proteins to a substrate include, but are not limited to, attachment of a protein to a charged surface, binding with specific antibodies, binding to a third unrelated interacting protein, etc.

Assays of agents (including cell extracts or a library pool) for competition for binding of one member of a complex (or derivatives thereof) with another member of the complex labeled by any means (e.g., those means described above) are provided to screen for competitors or enhancers of complex formation.

In specific embodiments, blocking agents to inhibit non-specific binding of reagents to other protein components, or absorptive losses of reagents to plastics, immobilization matrices, etc., are included in the assay mixture. Blocking agents include, but are not restricted to bovine serum albumin, beta-casein, nonfat dried milk, Denhardt's reagent, Ficoll, polyvinylpyrolidine, nonionic detergents (NP40, Triton X-100, Tween 20, Tween 80, etc.), ionic detergents (e.g., SDS, LDS, etc.), polyethylene glycol, etc. Appropriate blocking agent concentrations allow complex formation.

After binding is performed, unbound, labeled protein is removed in the supernatant, and the immobilized protein retaining any bound, labeled protein is washed extensively. The amount of bound label is then quantified using standard methods in the art to detect the label as described, supra.

Moreover, a number of polyadenylation assays are described in the prior art. Such assays can be found in Bienroth, S. E.; Wahle, C.; Suter-Crazzolara, C. and Keller, W. (1991), J. Biol. Chem. 266, 19768-19776; Edwards-Gilbert, G. and Milcarek, C. (1995), Mol. Cell. Biol. 15, 6420-6429; Wahle, E. (1991), Cell 66, 759-768; Christofori, G. and Keller, W. (Cell) 54, 875-889.

Exemplary assays useful to measure the 3′ end processing activity for mRNA of complex 162 include, but are not limited to those described in Kessler M M et al, 1996, J Biol. Chem. 271: 27167-75, and Butler, S. J. and Platt, T. (1988), Science 242, 1270-1274, and Moore, C. L. and Sharp, P. A. (1985), Cell 41, 845-855

Exemplary assays useful to measure the cleavage step in 3′ end processing activity of mRNA of complex 162 include, but are not limited to those described in Ruegsegger U et al., 1996, J Biol Chem 271: 6107-6113.

An exemplary RNA binding assay can be carried out by contacting a complex having RNA binding activity with a radioactive [32P] end-labeled RNA substrate, e.g. a poly (A) RNA, under appropriate conditions and detecting bound protein. The detection of bound protein can be carried out, e.g., by filtrating the solution through a nitrocellulose filter and determining the radioactivity bound to the filter. This assay is based on the retention of nucleic acid-protein complexes on Nitrocellulose whereas free nucleic acid can pass through the filter

(see e.g. Wahle, E., 1991, Methods 66: 759-68)

An exemplary RNA exonuclease assay can be carried out by contacting a complex having RNA exonuclease activity with a radioactivity [32 phosphate] end-labeled RNA substrate under appropriate conditions and detecting the release of free radioactive nucleotides. The detection of free radioactive nucleotides can be carried out, e.g., by adding 20% trichloroacetic acid, filtrating the solution through a filter and measuring the amount of acid-soluble radioactivity

(see e.g. Ross, J., 1999, Methods 17: 52-9)

An exemplary mRNA splicing assay can be carried out by contacting a complex having mRNA splicing activity with a radioactively labeled RNA substrate under appropriate conditions and detecting the release of spliced RNA species. The detection of spliced RNA species can be carried out, e.g., by fractionation of processed RNAs in a glycerol gradient and subsequent analysis by denaturing polyacrylamide gel elecrophoresis and visualization by autoradiography.

(see e.g. Schwer, B. and Gross, C H., 1998, Methods17: 2086-94)

An exemplary rRNA processing assay can be carried out by contacting a complex having rRNA processing activity with an pre-rRNA substrate under appropriate conditions and detecting the release of free processed rRNA species. The detection of processed rRNA species can be carried out, e.g., using a primer extension or northern blotting assay by measuring the size of the rRNA species.

(see e.g. Kressler, D. et al, 1997, Methods 17: 7283-94)

5.6.1. Candidate Molecules

Any molecule known in the art can be tested for its ability to modulate (increase or decrease) the amount of, activity of, or protein component composition of a complex of the present invention as detected by a change in the amount of, activity of, or protein component composition of, said complex. By way of example, a change in the amount of the complex can be detected by detecting a change in the amount of the complex that can be isolated from a cell expressing the complex machinery. For identifying a molecule that modulates complex activity, candidate molecules can be directly provided to a cell expressing the complex machinery, or, in the case of candidate proteins, can be provided by providing their encoding nucleic acids under conditions in which the nucleic acids are recombinantly expressed to produce the candidate proteins within the cell expressing the complex machinery, the complex is then isolated from the cell and the isolated complex is assayed for activity using methods well known in the art, not limited to those described, supra.

This embodiment of the invention is well suited to screen chemical libraries for molecules which modulate, e.g., inhibit, antagonize, or agonize, the amount of, activity of, or protein component composition of the complex. The chemical libraries can be peptide libraries, peptidomimetic libraries, chemically synthesized libraries, recombinant, e.g., phage display libraries, and in vitro translation-based libraries, other non-peptide synthetic organic libraries, etc.

Exemplary libraries are commercially available from several sources (ArQule, Tripos/PanLabs, ChemDesign, Pharmacopoeia). In some cases, these chemical libraries are generated using combinatorial strategies that encode the identity of each member of the library on a substrate to which the member compound is attached, thus allowing direct and immediate identification of a molecule that is an effective modulator. Thus, in many combinatorial approaches, the position on a plate of a compound specifies that compound's composition. Also, in one example, a single plate position may have from 1-20 chemicals that can be screened by administration to a well containing the interactions of interest. Thus, if modulation is detected, smaller and smaller pools of interacting pairs can be assayed for the modulation activity. By such methods, many candidate molecules can be screened.

Many diversity libraries suitable for use are known in the art and can be used to provide compounds to be tested according to the present invention. Alternatively, libraries can be constructed using standard methods. Chemical (synthetic) libraries, recombinant expression libraries, or polysome-based libraries are exemplary types of libraries that can be used.

The libraries can be constrained or semirigid (having some degree of structural rigidity), or linear or nonconstrained. The library can be a cDNA or genomic expression library, random peptide expression library or a chemically synthesized random peptide library, or non-peptide library. Expression libraries are introduced into the cells in which the assay occurs, where the nucleic acids of the library are expressed to produce their encoded proteins.

In one embodiment, peptide libraries that can be used in the present invention may be libraries that are chemically synthesized in vitro. Examples of such libraries are given in Houghten et al., 1991, Nature 354:84-86, which describes mixtures of free hexapeptides in which the first and second residues in each peptide were individually and specifically defined; Lam et al., 1991, Nature 354:82-84, which describes a “one bead, one peptide” approach in which a solid phase split synthesis scheme produced a library of peptides in which each bead in the collection had immobilized thereon a single, random sequence of amino acid residues; Medynski, 1994, Bio/Technology 12:709-710, which describes split synthesis and T-bag synthesis methods; and Gallop et al., 1994, J. Medicinal Chemistry 37(9):1233-1251. Simply by way of other examples, a combinatorial library may be prepared for use, according to the methods of Ohlmeyer et al., 1993, Proc. Natl. Acad. Sci. USA 90:10922-10926; Erb et al., 1994, Proc. Natl. Acad. Sci. USA 91:11422-11426; Houghten et al., 1992, Biotechniques 13:412; Jayawickreme et al., 1994, Proc. Natl. Acad. Sci. USA 91:1614-1618; or Salmon et al., 1993, Proc. Natl. Acad. Sci. USA 90:11708-11712. PCT Publication No. WO 93/20242 and Brenner and Lerner, 1992, Proc. Natl. Acad. Sci. USA 89:5381-5383 describe “encoded combinatorial chemical libraries,” that contain oligonucleotide identifiers for each chemical polymer library member.

In a preferred embodiment, the library screened is a biological expression library that is a random peptide phage display library, where the random peptides are constrained (e.g., by virtue of having disulfide bonding).

Further, more general, structurally constrained, organic diversity (e.g., nonpeptide) libraries, can also be used. By way of example, a benzodiazepine library (see e.g., Bunin et al., 1994, Proc. Natl. Acad. Sci. USA 91:4708-4712) may be used.

Conformationally constrained libraries that can be used include but are not limited to those containing invariant cysteine residues which, in an oxidizing environment, cross-link by disulfide bonds to form cystines, modified peptides (e.g., incorporating fluorine, metals, isotopic labels, are phosphorylated, etc.), peptides containing one or more non-naturally occurring amino acids, non-peptide structures, and peptides containing a significant fraction of -carboxyglutamic acid.

Libraries of non-peptides, e.g., peptide derivatives (for example, that contain one or more non-naturally occurring amino acids) can also be used. One example of these are peptoid libraries (Simon et al., 1992, Proc. Natl. Acad. Sci. USA 89:9367-9371). Peptoids are polymers of non-natural amino acids that have naturally occurring side chains attached not to the alpha carbon but to the backbone amino nitrogen. Since peptoids are not easily degraded by human digestive enzymes, they are advantageously more easily adaptable to drug use. Another example of a library that can be used, in which the amide functionalities in peptides have been permethylated to generate a chemically transformed combinatorial library, is described by Ostresh et al., 1994, Proc. Natl. Acad. Sci. USA 91:11138-11142).

The members of the peptide libraries that can be screened according to the invention are not limited to containing the 20 naturally occurring amino acids. In particular, chemically synthesized libraries and polysome based libraries allow the use of amino acids in addition to the 20 naturally occurring amino acids (by their inclusion in the precursor pool of amino acids used in library production). In specific embodiments, the library members contain one or more non-natural or non-classical amino acids or cyclic peptides. Non-classical amino acids include but are not limited to the D-isomers of the common amino acids, -amino isobutyric acid, 4-aminobutyric acid, Abu, 2-amino butyric acid; -Abu, -Ahx, 6-amino hexanoic acid; Aib, 2-amino isobutyric acid; 3-amino propionic acid; omithine; norleucine; norvaline, hydroxyproline, sarcosine, citrulline, cysteic acid, t-butylglycine, t-butylalanine, phenylglycine, cyclohexylalanine, β-alanine, designer amino acids such as β-methyl amino acids, C-methyl amino acids, N-methyl amino acids, fluoro-amino acids and amino acid analogs in general. Furthermore, the amino acid can be D (dextrorotary) or L (levorotary).

In a specific embodiment, fragments and/or analogs of complexes of the invention, or protein components thereof, especially peptidomimetics, are screened for activity as competitive or non-competitive inhibitors of complex activity or formation.

In another embodiment of the present invention, combinatorial chemistry can be used to identify modulators of a the complexes. Combinatorial chemistry is capable of creating libraries containing hundreds of thousands of compounds, many of which may be structurally similar. While high throughput screening programs are capable of screening these vast libraries for affinity for known targets, new approaches have been developed that achieve libraries of smaller dimension but which provide maximum chemical diversity. (See e.g., Matter, 1997, Journal of Medicinal Chemistry 40:1219-1229).

One method of combinatorial chemistry, affinity fingerprinting, has previously been used to test a discrete library of small molecules for binding affinities for a defined panel of proteins. The fingerprints obtained by the screen are used to predict the affinity of the individual library members for other proteins or receptors of interest (in the instant invention, the protein complexes of the present invention and protein components thereof.) The fingerprints are compared with fingerprints obtained from other compounds known to react with the protein of interest to predict whether the library compound might similarly react. For example, rather than testing every ligand in a large library for interaction with a complex or protein component, only those ligands having a fingerprint similar to other compounds known to have that activity could be tested. (See, e.g., Kauvar et al., 1995, Chemistry and Biology 2:107-118; Kauvar, 1995, Affinity fingerprinting, Pharmaceutical Manufacturing International. 8:25-28; and Kauvar, Toxic-Chemical Detection by Pattern Recognition in New Frontiers in Agrochemical Immunoassay, D. Kurtz. L. Stanker and J. H. Skerritt. Editors, 1995, AOAC: Washington, D.C., 305-312).

Kay et al., 1993, Gene 128:59-65 (Kay) discloses a method of constructing peptide libraries that encode peptides of totally random sequence that are longer than those of any prior conventional libraries. The libraries disclosed in Kay encode totally synthetic random peptides of greater than about 20 amino acids in length. Such libraries can be advantageously screened to identify complex modulators. (See also U.S. Pat. No. 5,498,538 dated Mar. 12, 1996; and PCT Publication No. WO 94/18318 dated Aug. 18, 1994).

A comprehensive review of various types of peptide libraries can be found in Gallop et al., 1994, J. Med. Chem. 37:1233-1251.

### 5.7. Pharmaceutical Compositions and Therapeutic/Prophylactic Administration

The invention provides methods of treatment (and prophylaxis) by administration to a subject of an effective amount of a Therapeutic of the invention. In a preferred aspect, the Therapeutic is substantially purified. The subject is preferably an animal including, but not limited to animals such as cows, pigs, horses, chickens, cats, dogs, etc., and is preferably a mammal, and most preferably human. In a specific embodiment, a non-human mammal is the subject.

Various delivery systems are known and can be used to administer a Therapeutic of the invention, e.g., encapsulation in liposomes, microparticles, and microcapsules: use of recombinant cells capable of expressing the Therapeutic, use of receptor-mediated endocytosis (e.g., Wu and Wu, 1987, J. Biol. Chem. 262:4429-4432); construction of a Therapeutic nucleic acid as part of a retroviral or other vector, etc. Methods of introduction include but are not limited to intradermal, intramuscular, intraperitoneal, intravenous, subcutaneous, intranasal, epidural, and oral routes. The compounds may be administered by any convenient route, for example by infusion, by bolus injection, by absorption through epithelial or mucocutaneous linings (e.g., oral, rectal and intestinal mucosa, etc.), and may be administered together with other biologically active agents. Administration can be systemic or local. In addition, it may be desirable to introduce the pharmaceutical compositions of the invention into the central nervous system by any suitable route, including intraventricular and intrathecal injection; intraventricular injection may be facilitated by an intraventricular catheter, for example, attached to a reservoir, such as an Ommaya reservoir. Pulmonary administration can also be employed, e.g., by use of an inhaler or nebulizer, and formulation with an aerosolizing agent.

In a specific embodiment, it may be desirable to administer the pharmaceutical compositions of the invention locally to the area in need of treatment. This may be achieved by, for example, and not by way of limitation, local infusion during surgery, topical application, e.g., in conjunction with a wound dressing after surgery, by injection, by means of a catheter, by means of a suppository, or by means of an implant, said implant being of a porous, non-porous, or gelatinous material, including membranes, such as sialastic membranes, or fibers. In one embodiment, administration can be by direct injection at the site (or former site) of a malignant tumor or neoplastic or pre-neoplastic tissue.

In another embodiment, the Therapeutic can be delivered in a vesicle, in particular a liposome (Langer, 1990, Science 249:1527-1533; Treat et al., 1989, In: Liposomes in the Therapy of Infectious Disease and Cancer, Lopez-Berestein and Fidler, eds., Liss, New York, pp. 353-365; Lopez-Berestein, ibid., pp. 317-327; see generally ibid.)

In yet another embodiment, the Therapeutic can be delivered via a controlled release system. In one embodiment, a pump may be used (Langer, supra; Sefton, 1987, CRC Crit. Ref. Biomed. Eng. 14:201-240; Buchwald et al., 1980, Surgery 88:507-516; Saudek et al., 1989, N. Engl. J. Med. 321:574-579). In another embodiment, polymeric materials can be used (Medical Applications of Controlled Release, Langer and Wise, eds., CRC Press, Boca Raton, Fla., 1974; Controlled Drug Bioavailability, Drug Product Design and Performance, Smolen and Ball, eds., Wiley, New York, 1984; Ranger and Peppas, 1983, Macromol. Sci. Rev. Macromol. Chem. 23:61; Levy et al., 1985, Science 228:190-192; During et al., 1989, Ann. Neurol. 25:351-356; Howard et al., 1989, J. Neurosurg. 71:858-863). In yet another embodiment, a controlled release system can be placed in proximity of the therapeutic target, i.e., the brain, thus requiring only a fraction of the systemic dose (e.g., Goodson, 1984, In: Medical Applications of Controlled Release, supra, Vol. 2, pp. 115-138). Other controlled release systems are discussed in the review by Langer (1990, Science 249:1527-1533).

In a specific embodiment where the Therapeutic is a nucleic acid encoding a protein Therapeutic, the nucleic acid can be administered in vivo to promote expression of its encoded protein, by constructing it as part of an appropriate nucleic acid expression vector and administering it so that it becomes intracellular, e.g., by use of a retroviral vector (U.S. Pat. No. 4,980,286), or by direct injection, or by use of microparticle bombardment (e.g., a gene gun; Biolistic, Dupont), or by coating it with lipids, cell-surface receptors or transfecting agents, or by administering it in linkage to a homeobox-like peptide which is known to enter the nucleus (e.g., Joliot et al., 1991, Proc. Natl. Acad. Sci. USA 88:1864-1868), etc. Alternatively, a nucleic acid Therapeutic can be introduced intracellularly and incorporated by homologous recombination within host cell DNA for expression.

The present invention also provides pharmaceutical compositions. Such compositions comprise a therapeutically effective amount of a Therapeutic, and a pharmaceutically acceptable carrier. In a specific embodiment, the term “pharmaceutically acceptable” means approved by a regulatory agency of the Federal or a state government or listed in the U.S. Pharmacopeia or other generally recognized pharmacopeia for use in animals, and more particularly, in humans. The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, including but not limited to peanut oil, soybean oil, mineral oil, sesame oil and the like. Water is a preferred carrier when the pharmaceutical composition is administered orally. Saline and aqueous dextrose are preferred carriers when the pharmaceutical composition is administered intravenously. Saline solutions and aqueous dextrose and glycerol solutions are preferably employed as liquid carriers for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsions, tablets, pills, capsules, powders, sustained-release formulations and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E. W. Martin. Such compositions will contain a therapeutically effective amount of the Therapeutic, preferably in purified form, together with a suitable amount of carrier so as to provide the form for proper administration to the patient. The formulation should suit the mode of administration.

In a preferred embodiment, the composition is formulated, in accordance with routine procedures, as a pharmaceutical composition adapted for intravenous administration to human beings. Typically, compositions for intravenous administration are solutions in sterile isotonic aqueous buffer. Where necessary, the composition may also include a solubilizing agent and a local anesthetic such as lidocaine to ease pain at the site of the injection. Generally, the ingredients are supplied either separately or mixed together in unit dosage form, for example, as a dry lyophilized powder or water-free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water or saline for injection can be provided so that the ingredients may be mixed prior to administration.

The Therapeutics of the invention can be formulated as neutral or salt forms. Pharmaceutically acceptable salts include those formed with free carboxyl groups such as those derived from hydrochloric, phosphoric, acetic, oxalic, tartaric acids, etc., those formed with free amine groups such as those derived from isopropylamine, triethylamine, 2-ethylamino ethanol, histidine, procaine, etc., and those derived from sodium, potassium, ammonium, calcium, and ferric hydroxides, etc.

The amount of the Therapeutic of the invention which will be effective in the treatment of a particular disorder or condition will depend on the nature of the disorder or condition, and can be determined by standard clinical techniques. In addition, in vitro assays may optionally be employed to help identify optimal dosage ranges. The precise dose to be employed in the formulation will also depend on the route of administration, and the seriousness of the disease or disorder, and should be decided according to the judgment of the practitioner and each patient's circumstances. However, suitable dosage ranges for intravenous administration are generally about 20-500 micrograms of active compound per kilogram body weight. Suitable dosage ranges for intranasal administration are generally about 0.01 pg/kg body weight to 1 mg/kg body weight. Effective doses may be extrapolated from dose-response curves derived from in vitro or animal model test systems.

Suppositories generally contain active ingredient in the range of 0.5% to 10% by weight; oral formulations preferably contain 10% to 95% active ingredient.

The invention also provides a pharmaceutical pack or kit comprising one or more containers filled with one or more of the ingredients of the pharmaceutical compositions of the invention. Optionally associated with such container(s) can be a notice in the form prescribed by a governmental agency regulating the manufacture, use or sale of pharmaceuticals or biological products, which notice reflects approval by the agency of manufacture, use or sale for human administration.

The invention also provides a pharmaceutical pack or kit comprising one or more containers filled with one or more of the ingredients of the pharmaceutical compositions of the invention. For example, the kit can comprise in one or more containers a first protein, or a functionally active fragment or functionally active derivative thereof, which first protein is selected from the group consisting of proteins listed in column A of table 1 of a given row; and a second protein, or a functionally active fragment or functionally active derivative thereof, which second protein is selected from the group consisting of proteins listed in column B of table 1 of said row. Alternatively, the kit can comprise in one or more containers, all proteins, functionally active fragments or functionally active derivatives thereof of from the group of proteins in column C of table 1.

The kits of the present invention can also contain expression vectors encoding the essential components of the complex machinery, which components after being expressed can be reconstituted in order to form a biologically active complex. Such a kit preferably also contains the required buffers and reagents. Optionally associated with such container(s) can be instructions for use of the kit and/or a notice in the form prescribed by a governmental agency regulating the manufacture, use or sale of pharmaceuticals or biological products, which notice reflects approval by the agency of manufacture, use or sale for human administration.

### 5.8 Animal Models

The present invention also provides animal models. In one embodiment, animal models for diseases and disorders involving the protein complexes of the present invention are provided. These animal models are well known in the art. These animal models include, but are not limited to those which are listed in the section 5.6 (supra) as exemplary animaid models to study any of the complexes provided in the invention. Such animals can be initially produced by promoting homologous recombination or insertional mutagenesis between genes encoding the protein components of the complexes in the chromosome, and exogenous genes encoding the protein components of the complexes that have been rendered biologically inactive or deleted (preferably by insertion of a heterologous sequence, e.g., an antibiotic resistance gene). In a preferred aspect, homologous recombination is carried out by transforming embryo-derived stem (ES) cells with one or more vectors containing one or more insertionally inactivated genes, such that homologous recombination occurs, followed by injecting the transformed ES cells into a blastocyst, and implanting the blastocyst into a foster mother, followed by the birth of the chimeric animal (“knockout animal”) in which a gene encoding a component protein from column A of table 1 of a given row, or a functionally active fragment or functionally active derivative thereof, and a gene encoding a component protein from column B of table 1 of said row, or a functionally active fragment or functionally active derivative thereof, has been inactivated or deleted (Capecchi, 1989, Science 244:1288-1292).

In another preferred aspect, homologous recombination is carried out by transforming embryo-derived stem (ES) cells with one or more vectors containing one or more insertionally inactivated genes, such that homologous recombination occurs, followed by injecting the transformed ES cells into a blastocyst, and implanting the blastocyst into a foster mother, followed by the birth of the chimeric animal (“knockout animal”) in which the genes of all component proteins from the group of proteins listed in column C of table 1 or of all proteins from the group of proteins listed in columb D of table 1 have been inactivated or deleted.

The chimeric animal can be bred to produce additional knockout animals. Such animals can be mice, hamsters, sheep, pigs, cattle, etc., and are preferably non-human mammals. In a specific embodiment, a knockout mouse is produced.

Such knockout animals are expected to develop, or be predisposed to developing, diseases or disorders associated with mutations involving the protein complexes of the present invention, and thus, can have use as animal models of such diseases and disorders, e.g., to screen for or test molecules (e.g., potential Therapeutics) for such diseases and disorders.

In a different embodiment of the invention, transgenic animals that have incorporated and express (or over-express or mis-express) a functional gene encoding a protein component of the complex, e.g. by introducing the a gene encoding one or more of the components of the complex under the control of a heterologous promoter (i.e., a promoter that is not the native promoter of the gene) that either over-expresses the protein or proteins, or expresses them in tissues not normally expressing the complexes or proteins, can have use as animal models of diseases and disorders characterized by elevated levels of the protein complexes. Such animals can be used to screen or test molecules for the ability to treat or prevent the diseases and disorders cited supra.

In one embodiment, the present invention provides a recombinant non-human animal in which an endogenous gene encoding a first protein, or a functionally active fragment or functionally active derivative thereof, which first protein is selected from the group of proteins of column A of table 2 of a given complex, and and endogenous gene encoding a second protein, or a functionally active fragment or functionally active derivative thereof, which second protein is selected from the group consisting of proteins of column B, of table 2 of said complex has been deleted or inactivated by homologous recombination or insertional mutagenesis of said animal or an ancestor thereof. In addition, the present invention provides a recombinant non-human animal in which the endogenous genes of all proteins, or functionally active fragments or functionally active derivatives thereof of one of the group of proteins listed in column C have been deleted or inactivated by homologous recombination or insertional mutagenesis of said animal or an ancestor thereof:

In another embodiment, the present invention provides a recombinant non-human animal in which an endogenous gene encoding a first protein, or a functionally active fragment or functionally active derivative thereof, which first protein is selected from the group consisting of proteins of column A of table 2 of a given complex, and endogenous gene encoding a second protein, or a functionally active fragment or functionally active derivative thereof, which second protein is selected from the group consisting of proteins of column B, of table 2 of said complex are recombinantly expressed in said animal or an ancestor thereof.

The following series of examples are presented by way of illustration and not by way of limitation on the scope of the invention.

### EXAMPLES

By applying the process according to the invention to the isolation of the polyadenylation/cleavage machinery from yeast, which is further described below, thirty-two new proteins could be identified in said yeast complex.

Purifications have been done using different proteins as bait according to the protocols stated further below.

Below is a more detailed list of the newly identified components of the polyadenylation complex (see also Tab. 1). The Accession-Number stated is the GenBank-Accession number for the protein.

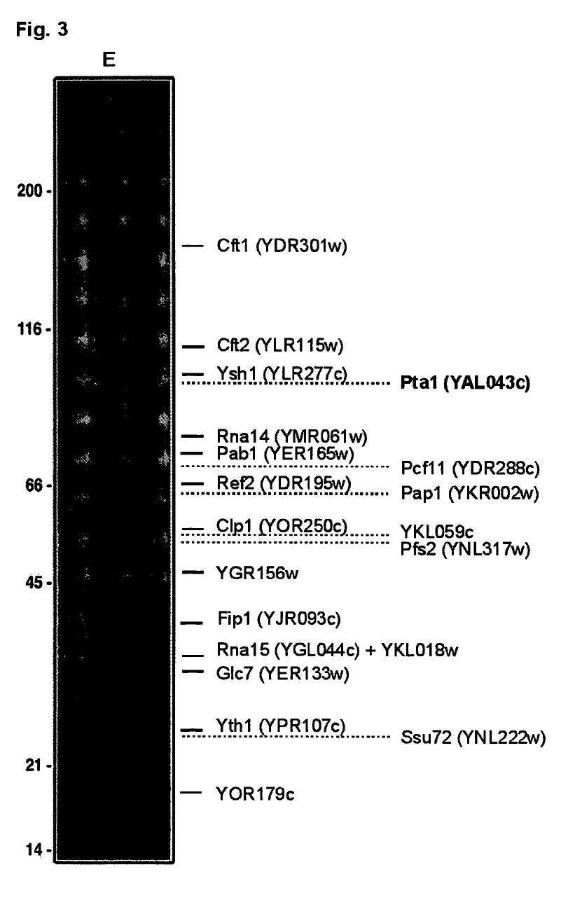

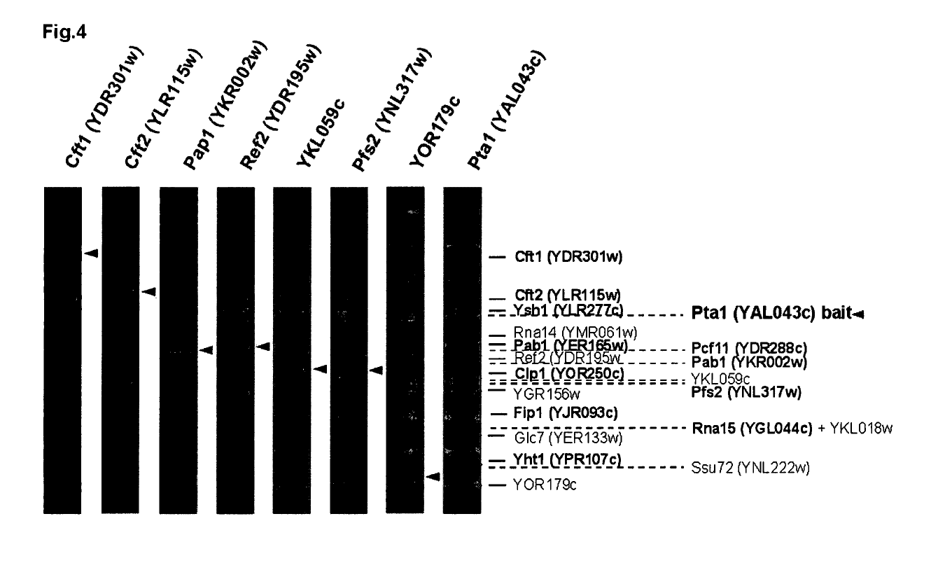

Protein patterns for some of the purifications are shown in FIGS. 3 and 4.

Act1: Is a known and essential protein (GenBank Acc. No. BAA21512.1), which has been shown to be involved in Pol II transcription and has been found to be associated with histone acetylation. It serves as a structural protein.

Cka1: Is a known and non-essential protein (GenBank Acc. No. CAA86916.1), which has been found to be involved in Polymerase III transcription and has been found to be associated with the Casein kinase II complex.

Eft2: The translation elongation factor EF-2 is a known protein involved in protein synthesis (GenBank AAB64827.1)

Eno2: Is a known and essential protein (GenBank Acc. No. AAB68019.1). It has been shown to have lyase activity and is known to be involved in carbohydrate metabolism.

Glc7 (YER1 33w) is also a known protein (GenBank Acc. No. AAC03231.1). It is also an essential protein and is a Type I protein serine threonine phosphatase which has been implicated in distinct cellular roles, such as carbohydrate metabolism, meiosis, mitosis and cell polarity. Its occurrence in the cleavage/polyadenylation machinery has not been known before.

Gpm1: This protein is a phosphoglycerate mutase that converts 2-phosphoglyvcerate to 3-phosphoglycerate in glycolysis. It is an essential protein (GenBank: CAA81994.1)

Hhf2: Is a known and non-essential protein (GenBank Acc. No. CAA95892.1) which has been shown to be involved in DNA-binding. It has previously been linked to Histone octamer and the RNA polymerase I upstream activation factor.

Hta1: Is a known and non-essential protein (GenBank Acc. No. CAA88505.1) which has DNA-binding capability and has been shown to be involved in polymerase II transcription.

Hsc82: Is a non-essential protein so far being associated with protein folding. (GenBank Acc. No: CAA89919.1)

Imd2: Is an Inosine-5′-monophosphate dehydrogenase so far being associated with nucleotide metabolism. It is non-essential. (GenBank Acc.-No.: AAB69728.1)

Imd4: Is a non-essential protein with similiarity to Imd2 so far being associated with nucleotide metabolism (GenBank Acc-No.: CAA86719.1)

Met6: Is a homocysteine methyltransferase so far being associated with amino-acid metabolism (GenBank Acc.-No.: AAB64646.1)

Pdc1: Is a pyruvate decarboxylase isozymel so far being associated with carbohydrate metabolism (GenBank Acc.-No.: CAA97573.1)

Pfk1: Is a known protein (GenBank Acc. No. CAA97268.1) which has previously been described as part of the phosphofructokinase complex.

Ref2 (YDR195w) is a known protein (GenBank Acc. No. CAA88708.1). It is a non-essential gene product. It has been shown to be involved in 3′-end formation prior to the final polyadenylation step. However, Ref2 has never been identified before as a component of the 3′-end processing machinery. Ref2 has been shown to interact with Glc7, another new component of the cleavage/polyadenylation machinery.

Sec13: Is a known and essential protein (GenBank Acc. No AAB67426.1).

Sec31: Is a known and essential protein (GenBank Acc. No. CAA98772.1)

Ssa3: Is a known and non-essential protein (GenBank Acc. No. CAA84896.1) which so far has been implicated with protein folding/protein transport.

Ssu72 (YNL222w) is also a known protein (GenBank Acc. No. CAA96125.1) and is an essential phylogenetically conserved protein which has been shown to interact with the general transcription factor TFIIB (Sua7). TFIIB is an essential component of the RNA polymerase II (RNAP II) core transcriptional machinery. It is thought that this interaction plays a role in the mechanism of start site selection by RNAP II. The finding according to the present invention that Ssu72 is associated with Pta1 is likely to be relevant since it is believed that mRNA 3′-end formation is linked with other nuclear processes like transcription, capping and splicing. Furthermore, Ssu 72 has also been clearly identified in a “reverse tagging experiment” as explained herein below by using some of the Pta1 associated proteins as bait. However, when Ssu72 itself was used as a bait associated proteins were not found most likely due to the fact that the addition of a C-terminal tag renders Ssu72 non-functional.

Taf60: Is a known and essential protein (GenBank Acc. No. CAA96819.1) which has been shown to be involved in Polymerase II transcription.

Tkl1: Is a non-essential transketolase so far being associated with amino-acid metabolism and carbohydrate metabolism (GenBank Acc-No.: CAA89191.1)

Tsa1: Translation initiation factor eIF5 which so far has been to shown to catalyze hydrolysis of GTP on the 40S ribosomal subunit-initiation complex followed by joining to 60S ribosomal subunit. (GenBankAcc.-No.: CAA92145.1)

Tye7: Is a known protein (GenBank Acc. No. CAA99671.1). It has been shown to be a basic helix-loop-helix transcription factor.

Vid24: Is a known and non-essential protein (GenBank Acc. No. CAA89320.1) which has previously been associated with protein degradation and vesicular transport.

Vps53: Is a known protein (GenBank Acc. No. CAA89320.1) which has been found to play a role in protein sorting.

YCL046w: Is a non-essential protein (GenBank Acc. No. CAA42371.1).

YGR156w is the protein product of an essential gene. This protein also contains a RNA binding motif. (GenBank Acc. No. CAA97170.1).

YHL035c: Is a known and non-essential protein (GenBank Acc. No. AAB65047.1). It is a member of the ATP-binding cassette superfamily.

YKL018w is also an essential protein containing a WD40 domain which is a typical protein binding domain. (GenBank Acc. No. CAA81853.1)

YLR221c: Is a protein of unknown function (GenBank Acc. No.AAB67410.1)

YML030w: Is a protein of unknown function (GenBank Acc. No. CAA86625.1)

YOR179c shows significant sequence similarity to Ysh1 (GenBank Acc. No. CAA99388.1)

Two further proteins for which binary interactions with members of the polyadenylation complex as known so far have been shown before have also been purified with the complex:

YKL059c: is the product of an essential gene and is a zinc binding protein containing a C2HC Zinc finger. The presence of this domain predicts a RNA binding function of YKL059c. We believe the corresponding gene product is identical to Pfs1, a protein which has been mentioned in several publications, but which has never been annotated in the databases (for review see Keller, W. and Minvielle-Sebastia (1997). Curr Opin Cell Biol 11: 352-357). (GenBank Acc. No. CAA81896.1)

Tif4632: Is a known and non-essential protein (GenBank Acc. No. CAA96751.1) which has been shown to have an RNA-binding/translation factor activity and is involved in protein synthesis.

Below is a description of the experimental steps and protocols as used herein:

The initial round of purification of the complex was carried out using Pta-1 as a bait as described below:

Construction of a Yeast Strain Expressing Tap-Tagged Pta1

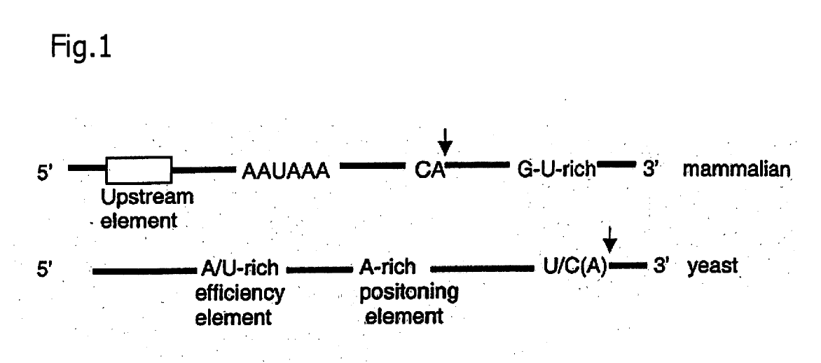

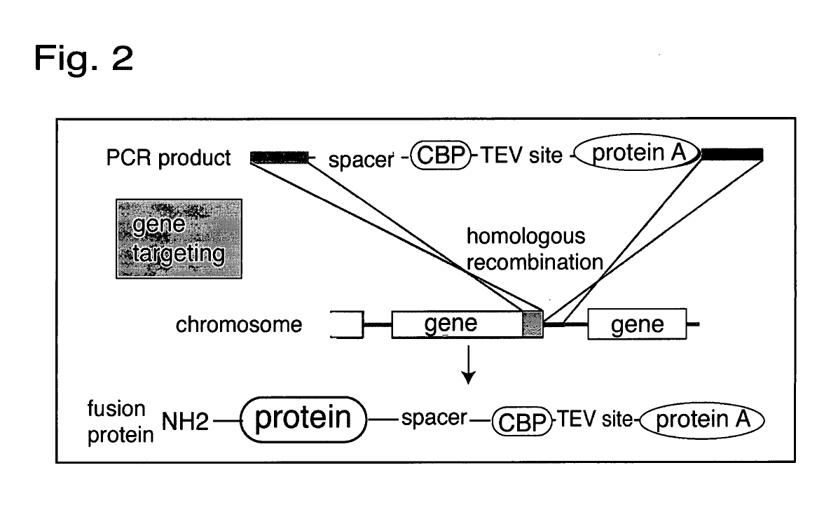

The construction of these strains is illustrated both in FIG. 2 and table 5.

Purification of Proteins Associated with PTA1

The TAP-technology, which is more fully described in WO/0009716 and in Rigaut, G. et. al. (1999), Nature Biotechnology. Vol. 17 (10): 1030-1032 respectively was used for protein complex purification. The Pta1 protein was C-terminally tagged with a TAP-tag which consists of calmodulin-binding peptide (CBP), a cleavage site for TEV protease followed by two IgG-binding units of protein A (Rigaut, G. et. al. (1999), Nature Biotechnology. Vol. 17 (10): 1030-1032). Pta1 is an essential protein which has been reported to be a component of PFI. Pta1-TAP was used as a bait to identify associated partners from cell lysates using the two-step TAP purification procedure. Proteins were separated by 1D gel electrophoresis and visualized by staining with Coomassie. More than a total of 20 bands could be detected on the gel (see FIG. 3). The identity of the proteins was determined by mass spectrometry. 13 of these are known components of the pre-mRNA processing machinery: Cft1, Cft2, Ysh, Pta1, Rna14, Pab1, Pcf11, Pap1, Clp1, Pfs2, Fip1, Rna15 and Yth1. It is to be noted that such a comprehensive number has never before been purified together in form of a complex. The remaining seven proteins have not previously been found associated with Pta1: Ref2, YK059c, YGR156w, YKL018w, Glc7, Ssu72 and YOR179c.

Validation of Interactions Found with Pta1

A reciprocal experiment to the one described above was performed. For this purpose a subset of the interactors found in the above described Pta purification (both known and novel interactors) were chosen as a bait for a further round of purification (the baits used herein are listed in the first column of Table 1). In the case of some proteins the C-terminally tagged versions could not be recovered. The likely reason for this is that the addition of the TAP tag at the C-terminus interferes with the function of these proteins. An important fact is that almost all of the known components involved in 3′-end formation and five of the seven novel proteins identified herein are essential for cell viability. The protein pattern obtained in some of those experiments is shown in FIG. 4. The construction of the strains was carried out as described for the strain expressing the TAP-tagged Pta-1.

Sequence Analysis of Members of the Complex

The process of mRNA processing is highly conserved in eukaryots. Accordingly, for a number of the yeast proteins human orthologues could be found (see Table 2). This illustrates that many of the functions found in the yeast complex can be transferred to humans. Also the enzymatic activity of this complex has long been known, the enzymatically active member could not yet be unraveled. Using extensive sequence similarity searches it could be shown that Ysh1 is homologous to a class of bacterial beta-lactamases. The active center of this protein family contains 2 zinc ions which are bound by histidines. As these residues are conserved in Ysh1 and it was shown that enzymatic activity of the yeast complex is zinc dependent predicted that Ysh1 is responsible for the catalytic activity of the complex. Two other proteins found in the complex, Cft2 and YOR179c, are homologous to the Ysh1 N- and C-terminus, respectively. Though Cft2 is homologous to the enzymatic region of Ysh1 it misses the zinc binding histidines indicating that it lacks enzymatic activity. Thus, Cft 2 and YOR179c could compete with Ysh1 for the same binding slot of the complex, suggesting a novel type of regulation of polyadenylation. A similar way of regulation might be used in the case of Pfs2 and YKL018w, which both consist of multiple WD40 domains.

Prediction of Mammalian Proteins

To allow the transfer of function information from yeast to human proteins, we did not only use an identity cutoff, but also the ‘orthology’ concept. Orthology defines genes which arose via a speciation event, in contrast to genes which arose via gene duplication. Orthologue genes are supposed to perform the same function in different organisms, therefore more detailed function information can be transferred. The algorithm for the detection of orthologous gene pairs from yeast and human uses the whole genome of these organisms. First, pairwise best hits were retrieved, using a full Smith-Waterman alignment of predicted proteins. To further improve reli-ability, these pairs were clustered with pairwise best hits involving Drosophila melanogaster and Caenorhabditis elegans proteins. See “Initial sequencing and analysis of the human genome”, Nature 2001 Feb 15; 409(6822):860-921 for a detailed description of the analysis.

Bioinformatic Analysis of the Complex:

Functional domains of all members of the complex were analyzed using SMART (SMART: a web-based tool for the study of genetically mobile domains. Nucleic Acids Res 2000 Jan 1; 28(1):231-4) and Pfam (Pfam: protein families database, Nucleic Acids Res 2000 Jan 1; 28(1):263-6).

Comparison of the Yeast and Mammalian Cleavage/Polyadenylation Machinery

The sequence of many of the polypeptides involved in 3′-end formation are conserved form yeast to mammals, although the sequence elements on the substrate pre-mRNA differ (see FIG. 1).

The detailed experimental protocols for the example stated herein are given below:

### Protocols

Isolation of Protein Complexes:

a) Isolation FO Complexes from Yeast:

Yeast Strain Construction:

Yeast strains expressing TAP-tagged ORFs were constructed in a semi-automated way essentially according to Rigaut et. al. (Rigaut, G. et. al. Nat Biotechnol 17, 1030-2 (1999)) and Puig et al. (Puig, O. et al. Methotds 24, 218-19. (2001)) (See also FIG. 2 and Table 5)

TAP-purification using the Pta-1-tagged Strains:

Pta1-tagged strain was cultured in 4 l of YPD medium to an OD600 of 2.

After harvesting, the cell pellet was frozen in liquid nitrogen and stored at −80° C. All further manipulations were done at 4° C. except where noted. For preparation of protein lysates the cells were resuspended in lysis buffer (50 mM Tris-HCl pH 7.5, 100 mM NaCl, 0.15% NP-40, 1.5 mM MgCl2, 0.5 mM DTT, protease inhibitors) and subjected to mechanical disruption with glass beads. Lysates were clarified by two successive centrifugation steps at 20.000×g for 10 min and 100.000×g for 1 hour. After addition of glycerol to 5% final concentration the lysates were frozen in liquid nitrogen and stored at −80° C.

For the first purification step 500 μl of rabbit IgG-Agarose (50:50 slurry, Sigma A2909) pre-equilibrated in lysis buffer were added to the lysate and the sample was rotated for 2 hours. The unbound fraction was discarded and the beads with the bound material were transferred to a 0.8 ml column (MoBiTec M1002, 90 μm filter). The beads were washed with 10 ml of lysis buffer followed by 5 ml of TEV cleavage buffer (10 mM Tris-HCl pH 8.0, 100 mM NaCl, 0.1% NP-40, 0.5 mM EDTA, 1 mM DTT).

150 μl of TEV cleavage buffer and 4 μl of TEV protease were added to the column and the sample was incubated on a shaker at 16° C. for 2 hours. The eluate was recovered by pressing with a syringe.

150 μl of Calmodulin dilution buffer (10 mM Tris-HCl pH 8.0, 100 mM NaCl, 0.1% NP-40, 2 mM MgAc, 2 mM imidazole, 4 mM CaCl2, 1 mM DTT) was added to the previous eluate and this mixture was transferred to a MoBiTec column containing 300 μl (bead volume) of Calmodulin affinity resin (Stratagene #214303) which was prewashed in Calmodulin wash buffer (10 mM Tris-HCl pH 8.0, 100 mM NaCl, 0.1% NP-40, 1 mM MgAc, 1 mM imidazole, 2 mM CaCl2, 1 mM DTT). The samples were rotated for 1 hour at 4° C.

After washing of the beads with 10 ml of Calmodulin wash buffer, protein complexes were eluted with 600 μl of elution buffer (10 mM Tris-HCl pH 8.0, 5 mM EDTA). The samples were concentrated in siliconised tubes in a speed vac to a final volume of 10-20 μl. Proteins were detected by polyacrylamide gel electrophoresis followed by staining with colloidal Coomassie blue.

General TAP-Purification Protocol for Soluble Proteins:

TAP-Purification of Soluble Proteins:

The purification was done from 2 liters of yeast cells grown to late log phase (OD₆₀₀ ˜3-4). Cells were harvested and the pellet was frozen in liquid nitrogen and stored at −80° C. All steps were done at 4° C. For preparation of protein lysates the cells were resuspended in buffer A (50 mM Tris-HCl pH 7.5, 100 mM NaCl, 0.15% NP-40, 1.5 mM MgCl₂, 0.5 mM DTT, protease inhibitors) and subjected to mechanical disruption with glass beads. Lysates were clarified by two successive centrifugation steps at 20.000×g for 10 min and 100,000×g for 1 hour. After addition of glycerol to 5% final concentration the lysates were frozen in liquid nitrogen and stored at −80° C.

For the first purification step 500 μl of rabbit IgG-Agarose (50:50 slurry, Sigma A2909) pre-equilibrated in buffer A were added to the lysate and the sample was rotated for 1 hour. The unbound fraction was discarded and the beads with the bound material were transferred to a 0.8 ml column (MoBiTec M1002, 90 μm filter). The beads were washed with 10 ml of buffer A.

150 μl of buffer A and 4 μl of TEV protease (1 mg/ml) were added to the column and the sample was incubated on a shaker at 16° C. for 1 hour. The eluate was recovered by pressing with a syringe.

150 μl of buffer A containing 4 mM CaCl₂ was added to the previous eluate and this mixture was transferred to a MoBiTec column containing 300 μl (bead volume) of Calmodulin affinity resin (Stratagene #214303) which was prewashed in buffer A containing 2 mM CaCl₂. The samples were rotated for 1 hour at 4° C.

After washing of the beads with 5 ml of buffer A containing 2 mM CaCl₂, protein complexes were eluted with 600 μl of elution buffer (10 mM Tris-HCl pH 8.0, 5 mM EGTA). The samples were concentrated in siliconized tubes in a speed vac. Proteins were detected by polyacrylamide gel electrophoresis followed by staining with colloidal Coomassie blue.

TAP-Purification of Membrane Proteins:

The purification was done from 2 liters of yeast cells grown to late log phase (OD₆₀₀ ˜3-4). Cells were harvested and the pellet was frozen in liquid nitrogen and stored at −80° C. All steps were done at 4° C. For the purification of TAP-tagged membrane proteins cells were lysed in buffer containing 50 mM Hepes/KOH pH 7.5, 150 mM KCl, 0.25% NP40, 2 mM MgCl₂, 2 mM EDTA, 0.5 mM DTT and protease inhibitors. The extracts were spun at 20,000×g for 10 min and the resulting supernatant was adjusted to 1.5% NP-40 and 5% glycerol. Samples were incubated for 30 min with end-over-end shaking and then centrifuged at 180,000×g for 30 min. The resulting supernatant was immediately used for TAP-purification.

For the first purification step 500 μl of rabbit IgG-Agarose (50:50 slurry, Sigma A2909) pre-equilibrated in buffer B (50 mM Tris-HCl pH 7.5, 100 mM NaCl, 0.5% NP-40, 1.5 mM MgCl₂, 0.5 mM DTT, protease inhibitors) was added to the lysate and the sample was rotated for 1 hour. The unbound fraction was discarded and the beads with the bound material were transferred to a 0.8 ml column (MoBiTec M1002, 90 μm filter). The beads were washed with 10 ml of buffer B.

150 μl of buffer B and 8 μl of TEV protease (1 mg/ml) were added to the column and the sample was incubated on a shaker at 16° C. for 1 hour. The eluate was recovered by pressing with a syringe.

150 μl of buffer B containing 4 mM CaCl₂ was added to the previous eluate and this mixture was transferred to a MoBiTec column containing 300 μl (bead volume) of Calmodulin affinity resin (Stratagene #214303) which was prewashed in buffer B containing 2 mM CaCl₂. The samples were rotated for 1 hour at 4° C. After washing of the beads with 5 ml of buffer B containing 2 mM CaCl₂, protein complexes were eluted with 600 μl of elution buffer (10 mM Tris-HCl pH 8.0, 5 mM EGTA). The samples were concentrated in siliconized tubes in a speed vac. Proteins were detected by polyacrylamide gel electrophoresis followed by staining with colloidal Coomassie blue.

b) Isolation of Complexes from Mammalian Cells

Cells:

Retroviral transduction vectors containing the TAP-cassette were generated by directional cloning of PCR-amplified ORFs into a modified version of a MmoLV-based vector via the Gateway site-specific recombination sstem (Life Technologies). Virus stocks were generated in a HEK293 gag-pol packaging cell line by pseudotyping with VSV-G. Cells were infected and complexe were purified after cell expansion and cultivation of 3-5 using a modified TAP-protocoll

Standard Lysis Protocol:

The medium was removed from the culture dish and the cells were scraped directly from the plate with help of a rubber policeman. The cells were collected on ice washed 3 times with PBS and resuspended in lysis buffer (50 mM Tris, pH: 7.5; 5% glycerol; 0,2% IGEPAL; 1.5 mM MgCl2; 1 mM DTT; 100 mM NaCl; 50 mM NaF; 1 mM Na3VO4+protease inhibitors). The cells were lysed for 30 min on ice, spun for 10 min. at 20,000 g and re-spun for 1 h at 100,000 g. The supernatant was recovered, rapidly frozen in liquid nitrogen and stored at −80° C. For pre-clearing the thawed lysate was incubated with 500 μl sepharose CL-4B beads (Amersham Pharmacia) for 1 h shaking and finally processed according the TAP protocol.

Nuclear Lysis Protocol:

The medium was removed from the culture dish and the cells were scraped directly from the plate with help of a rubber policeman. The cells were collected on ice washed 3 times with PBS and resuspended in buffer A (10 mM Tris-Cl, pH 7.5; 1, 5 mM MgCl2; 10 mM KCl; 1 mM DTT, 50 mM NaF; 1 mM Na3VO4). To isolate the nuclei the lysate was dounced with a tight fitted pestle in a dounce homogenizer for 15 strokes. The nuclei were harvested by centrifugation (10 min. at 2000 g and 20 min. at 16,000 g) and lysed in buffer B (50 mM Tris-Cl, pH: 7.5; 1.5 mM MgCl; 20% glycerol; 420 mM NaCl; 1 mM DTT; 50 mM NaF; 1 mM Na3VO4) for 30 min. on ice with frequent shaking. The protein lysate was cleared by centrifugation (30 min at 100,000 g) and 1:4 diluted with buffer C (50 mM Tris-Cl, pH: 7.5; 1 mM DTT; 0.26% NP40; 1.5 mM MgCl; 50 mM NaF; 1 mM Na3VO4). After 30 min incubation on ice the lysate was re-spun for 30 min at 100,000 g, quickly frozen in liquid nitrogen and stored at −80° C. For pre-clearing the thawed lysate was incubated with 500 μl sepharose CL-4B beads (Amersham Pharmacia) for 1 h shaking and finally processed according the TAP protocol.

Mass Spectrometric Analysis

Protein Digestion Prior to Mass Spectrometric Analysis:

Gel-separated proteins were reduced, alkylated and digested in gel essentially following the procedure described by Shevchenko et al. (Shevchenko, A., Wilm, M., Vorm, O., Mann, M. Anal Chem 1996, 68, 850-858). Briefly, gel-separated proteins were excised from the gel using a clean scalpel, reduced using 10 mM DTT (in 5 mM ammonium bicarbonate, 54° C., 45 min) and subsequently alkylated with 55 mM iodoacetamid (in 5 mM ammonium bicarbonate) at room temperature in the dark (30 min). Reduced and alkylated proteins were digested in gel with porcine trypsin (Promega) at a protease concentration of 12.5 ng/μl in 5 mM ammonium bicarbonate. Digestion was allowed to proceed for 4 hours at 37° C. and the reaction was subsequently stopped using 2 μl 25% TFA.

Desalting and Concentration of Peptides Produced By In-Gel Digestion of Gel-Separated Proteins:

Peptides were desalted and concentrated using a prefabricated uZipTip (Millipore) reversed phase column. Peptides were eluted directly onto stainless steel MS sample holders using 2 μl eluent (70% acetonitrile in 5% TFA containing 2 mg/ml alpha-Cyano-4-hydroxy-cinnamic acid and two standard peptides for internal calibration of mass spectra).

Mass Spectrometric Data Acquisition:

Matrix-assisted laser desorption/ionisation (MALDI) time-of-flight (TOF) mass spectra were acquired in delayed extraction mode on a Voyager DE-STR PRO MALDI mass spectrometer (Applied Biosystems) equipped with a 337 nm nitrogen laser. 500 laser shots were averaged in order to produce final spectra. Spectra were automatically internally calibrated using two standard peptides. The monoisotopic masses for all peptide ion signals detected in the acquired spectra were determined and used for database searching.

Protein Sequence Database Searching Using Peptide Mass Fingerprinting (PMF) Data:

The list of monoisotopic peptide masses obtained from the MALDI mass spectrum was used to query a fasta formatted protein sequence database that contained all protein sequences from S. cerevesia. Proteins were identified by peptide mass fingerprinting (Mann, M., Højrup, P., Roep-storff, P. Biol Mass Spectrom 1993, 22, 338-345; Pappin, D., Højrup, P, Bleasby, AJ Curr. Biol. 1993, 3, 327-33; Henzel, W. J., Billeci, T. M., Stults, J. T., Wong, S. C., Grimley, C., Watanabe, C. Proc Natl Acad Sci U S A 1993, 90, 5011-5015; Yates, J. R., Speicher, S., Griffin, P. R., Hunkapiller, T. Anal Biochem 1993, 214, 397-408; James, P., Quadroni, M., Carafoli, E., Gonnet, G. Biochem Biophys Res Commun 1993, 195, 58-64) using the software tool Profound (Proteometrics). In PMF, a protein is identified by correlating the measured peptide masses with theoretical digests of all proteins present in the database. Search criteria included: tryptic protein cleavage, monoisotopic masses, 30 ppm mass accuracy. No restrictions on protein size or isoelectric point were imposed.

Bioinformatics

Functional and localization information about yeast proteins was retrieved from the Yeast Protein Database (YPD (Constanzo, M. C. et al., 2001, Nucl. Acid Res, 29: 75-9; Hodges, P. E. et al., 1999, Nucl. Acids Res 27: 69-73)) released in August 2001. In order to get a more concise classification for localization and function, YPD classes were merged. Protein domain analysis was performed using SMART (Schultz, J., Copley, R. R., Doerks, T., Ponting, C. P. & Bork, P. Schultz, J., Copley, R. R., Doerks, T., Ponting, C. P. & Bork, P. SMART, Nucleic Acids Res 28, 231-4. (2000)). PsiBlast (Altschul, S. F. et al. Gapped BLAST and PSI-BLAST: Nucleic Acids Res 25, 3389-402. (1997)) was used for homology analysis. All additional analysis software has been developed in house, using Perl and Python.

### Assays for Assaying the Activities of the Complexes Presented in the Invention

An exemplary RNA binding assay can be carried out by contacting a complex having RNA binding activity with a radioactive [32P] end-labeled RNA substrate, e.g. a poly (A) RNA, under appropriate conditions and detecting bound protein. The detection of bound protein can be carried out, e.g., by filtrating the solution through a nitrocellulose filter and determining the radioactivity bound to the filter. This assay is based on the retention of nucleic acid-protein complexes on Nitrocellulose whereas free nucleic acid can pass through the filter

(see e.g. Wahle, E., 1991, Methods 66: 759-68)

An exemplary RNA exonuclease assay can be carried out by contacting a complex having RNA exonuclease activity with a radioactivity [32 phosphate] end-labeled RNA substrate under appropriate conditions and detecting the release of free radioactive nucleotides. The detection of free radioactive nucleotides can be carried out, e.g., by adding 20% trichloroacetic acid, filtrating the solution through a filter and measuring the amount of acid-soluble radioactivity

(see e.g. Ross, J., 1999, Methods 17: 52-9)

An exemplary mRNA splicing assay can be carried out by contacting a complex having mRNA splicing activity with a radioactively labeled RNA substrate under appropriate conditions and detecting the release of spliced RNA species. The detection of spliced RNA species can be carried out, e.g., by fractionation of processed RNAs in a glycerol gradient and subsequent analysis by denaturing polyacrylamide gel elecrophoresis and visualization by autoradiography.

(see e.g. Schwer, B. and Gross, C H., 1998, Methods17: 2086-94)

An exemplary rRNA processing assay can be carried out by contacting a complex having rRNA processing activity with an pre-rRNA substrate under appropriate conditions and detecting the release of free processed rRNA species. The detection of processed rRNA species can be carried out, e.g., using a primer extension or northern blotting assay by measuring the size of the rRNA species.

(see e.g. Kressler, D. et al, 1997, Methods 17: 7283-94)

| | | | | | | | Column | |
|---|---|---|---|---|---|---|---|---|
| | | | COLUMN A: | | COLUMN C | | D: | |
| | | | known | | Cleavage/ | | Proteins | |
| | | | components | COLUMN B: | poly- | | of | |
| Entry | | Proteins | of the | novel | adenylation | Activity of | unknown | |
| point | Interactions | found | complex | proteins | machinery | the complex | function | local. |
| Cft1 | Cft1, Cft2, Pta1, | Act1 Cft1 | Cft1 Cft2 Clp1 | Act1 Cka1 | Act1 Cft1 Cft2 | 3′ end | Ycl046w | c n u e |
| Cft2 | Ysh1 coippt; | Cft2 Cka1 | Fip1 Pab1 | Eft2 Eno2 | Cka1 Clp1 | processing | Ygr156w | |
| Clp1 | Cft1, Cft2, Fip1, | Clp1 Eft2 | Pap1 Pcf11 | Glc7 Gpm1 | Eft2 Eno2 | activity for | Yhl035c | |
| Fip1 | Pap1, Pta1, | Eno2 Fip1 | Pfs2 Pta1 | Hhf2 Hta1 | Fip1 Glc7 | mRNA | Ykl018w | |
| Pap1 | Ykl059c, Ysh1, | Glc7 | Rna14 Rna15 | Hsc82 Imd2 | Gpm1 Hhf2 | | Ylr221c | |
| Pcf1 | Yth1 coippt; | Gpm1 | Tif4632 | Imd4 Met6 | Hsc82 Hta1 | | Yml030w | |
| 1 | Fip1, Yth1 2- | Hhf2 | Ykl059c Ysh1 | Pdc1 Pfk1 | Imd2 Imd4 | | Yor179c | |
| Pfs2 | hybrid, coippt, | Hsc82 | Yth1 | Ref2 Sec13 | Met6 Pab1 | | | |
| Pta1 | high-throughput | Hta1 Imd2 | | Sec31 Ssa3 | Pap1 Pcf11 | | | |
| Ref2 | 2-hybrid, in vitro | Imd4 Met6 | | Ssu72 Taf60 | Pdc1 Pfk1 | | | |
| Rna1 | binding; Glc7, | Pab1 | | Tkl1 Tsa1 | Pfs2 Pta1 | | | |
| 4 | Ref2; Fip1, | Pap1 | | Tye7 Vid24 | Ref2 Rna14 | | | |
| Rna1 | Rna14 2-hybrid; | Pcf11 | | Vps53 | Rna15 Sec13 | | | |
| 5 | Rna14, Rna15 | Pdc1 Pfk1 | | Ycl046w | Sec31 Ssa3 | | | |
| Ykl0 | 2-hybrid; Clp1, | Pfs2 Pta1 | | Ygr156w | Ssu72 Taf60 | | | |
| 59c | Pcf11 coippt, | Ref2 | | Yhl035c | Tif4632 Tkl1 | | | |
| Yor1 | high-throughput | Rna14 | | Ykl018w | Tsa1 TYe7 | | | |
| 79c | 2-hybrid; Fip1, | Rna15 | | Ylr221c | Vid24 Vps53 | | | |
| Ysh1 | Pap1 2-hybrid, | Sec13 | | Yml030w | Ycl046w | | | |
| Yth1 | coippt; Fip1, | Sec31 | | Yor179c | Ygr156w | | | |
| | Ysh1 coippt, | Ssa3 | | | Yhl035c | | | |
| | copurification; | Ssu72 | | | Ykl018w | | | |
| | Hhf2, Hta1 | Taf60 | | | Ykl059c | | | |
| | affinity column, | Tif4632 | | | Ylr221c | | | |
| | coippt; Sec13, | Tkl1 Tsa1 | | | Yml030w | | | |
| | Sec31 2-hybrid, | TYe7 | | | Yor179c Ysh1 | | | |
| | affinity column, | Vid24 | | | Yth1 | | | |
| | copurification, | Vps53 | | | | | | |
| | high-throughput | Ycl046w | | | | | | |
| | 2-hybrid; Pab1, | Ygr156w | | | | | | |
| | Pcf11p, | Yhl035c | | | | | | |
| | Rna14p, | Ykl018w | | | | | | |
| | Rna15p | Ykl059c | | | | | | |
| | copurification; | Ylr221c | | | | | | |
| | Pab1, Tif4632 | Yml030w | | | | | | |
| | copurification, in | Yor179c | | | | | | |
| | vitro binding; | Ysh1 Yth1 | | | | | | |
| | Pcf11, Rna14, | | | | | | | |
| | Rna15 | | | | | | | |
| | copurification; | | | | | | | |
| | Pcf11, Rna14 2- | | | | | | | |
| | hybrid, coippt, | | | | | | | |
| | copurification, | | | | | | | |
| | high-throughput | | | | | | | |
| | 2-hybrid; Pcf11, | | | | | | | |
| | Rna15 2-hybrid, | | | | | | | |
| | copurification, | | | | | | | |
| | high-throughput | | | | | | | |
| | 2-hybrid; Pfs2, | | | | | | | |
| | Rna14 2-hybrid, | | | | | | | |
| | coippt; Pfs2, | | | | | | | |
| | Ysh1 coippt; | | | | | | | |
| | Fip1, Pfs2 | | | | | | | |
| | coippt; Pap1, | | | | | | | |
| | Yth1 coippt; | | | | | | | |
| | Clp1, Rna14 | | | | | | | |
| | coippt; Clp1, | | | | | | | |
| | Rna15; Ysh1, | | | | | | | |
| | Yth1 in vitro | | | | | | | |
| | binding; Sec13, | | | | | | | |
| | Sec31 | | | | | | | |
| | Coatomer | | | | | | | |
| | COPII complex; | | | | | | | |
| | Cft1, Cft2, Pta1, | | | | | | | |
| | Ysh1 Pre- | | | | | | | |
| | mRNA cleavage | | | | | | | |
| | factor II; Hhf2, | | | | | | | |
| | Hta1 Histone | | | | | | | |
| | octamer; Cft1, | | | | | | | |
| | Cft2, Fip1, | | | | | | | |
| | Pap1, Pta1, | | | | | | | |
| | Ykl059w, Ysh1, | | | | | | | |
| | Yth1 | | | | | | | |
| | Polyadenylation | | | | | | | |
| | Factor I (PFI); | | | | | | | |
| | Cft1, Cft2, Fip1, | | | | | | | |
| | Pap1, Pfs2, | | | | | | | |
| | Pta1, Ykl059w, | | | | | | | |
| | Ysh1, Yth1 | | | | | | | |
| | polyadenylation | | | | | | | |
| | factor I (PF I); | | | | | | | |
| | Clp1, Pab1, | | | | | | | |
| | Pcf11, Rna14, | | | | | | | |
| | Rna15 Pre- | | | | | | | |
| | mRNA cleavage | | | | | | | |
| | and | | | | | | | |
| | polyadenylation | | | | | | | |
| | factor IA; Fip1, | | | | | | | |
| | Pap1, Ysh1 | | | | | | | |
| | Pre-mRNA | | | | | | | |
| | polyadenylation | | | | | | | |
| | factor I; Sec13, | | | | | | | |
| | Sec31 Sec13p- | | | | | | | |
| | Sec31p | | | | | | | |

TABLE 2
INDIVIDUAL YEAST PROTEINS OF THE COMPLEXES
A)

| Yeast | | | | | | | | |
|---|---|---|---|---|---|---|---|---|
| proteins | | | | | | Human | C. elegans | Drosophila |
| listed in | SEQ | | SWISS- | | | orthologue | orthologue | orthologue |
| table 2 | ID | MIPS | PROT | SGD | Genbank | in GenBank | in GenBank | in GenBank |
| ACT1 | 1 | ATBY | P02579 | S0001855 | D50617 | gi4501885 | ACT4_CAEE | Q9W460 |
| | | | | | | | L | |
| CFT1 | 3 | S61187 | | S0002709 | U28374 | | | |
| CFT2 | 5 | S64952 | | S0004105 | Z73287 | | | |
| CKA1 | 7 | A31564 | P15790 | S0001297 | Z46861 | | | |
| CLP1 | 9 | S67147 | | S0005776 | Z75158 | gi5803029 | YMI4_CAEE | Q9V6Q1 |
| | | | | | | | L | |
| EFT2 | 11 | | P32324 | S0002793 | AAB64827.1 | gi: 4503483 | EF2_{—}CAEEL | Q9V9RO |
| ENO2 | 13 | NOBY2 | P00925 | S0001217 | U00027 | gi: 4503571 | ENO_CAEE | Q9VQ38 |
| | | | | | | | L | |
| GLC7 | 15 | S32595 | P32598 | S0000935 | U18916 | gi4506003 | YME1_CAE | Q9VC69 |
| | | | | | | | EL | |
| GPM1 | 17 | PMBYY | P00950 | S0001635 | Z28152 | | | |
| Fip1 | 19 | A56545 | P45976 | S0003853 | Z49593 | | | |
| HHF2 | 21 | | P02309 | S0004975 | Z71306 | | | |
| HTA1 | 23 | HSBYA1 | P04911 | S0002633 | Z48612 | | | |
| HSC82 | 25 | S55133 | P15108 | S0004798 | CAA89919.1 | gi6680307 | gi3875041 | gi7292327 |
| IMD2 | 27 | S48997 | P38697 | S0001259 | AAB69728.1 | gi4504689 | gi18030187 | gi7291188 |
| IMD4 | 29 | S50890 | P50094 | S0004520 | CAA86719.1 | gi4504688 | gi18030187 | gi7291188 |
| MET6 | 31 | S50594 | P05694 | S0000893 | AAB64646.1 | | | |
| PAB1 | 33 | DNBYPA | P04147 | S0000967 | U18922 | F15P000000 | Q9U302 | Q9V8C3 |
| | | | | | | 84557 | | |
| PAP1 | 35 | S19031 | P29468 | S0001710 | Z28227 | P51003 | Q20370 | Q9V8X7 |
| PCF11 | 37 | S59435 | P39081 | S0002636 | Z48612 | gi7706224 | YRR2_CAEE | Q9V768 |
| | | | | | | | L | |
| PDC1 | 39 | DCBYP | P06169 | S0004034 | Z73216 | | | |
| PFK1 | 41 | JQ0016 | P16861 | S0003472 | Z73025 | | | |
| PFS2 | 43 | S51295 | P42841 | S0005261 | Z71593 | F15P000000 | CAB76722 | Q9VNG2 |
| | | | | | | 84546 | | |
| PTA1 | 45 | S31299 | Q01329 | S0000041 | U12980 | | | |
| REF2 | 47 | S52702 | P42073 | S0002603 | Z48784 | | | |
| RNA14 | 49 | S54561 | P25298 | S0004665 | Z49703 | | | |
| RNA15 | 51 | B40257 | P25299 | S0003012 | Z72566 | F15P000000 | O45577 | Q9VE52 |
| | | | | | | 61276 | | |
| SEC13 | 53 | A45442 | Q04491 | S0004198 | U14913 | P55735 | AAF36082 | Q9V3J4 |
| SEC31 | 55 | S58782 | P38968 | S0002354 | Z74243 | gi7662370 | O45604 | Q9V4Z0 |
| SSA3 | 57 | S36753 | P09435 | S0000171 | Z35836 | gi5729877 | Q93601 | Q9VFB0 |
| SSU72 | 59 | S63180 | P53538 | S0005166 | Z71498 | | | |
| TAF60 | 61 | S64120 | P53040 | S0003080 | Z72634 | gi5032147 | O17279 | Q9VW16 |
| TIF4632 | 63 | B48086 | P39936 | S0003017 | Z72571 | | | |
| TKL1 | 65 | XJBYTK | P23254 | S0006278 | Z49219 | gi4507521 | O17759 | Q9VHN7 |
| TSA1 | 67 | A47362 | P34760 | S0004490 | Z46659 | | | |
| TYE7 | 69 | S48252 | P33122 | S0005871 | Z75252 | | | |
| VID24 | 71 | S48270 | P38263 | S0000309 | Z35974 | | | |
| VPS53 | 73 | S56801 | P47061 | S0003566 | Z49304 | F15000000 | YNP8_CAEE | Q9VQY8 |
| | | | | | | 87184 | L | |
| YSH1 | 75 | S51413 | | S0004267 | U17245 | | | |
| YTH1 | 77 | S59772 | | S0006311 | U32445 | | | |
| YCL046W | 79 | S19375 | P25575 | S0000551 | X59720 | | | |
| YGR156W | 81 | S60446 | P39927 | S0003388 | | | | |
| YHL035C | 83 | S48933 | P38735 | S0001027 | U11583 | | | |
| YKL018W | 85 | S37831 | P36104 | S0001501 | Z28018 | F15P000000 | Q18403 | Q9VLN1 |
| | | | | | | 64108 | | |
| YLR221C | 87 | S51444 | | S0004211 | U19027 | | | |
| YKL059C | 89 | S37881 | P35728 | S0001542 | Z28059 | | | |
| YML030W | 91 | S49749 | Q03713 | S0004492 | Z46659 | | | |
| YOR179C | 93 | S67071 | | S0005705 | Z75087 | | | |

B)

| Yeast | | | |
|---|---|---|---|
| proteins | | Biochemical | |
| listed in | | function | Cellular function |
| table 2 | YPD description | from YPD | from YPD |
| ACT1 | Actin, involved in cell | Structural protein | Cell polarity; Cell |
| | polarization, endocytosis, | | structure; |
| | and other cytoskeletal | | Chromatin/chromosome |
| | functions | | structure; Mating |
| | | | response; Pol II |
| | | | transcription; Vesicular |
| | | | transport |
| CFT1 | Component of pre-mRNA | Hydrolase; Nuclease | RNA |
| | cleavage factor II | [endo, exo, ribo, | processing/modification |
| | | deoxyribo] | |
| CFT2 | Component of pre-mRNA | RNA-binding protein | RNA |
| | cleavage factor II | | processing/modification |
| CKA1 | Casein kinase II (protein | Protein kinase; | Pol III transcription |
| | kinase CK2), catalytic | Transferase | |
| | (alpha) subunit | | |
| CLP1 | Subunit of cleavage and | Nuclease [endo, | RNA |
| | polyadenylation factor IA, | exo, ribo, deoxyribo] | processing/modification |
| | required for 3′-end | | |
| | processing of pre-mRNA | | |
| EFT2 | Translation elongation | Translation factor | Protein synthesis |
| | factor EF-2, identical | | |
| | to Eft1p, contains | | |
| | diphthamide which is not | | |
| | essential for its activity | | |
| ENO2 | Enolase 2 (2-phosphoglycerate | Lyase | Carbohydrate metabolism |
| | dehydratase); converts 2-phospho-D- | | |
| | glycerate to phosphoenolpyruvate | | |
| | in glycolysis | | |
| FIP1 | Component of polyadenylation | RNA polymerase | RNA |
| | factor that interacts with poly(A) | subunit; RNA- | processing/modification |
| | polymerase | binding protein; | |
| | | Regulatory subunit | |
| GLC7 | Protein serine/threonine phosphatase | Hydrolase; Protein | Carbohydrate |
| | PP1 required for glucose | phosphatase | metabolism; Cell polarity; |
| | repression, membrane bilayer mixing, | | Cell stress; Meiosis; |
| | and ER-to-Golgi and endocytic | | Mitosis |
| | vesicular trafficking | | |
| GPM1 | Phosphoglycerate mutase that | Isomerase | Carbohydrate |
| | converts 2-phosphoglycerate to 3- | | metabolism; Energy |
| | phosphoglycerate in glycolysis | | generation |
| HHF2 | Histone H4, identical to Hhf1p | DMA-binding protein | Chromatin/chromosome |
| | | | structure; Pol I |
| | | | transcription |
| HTA1 | Histone H2A, nearly identical | DMA-binding protein | Cell stress; |
| | to Hta2p | | Chromatin/chromosome |
| | | | structure; Pol II |
| | | | transcription |
| HSC82 | Chaperonin homologous to E. coli | Heat shock protein; | Protein folding; Cell stress |
| | HtpG and mammalian HSP90 | Hydrolase; ATPase; | |
| | | Chaperones | |
| IMD2 | Inosine-5′-monophosphate | Oxidoreductase | Nucleotide metabolism |
| | dehydrogenase, catalyzes the | | |
| | conversion of inosine | | |
| | 5′-phosphate and NAD(+) | | |
| | to xanthosine 5′-phosphate | | |
| | and NADH, the first reaction unique | | |
| | to GMP biosynthesis | | |
| IMD4 | Protein with similarity to | Oxidoreductase | Nucleotide metabolism |
| | inosine-5′-monophosphate | | |
| | dehydrogenase | | |
| MET6 | Homocysteine methyltransferase | Transferase | Amino-acid metabolism |
| | (5-methyltetrahydropteroyl | | |
| | triglutamate-homocysteine | | |
| | methyltransferase), | | |
| | methionine synthase, cobalamin- | | |
| | independent | | |
| PAB1 | Poly(A)-binding protein of | RNA-binding protein; | Protein synthesis; RNA |
| | cytoplasm and nucleus, part | Translation factor | processing/modification; |
| | of the 3′-end RNA- | | RNA turnover |
| | processing complex (cleavage | | |
| | factor I), has 4 RNA recognition | | |
| | (RRM) domains | | |
| PAP1 | Poly(A) polymerase, required | RNA polymerase | RNA |
| | for mRNA 3′ end formation, | subunit; RNA- | processing/modification |
| | has a poorly conserved RNA | binding protein; | |
| | recognition (RRM) domain | Transferase | |
| PCF11 | Component of pre-mRNA cleavage | Nuclease [endo, | RNA |
| | and polyadenylation factor I, | exo, ribo, deoxyribo]; | processing/modification |
| | interacts with Rna14p and Rna15p | RNA-binding protein | |
| PDC1 | Pyruvate decarboxylase isozyme 1 | Lyase | Carbohydrate metabolism |
| PFK1 | Phosphofructokinase alpha subunit, | Other kinase; | Carbohydrate metabolism |
| | part of a complex with Pfk2p which | Transferase | |
| | catalyze ATP-dependent conversion | | |
| | of fructose-6-phosphate to fructose- | | |
| | 1,6-bisphosphate, a key regulatory | | |
| | step in glycolysis | | |
| PFS2 | Polyadenylation factor I | Unknown | Protein complex |
| | subunit 2 required for mRNA | | assembly; RNA |
| | 3′-end processing, | | processing/modification |
| | bridges two mRNA 3′-end | | |
| | processing factors, has WD | | |
| | (WD-40) repeats | | |
| PTA1 | Component of pre-mRNA cleavage | Hydrolase; Nuclease | RNA |
| | factor II (CFII), required for | [endo, exo, ribo, | processing/modification |
| | both cleavage and polyadenylation | deoxyribo] | |
| | of mRNA precursor | | |
| REF2 | Protein involved in mRNA | RNA-binding protein | RNA |
| | 3′-end formation before | | processing/modification |
| | polyadenylation, mutant | | |
| | displays significantly lower | | |
| | usage of weak poly(A) sites | | |
| RNA14 | Component of pre-mRNA cleavage | Hydrolase; Nuclease | RNA |
| | and polyadenylation factor I | [endo, exo, ribo, | processing/modification |
| | (CFI) involved in poly(A) site | deoxyribo] | |
| | choice, interacts with Rna15p, | | |
| | Fip1p, Pap1p, and Pcf11p | | |
| RNA15 | Component of pre-mRNA cleavage | Nuclease [endo, | RNA |
| | and polyadenylation factor I | exo, ribo, deoxyribo]; | processing/modification |
| | (CFI), involved in poly(A) site | RNA-binding protein | |
| | choice, interacts with Rna14p, | | |
| | Pap1p, and Pcf11p, contains one | | |
| | RNA recognition (RRM) domain | | |
| SEC13 | Component of the COPII coat of | Unknown | Small molecule transport; |
| | vesicles involved in endoplasmic | | Vesicular transport |
| | reticulum to Golgi transport, | | |
| | contains six WD (WD-40) repeats | | |
| SEC31 | Component (p150) of the COPII | Vesicle coat protein | Vesicular transport |
| | coat of secretory pathway vesicles | | |
| | involved in endoplasmic reticulum | | |
| | to Golgi transport, associated with | | |
| | Sec13p, member of WD (WD-40) repeat | | |
| | family | | |
| SSA3 | Chaperone of the HSP70 family, | Chaperones; Heat | Cell stress; Protein |
| | heat-induced cytoplasmic form not | shock protein | folding; Protein |
| | expressed under optimal conditions | | translocation |
| SSU72 | Protein that interacts with | Complex assembly | Pol II transcription |
| | TFIIB (Sua7p) and influences | protein | |
| | RNA polymerase II start-site | | |
| | selection in sua7 mutants | | |
| TAF60 | Component of TAF(II) complex | Transcription factor | Pol II transcription |
| | (TBP-associated protein complex) | | |
| | and SAGA complex | | |
| | (Spt-Ada-Gcn5-acetyltransferase), | | |
| | required for activated | | |
| | transcription by RNA polymerase II | | |
| TIF4632 | mRNA cap-binding protein | RNA-binding protein; | Protein synthesis |
| | (elF4F) 130K subunit | Translation factor | |
| TKL1 | Transketolase 1 | Transferase | Amino-acid metabolism; |
| | | | Carbohydrate metabolism |
| TSA1 | Thioredoxin peroxidase, abundant | Oxidoreductase | Cell stress |
| | thiol-specific antioxidant protein | | |
| | that prevents formation of | | |
| | sulfur-containing radicals | | |
| TYE7 | Basic helix-loop-helix transcription | Transcription factor | Pol II transcription |
| | factor that can suppress the Gcr1p | | |
| | requirement for glycolytic gene | | |
| | expression | | |
| VID24 | Protein required for vacuolar import | Unknown | Protein degradation; |
| | and degradation of Fbp1p (fructose- | | Vesicular transport |
| | 1,6-bisphosphatase) | | |
| VPS53 | Subunit of the Vps52p-Vps53p-Vps54p | Docking protein | Vesicular transport |
| | complex, involved in protein | | |
| | sorting in the late Golgi | | |
| YCL046W | Protein of unknown function | Unknown | Unknown |
| YGR156W | | Unknown | Unknown |
| YHL035C | Member of the ATP-binding | ATP-binding | Small molecule transport |
| | cassette (ABC) superfamily | cassette; ATPase; | |
| | | Active transporter, | |
| | | primary; Hydrolase; | |
| | | Transporter | |
| YKL018W | Protein of unknown function | Unknown | Unknown |
| YLR221C | Protein of unknown function | Unknown | Unknown |
| YKL059C | Protein with similarity to | Unknown | Unknown |
| | members of the chaperonin- | | |
| | containing T-complex | | |
| YML030W | Protein of unknown function, | Unknown | Energy generation |
| | may be involved in mitochondrial | | |
| | translation | | |
| YOR179C | Protein with similarity to Ysh1p | Unknown | Unknown |
| YSH1 | Component of pre-mRNA cleavage | RNA-binding protein | RNA |
| | factor II (CFII), required for | | processing/modification |
| | processing of mRNA 3′ end | | |
| YTH1 | Component of polyadenylation | Unknown | RNA |
| | factor, required for both | | processing/modification |
| | cleavage and polyadenylation | | |
| | of pre-mRNA | | |

TABLE 3
MEDICAL APPLICATION OF THE COMPLEX

| Name of | Cellular | |
|---|---|---|
| complex | role | Medical applications |
| Poly- | RNA | infectious diseases; viral |
| adenylation- | processing/ | infections such as herpes |
| complex | modifi- | simplex infections, Epstein- |
| | cation | Barr-infections, influenza; |
| | | metabolic disease such as |
| | | metachromatic leukodystrophy; |
| | | neurodegenerative disorders |
| | | such as amyotrophic lateral |
| | | sclerosis; cancer |

TABLE 4
CHARACTERIZATION OF PREVIOUSLY UNDESCRIBED PROTEINS

| List of | | | |
|---|---|---|---|
| proteins of | | Motifs found | predicted |
| unknown | | by sequence | known |
| functions | Putative function | analysis | orthologues |
| YCL046W | | — | |
| YGR156W | | RRM | |
| YHL035C | multispecific organic | 3x TM, | no ortholog, |
| | anion transporter 1, | 2x PFAM: | but high |
| | multidrug resistance- | ABC-membrane | identity to |
| | associated protein 2) | domain, | MRP1-HUMAN |
| | | 2x AAA | (P33527), |
| | | | MRP3-HUMAN |
| | | | (O15438), |
| | | | MRP2-HUMAN |
| | | | (Q92887) |
| YKL018W | guanine nucleotide | 4x WD40 | |
| | binding protein | | |
| YLR221C | | — | |
| YML030W | | 2x trans- | Q9P297 (CLST |
| | | membrane | 11240 protein) |
| YOR179C | polyadenylation | — | |

| | |
|---|---|
| Legend | |
| AAA | ATPases associated with a variety of cellular activities |
| ABC-membrane | unit of six transmembrane helices; many members of ABC |
| domain | transporter family have two such regions |
| RRM | RNA recognition motif |
| WD40 | 40 residues repeat first identified in beta transducin |

### Table 5

### Overview on Experimental Steps

### High Troughput Production of TAP Tagged Yeast ORFs

1) Oligo Design

The oligo are semi-automatically designed in FileMaker Pro. The 3′ end of the ORF (forward oligo, 51 nucleotides), the 3′ UTR (100 nucleotides used to design the reverse oligo) and sequence 500 nucleotides upstream of the stop codon (40 nucleotides, check oligos) are directly extracted from database (SDG). For the reverse oligo, the program automatically gives the antiparallel sequence. The 17 constant nucleotide sequences used to prime the PCR reaction are automatically added at the 3′ end of the oligo

2) Target PCR

Fully Automated (Tecan Robot)

Materials: target plasmid: pBS1539 (described in Rigaut G, et al., Nat Biotechnol. 1999 Oct;17(10):1030-2.)

oligos: purchased from MWG, forwards and reverse primers are pre-mixed to a final concentration of 10 micromolar and delivered in a 96 well plate format

| | | tempera- | |
|---|---|---|---|
| step | volume | ture | Device |
| prepare master | up to | 4 C. | cooling |
| mix (AmpliTaq, | 2.5 ml | master | platform 1 |
| Perkin Elmer), | | mix | |
| containing the | | | |
| TAP cassette | | | |
| vector (1.5 ng/25 | | | |
| microliter reaction) | | | |
| Dispense the | 23.5 | 4 C. | cooling |
| master mix in 96 | microliters | master | platforms |
| well plate = | | mix + | 1 + 2 |
| reaction plate | | reaction | |
| (96 well plate) | | plate | |
| Add 1.0 microliters | 1.0 | 4 C. | cooling |
| of oligo (final | microliters | reaction | platform 2 |
| concentration: 0.4 | | plate | |
| microM) to reaction | | | |
| plate | | | |
| Cycle (30 cycles) | | | MJ |
| the PCR reaction | | | thermocyclers |
| Pipette 2 microliters | 2 | rt | Pharmacia |
| from the reaction | microliters | | ready |
| plate to a 96 well gel | | | to run gels |
| (Pharmacia) | | | |
| run gel 5 minutes | | | Pharmacia, |
| | | | Ready-To-Run |

3) Yeast Transformation

General considerations: Procedure partially automated

Materials: the haploid yeast strain is MGD453-13D: MATa, ade2, arg4, leu2-3,112, trp1-289, ura3-52.

| | | | | “novelties” |
|---|---|---|---|---|
| | | | | verus |
| | | tempera- | | original |
| step | volume | ture | device | protocole. |
| The day before, | 200 ml | 30 C. | shaking | |
| inoculate 200 ml | | | incubator | |
| of YPD with ˜100 | | | | |
| microliters of | | | | |
| freshly growing | | | | |
| yeast culture | | | | |
| When the culture | 2 ml | rt | | uses of 24 |
| reaches an OD600 | | | | well plates |
| of 1.0, dispense | | | | (Qiagen) |
| 2 ml of yeast | | | | |
| culture in 24 | | | | |
| well plate = | | | | |
| transformation | | | | |
| plate (4) | | | | |
| spin | | | heraus | |
| | | rt | centrifuge | |
| aspirate the sup | 2 ml | rt | | |
| ressuspend cells | | rt | shaker | no washing |
| in the remaining | | | | steps of |
| of media | | | | the cells |
| dispense 5 | 5 | rt | | |
| microliters | microliters | | | |
| of carrier | | | | |
| DNA, (Herring | | | | |
| testes Carrier | | | | |
| DNA; 10 mg/ml; | | | | |
| Clontech) | | | | |
| mix | | rt | shaker | |
| transfer | 20 | rt | | no cleaning |
| remaining | microliters | | | of the PCR |
| of PCR from | | | | reaction |
| reaction plate | | | | |
| to the trans- | | | | |
| formation plate | | | | |
| mix | | rt | shaker | |
| add 500 | 500 | rt | | |
| microliters | microliters | | | |
| PEG/LiAc | (viscous) | | | |
| mix | | rt | shaker | |
| add 55 | 50 | rt | shaker | |
| microliters | microliters | | | |
| DMSO, mix | | | | |
| incubate 15′ rt | | rt | | |
| incubate 15′ | | 42 C. | heating | |
| at 42 C. | | | platform | |
| | | | with custom | |
| | | | made | |
| | | | Pelletier | |
| | | | elements | |
| transfer at | 700 | rt | | |
| rt + 700 | microliters | | | |
| microliters | | | | |
| TE | | | | |
| spin | | rt | | |
| aspirate sup | | rt | | |
| add 800 | 800 | rt | | |
| microliters | microliters | | | |
| YPD | | | | |
| incubate 30′ | | 30 C. | heating | |
| at 30 C + | | | platform + | |
| shake | | | shaker | |
| add 1 ml of | | rt | | |
| TE | | | | |
| spin | | rt | | |
| aspirate sup | | rt | | |
| add 50 | 50 | rt | 8 tips | |
| microliters | microliters | | | |
| TE | | | | |
| mix | | rt | shaker | |
| plate + incubate | | | | |
| for ˜3 days at | | | | |
| 30 C. (10 cm | | | | |
| Petri dishes | | | | |
| or 12-24 | | | | |
| wellplates) | | | | |

4) Check PCR

general considerations: fully automated. According to results of the transformation 0 to 6 colonies are tested for homologous recombination. These results are filed in an excell file directly linked to the robot program.

Material: the forward oligos are specific for each ORF (for te sequence cf 1); purchased from MWG at 10 micromolar in 96 well plates. The reverse oligo is constant for all ORFs and annealed in the TAP sequense

| | | | | |
|---|---|---|---|---|
| dispense 20 | 20 | microliters | rt | |
| microliters | | | | |
| NaOH (20 mM) | | | | |
| in 96 well | | | | |
| plates | | | | |
| pickup colonies | | | | |
| (0 to 6) in the | | | | |
| 96 well plate | | | | |
| containing the | | | | |
| NaOH = DNA | | | | |
| plate (96 well) | | | | |
| Boil 2′ | | | | |
| add 140 | | | | |
| microliters | | | | |
| of TE pH 7.5 | | | | |
| (8x dilution) | | | | |
| spin at 2000 rpm | | | | |
| Prepare master | up to 9 ml | microliters | 4 C. | cooling |
| mix (PCR master, | | | | platform |
| Roche) | | | | 1 |
| dispense 14.5 | 14.5 | microliters | 4 C. | cooling |
| microliters in | | | | platform |
| reaction plate | | | | 2 |
| (96 well plate) | | | | |
| add 2.5 | 2.5 | microliters | 4 C. | cooling |
| microliters | | | reaction | platform |
| of oligo (final | | | plate | 1 |
| concentration | | | | |
| 1 microM) to | | | | |
| reaction plate | | | | |
| transfer 8 | 8 | microliters | 4 C. | cooling |
| microliters | | | reaction | platform |
| DNA from DNA | | | plate | 1 |
| plate to | | | | |
| reaction plate | | | | |
| Cycle (30 cycles) | | | | MJ |
| the PCR reaction | | | | thermo- |
| | | | | cyclers |
| add 1 microliters | 5 | microliters | rt | |
| of LB | | | | |
| load 10 | 10 | microliters | | Pharmacia |
| microliters | | | | ready to |
| on a 96 well | | | | run gels |
| agarose gel | | | | |
| run gel 5 | | | | Pharmacia, |
| minutes | | | | Ready-To-Run |

5) Dot Blot Analysis

| | |
|---|---|
| the remaining of the check PCR | 30 C. incubator |
| positive colonies are restreacked | |
| on -ura plates. Plates are incubated | |
| at 30 C. | |
| the next day, the restreack is used to | 30 C. shaker |
| inoculate 2 ml of YPD in 24 well plates | |
| (Qiagen). Plates are incubated over | |
| night at 30 C. | |
| Culture plates are spin | |
| remove the supernatant | |
| add 100 microliters of waters | mix shaker |
| add 100 microliters of 0.2 M NaOH | mix shaker |
| incubate 3 minutes at room temperature | |
| spin | |
| remove the supernatant | |
| add 50 microliters of 2× sample buffer | |
| boil 3 minutes | |
| spin | |
| load on a 96 dot blot apparatus | Biodot, BioRad |
| dot blot on a nitrocellulose membranne | Protran, Schleicher |
| | and Schuell |
| detect TAP tagged protein by ECL using | |
| peroxidase anti-peroxidase complex | |

TABLE 6
KNOWN COMPONENTS OF THE YEAST mRNA
3′-END PROCESSING MACHINERY

| | | Poly- | | |
|---|---|---|---|---|
| | | peptide | | |
| | | comp. | Gene product/ | |
| Factor | Function | (kDa) | (ORF) | Sequence motifs |
| CF IA | Cleavage and | 79.8 | Rna14 | 8xHAT domains |
| | polyadenylation | 71.9 | (YMR061w) | |
| | | 50.0 | Pcf11 (YDR228c) | |
| | | 32.8 | Clp1 (YOR250c) | RRM |
| | | | Rna15 (YGL044c) | |
| | Polyadenylation | 64.2 | | 4×RRM 1xPolyA |
| | | | Pab1 (YER165w) | |
| CF IB | Cleavage site | 73 | Hrp1 (YOL123w) | |
| | selection and | | | |
| | polyadenylation | | | |
| CF II/ | Cleavage and | 153.4 | Cft1/Yhh1 (YDR301w) | |
| PF I | polyadenylation | 96.1 | Cft2/Ydh1 (YLR115w) | Lactamase |
| (= CPF) | | 87.6 | Ysh1/Brr5 (YLR277c) | NTP_transfer_2 |
| | | 88.3 | Pta1 (YAL043c) | |
| | | 64.4 | Pap1 (YKR002w) | 7xWD40 |
| | | | Pfs1 | 5xZnF_C3H1 |
| | | 53.1 | Pfs2 (YNL317w) | |
| | | 35.6 | Fip1 (YJR093c) | |
| | | 24.4 | Yth1 (YPR107c) | |

NOVEL COMPLEX MEMBERS

| | | | Gene product | |
|---|---|---|---|---|
| Factor | Function | kDA | (OKE) | Seq. Motifs |
| | | 59.7 | Ref2 (YDR195w) | — |
| | | 59.5 | YKL059c = Pfs1? | ZnF_C2HC |
| | | 46.8 | YGR156w | RRM |
| | | 37.0 | YKL018w | 4xWD40 |
| | | 35.9 | Glc7 (YER133w) | PP2Ac |
| | | 23.3 | Ssu72 (YNL222w) | — |
| | | 20.9 | YOR179c | similarity |
| | | | | to Ysh1 |
| | | | Act1 | Actin |
| | | | Cka1 | Kinase |
| | | | Eft2 | |
| | | | Eno2 | Enolase |
| | | | Gpm1 | |
| | | | Hhf2 | Core Histone |
| | | | Hsc82 | |
| | | | Hta1 | Core Histone |
| | | | Imd2 | |
| | | | Imd4 | |
| | | | Met6 | |
| | | | Pdc1 | |
| | | | Pfk1 | Phosphofrukto- |
| | | | | kinase |
| | | | Sec13 | WD domain, |
| | | | | G-beta repeat |
| | | | Sec31 | WD domain, |
| | | | | G-beta repeat |
| | | | Ssa3 | Hsp70 protein |
| | | | | domain |
| | | | Taf60 | — |
| | | | Tif4632 | Helix-loop-Helix |
| | | | | DNA-bind. |
| | | | Tsa1 | |
| | | | Tye7 | — |
| | | | Vid24 | — |
| | | | Vps53 | — |
| | | | YCL046w | ABC transporter |
| | | | | transmemb reg |
| | | | YHL035c | — |
| | | | YLR221c | — |
| | | | YML030w | |

CF: cleavage factor
PF I: polyadenylation factor
CstF: cleavage and stimulation factor
CPSF: cleavage and polyadenylation specificity factor
YGR156w: has RNA-binding domain
Glc7: was found in Y2H using Ref2 as bait (Uetz screen)
YOR179c: similar to Ysh1 (PF I complex) (37% identity, 56% similarity)

The present invention is not to be limited in scope by the specific embodiments described herein. Indeed, various modifications of the invention in addition to those described herein will become apparent to those skilled in the art from the foregoing description and accompanying figures. Such modifications are intended to fall within the scope of the appended claims.

Various publications are cited herein, the disclosures of which are incorporated by reference in their entireties.

1. An isolated complex selected from complex (I) and comprising
(a) a first protein, or a functionally active fragment or functionally active derivative thereof, which first protein is selected from the group consisting of:
(i) Cft1 (SEQ ID NO:3), or a mammalian homolog thereof, or a variant of Cft1 encoded by a nucleic acid that hybridizes to the Cft1 nucleic acid (SEQ ID NO:4) or its complement under low stringency conditions,
(ii) Cft2 (SEQ ID NO:5), or a mammalian homolog thereof, or a variant of Cft2 encoded by a nucleic acid that hybridizes to the Cft2 nucleic acid (SEQ ID NO:6) or its complement under low stringency conditions,
(iii) Clp1 (SEQ ID NO:9), or a mammalian homolog thereof, or a variant of Clp1 encoded by a nucleic acid that hybridizes to the Clp1 nucleic acid (SEQ ID NO:10) or its complement under low stringency conditions,
(iv) Fip1 (SEQ ID NO:19), or a mammalian homolog thereof, or a variant of Fip1 encoded by a nucleic acid that hybridizes to the Fip1 nucleic acid (SEQ ID NO:20) or its complement under low stringency conditions,
(v) Pab1 (SEQ ID NO:33), or a mammalian homolog thereof, or a variant of Pab1 encoded by a nucleic acid that hybridizes to the Pab1 nucleic acid (SEQ ID NO:34) or its complement under low stringency conditions,
(vi) Pap1 (SEQ ID NO:35), or a mammalian homolog thereof, or a variant of Pap1 encoded by a nucleic acid that hybridizes to the Pap1 nucleic acid (SEQ ID NO:36) or its complement under low stringency conditions,
(vii) Pcf11 (SEQ ID NO:37), or a mammalian homolog thereof, or a variant of Pcf11 encoded by a nucleic acid that hybridizes to the Pcf11 nucleic acid (SEQ ID NO:38) or its complement under low stringency conditions,
(viii) Pfs2 (SEQ ID NO:43), or a mammalian homolog thereof, or a variant of Pfs2 encoded by a nucleic acid that hybridizes to the Pfs2 nucleic acid (SEQ ID NO:44) or its complement under low stringency conditions,
(ix) Pta1 (SEQ ID NO:45), or a mammalian homolog thereof, or a variant of Pta1 encoded by a nucleic acid that hybridizes to the Pta1 nucleic acid (SEQ ID NO:46) or its complement under low stringency conditions,
(x) Rna14 (SEQ ID NO:49), or a mammalian homolog thereof, or a variant of Rna14 encoded by a nucleic acid that hybridizes to the Rna14 nucleic acid (SEQ ID NO:50) or its complement under low stringency conditions,
(xi) Rna15 (SEQ ID NO:51), or a mammalian homolog thereof, or a variant of Rna15 encoded by a nucleic acid that hybridizes to the Rna15 nucleic acid (SEQ ID NO:52) or its complement under low stringency conditions,
(xii) Tif4632 (SEQ ID NO:63), or a mammalian homolog thereof, or a variant of Tif4632 encoded by a nucleic acid that hybridizes to the Tif4632 nucleic acid (SEQ ID NO:64) or its complement under low stringency conditions,
(xiii) Ykl059c (SEQ ID NO:89), or a mammalian homolog thereof, or a variant of Ykl059c encoded by a nucleic acid that hybridizes to the Ykl059c nucleic acid (SEQ ID NO:90) or its complement under low stringency conditions,
(xiv) Ysh1 (SEQ ID NO:75), or a mammalian homolog thereof, or a variant of Ysh1 encoded by a nucleic acid that hybridizes to the Ysh1 nucleic acid (SEQ ID NO:76) or its complement under low stringency conditions, and
(xv) Yth1 (SEQ ID NO:77), or a mammalian homolog thereof, or a variant of Yth1 encoded by a nucleic acid that hybridizes to the Yth1 nucleic acid (SEQ ID NO:78) or its complement under low stringency conditions; and
(b) a second protein, or a functionally active fragment or functionally active derivative thereof, which second protein is selected from the group consisting of:
(i) Act1 (SEQ ID NO:1), or a mammalian homolog thereof, or a variant of Act1 encoded by a nucleic acid that hybridizes to the Act1 nucleic acid (SEQ ID NO:2) or its complement under low stringency conditions,
(ii) Cka1 (SEQ ID NO:7), or a mammalian homolog thereof, or a variant of Cka1 encoded by a nucleic acid that hybridizes to the Cka1 nucleic acid (SEQ ID NO:8) or its complement under low stringency conditions,
(iii) Eft2 (SEQ ID NO:11), or a mammalian homolog thereof, or a variant of Eft2 encoded by a nucleic acid that hybridizes to the Eft2 nucleic acid (SEQ ID NO:12) or its complement under low stringency conditions,
(iv) Eno2 (SEQ ID NO:13), or a mammalian homolog thereof, or a variant of Eno2 encoded by a nucleic acid that hybridizes to the Eno2 nucleic acid (SEQ ID NO:14) or its complement under low stringency conditions,
(v) Glc7 (SEQ ID NO:15), or a mammalian homolog thereof, or a variant of Glc7 encoded by a nucleic acid that hybridizes to the Glc7 nucleic acid (SEQ ID NO:16) or its complement under low stringency conditions,
(vi) Gpm1 (SEQ ID NO:17), or a mammalian homolog thereof, or a variant of Gpm1 encoded by a nucleic acid that hybridizes to the Gpm1 nucleic acid (SEQ ID NO:18) or its complement under low stringency conditions,
(vii) Hhf2 (SEQ ID NO:21), or a mammalian homolog thereof, or a variant of Hhf2 encoded by a nucleic acid that hybridizes to the Hhf2 nucleic acid (SEQ ID NO:22) or its complement under low stringency conditions,
(viii) Hta1 (SEQ ID NO:23), or a mammalian homolog thereof, or a variant of Hta1 encoded by a nucleic acid that hybridizes to the Hta1 nucleic acid (SEQ ID NO:24) or its complement under low stringency conditions,
(ix) Hsc82 (SEQ ID NO:25), or a mammalian homolog thereof, or a variant of Hsc82 encoded by a nucleic acid that hybridizes to the Hsc82 nucleic acid (SEQ ID NO:26) or its complement under low stringency conditions,
(x) Imd2 (SEQ ID NO:27), or a mammalian homolog thereof, or a variant of Imd2 encoded by a nucleic acid that hybridizes to the Imd2 nucleic acid (SEQ ID NO:28) or its complement under low stringency conditions,
(xi) Imd4 (SEQ ID NO:29), or a mammalian homolog thereof, or a variant of Imd4 encoded by a nucleic acid that hybridizes to the Imd4 nucleic acid (SEQ ID NO:30) or its complement under low stringency conditions,
(xii) Met6 (SEQ ID NO:31), or a mammalian homolog thereof, or a variant of Met6 encoded by a nucleic acid that hybridizes to the Met6 nucleic acid (SEQ ID NO:32) or its complement under low stringency conditions,
(xiii) Pdc1 (SEQ ID NO:39), or a mammalian homolog thereof, or a variant of Pdc1 encoded by a nucleic acid that hybridizes to the Pdc1 nucleic acid (SEQ ID NO:40) or its complement under low stringency conditions,
(xiv) Pfk1 (SEQ ID NO:41), or a mammalian homolog thereof, or a variant of Pfk1 encoded by a nucleic acid that hybridizes to the Pfk1 nucleic acid (SEQ ID NO:42) or its complement under low stringency conditions,
(xv) Ref2 (SEQ ID NO:47), or a mammalian homolog thereof, or a variant of Ref2 encoded by a nucleic acid that hybridizes to the Ref2 nucleic acid (SEQ ID NO:48) or its complement under low stringency conditions,
(xvi) Sec13 (SEQ ID NO:53), or a mammalian homolog thereof, or a variant of Sec13 encoded by a nucleic acid that hybridizes to the Sec13 nucleic acid (SEQ ID NO:54) or its complement under low stringency conditions,
(xvii) Sec31 (SEQ ID NO:55), or a mammalian homolog thereof, or a variant of Sec31 encoded by a nucleic acid that hybridizes to the Sec31 nucleic acid (SEQ ID NO:56) or its complement under low stringency conditions,
(xviii) Ssa3 (SEQ ID NO:57), or a mammalian homolog thereof, or a variant of Ssa3 encoded by a nucleic acid that hybridizes to the Ssa3 nucleic acid (SEQ ID NO:58) or its complement under low stringency conditions,
(xix) Ssu72 (SEQ ID NO:59), or a mammalian homolog thereof, or a variant of Ssu72 encoded by a nucleic acid that hybridizes to the Ssu72 nucleic acid (SEQ ID NO:60) or its complement under low stringency conditions,
(xx) Taf60 (SEQ ID NO:61), or a mammalian homolog thereof, or a variant of Taf60 encoded by a nucleic acid that hybridizes to the Taf60 nucleic acid (SEQ ID NO:62) or its complement under low stringency conditions,
(xxi) Tkl1 (SEQ ID NO:65), or a mammalian homolog thereof, or a variant of Tkl1 encoded by a nucleic acid that hybridizes to the Tkl1 nucleic acid (SEQ ID NO:66) or its complement under low stringency conditions,
(xxii) Tsa1 (SEQ ID NO:67), or a mammalian homolog thereof, or a variant of Tsa1 encoded by a nucleic acid that hybridizes to the Tsa1 nucleic acid (SEQ ID NO:68) or its complement under low stringency conditions,
(xxiii) Tye7 (SEQ ID NO:69), or a mammalian homolog thereof, or a variant of Tye7 encoded by a nucleic acid that hybridizes to the Tye7 nucleic acid (SEQ ID NO:70) or its complement under low stringency conditions,
(xxiv) Vid24 (SEQ ID NO:71), or a mammalian homolog thereof, or a variant of Vid24 encoded by a nucleic acid that hybridizes to the Vid24 nucleic acid (SEQ ID NO:72) or its complement under low stringency conditions,
(xxv) Vps53 (SEQ ID NO:73), or a mammalian homolog thereof, or a variant of Vps53 encoded by a nucleic acid that hybridizes to the Vps53 nucleic acid (SEQ ID NO:74) or its complement under low stringency conditions,
(xxvi) Ycl046w (SEQ ID NO:79), or a mammalian homolog thereof, or a variant of Ycl046w encoded by a nucleic acid that hybridizes to the Ycl046w nucleic acid (SEQ ID NO:80) or its complement under low stringency conditions,
(xxvii) Ygr156w (SEQ ID NO:81), or a mammalian homolog thereof, or a variant of Ygr156w encoded by a nucleic acid that hybridizes to the Ygr156w nucleic acid (SEQ ID NO:82) or its complement under low stringency conditions,
(xxviii) Yhl035c (SEQ ID NO:83), or a mammalian homolog thereof, or a variant of Yhl035c encoded by a nucleic acid that hybridizes to the Yhl035c nucleic acid (SEQ ID NO:84) or-its complement under low stringency conditions,
(xxix) Ykl018w (SEQ ID NO:85), or a mammalian homolog thereof, or a variant of Ykl018w encoded by a nucleic acid that hybridizes to the Ykl018w nucleic acid (SEQ ID NO:86) or its complement under low stringency conditions,
(xxx) Ylr221c (SEQ ID NO:87), or a mammalian homolog thereof, or a variant of Ylr221c encoded by a nucleic acid that hybridizes to the Ylr221c nucleic acid (SEQ ID NO:88) or its complement under low stringency conditions,
(xxxi) Yml030w (SEQ ID NO:91), or a mammalian homolog thereof, or a variant of Yml030w encoded by a nucleic acid that hybridizes to the Yml030w nucleic acid (SEQ ID NO:92) or its complement under low stringency conditions, and
(xxxii) Yor179c (SEQ ID NO:93), or a mammalian homolog thereof, or a variant of Yor179c encoded by a nucleic acid that hybridizes to the Yor179c nucleic acid (SEQ ID NO:94) or its complement under low stringency conditions,
wherein said first protein and said second protein are members of a native cellular Polyadenylation-complex, and wherein said low stringency conditions comprise hybridization in a buffer comprising 35% formamide, 5×SSC, 50 mM Tris-HCl (pH 7.5), 5 mM EDTA, 0.02% PVP, 0.02% Ficoll, 0.2% BSA, 100 ug/ml denatured salmon sperm DNA, and 10% (wt/vol) dextran sulfate for 18-20 hours at 40° C., washing in a buffer consisting of 2×SSC, 25 mM Tris-HCl (pH 7.4), 5 mM EDTA, and 0.1% SDS for 1.5 hours at 55° C., and washing in a buffer consisting of 2×SSC, 25 mM Tris-HCl (pH 7.4), 5 mM EDTA, and 0.1% SDS for 1.5 hours at 60° C., and a complex (II) comprising at least two second proteins.

2. An isolated complex comprising the following proteins:
(i) Act1 (SEQ ID NO:1), or a mammalian homolog thereof, or a variant of Act1 encoded by a nucleic acid that hybridizes to the Act1 nucleic acid (SEQ ID NO:2) or its complement under low stringency conditions,
(ii) Cft1 (SEQ ID NO:3), or a mammalian homolog thereof, or a variant of Cft1 encoded by a nucleic acid that hybridizes to the Cft1 nucleic acid (SEQ ID NO:4) or its complement under low stringency conditions,
(iii) Cft2 (SEQ ID NO:5), or a mammalian homolog thereof, or a variant of Cft2 encoded by a nucleic acid that hybridizes to the Cft2 nucleic acid (SEQ ID NO:6) or its complement under low stringency conditions,
(iv) Cka1 (SEQ ID NO:7), or a mammalian homolog thereof, or a variant of Cka1 encoded by a nucleic acid that hybridizes to the Cka1 nucleic acid (SEQ ID NO:8) or its complement under low stringency conditions,
(v) Clp1 (SEQ ID NO:9), or a mammalian homolog thereof, or a variant of Clp1 encoded by a nucleic acid that hybridizes to the Clp1 nucleic acid (SEQ ID NO:10) or its complement under low stringency conditions,
(vi) Eft2 (SEQ ID NO:11), or a mammalian homolog thereof, or a variant of Eft2 encoded by a nucleic acid that hybridizes to the Eft2 nucleic acid (SEQ ID NO:12) or its complement under low stringency conditions,
(vii) Eno2 (SEQ ID NO:13), or a mammalian homolog thereof, or a variant of Eno2 encoded by a nucleic acid that hybridizes to the Eno2 nucleic acid (SEQ ID NO:14) or its complement under low stringency conditions,
(viii) Glc7 (SEQ ID NO:15), or a mammalian homolog thereof, or a variant of Glc7 encoded by a nucleic acid that hybridizes to the Glc7 nucleic acid (SEQ ID NO:16) or its complement under low stringency conditions,
(ix) Gpm1 (SEQ ID NO:17), or a mammalian homolog thereof, or a variant of Gpm1 encoded by a nucleic acid that hybridizes to the Gpm1 nucleic acid (SEQ ID NO:18) or its complement under low stringency conditions,
(x) Fip1 (SEQ ID NO:19), or a mammalian homolog thereof, or a variant of Fip1 encoded by a nucleic acid that hybridizes to the Fip1 nucleic acid (SEQ ID NO:20) or its complement under low stringency conditions,
(xi) Hhf2 (SEQ ID NO:21), or a mammalian homolog thereof, or a variant of Hhf2 encoded by a nucleic acid that hybridizes to the Hhf2 nucleic acid (SEQ ID NO:22) or its complement under low stringency conditions,
(xii) Hta1 (SEQ ID NO:23), or a mammalian homolog thereof, or a variant of Hta1 encoded by a nucleic acid that hybridizes to the Hta1 nucleic acid (SEQ ID NO:24) or its complement under low stringency conditions,
(xiii) Hsc82 (SEQ ID NO:25), or a mammalian homolog thereof, or a variant of Hsc82 encoded by a nucleic acid that hybridizes to the Hsc82 nucleic acid (SEQ ID NO:26) or its complement under low stringency conditions,
(xiv) Imd2 (SEQ ID NO:27), or a mammalian homolog thereof, or a variant of Imd2 encoded by a nucleic acid that hybridizes to the Imd2 nucleic acid (SEQ ID NO:28) or its complement under low stringency conditions,
(xv) Imd4 (SEQ ID NO:29), or a mammalian homolog thereof, or a variant of Imd4 encoded by a nucleic acid that hybridizes to the Imd4 nucleic acid (SEQ ID NO:30) or its complement under low stringency conditions,
(xvi) Met6 (SEQ ID NO:31), or a mammalian homolog thereof, or a variant of Met6 encoded by a nucleic acid that hybridizes to the Met6 nucleic acid (SEQ ID NO:32) or its complement under low stringency conditions,
(xvii) Pab1 (SEQ ID NO:33), or a mammalian homolog thereof, or a variant of Pab1 encoded by a nucleic acid that hybridizes to the Pab1 nucleic acid (SEQ ID NO:34) or its complement under low stringency conditions,
(xviii) Pap1 (SEQ ID NO:35), or a mammalian homolog thereof, or a variant of Pap1 encoded by a nucleic acid that hybridizes to the Pap1 nucleic acid (SEQ ID NO:36) or its complement under low stringency conditions,
(xix) Pcf11 (SEQ ID NO:37), or a mammalian homolog thereof, or a variant of Pcf11 encoded by a nucleic acid that hybridizes to the Pcf11 nucleic acid (SEQ ID NO:38) or its complement under low stringency conditions,
(xx) Pdc1 (SEQ ID NO:39), or a mammalian homolog thereof, or a variant of Pdc1 encoded by a nucleic acid that hybridizes to the Pdc1 nucleic acid (SEQ ID NO:40) or its complement under low stringency conditions,
(xxi) Pfk1 (SEQ ID NO:41), or a mammalian homolog thereof, or a variant of Pfk1 encoded by a nucleic acid that hybridizes to the Pfk1 nucleic acid (SEQ ID NO:42) or its complement under low stringency conditions,
(xxii) Pfs2 (SEQ ID NO:43), or a mammalian homolog thereof, or a variant of Pfs2 encoded by a nucleic acid that hybridizes to the Pfs2 nucleic acid (SEQ ID NO:44) or its complement under low stringency conditions,
(xxiii) Pta1 (SEQ ID NO:45), or a mammalian homolog thereof, or a variant of Pta1 encoded by a nucleic acid that hybridizes to the Pta1 nucleic acid (SEQ ID NO:46) or its complement under low stringency conditions,
(xxiv) Ref2 (SEQ ID NO:47), or a mammalian homolog thereof, or a variant of Ref2 encoded by a nucleic acid that hybridizes to the Ref2 nucleic acid (SEQ ID NO:48) or its complement under low stringency conditions,
(xxv) Rna14 (SEQ ID NO:49), or a mammalian homolog thereof, or a variant of Rna14 encoded by a nucleic acid that hybridizes to the Rna14 nucleic acid (SEQ ID NO:50) or its complement under low stringency conditions,
(xxvi) Rna15 (SEQ ID NO:51), or a mammalian homolog thereof, or a variant of Rna15 encoded by a nucleic acid that hybridizes to the Rna15 nucleic acid (SEQ ID NO:52) or its complement under low stringency conditions,
(xxvii) Sec13 (SEQ ID NO:53), or a mammalian homolog thereof, or a variant of Sec13 encoded by a nucleic acid that hybridizes to the Sec13 nucleic acid (SEQ ID NO:54) or its complement under low stringency conditions,
(xxviii) Sec31 (SEQ ID NO:55), or a mammalian homolog thereof, or a variant of Ser31 encoded by a nucleic acid that hybridizes to the Sec31 nucleic acid (SEQ ID NO:56) or its complement under low stringency conditions,
(xxix) Ssa3 (SEQ ID NO:57), or a mammalian homolog thereof, or a variant of Ssa3 encoded by a nucleic acid that hybridizes to the Ssa3 nucleic acid (SEQ ID NO:58) or its complement under low stringency conditions,
(xxx) Ssu72 (SEQ ID NO:59), or a mammalian homolog thereof, or a variant of Ssu72 encoded by a nucleic acid that hybridizes to the Ssu72 nucleic acid (SEQ ID NO:60) or its complement under low stringency conditions,
(xxxi) Taf60 (SEQ ID NO:61), or a mammalian homolog thereof, or a variant of Taf60 encoded by a nucleic acid that hybridizes to the Taf60 nucleic acid (SEQ ID NO:62) or its complement under low stringency conditions,
(xxxii) Tif4632 (SEQ ID NO:63), or a mammalian homolog thereof, or a variant of Tif4632 encoded by a nucleic acid that hybridizes to the Tif4632 nucleic acid (SEQ ID NO:64) or its complement under low stringency conditions,
(xxxiii) Tkl1 (SEQ ID NO:65), or a mammalian homolog thereof, or a variant of Tkl1 encoded by a nucleic acid that hybridizes to the Tkl1 nucleic acid (SEQ ID NO:66) or its complement under low stringency conditions,
(xxxiv) Tsa1 (SEQ ID NO:67), or a mammalian homolog thereof, or a variant of Tsa1 encoded by a nucleic acid that hybridizes to the Tsa1 nucleic acid (SEQ ID NO:68) or its complement under low stringency conditions,
(xxxv) Tye7 (SEQ ID NO:69), or a mammalian homolog thereof, or a variant of Tye7 encoded by a nucleic acid that hybridizes to the Tye7 nucleic acid (SEQ ID NO:70) or its complement under low stringency conditions,
(xxxvi) Vid24 (SEQ ID NO:71), or a mammalian homolog thereof, or a variant of Vid24 encoded by a nucleic acid that hybridizes to the Vid24 nucleic acid (SEQ ID NO:72) or its complement under low stringency conditions,
(xxxvii) Vps53 (SEQ ID NO:73), or a mammalian homolog thereof, or a variant of Vps53 encoded by a nucleic acid that hybridizes to the Vps53 nucleic acid (SEQ ID NO:74) or its complement under low stringency conditions,
(xxxviii) Ysh1 (SEQ ID NO:75), or a mammalian homolog thereof, or a variant of Ysh1 encoded by a nucleic acid that hybridizes to the Ysh1 nucleic acid (SEQ ID NO:76) or its complement under low stringency conditions,
(xxxix) Yth1 (SEQ ID NO:77), or a mammalian homolog thereof, or a variant of Yth1 encoded by a nucleic acid that hybridizes to the Yth1 nucleic acid (SEQ ID NO:78) or its complement under low stringency conditions,
(xl) Ycl046w (SEQ ID NO:79), or a mammalian homolog thereof, or a variant of Ycl046w encoded by a nucleic acid that hybridizes to the Ycl046w nucleic acid (SEQ ID NO:80) or its complement under low stringency conditions,
(xli) Ygr156w (SEQ ID NO:81), or a mammalian homolog thereof, or a variant of Ygr156w encoded by a nucleic acid that hybridizes to the Ygr156w nucleic acid (SEQ ID NO:82) or its complement under low stringency conditions,
(xlii) Yhl035c (SEQ ID NO:83), or a mammalian homolog thereof, or a variant of Yhl035c encoded by a nucleic acid that hybridizes to the Yhl035c nucleic acid (SEQ ID NO:84) or its complement under low stringency conditions,
(xliii) Ykl018w (SEQ ID NO:85), or a mammalian homolog thereof, or a variant of Ykl018w encoded by a nucleic acid that hybridizes to the Ykl018w nucleic acid (SEQ ID NO:86) or its complement under low stringency conditions,
(xliv) Ylr221c (SEQ ID NO:87), or a mammalian homolog thereof, or a variant of Ylr221c encoded by a nucleic acid that hybridizes to the Ylr221c nucleic acid (SEQ ID NO:88) or its complement under low stringency conditions,
(xlv) Ykl059c (SEQ ID NO:89), or a mammalian homolog thereof, or a variant of Ykl059c encoded by a nucleic acid that hybridizes to the Ykl059c nucleic acid (SEQ ID NO:90) or its complement under low stringency conditions,
(xlvi) Yml030w (SEQ ID NO:91), or a mammalian homolog thereof, or a variant of Yml030w encoded by a nucleic acid that hybridizes to the Yml030w nucleic acid (SEQ ID NO:92) or its complement under low stringency conditions, and
(xlvii) Yor179c (SEQ ID NO:93), or a mammalian homolog thereof, or a variant of Yor179c encoded by a nucleic acid that hybridizes to the Yor179c nucleic acid (SEQ ID NO:94) or its complement under low stringency conditions,
wherein said proteins are members of a native cellular Polyadenylation-complex, and
wherein said low stringency conditions comprise hybridization in a buffer comprising 35% formamide, 5×SSC, 50 mM Tris-HCl (pH 7.5), 5 mM EDTA, 0.02% PVP, 0.02% Ficoll, 0.2% BSA, 100 ug/ml denatured salmon sperm DNA, and 10% (wt/vol) dextran sulfate for 18-20 hours at 40° C., washing in a buffer consisting of 2×SSC, 25 mM Tris-HCl (pH 7.4), 5 mM EDTA, and 0.1% SDS for 1.5 hours at 55° C., and washing in a buffer consisting of 2×SSC, 25 mM Tris-HCl (pH 7.4), 5 mM EDTA, and 0.1% SDS for 1.5 hours at 60° C.

3. An isolated complex that comprises all but 2, 3, 4, 5, 6, 7, 8, 9, 10, 11, 12, 13, 14, 15, 16, 17,18,19,20,21,22,23,24,25,26,27 or 28 of the following 47 proteins:
(i) Act1 (SEQ ID NO:1), or a mammalian homolog thereof, or a variant of Act1 encoded by a nucleic acid that hybridizes to the Act1 nucleic acid (SEQ ID NO:2) or its complement under low stringency conditions,
(ii) Cft1 (SEQ ID NO:3), or a mammalian homolog thereof, or a variant of Cft1 encoded by a nucleic acid that hybridizes to the Cft1 nucleic acid (SEQ ID NO-4) or its complement under low stringency conditions,
(iii) Cft2 (SEQ ID NO:5), or a mammalian homolog thereof, or a variant of Cft2 encoded by a nucleic acid that hybridizes to the Cft2 nucleic acid (SEQ ID NO:6) or its complement under low stringency conditions,
(iv) Cka1 (SEQ ID NO:7), or a mammalian homolog thereof, or a variant of Cka1 encoded by a nucleic acid that hybridizes to the Cka1 nucleic acid (SEQ ID NO:8) or its complement under low stringency conditions,
(v) Clp1 (SEQ ID NO:9), or a mammalian homolog thereof, or a variant of Clp1 encoded by a nucleic acid that hybridizes to the Clp1 nucleic acid (SEQ ID NO:10) or its complement under low stringency conditions,
(vi) Eft2 (SEQ ID NO:11), or a mammalian homolog thereof, or a variant of Eft2 encoded by a nucleic acid that hybridizes to the Eft2 nucleic acid (SEQ ID NO:12) or its complement under low stringency conditions,
(vii) Eno2 (SEQ ID NO:13), or a mammalian homolog thereof, or a variant of Eno2 encoded by a nucleic acid that hybridizes to the Eno2 nucleic acid (SEQ ID NO:14) or its complement under low stringency conditions,
(viii) Glc7 (SEQ ID NO:15), or a mammalian homolog thereof, or a variant of Glc7 encoded by a nucleic acid that hybridizes to the Glc7 nucleic acid (SEQ ID NO:16) or its complement under low stringency conditions,
(ix) Gpm1 (SEQ ID NO:17), or a mammalian homolog thereof, or a variant of Gpm1 encoded by a nucleic acid that hybridizes to the Gpm1 nucleic acid (SEQ ID NO:18) or its complement under low stringency conditions,
(x) Fip1 (SEQ ID NO:19), or a mammalian homolog thereof, or a variant of Fip1 encoded by a nucleic acid that hybridizes to the Fip1 nucleic acid (SEQ ID NO:20) or its complement under low stringency conditions,
(xi) Hhf2 (SEQ ID NO:21), or a mammalian homolog thereof, or a variant of Hhf2 encoded by a nucleic acid that hybridizes to the Hhf2 nucleic acid (SEQ ID NO:22) or its complement under low stringency conditions,
(xii) Hta1 (SEQ ID NO:23), or a mammalian homolog thereof, or a variant of Hta1 encoded by a nucleic acid that hybridizes to the Hta1 nucleic acid (SEQ ID NO:24) or its complement under low stringency conditions,
(xiii) Hsc82 (SEQ ID NO:25), or a mammalian homolog thereof, or a variant of Hsc82 encoded by a nucleic acid that hybridizes to the Hsc82 nucleic acid (SEQ ID NO:26) or its complement under low stringency conditions,
(xiv) Imd2 (SEQ ID NO:27), or a mammalian homolog thereof, or a variant of Imd2 encoded by a nucleic acid that hybridizes to the Imd2 nucleic acid (SEQ ID NO:28) or its complement under low stringency conditions,
(xv) Imd4 (SEQ ID NO:29), or a mammalian homolog thereof, or a variant of Imd4 encoded by a nucleic acid that hybridizes to the Imd4 nucleic acid (SEQ ID NO:30) or its complement under low stringency conditions,
(xvi) Met6 (SEQ ID NO:31), or a mammalian homolog thereof, or a variant of Met6 encoded by a nucleic acid that hybridizes to the Met6 nucleic acid (SEQ ID NO:32) or its complement under low stringency conditions,
(xvii) Pab1 (SEQ ID NO:33), or a mammalian homolog thereof, or a variant of Pab1 encoded by a nucleic acid that hybridizes to the Pab1 nucleic acid (SEQ ID NO:34) or its complement under low stringency conditions,
(xviii) Pap1 (SEQ ID NO:35), or a mammalian homolog thereof, or a variant of Pap1 encoded by a nucleic acid that hybridizes to the Pap1 nucleic acid (SEQ ID NO:36) or its complement under low stringency conditions,
(xix) Pcf11 (SEQ ID NO:37), or a mammalian homolog thereof, or a variant of Pcf11 encoded by a nucleic acid that hybridizes to the Pcf11 nucleic acid (SEQ ID NO:38) or its complement under low stringency conditions,
(xx) Pdc1 (SEQ ID NO:39), or a mammalian homolog thereof, or a variant of Pdc1 encoded by a nucleic acid that hybridizes to the Pdc1 nucleic acid (SEQ ID NO:40) or its complement under low stringency conditions,
(xxi) Pfk1 (SEQ ID NO:41), or a mammalian homolog thereof, or a variant of Pfk1 encoded by a nucleic acid that hybridizes to the Pfk1 nucleic acid (SEQ ID NO:42) or its complement under low stringency conditions,
(xxii) Pfs2 (SEQ ID NO:43), or a mammalian homolog thereof, or a variant of Pfs2 encoded by a nucleic acid that hybridizes to the Pfs2 nucleic acid (SEQ ID NO:44) or its complement under low stringency conditions,
(xxiii) Pta1 (SEQ ID NO:45), or a mammalian homolog thereof, or a variant of Pta1 encoded by a nucleic acid that hybridizes to the Pta1 nucleic acid (SEQ ID NO:46) or its complement under low stringency conditions,
(xxiv) Ref2 (SEQ ID NO:47), or a mammalian homolog thereof, or a variant of Ref2 encoded by a nucleic acid that hybridizes to the Ref2 nucleic acid (SEQ ID NO:48) or its complement under low stringency conditions,
(xxv) Rna14 (SEQ ID NO:49), or a mammalian homolog thereof, or a variant of Rna14 encoded by a nucleic acid that hybridizes to the Rna14 nucleic acid (SEQ ID NO:50) or its complement under low stringency conditions,
(xxvi) Rna15 (SEQ ID NO:51), or a mammalian homolog thereof, or a variant of Rna15 encoded by a nucleic acid that hybridizes to the Rna15 nucleic acid (SEQ ID NO:52) or its complement under low stringency conditions,
(xxvii) Sec13 (SEQ ID NO:53), or a mammalian homolog thereof, or a variant of Sec13 encoded by a nucleic acid that hybridizes to the Sec13 nucleic acid (SEQ ID NO:54) or its complement under low stringency conditions,
(xxviii) Sec31 (SEQ ID NO:55), or a mammalian homolog thereof, or a variant of Sec31 encoded by a nucleic acid that hybridizes to the Sec31 nucleic acid (SEQ ID NO:56) or its complement under low stringency conditions,
(xxix) Ssa3 (SEQ ID NO:57), or a mammalian homolog thereof, or a variant of Ssa3 encoded by a nucleic acid that hybridizes to the Ssa3 nucleic acid (SEQ ID NO:58) or its complement under low stringency conditions,
(xxx) Ssu72 (SEQ ID NO:59), or a mammalian homolog thereof, or a variant of Ssu72 encoded by a nucleic acid that hybridizes to the Ssu72 nucleic acid (SEQ ID NO:60) or its complement under low stringency conditions,
(xxxi) Taf60 (SEQ ID NO:61), or a mammalian homolog thereof, or a variant of Taf60 encoded by a nucleic acid that hybridizes to the Taf60 nucleic acid (SEQ ID NO:62) or its complement under low stringency conditions,
(xxxii) Tif4632 (SEQ ID NO:63), or a mammalian homolog thereof, or a variant of Tif4632 encoded by a nucleic acid that hybridizes to the Tif4632 nucleic acid (SEQ ID NO:64) or its complement under low stringency conditions,
(xxxiii) Tkl1 (SEQ ID NO:65), or a mammalian homolog thereof, or a variant of Tkl1 encoded by a nucleic acid that hybridizes to the Tkl1 nucleic acid (SEQ ID NO:66) or its complement under low stringency conditions,
(xxxiv) Tsa1 (SEQ ID NO:67), or a mammalian homolog thereof, or a variant of Tsa1 encoded by a nucleic acid that hybridizes to the Tsa1 nucleic acid (SEQ ID NO:68) or its complement under low stringency conditions,
(xxxv) Tye7 (SEQ ID NO:69), or a mammalian homolog thereof, or a variant of Tye7 encoded by a nucleic acid that hybridizes to the Tye7 nucleic acid (SEQ ID NO:70) or its complement under low stringency conditions,
(xxxvi) Vid24 (SEQ ID NO:71), or a mammalian homolog thereof, or a variant of Vid24 encoded by a nucleic acid that hybridizes to the Vid24 nucleic acid (SEQ ID NO:72) or its complement under low stringency conditions,
(xxxvii) Vps53 (SEQ ID NO:73), or a mammalian homolog thereof, or a variant of Vps53 encoded by a nucleic acid that hybridizes to the Vps53 nucleic acid (SEQ ID NO:74) or its complement under low stringency conditions,
(xxxviii) Ysh1 (SEQ ID NO:75), or a mammalian homolog thereof, or a variant of Ysh1 encoded by a nucleic acid that hybridizes to the Ysh1 nucleic acid (SEQ ID NO:76) or its complement under low stringency conditions,
(xxxix) Yth1 (SEQ ID NO:77), or a mammalian homolog thereof, or a variant of Yth1 encoded by a nucleic acid that hybridizes to the Yth1 nucleic acid (SEQ ID NO:78) or its complement under low stringency conditions,
(xl) Tcl046w (SEQ ID NO:79), or a mammalian homolog thereof, or a variant of Ycl046w encoded by a nucleic acid that hybridizes to the Tcl046w nucleic acid (SEQ ID NO:80) or its complement under low stringency conditions,
(xli) Ygr156w (SEQ ID NO:81), or a mammalian homolog thereof, or a variant of Ygr156w encoded by a nucleic acid that hybridizes to the Ygr156w nucleic acid (SEQ ID NO:82) or its complement under low stringency conditions,
(xlii) Yhl035c (SEQ ID NO:83), or a mammalian homolog thereof, or a variant of Yhl035c encoded by a nucleic acid that hybridizes to the Yhl035c nucleic acid (SEQ ID NO:84) or its complement under low stringency conditions,
(xliii) Ykl018w (SEQ ID NO:85), or a mammalian homolog thereof, or a variant of Ykl018w encoded by a nucleic acid that hybridizes to the Ykl018w nucleic acid (SEQ ID NO:86) or its complement under low stringency conditions,
(xliv) Ylr221c (SEQ ID NO:87), or a mammalian homolog thereof, or a variant of Ylr221c encoded by a nucleic acid that hybridizes to the Ylr221c nucleic acid (SEQ ID NO:88) or its complement under low stringency conditions,
(xlv) Ykl059c (SEQ ID NO:89), or a mammalian homolog thereof, or a variant of Ykl059c encoded by a nucleic acid that hybridizes to the Ykl059c nucleic acid (SEQ ID NO:90) or its complement under low stringency conditions,
(xlvi) Yml030w (SEQ ID NO:91), or a mammalian homolog thereof, or a variant of Yml030w encoded by a nucleic acid that hybridizes to the Yml030w nucleic acid (SEQ ID NO:92) or its complement under low stringency conditions, and
(xlvii) Yor179c (SEQ ID NO:93), or a mammalian homolog thereof, or a variant of Yor179c encoded by a nucleic acid that hybridizes to the Yor179c nucleic acid (SEQ ID NO:94) or its complement under low stringency conditions,
wherein said proteins are members of a native cellular Polyadenylation-complex, and
wherein said low stringency conditions comprise hybridization in a buffer comprising 35% formamide, 5×SSC, 50 mM Tris-HCl (pH 7.5), 5 mM EDTA, 0.02% PVP, 0.02% Ficoll, 0.2% BSA, 100 ug/ml denatured salmon sperm DNA, and 10% (wt/vol) dextran sulfate for 18-20 hours at 40° C., washing in a buffer consisting of 2×SSC, 25 mM Tris-HCl (pH 7.4), 5 mM EDTA, and 0.1% SDS for 1.5 hours at 55° C., and washing in a buffer consisting of 2×SSC, 25 mM Tris-HCl (pH 7.4), 5 mM EDTA, and 0.1% SDS for 1.5 hours at 60° C.

4. The complex according to claim 2, which comprises all but 1 of the 47 proteins.

5. The complex of claim 1, 2, 3 or 4 comprising a functionally active derivative of said first protein and/or a functionally active derivative of said second protein, wherein the functionally active derivative is a fusion protein comprising said first protein or said second protein fused to an amino acid sequence different from the first protein or second protein, respectively.

6. The complex of claim 1, 2, 3 or 4 comprising a fragment of said first protein and/or a fragment of said second protein, which fragment binds to another protein component of said complex.

7. The complex of claim 1, 2, 3, 4, 5 or 6 that is involved in the 3′ end processing activity for mRNA.

8. The complex of claim 5 wherein the functionally active derivative is a fusion protein comprising said first protein or said second protein fused to an affinity tag or label.

9. An antibody or a fragment of said antibody containing the binding domain thereof, which binds the complex of claim 1, 2, 3, 4, 5, 6 or 7 and which does not bind the first protein when uncomplexed or the second protein when uncomplexed.

10. A process for processing RNA comprising the step of bringning into contact a product to any of claims 1-8 with RNA, such that the RNA is processed.

11. A pharmaceutical composition comprising the protein complex of claim 1, 2, 3, 4, 5, 6, 7 or 8; and a pharmaceutically acceptable carrier.

12. A method for screening for a molecule that modulates directly or indirectly the function, activity, composition or formation of the complex of any one of claims 1-8 comprising the steps of:
(a) exposing said complex, or a cell or organism containing said Polyadenylation-complex to one or more candidate molecules; and
(b) determining the amount of 3′ end processing activity for mRNA of, or protein components of, said complex, wherein a change in said amount, activity, or protein components relative to said amount, activity or protein components in the absence of said candidate molecules indicates that the molecules modulate function, activity or composition of said complex.

13. The method of claim 12, wherein the amount of said complex is determined.

14. The method of claim 12, wherein the activity of said complex is determined.

15. The method of claim 14, wherein said determining step comprises isolating from the cell or organism said Polyadenylation-complex to produce said isolated complex and contacting said isolated complex with a RNA molecule such that the complex binds to the RNA.

16. The method of claim 12, wherein the protein components of said complex are determined.

17. The method of claim 16, wherein said determining step comprises determining whether
(i) Act1 (SEQ ID NO:1), or a mammalian homolog thereof, or a variant of Act1 encoded by a nucleic acid that hybridizes to the Act1 nucleic acid (SEQ ID NO:2) or its complement under low stringency conditions,
(ii) Cka1 (SEQ ID NO:7), or a mammalian homolog thereof, or a variant of Cka1 encoded by a nucleic acid that hybridizes to the Cka1 nucleic acid (SEQ ID NO:8) or its complement under low stringency conditions,
(iii) Eft2 (SEQ ID NO:11), or a mammalian homolog thereof, or a variant of Eft2 encoded by a nucleic acid that hybridizes to the Eft2 nucleic acid (SEQ ID NO:12) or its complement under low stringency conditions,
(iv) Eno2 (SEQ ID NO:13), or a mammalian homolog thereof, or a variant of Eno2 encoded by a nucleic acid that hybridizes to the Eno2 nucleic acid (SEQ ID NO:14) or its complement under low stringency conditions,
(v) Glc7 (SEQ ID NO:15), or a mammalian homolog thereof, or a variant of Glc7 encoded by a nucleic acid that hybridizes to the Glc7 nucleic acid (SEQ ID NO:16) or its complement under low stringency conditions,
(vi) Gpm1 (SEQ ID NO:17), or a mammalian homolog thereof, or a variant of Gpm1 encoded by a nucleic acid that hybridizes to the Gpm1 nucleic acid (SEQ ID NO:18) or its complement under low stringency conditions,
(vii) Hhf2 (SEQ ID NO:21), or a mammalian homolog thereof, or a variant of Hhf2 encoded by a nucleic acid that hybridizes to the Hhf2 nucleic acid (SEQ ID NO:22) or its complement under low stringency conditions,
(viii) Hta1 (SEQ ID NO:23), or a mammalian homolog thereof, or a variant of Hta1 encoded by a nucleic acid that hybridizes to the Hta1 nucleic acid (SEQ ID NO:24) or its complement under low stringency conditions,
(ix) Hsc82 (SEQ ID NO:25), or a mammalian homolog thereof, or a variant of Hsc82 encoded by a nucleic acid that hybridizes to the Hsc82 nucleic acid (SEQ ID NO:26) or its complement under low stringency conditions,
(x) Imd2 (SEQ ID NO:27), or a mammalian homolog thereof, or a variant of Imd2 encoded by a nucleic acid that hybridizes to the Imd2 nucleic acid (SEQ ID NO:28) or its complement under low stringency conditions,
(xi) Imd4 (SEQ ID NO:29), or a mammalian homolog thereof, or a variant of Imd4 encoded by a nucleic acid that hybridizes to the Imd4 nucleic acid (SEQ ID NO:30) or its complement under low stringency conditions,
(xii) Met6 (SEQ ID NO:31), or a mammalian homolog thereof, or a variant of Met6 encoded by a nucleic acid that hybridizes to the Met6 nucleic acid (SEQ ID NO:32) or its complement under low stringency conditions,
(xiii) Pdc1 (SEQ ID NO:39), or a mammalian homolog thereof, or a variant of Pdc1 encoded by a nucleic acid that hybridizes to the Pdc1 nucleic acid (SEQ ID NO:40) or its complement under low stringency conditions,
(xiv) Pfk1 (SEQ ID NO:41), or a mammalian homolog thereof, or a variant of Pfk1 encoded by a nucleic acid that hybridizes to the Pfk1 nucleic acid (SEQ ID NO:42) or its complement under low stringency conditions,
(xv) Ref2 (SEQ ID NO:47), or a mammalian homolog thereof, or a variant of Ref2 encoded by a nucleic acid that hybridizes to the Ref2 nucleic acid (SEQ ID NO:48) or its complement under low stringency conditions,
(xvi) Sec13 (SEQ ID NO:53), or a mammalian homolog thereof, or a variant of Sec13 encoded by a nucleic acid that hybridizes to the Sec13 nucleic acid (SEQ ID NO:54) or its complement under low stringency conditions,
(xvii) Sec31 (SEQ ID NO:55), or a mammalian homolog thereof, or a variant of Sec31 encoded by a nucleic acid that hybridizes to the Sec31 nucleic acid (SEQ ID NO:56) or its complement under low stringency conditions,
(xviii) Ssa3 (SEQ ID NO:57), or a mammalian homolog thereof, or a variant of Ssa3 encoded by a nucleic acid that hybridizes to the Ssa3 nucleic acid (SEQ ID NO:58) or its complement under low stringency conditions,
(xix) Ssu72 (SEQ ID NO:59), or a mammalian homolog thereof, or a variant of Ssu72 encoded by a nucleic acid that hybridizes to the Ssu72 nucleic acid (SEQ ID NO:60) or its complement under low stringency conditions,
(xx) Taf60 (SEQ ID NO:61), or a mammalian homolog thereof, or a variant of Taf60 encoded by a nucleic acid that hybridizes to the Taf60 nucleic acid (SEQ ID NO:62) or its complement under low stringency conditions,
(xxi) Tkl1 (SEQ ID NO:65), or a mammalian homolog thereof, or a variant of Tkl1 encoded by a nucleic acid that hybridizes to the Tkl1 nucleic acid (SEQ ID NO:66) or its complement under low stringency conditions,
(xxii) Tsa1 (SEQ ID NO:67), or a mammalian homolog thereof, or a variant of Tsa1 encoded by a nucleic acid that hybridizes to the Tsa1 nucleic acid (SEQ ID NO:68) or its complement under low stringency conditions,
(xxiii) Tye7 (SEQ ID NO:69), or a mammalian homolog thereof, or a variant of Tye7 encoded by a nucleic acid that hybridizes to the Tye7 nucleic acid (SEQ ID NO:70) or its complement under low stringency conditions,
(xxiv) Vid24 (SEQ ID NO:71), or a mammalian homolog thereof, or a variant of Vid24 encoded by a nucleic acid that hybridizes to the Vid24 nucleic acid (SEQ ID NO:72) or its complement under low stringency conditions,
(xxv) Vps53 (SEQ ID NO:73), or a mammalian homolog thereof, or a variant of Vps53 encoded by a nucleic acid that hybridizes to the Vps53 nucleic acid (SEQ ID NO:74) or its complement under low stringency conditions,
(xxvi) Ycl046w (SEQ ID NO:79), or a mammalian homolog thereof, or a variant of Ycl046w encoded by a nucleic acid that hybridizes to the Ycl046w nucleic acid (SEQ ID NO:80) or its complement under low stringency conditions,
(xxvii) Ygr156w (SEQ ID NO:81), or a mammalian homolog thereof, or a variant of Ygr156w encoded by a nucleic acid that hybridizes to the Ygr156w nucleic acid (SEQ ID NO:82) or its complement under low stringency conditions,
(xxviii) Yhl035c (SEQ ID NO:83), or a mammalian homolog thereof, or a variant of Yhl035c encoded by a nucleic acid that hybridizes to the Yhl035c nucleic acid (SEQ ID NO:84) or its complement under low stringency conditions,
(xxix) Ykl018w (SEQ ID NO:85), or a mammalian homolog thereof, or a variant of Ykl018w encoded by a nucleic acid that hybridizes to the Ykl018w nucleic acid (SEQ ID NO:86) or its complement under low stringency conditions,
(xxx) Ylr221c (SEQ ID NO:87), or a mammalian homolog thereof, or a variant of Ylr221c encoded by a nucleic acid that hybridizes to the Ylr221c nucleic acid (SEQ ID NO:88) or its complement under low stringency conditions,
(xxxi) Yml030w (SEQ ID NO:91), or a mammalian homolog thereof, or a variant of Yml030w encoded by a nucleic acid that hybridizes to the Yml030w nucleic acid (SEQ ID NO:92) or its complement under low stringency conditions, and
(xxxii) Yor179c (SEQ ID NO:93), or a mammalian homolog thereof, or a variant of Yor179c encoded by a nucleic acid that hybridizes to the Yor179c nucleic acid (SEQ ID NO:94) or its complement under low stringency conditions, is present in the complex,
wherein said low stringency conditions comprise hybridization in a buffer comprising 35% formamide, 5×SSC, 50 mM Tris-HCl (pH 7.5), 5 mM EDTA, 0.02% PVP, 0.02% Ficoll, 0.2% BSA, 100 ug/ml denatured salmon sperm DNA, and 10% (wt/vol) dextran sulfate for 18-20 hours at 40° C., washing in a buffer consisting of 2×SSC, 25 mM Tris-HCl (pH 7.4), 5 mM EDTA, and 0.1% SDS for 1.5 hours at 55° C., and washing in a buffer consisting of 2×SSC, 25 mM Tris-HCl (pH 7.4), 5 mM EDTA, and 0.1% SDS for 1.5 hours at 60° C.

18. The method of any of claim 12 to 17, wherein said method is a method of screening for a drug for treatment or prevention of a disease or disorder such as infectious diseases; viral infections such as herpes simplex infections, Epstein-Barr-infections, influenza; metabolic disease such as metachromatic leukodystrophy; neurodegenerative disorders such as amyotrophic lateral sclerosis; cancer.

19. A method for screening for a molecule that binds the complex of anyone of claim 1-8 comprising the following steps:
(a) exposing said complex, or a cell or organism containing said Polyadenylation-complex, to one or more candidate molecules; and
(b) determining whether said complex is bound by any of said candidate molecules.

20. A method for diagnosing or screening for the presence of a disease or disorder or a predisposition for developing a disease or disorder in a subject, which disease or disorder is characterized by an aberrant amount of 3′ end processing activity for mRNA of, or component composition of, the complex of any one of the claim 1-8, comprising determining the amount of, 3′ end processing activity for mRNA of, or protein components of, said complex in a sample derived from a subject, wherein a difference in said amount, activity, or protein components of, said complex in an analogous sample from a subject not having the disease or disorder or predisposition indicates the presence in the subject of the disease or disorder or predisposition.

21. The method of claim 20, wherein the amount of said complex is determined.

22. The method of claim 20, wherein the activity of said complex is determined.

23. The method of claim 22, wherein said determining step comprises isolating from the subject said Polyadenylation-complex to produce said isolated complex and contacting said isolated complex with a RNA molecule such that the complex binds to the RNA.

24. The method of claim 20, wherein the protein components of said complex are determined.

25. The method of claim 24, wherein said determining step comprises determining whether
(i) Act1 (SEQ ID NO:1), or a mammalian homolog thereof, or a variant of Act1 encoded by a nucleic acid that hybridizes to the Act1 nucleic acid (SEQ ID NO:2) or its complement under low stringency conditions,
(ii) Cka1 (SEQ ID NO:7), or a mammalian homolog thereof, or a variant of Cka1 encoded by a nucleic acid that hybridizes to the Cka1 nucleic acid (SEQ ID NO:8) or its complement under low stringency conditions,
(iii) Eft2 (SEQ ID NO:11), or a mammalian homolog thereof, or a variant of Eft2 encoded by a nucleic acid that hybridizes to the Eft2 nucleic acid (SEQ ID NO:12) or its complement under low stringency conditions,
(iv) Eno2 (SEQ ID NO:13), or a mammalian homolog thereof, or a variant of Eno2 encoded by a nucleic acid that hybridizes to the Eno2 nucleic acid (SEQ ID NO:14) or its complement under low stringency conditions,
(v) Glc7 (SEQ ID NO:15), or a mammalian homolog thereof, or a variant of Glc7 encoded by a nucleic acid that hybridizes to the Glc7 nucleic acid (SEQ ID NO:16) or its complement under low stringency conditions,
(vi) Gpm1 (SEQ ID NO:17), or a mammalian homolog thereof, or a variant of Gpm1 encoded by a nucleic acid that hybridizes to the Gpm1 nucleic acid (SEQ ID NO:18) or its complement under low stringency conditions,
(vii) Hhf2 (SEQ ID NO:21), or a mammalian homolog thereof, or a variant of Hhf2 encoded by a nucleic acid that hybridizes to the Hhf2 nucleic acid (SEQ ID NO:22) or its complement under low stringency conditions,
(viii) Hta1 (SEQ ID NO:23), or a mammalian homolog thereof, or a variant of Hta1 encoded by a nucleic acid that hybridizes to the Hta1 nucleic acid (SEQ ID NO:24) or its complement under low stringency conditions,
(ix) Hsc82 (SEQ ID NO:25), or a mammalian homolog thereof, or a variant of Hsc82 encoded by a nucleic acid that hybridizes to the Hsc82 nucleic acid (SEQ ID NO:26) or its complement under low stringency conditions,
(x) Imd2 (SEQ ID NO:27), or a mammalian homolog thereof, or a variant of Imd2 encoded by a nucleic acid that hybridizes to the Imd2 nucleic acid (SEQ ID NO:28) or its complement under low stringency conditions,
(xi) Imd4 (SEQ ID NO:29), or a mammalian homolog thereof, or a variant of Imd4 encoded by a nucleic acid that hybridizes to the Imd4 nucleic acid (SEQ ID NO:30) or its complement under low stringency conditions,
(xii) Met6 (SEQ ID NO:31), or a mammalian homolog thereof, or a variant of Met6 encoded by a nucleic acid that hybridizes to the Met6 nucleic acid (SEQ ID NO:32) or its complement under low stringency conditions,
(xiii) Pdc1 (SEQ ID NO:39), or a mammalian homolog thereof, or a variant of Pdc1 encoded by a nucleic acid that hybridizes to the Pdc1 nucleic acid (SEQ ID NO:40) or its complement under low stringency conditions,
(xiv) Pfk1 (SEQ ID NO:41), or a mammalian homolog thereof, or a variant of Pfk1 encoded by a nucleic acid that hybridizes to the Pfk1 nucleic acid (SEQ ID NO:42) or its complement under low stringency conditions,
(xv) Ref2 (SEQ ID NO:47), or a mammalian homolog thereof, or a variant of Ref2 encoded by a nucleic acid that hybridizes to the Ref2 nucleic acid (SEQ ID NO:48) or its complement under low stringency conditions,
(xvi) Sec13 (SEQ ID NO:53), or a mammalian homolog thereof, or a variant of Sec13 encoded by a nucleic acid that hybridizes to the Sec13 nucleic acid (SEQ ID NO:54) or its complement under low stringency conditions,
(xvii) Sec31 (SEQ ID NO:55), or a mammalian homolog thereof, or a variant of Sec31 encoded by a nucleic acid that hybridizes to the Sec31 nucleic acid (SEQ ID NO:56) or its complement under low stringency conditions,
(xviii) Ssa3 (SEQ ID NO:57), or a mammalian homolog thereof, or a variant of Ssa3 encoded by a nucleic acid that hybridizes to the Ssa3 nucleic acid (SEQ ID NO:58) or its complement under low stringency conditions,
(xix) Ssu72 (SEQ ID NO:59), or a mammalian homolog thereof, or a variant of Ssu72 encoded by a nucleic acid that hybridizes to the Ssu72 nucleic acid (SEQ ID NO:60) or its complement under low stringency conditions,
(xx) Taf60 (SEQ ID NO:61), or a mammalian homolog thereof, or a variant of Taf60 encoded by a nucleic acid that hybridizes to the Taf60 nucleic acid (SEQ ID NO:62) or its complement under low stringency conditions,
(xxi) Tkl1 (SEQ ID NO:65), or a mammalian homolog thereof, or a variant of Tkl1 encoded by a nucleic acid that hybridizes to the Tkl1 nucleic acid (SEQ ID NO:66) or its complement under low stringency conditions,
(xxii) Tsa1 (SEQ ID NO:67), or a mammalian homolog thereof, or a variant of Tsa1 encoded by a nucleic acid that hybridizes to the Tsa1 nucleic acid (SEQ ID NO:68) or its complement under low stringency conditions,
(xxiii) Tye7 (SEQ ID NO:69), or a mammalian homolog thereof, or a variant of Tye7 encoded by a nucleic acid that hybridizes to the Tye7 nucleic acid (SEQ ID NO:70) or its complement under low stringency conditions,
(xxiv) Vid24 (SEQ ID NO:71), or a mammalian homolog thereof, or a variant of Vid24 encoded by a nucleic acid that hybridizes to the Vid24 nucleic acid (SEQ ID NO:72) or its complement under low stringency conditions,
(xxv) Vps53 (SEQ ID NO:73), or a mammalian homolog thereof, or a variant of Vps53 encoded by a nucleic acid that hybridizes to the Vps53 nucleic acid (SEQ ID NO:74) or its complement under low stringency conditions,
(xxvi) Tcl046w (SEQ ID NO:79), or a mammalian homolog thereof, or a variant of Tcl046w encoded by a nucleic acid that hybridizes to the Tcl046w nucleic acid (SEQ ID NO:80) or its complement under low stringency conditions,
(xxvii) Ygr156w (SEQ ID NO:81), or a mammalian homolog thereof, or a variant of Ygr156w encoded by a nucleic acid that hybridizes to the Ygr156w nucleic acid (SEQ ID NO:82) or its complement under low stringency conditions,
(xxviii) Yhl035c (SEQ ID NO:83), or a mammalian homolog thereof, or a variant of Yhl035c encoded by a nucleic acid that hybridizes to the Yhl035c nucleic acid (SEQ ID NO:84) or its complement under low stringency conditions,
(xxix) Ykl018w (SEQ ID NO:85), or a mammalian homolog thereof, or a variant of Ykl018w encoded by a nucleic acid that hybridizes to the Ykl018w nucleic acid (SEQ ID NO:86) or its complement under low stringency conditions,
(xxx) Ylr221c (SEQ ID NO:87), or a mammalian homolog thereof, or a variant of Ylr221c encoded by a nucleic acid that hybridizes to the Ylr221c nucleic acid (SEQ ID NO:88) or its complement under low stringency conditions,
(xxxi) Yml030w (SEQ ID NO:91), or a mammalian homolog thereof, or a variant of Yml030w encoded by a nucleic acid that hybridizes to the Yml030w nucleic acid (SEQ ID NO:92) or its complement under low stringency conditions, and
(xxxii) Yor179c (SEQ ID NO:93), or a mammalian homolog thereof, or a variant of Yor179c encoded by a nucleic acid that hybridizes to the Yor179c nucleic acid (SEQ ID NO:94) or its complement under low stringency conditions, is present in the complex,
wherein said low stringency conditions comprise hybridization in a buffer comprising 35% formamide, 5×SSC, 50 mM Tris-HCl (pH 7.5), 5 mM EDTA, 0.02% PVP, 0.02% Ficoll, 0.2% BSA, 100 ug/ml denatured salmon sperm DNA, and 10% (wt/vol) dextran sulfate for 18-20 hours at 40° C., washing in a buffer consisting of 2×SSC, 25 mM Tris-HCl (pH 7.4), 5 mM EDTA, and 0.1% SDS for 1.5 hours at 55° C., and washing in a buffer consisting of 2×SSC, 25 mM Tris-HCl (pH 7.4), 5 mM EDTA, and 0.1% SDS for 1.5 hours at 60° C.

26. A method for treating or preventing a disease or disorder characterized by an aberrant amount of, 3′ end processing activity for mRNA of, or component composition of, the complex of anyone of claim 1-8, comprising administering to a subject in need of such treatment or prevention a therapeutically effective amount of one or more molecules that modulate the amount of, 3′ end processing activity for mRNA of, or protein components of, said complex.

27. The method according to claim 26, wherein said disease or disorder involves decreased levels of the amount or activity of said complex.

28. The method according to claim 26, wherein said disease or disorder involves increased levels of the amount or activity of said complex.

29. Use of a molecule that modulates the amount of, 3′ end processing activity for mRNA of, or the protein components of the complex of any one of claim 1-8 for the manufacture of a medicament for the treatment or prevention of a disease or disorder such as infectious diseases; viral infections such as herpes simplex infections, Epstein-Barr-infections, influenza; metabolic disease such as metachromatic leukodystrophy; neurodegenerative disorders such as amyotrophic lateral sclerosis; cancer.

30. A kit comprising in one or more containers
(a) an isolated first protein, or a functionally active fragment or functionally active derivative thereof, which first protein is selected from the group consisting of:
(i) Cft1 (SEQ ID NO:3), or a mammalian homolog thereof, or a variant of Cft1 encoded by a nucleic acid that hybridizes to the Cft1 nucleic acid (SEQ ID NO:4) or its complement under low stringency conditions,
(ii) Cft2 (SEQ ID NO:5), or a mammalian homolog thereof, or a variant of Cft2 encoded by a nucleic acid that hybridizes to the Cft2 nucleic acid (SEQ ID NO:6) or its complement under low stringency conditions,
(iii) Clp1 (SEQ ID NO:9), or a mammalian homolog thereof, or a variant of Clp1 encoded by a nucleic acid that hybridizes to the Clp1 nucleic acid (SEQ ID NO:10) or its complement under low stringency conditions,
(iv) Fip1 (SEQ ID NO:19), or a mammalian homolog thereof, or a variant of Fip1 encoded by a nucleic acid that hybridizes to the Fip1 nucleic acid (SEQ ID NO:20) or its complement under low stringency conditions,
(v) Pab1 (SEQ ID NO:33), or a mammalian homolog thereof, or a variant of Pab1 encoded by a nucleic acid that hybridizes to the Pab1 nucleic acid (SEQ ID NO:34) or its complement under low stringency conditions,
(vi) Pap1 (SEQ ID NO:35), or a mammalian homolog thereof, or a variant of Pap1 encoded by a nucleic acid that hybridizes to the Pap1 nucleic acid (SEQ ID NO:36) or its complement under low stringency conditions,
(vii) Pcf11 (SEQ ID NO:37), or a mammalian homolog thereof, or a variant of Pcf11 encoded by a nucleic acid that hybridizes to the Pcf11 nucleic acid (SEQ ID NO:38) or its complement under low stringency conditions,
(viii) Pfs2 (SEQ ID NO:43), or a mammalian homolog thereof, or a variant of Pfs2 encoded by a nucleic acid that hybridizes to the Pfs2 nucleic acid (SEQ ID NO:44) or its complement under low stringency conditions,
(ix) Pta1 (SEQ ID NO:45), or a mammalian homolog thereof, or a variant of Pta1 encoded by a nucleic acid that hybridizes to the Pta1 nucleic acid (SEQ ID NO:46) or its complement under low stringency conditions,
(x) Rna14 (SEQ ID NO:49), or a mammalian homolog thereof, or a variant of Rna14 encoded by a nucleic acid that hybridizes to the Rna14 nucleic acid (SEQ ID NO:50) or its complement under low stringency conditions,
(xi) Rna15 (SEQ ID NO:51), or a mammalian homolog thereof, or a variant of Rna15 encoded by a nucleic acid that hybridizes to the Rna15 nucleic acid (SEQ ID NO:52) or its complement under low stringency conditions,
(xii) Tif4632 (SEQ ID NO:63), or a mammalian homolog thereof, or a variant of Tif4632 encoded by a nucleic acid that hybridizes to the Tif4632 nucleic acid (SEQ ID NO:64) or its complement under low stringency conditions,
(xiii) Ykl059c (SEQ ID NO:89), or a mammalian homolog thereof, or a variant of Ykl059c encoded by a nucleic acid that hybridizes to the Ykl059c nucleic acid (SEQ ID NO:90) or its complement under low stringency conditions,
(xiv) Ysh1 (SEQ ID NO:75), or a mammalian homolog thereof, or a variant of Ysh1 encoded by a nucleic acid that hybridizes to the Ysh1 nucleic acid (SEQ ID NO:76) or its complement under low stringency conditions, and
(xv) Yth1 (SEQ ID NO:77), or a mammalian homolog thereof, or a variant of Yth1 encoded by a nucleic acid that hybridizes to the Yth1 nucleic acid (SEQ ID NO:78) or its complement under low stringency conditions; and
(b) a second protein, or a functionally active fragment or functionally active derivative thereof, which second protein is selected from the group consisting of:
(i) Act1 (SEQ ID NO:1), or a mammalian homolog thereof, or a variant of Act1 encoded by a nucleic acid that hybridizes to the Act1 nucleic acid (SEQ ID NO:2) or its complement under low stringency conditions,
(ii) Cka1 (SEQ ID NO:7), or a mammalian homolog thereof, or a variant of Cka1 encoded by a nucleic acid that hybridizes to the Cka1 nucleic acid (SEQ ID NO:8) or its complement under low stringency conditions,
(iii) Eft2 (SEQ ID NO:11), or a mammalian homolog thereof, or a variant of Eft2 encoded by a nucleic acid that hybridizes to the Eft2 nucleic acid (SEQ ID NO:12) or its complement under low stringency conditions,
(iv) Eno2 (SEQ ID NO:13), or a mammalian homolog thereof, or a variant of Eno2 encoded by a nucleic acid that hybridizes to the Eno2 nucleic acid (SEQ ID NO:14) or its complement under low stringency conditions,
(v) Glc7 (SEQ ID NO:15), or a mammalian homolog thereof, or a variant of Glc7 encoded by a nucleic acid that hybridizes to the Glc7 nucleic acid (SEQ ID NO:16) or its complement under low stringency conditions,
(vi) Gpm1 (SEQ ID NO:17), or a mammalian homolog thereof, or a variant of Gpm1 encoded by a nucleic acid that hybridizes to the Gpm1 nucleic acid (SEQ ID NO:18) or its complement under low stringency conditions,
(vii) Hhf2 (SEQ ID NO:21), or a mammalian homolog thereof, or a variant of Hhf2 encoded by a nucleic acid that hybridizes to the Hhf2 nucleic acid (SEQ ID NO:22) or its complement under low stringency conditions,
(viii) Hta1 (SEQ ID NO:23), or a mammalian homolog thereof, or a variant of Hta1 encoded by a nucleic acid that hybridizes to the Hta1 nucleic acid (SEQ ID NO:24) or its complement under low stringency conditions,
(ix) Hsc82 (SEQ ID NO:25), or a mammalian homolog thereof, or a variant of Hsc82 encoded by a nucleic acid that hybridizes to the Hsc82 nucleic acid (SEQ ID NO:26) or its complement under low stringency conditions,
(x) Imd2 (SEQ ID NO:27), or a mammalian homolog thereof, or a variant of Imd2 encoded by a nucleic acid that hybridizes to the Imd2 nucleic acid (SEQ ID NO:28) or its complement under low stringency conditions,
(xi) Imd4 (SEQ ID NO:29), or a mammalian homolog thereof, or a variant of Imd4 encoded by a nucleic acid that hybridizes to the Imd4 nucleic acid (SEQ ID NO:30) or its complement under low stringency conditions,
(xii) Met6 (SEQ ID NO:31), or a mammalian homolog thereof, or a variant of Met6 encoded by a nucleic acid that hybridizes to the Met6 nucleic acid (SEQ ID NO:32) or its complement under low stringency conditions,
(xiii) Pdc1 (SEQ ID NO:39), or a mammalian homolog thereof, or a variant of Pdc1 encoded by a nucleic acid that hybridizes to the Pdc1 nucleic acid (SEQ ID NO:40) or its complement under low stringency conditions,
(xiv) Pfk1 (SEQ ID NO:41), or a mammalian homolog thereof, or a variant of Pfk1 encoded by a nucleic acid that hybridizes to the Pfk1 nucleic acid (SEQ ID NO:42) or its complement under low stringency conditions,
(xv) Ref2 (SEQ ID NO:47), or a mammalian homolog thereof, or a variant of Ref2 encoded by a nucleic acid that hybridizes to the Ref2 nucleic acid (SEQ ID NO:48) or its complement under low stringency conditions,
(xvi) Sec13 (SEQ ID NO:53), or a mammalian homolog thereof, or a variant of Sec13 encoded by a nucleic acid that hybridizes to the Sec13 nucleic acid (SEQ ID NO:54) or its complement under low stringency conditions,
(xvii) Sec31 (SEQ ID NO:55), or a mammalian homolog thereof, or a variant of Sec31 encoded by a nucleic acid that hybridizes to the Sec31 nucleic acid (SEQ ID NO:56) or its complement under low stringency conditions,
(xviii) Ssa3 (SEQ ID NO:57), or a mammalian homolog thereof, or a variant of Ssa3 encoded by a nucleic acid that hybridizes to the Ssa3 nucleic acid (SEQ ID NO:58) or its complement under low stringency conditions,
(xix) Ssu72 (SEQ ID NO:59), or a mammalian homolog thereof, or a variant of Ssu72 encoded by a nucleic acid that hybridizes to the Ssu72 nucleic acid (SEQ ID NO:60) or its complement under low stringency conditions,
(xx) Taf60 (SEQ ID NO:61), or a mammalian homolog thereof, or a variant of Taf60 encoded by a nucleic acid that hybridizes to the Taf60 nucleic acid (SEQ ID NO:62) or its complement under low stringency conditions,
(xxi) Tkl1 (SEQ ID NO:65), or a mammalian homolog thereof, or a variant of Tkl1 encoded by a nucleic acid that hybridizes to the Tkl1 nucleic acid (SEQ ID NO:66) or its complement under low stringency conditions,
(xxii) Tsa1 (SEQ ID NO:67), or a mammalian homolog thereof, or a variant of Tsa1 encoded by a nucleic acid that hybridizes to the Tsa1 nucleic acid (SEQ ID NO:68) or its complement under low stringency conditions,
(xxiii) Tye7 (SEQ ID NO:69), or a mammalian homolog thereof, or a variant of Tye7 encoded by a nucleic acid that hybridizes to the Tye7 nucleic acid (SEQ ID NO:70) or its complement under low stringency conditions,
(xxiv) Vid24 (SEQ ID NO:71), or a mammalian homolog thereof, or a variant of Vid24 encoded by a nucleic acid that hybridizes to the Vid24 nucleic acid (SEQ ID NO:72) or its complement under low stringency conditions,
(xxv) Vps53 (SEQ ID NO:73), or a mammalian homolog thereof, or a variant of Vps53 encoded by a nucleic acid that hybridizes to the Vps53 nucleic acid (SEQ ID NO:74) or its complement under low stringency conditions,
(xxvi) Ycl046w (SEQ ID NO:79), or a mammalian homolog thereof, or a variant of Ycl046w encoded by a nucleic acid that hybridizes to the Ycl046w nucleic acid (SEQ ID NO:80) or its complement under low stringency conditions,
(xxvii) Ygr156w (SEQ ID NO:81), or a mammalian homolog thereof, or a variant of Ygr156w encoded by a nucleic acid that hybridizes to the Ygr156w nucleic acid (SEQ ID NO:82) or its complement under low stringency conditions,
(xxviii) Yhl035c (SEQ ID NO:83), or a mammalian homolog thereof, or a variant of Yhl035c encoded by a nucleic acid that hybridizes to the Yhl035c nucleic acid (SEQ ID NO:84) or its complement under low stringency conditions,
(xxix) Ykl018w (SEQ ID NO:85), or a mammalian homolog thereof, or a variant of Ykl018w encoded by a nucleic acid that hybridizes to the Ykl018w nucleic acid (SEQ ID NO:86) or its complement under low stringency conditions,
(xxx) Ylr221c (SEQ ID NO:87), or a mammalian homolog thereof, or a variant of Ylr221c encoded by a nucleic acid that hybridizes to the Ylr221c nucleic acid (SEQ ID NO:88) or its complement under low stringency conditions,
(xxxi) Yml030w (SEQ ID NO:91), or a mammalian homolog thereof, or a variant of Yml030w encoded by a nucleic acid that hybridizes to the Yml030w nucleic acid (SEQ ID NO:92) or its complement under low stringency conditions, and
(xxxii) Yor179c (SEQ ID NO:93), or a mammalian homolog thereof, or a variant of Yor179c encoded by a nucleic acid that hybridizes to the Yor179c nucleic acid (SEQ ID NO:94) or its complement under low stringency conditions,
wherein said first protein and said second protein are members of a native cellular Polyadenylation-complex, and wherein said low stringency conditions comprise hybridization in a buffer comprising 35% formamide, 5×SSC, 50 mM Tris-HCl (pH 7.5), 5 mM EDTA, 0.02% PVP, 0.02% Ficoll, 0.2% BSA, 100 ug/ml denatured salmon sperm DNA, and 10% (wt/vol) dextran sulfate for 18-20 hours at 40° C., washing in a buffer consisting of 2×SSC, 25 mM Tris-HCl (pH 7.4), 5 mM EDTA, and 0.1% SDS for 1.5 hours at 55° C., and washing in a buffer consisting of 2×SSC, 25 mM Tris-HCl (pH 7.4), 5 mM EDTA, and 0.1% SDS for 1.5 hours at 60° C.

31. A kit comprising in a container the isolated complex of any one of claim 1-8 or the antibody of claim 9.

32. A kit for processing RNA comprising in a container the isolated complex of any of claims 1-8 optionally together with further components such as reagents and working instructions.

33. A kit for the diagnosis of a disease of mammals, preferentially for a disease or disorder such as infectious diseases; viral infections such as herpes simplex infections, Epstein-Barr-infections, influenza; metabolic disease such as metachromatic leukodystrophy; neurodegenerative disorders such as amyotrophic lateral sclerosis or cancer, comprising a product according to any of the claims 1-8 optionally together with further components such as reagents and working instructions.

34. The complex of any one of claim 1-8, or the antibody or fragment of claim 9, for use in a method of diagnosing a disease or disorder such as infectious diseases; viral infections such as herpes simplex infections, Epstein-Barr-infections, influenza; metabolic disease such as metachromatic leukodystrophy; neurodegenerative disorders such as amyotrophic lateral sclerosis; cancer.

35. A method for the production of a pharmaceutical composition comprising carrying out the method of claim 12 or 19 to identify a molecule that modulates the function, activity, composition or formation of said complex, and further comprising mixing the identified molecule with a pharmaceutically acceptable carrier.

36. A process for preparing complex of claim 1-8 and optionally the components thereof comprising the following steps:
expressing such a protein in a target cell,
isolating the protein complex which is attached to the tagged protein, and optionally disassociating the protein complex and isolating the individual complex members.

37. The process according to claim 36 characterized in that the tagged protein comprises two different tags which allow two separate affinity purification steps.

38. The process according to any of claim 36-37 characterized in that two tags are separated by a cleavage site for a protease.

39. Component of the Polyadenylation-complex obtainable by a process according to any of claim 36-38.

40. Complex of claim 1-8 and/or protein thereof as a target for an active agent of a pharmaceutical, preferably a drug target in the treatment or prevention of a disease or disorder such as infectious diseases; viral infections such as herpes simplex infections, Epstein-Barr-infections, influenza; metabolic disease such as metachromatic leukodystrophy; neurodegenerative disorders such as amyotrophic lateral sclerosis; cancer.

41. Component of the Polyadenylation-complex selected from
a) yeast proteins
(i) Tcl046w (SEQ ID NO:59),
(ii) Ygr156w (SEQ ID NO:61),
(iii) Yhl035c (SEQ ID NO:63),
(iv) Ykl018w (SEQ ID NO:179),
(v) Ylr221c (SEQ ID NO:67),
(vi) Yml030w (SEQ ID NO:69), and
(vii) Yor179c (SEQ ID NO:71).
b) the mammalian homologs/orthologs of the proteins of (a), and
c) a functionally active fragment or functionally active derivate of the proteins according to (a) and (b) carrying one or more amino acid substitutions, deletions and/or additions.

42. Component as described in claim 41, characterized in that it is encoded by a nucleic acid sequence which hybridizes to a nucleic acid sequence encoding any of the yeast proteins listed in claim 41 under low stringency conditions, wherein said low stringency conditions comprise hybridization in a buffer comprising 35% formamide, 5×SSC, 50 mM Tris-HCl (pH 7.5), 5 mM EDTA, 0.02% PVP, 0.02% Ficoll, 0.2% BSA, 100 ug/ml denatured salmon sperm DNA, and 10% (wt/vol) dextran sulfate for 18-20 hours at 40° C., washing in a buffer consisting of 2×SSC, 25 mM Tris-HCl (pH 7.4), 5 mM EDTA, and 0.1% SDS for 1.5 hours at 55° C., and washing in a buffer consisting of 2×SSC, 25 mM Tris-HCl (pH 7.4), 5 mM EDTA, and 0.1% SDS for 1.5 hours at 60° C.

43. Nucleic acid encoding a component to any of claims 41 and 42.

44. Construct, preferably a vector construct, comprising
(a) a nucleic acid according to claim 41 and at least one further nucleic acid which is normally not associated with the nucleic acid according to claim 43, or
(b) at least two separate nucleic acid sequences each encoding a different protein, or a functionally active fragment or a functionally active derivative thereof at least one of said proteins, or functionally active fragments or functionally active derivative thereof selected from the first group of proteins according to claim 1 (a) and at least one of said proteins, or functionally active fragments or functionally active derivative thereof selected from the second group of proteins according to claim 1 (b).

45. Host cell containing a nucleic acid of claim 43 and/or a construct of claim 44 or containing several vectors comprising on different vectors the nucleic acid sequence encoding at least one of the proteins, or functionally active fragments or functionally active derivatives thereof selected from the first group of proteins according to claim 1 (a) and at least one of the proteins, or functionally active fragments or functionally active derivatives thereof selected from the second group of proteins according to claim 1 (b).
